(19) United States
(12) Patent Application Publication
(10) Pub. No.: US 2026/0103735 A1
(43) Pub. Date: Apr. 16, 2026

(54) MICROORGANISMS PRODUCING LOW LINOLEIC ACID OILS AND METHODS OF USE

(71) Applicant: Cemvita Factory, Inc., Houston, TX (US)

(72) Inventors: Ryan GEORGIANNA, San Mateo, CA (US); Stephen B. DEL CARDAYRE, Belmont, CA (US); Peggy LAI, Oakland, CA (US); Kelly MILLER, San Francisco, CA (US); Eduardo J.L. COELHO, Braga (PL); Megan Hsi, Redwood City, CA (US); Julia Khandurina, San Mateo, CA (US); Evan Sims, Oakland, CA (US); Bonnie MCNEIL, San Mateo, CA (US)

(21) Appl. No.: 19/080,244

(22) Filed: Mar. 14, 2025

Related U.S. Application Data
(63) Continuation of application No. PCT/US2023/074390, filed on Sep. 15, 2023.
(60) Provisional application No. 63/376,026, filed on Sep. 16, 2022.
(60) Provisional application No. 63/479,937, filed on Jan. 13, 2023.
(60) Provisional application No. 63/516,690, filed on Jul. 31, 2023.

(51) Int. Cl. C12P 7/6431; C12N 9/02; C12N 9/04; C12N 9/88; C12N 15/81

(52) U.S. Cl. CPC C12P 7/6431; C12N 9/0006; C12N 9/0071; C12N 9/88; C12N 15/815; C12Y 101/01038; C12Y 114/19001; C12Y 114/19003; C12Y 401/02009; C12N 2800/102

(57) ABSTRACT

Some aspects of this invention provide engineered microbes for oil production. Methods for microbe engineering and for use of engineered microbes are also provided herein. In some embodiments, microbes provided are engineered to produce oils with low linoleic acids. In some embodiments, microbes provided are engineered to increase the production of lipids by the microbes. In some embodiments, microbes provided are engineered to decrease the viscosity of a liquid culture comprising the microbes. Also disclosed herein are methods of processing, culturing, separating, and extracting products from engineered microbes for oil production.

### CROSS REFERENCE TO RELATED APPLICATIONS

This application is a continuation of International Patent Application No. PCT/US2023/074390, filed on Sep. 15, 2023, which claims benefit of U.S. Provisional Patent Application No. 63/367,026, filed on Sep. 16, 2022, U.S. Provisional Patent Application No. 63/479,937, filed on Jan. 13, 2023, and U.S. Provisional Patent Application No. 63/516,690, filed on Jul. 31, 2023, each of which is incorporated herein by reference in its entirety.

### REFERENCE TO A SEQUENCE LISTING

This application contains a Sequence Listing which has been submitted electronically in XML format and is hereby incorporated by reference in its entirety. Said XML copy, created on Sep. 14, 2023, is named 59879-712.301.xml and is 39.583 bytes in size.

### BACKGROUND

The production of oils and fats by single cell microorganisms, such as yeast and microalgae, has been commercially pursued for close to a century. Most efforts have failed because the cost of manufacturing these oils and fats by fermentation could not compete with the cost of manufacturing equivalent oils from plants and animals. Only unique, high value oils, such as those rich in the essential omega-3-fatty acids, DHA and EPA, have been commercially successful and are used in infant formula, prenatal vitamins, and recently in fish feed. In order to be commercially successful and competitive with the environmentally destructive seed oils, such as soy, sunflower, safflower, rapeseed, and canola; microbial oils and fats will need to be of higher quality and less costly. There exists limited to no guidance within the art as to selection of particular genetic modifications for genetically engineering microorganisms which efficiently produce lipids with improved fatty acid profile at commercially viable efficiencies.

Microorganisms capable of producing oils are often difficult to separate from the oil that is produced. Various techniques have been employed to separate out the microorganisms from the oil but often rely on first lysing the microorganism. Further complications can arise as the microorganism is often cultured in a fermentation broth that may have similar or lower densities to the oil (e.g., lipids) generated.

### SUMMARY

Responsive to the unmet need within the art for microorganisms which efficiently produce lipids with improved fatty acid profile at commercially feasible rates and efficiencies, described here are novel microbial strains that produce novel compositions of high value fats and oils, in cultures with reduced viscosity, low linoleic acid content, and/or increased lipid production rates; and methods developed to create these strains, and the processes for their competitive commercial production, for example, separation processes allowing for removal of oil containing cells from a fermentation broth or cell culture.

In some aspects, provided herein are oleaginous microorganisms. In some embodiments, the oleaginous microorganism comprises one or more genetic modifications. In some embodiments, the microorganism produces a liquid culture comprising a lower viscosity and a lower linoleic acid content than a control oleaginous microorganism that does not have the one or more genetic modifications. In some embodiments, the oleaginous microorganism has greater than 50% by weight of triacylglycerides (TAGs). In some embodiments, the microorganism is genetically diploid. In some embodiments, the genetic modification comprises a targeted deletion or replacement of genomic DNA as compared to a wild-type oleaginous microorganism.

In some embodiments, the microorganism produces a liquid culture comprising a lower viscosity than a control microorganism that does not have the one or more genetic modifications. In some embodiments, the genetic modification comprises at least one of the following genetic modifications: a MNT1 deletion, under-production, under-expression, inactivation, or negative attenuation; a deletion, under-production, under-expression, inactivation, or attenuation of a MNT1 homologue; a deletion, under-production, under-expression, inactivation, or negative attenuation of a protein comprising an amino acid sequence set forth in SEQ ID NO. 27, SEQ ID NO. 72, or SEQ ID NO. 73; a deletion, under-production, under-expression, inactivation, or negative attenuation of a protein comprising an amino acid sequence which is at least 70%, 75%, 80%, 85%, 90%, or 95% similar to SEQ ID NO. 27, SEQ ID NO. 72, or SEQ ID NO. 73; or a deletion, under-production, under-expression, inactivation, or negative attenuation of a protein which catalyzes a reaction defined by EC 2.4.1; an AGS1 deletion, under-production, under-expression, inactivation, or negative attenuation; a deletion, under-production, under-expression, inactivation, or negative attenuation of an AGS1 homologue; a deletion, under-production, under-expression, inactivation, or negative attenuation of a protein comprising an amino acid sequence set forth in SEQ ID NO. 28, SEQ ID NO. 74, SEQ ID NO. 75, or SEQ ID NO. 76; a deletion, under-production, under-expression, inactivation, or negative attenuation of a protein comprising an amino acid sequence which is at least 70%, 75%, 80%, 85%, 90%, or 95% similar to SEQ ID NO. 28; or a deletion, under-production, under-expression, inactivation, or negative attenuation of a protein which catalyzes a reaction defined by EC 2.4.1.183; a VEL1 overexpression, overproduction, activation, or positive attenuation; an overexpression, overproduction, activation, or positive attenuation of a VEL1 homologue; overexpression, overproduction, activation, or positive attenuation of a protein comprising an amino acid sequence set forth in SEQ ID NO. 59, SEQ ID NO. 77, SEQ ID NO. 78, SEQ ID NO. 79, or SEQ ID NO. 80; or an overexpression, overproduction, activation, or positive attenuation of a protein comprising an amino acid sequence which is at least 70%, 75%, 80%, 85%, 90%, or 95% similar SEQ ID NO. 59, SEQ ID NO. 77, SEQ ID NO. 78, SEQ ID NO. 79, or SEQ ID NO. 80; or a VEL4 overexpression, overproduction, activation, or positive attenuation; an overexpression, overproduction, activation, or positive attenuation of a VEL4 homologue; an overexpression, overproduction, activation, or positive attenuation of a protein comprising an amino acid sequence set forth in SEQ ID NO. 58, or SEQ ID NO. 81, SEQ ID NO. 82, SEQ ID NO. 83, or SEQ ID NO. 84; or an overexpression, overproduction, activation, or positive attenuation of a protein comprising an amino acid sequence which is at least 70%, 75%, 80%, 85%, 90%, or 95% similar SEQ ID NO. 58, or SEQ ID NO. 81, SEQ ID NO. 82, SEQ ID NO. 83, or SEQ ID NO. 84. In some embodiments, the deletion, under-production, under-expression, inactivation, or negative attenuation comprises: gene deletion, replacement, mutation, or knockdown of a gene encoding a protein in a. or b. In some embodiments, the overexpression, overproduction, activation, or positive attenuation comprises: heterologous expression of a gene encoding a protein in c. or d, modification of a regulatory element coupled to a gene encoding a protein in c. or d., modification of a nucleotide sequence of a gene encoding a protein in c. or d., or a mutation to an amino acid sequence of a protein in c. or d. In some embodiments, the microorganism has a higher specific productivity in comparison to a control non-genetically modified microorganism. In some embodiments, the genetic modification comprises at least one of the following genetic modifications: a MNT1 deletion, under-production, under-expression, inactivation, or negative attenuation; a deletion, under-production, under-expression, inactivation, or attenuation of a MNT1 homologue; a deletion, under-production, under-expression, inactivation, or negative attenuation of a protein comprising an amino acid sequence set forth in SEQ ID NO. 27, SEQ ID NO. 72, or SEQ ID NO. 73; a deletion, under-production, under-expression, inactivation, or negative attenuation of a protein comprising an amino acid sequence which is at least 70%, 75%, 80%, 85%, 90%, or 95% similar to SEQ ID NO. 27, SEQ ID NO. 72, or SEQ ID NO. 73; or a deletion, under-production, under-expression, inactivation, or negative attenuation of a protein which catalyzes a reaction defined by EC 2.4.1; an AGS1 deletion, under-production, under-expression, inactivation, or negative attenuation; a deletion, under-production, under-expression, inactivation, or negative attenuation of an AGS1 homologue; a deletion, under-production, under-expression, inactivation, or negative attenuation of a protein comprising an amino acid sequence set forth in SEQ ID NO. 28, SEQ ID NO. 74, SEQ ID NO. 75, or SEQ ID NO. 76; a deletion, under-production, under-expression, inactivation, or negative attenuation of a protein comprising an amino acid sequence which is at least 70%, 75%, 80%, 85%, 90%, or 95% similar to SEQ ID NO. 28; or a deletion, under-production, under-expression, inactivation, or negative attenuation of a protein which catalyzes a reaction defined by EC 2.4.1.183; a VEL1 overexpression, overproduction, activation, or positive attenuation; an overexpression, overproduction, activation, or positive attenuation of a VEL1 homologue; overexpression, overproduction, activation, or positive attenuation of a protein comprising an amino acid sequence set forth in SEQ ID NO. 59, SEQ ID NO. 77, SEQ ID NO. 78, SEQ ID NO. 79, or SEQ ID NO. 80; or an overexpression, overproduction, activation, or positive attenuation of a protein comprising an amino acid sequence which is at least 70%, 75%, 80%, 85%, 90%, or 95% similar SEQ ID NO. 59, SEQ ID NO. 77, SEQ ID NO. 78, SEQ ID NO. 79, or SEQ ID NO. 80; or a VEL4 overexpression, overproduction, activation, or positive attenuation; an overexpression, overproduction, activation, or positive attenuation of a VEL4 homologue; an overexpression, overproduction, activation, or positive attenuation of a protein comprising an amino acid sequence set forth in SEQ ID NO. 58, or SEQ ID NO. 81, SEQ ID NO. 82, SEQ ID NO. 83, or SEQ ID NO. 84; or an overexpression, overproduction, activation, or positive attenuation of a protein comprising an amino acid sequence which is at least 70%, 75%, 80%, 85%, 90%, or 95% similar SEQ ID NO. 58, or SEQ ID NO. 81, SEQ ID NO. 82, SEQ ID NO. 83, or SEQ ID NO. 84. In some embodiments, the genetic modification comprises at least one of the following genetic modifications: a MNT1 deletion; an AGS1 deletion; a VEL1 overexpression; or a VEL4 overexpression. In some embodiments, the microorganism has a genetic modification comprising at least one of the following genetic modifications: a FAD2A deletion, under-production, under-expression, inactivation, or negative attenuation; a deletion, under-production, under-expression, inactivation, or negative attenuation of an FAD2A homologue; a deletion, under-production, under-expression, inactivation, or negative attenuation of a protein comprising an amino acid sequence set forth in SEQ ID NO. 1; a deletion, under-production, under-expression, inactivation, or negative attenuation of a protein comprising an amino acid sequence which is at least 70%, 75%, 80%, 85%, 90%, or 95% similar to SEQ ID NO. 1; or a deletion, under-production, under-expression, inactivation, or negative attenuation of a protein which catalyzes a reaction defined by EC 1.14.19.6; a VEL4 overexpression, overproduction, activation, or positive attenuation; an overexpression, overproduction, activation, or positive attenuation of a VEL4 homologue; an overexpression, overproduction, activation, or positive attenuation of a protein comprising an amino acid sequence set forth in SEQ ID NO. 58, or SEQ ID NO. 81, SEQ ID NO. 82, SEQ ID NO. 83, or SEQ ID NO. 84; or an overexpression, overproduction, activation, or positive attenuation of a protein comprising an amino acid sequence which is at least 70%, 75%, 80%, 85%, 90%, or 95% similar SEQ ID NO. 58, or SEQ ID NO. 81, SEQ ID NO. 82, SEQ ID NO. 83, or SEQ ID NO. 84; a overexpression, overproduction, activation, or positive attenuation of a XPK gene encoding a phosphoketolase; overexpression, overproduction, activation, or positive attenuation of a protein comprising an amino acid sequence set forth in SEQ ID NO. 40, or overexpression of a protein comprising an amino acid sequence which is at least 70%, 75%, 80%, 85%, 90%, or 95% similar SEQ ID NO. 40, overexpression of a protein which catalyzes a reaction defined by EC 4.1.2.9 or EC 4.1.2.22; or an overexpression, overproduction, activation, or positive attenuation of a ME2 gene encoding a Malic Enzyme; overexpression, overproduction, activation, or positive attenuation of a protein comprising an amino acid sequence set forth in SEQ ID NO. 37; or overexpression, overproduction, activation, or positive attenuation of a protein comprising an amino acid sequence which is at least 70%, 75%, 80%, 85%, 90%, or 95% similar SEQ ID NO. 37, overexpression of a protein which catalyzes a reaction defined by EC 1.1.1.40. In some embodiments, the gene XPK encoding the phosphoketolase is from Clostridium acetylbutilicum. In some embodiments, the Malic Enzyme is from C. oleaginosus (ME2_Co). In some embodiments, the microorganism comprises a deletion, under-production, under-expression, inactivation, or negative attenuation in the gene encoding FAD2A. In some embodiments, the microorganism comprises at least 2, 3, 4, 5, or 6 of the genetic modifications. In some embodiments, the microorganism comprises each of the genetic modifications. In some embodiments, the microorganism has genetic modifications comprising a deletion, under-production, under-expression, inactivation, or negative attenuation in the gene encoding FAD2A, overexpression, overproduction, activation, or positive attenuation of a phosphoketolase (XPK) encoding gene, and overexpression, overproduction, activation, or positive attenuation of a Malic Enzyme (ME2) encoding gene. In some embodiments, the microorganism has an additional genetic modification comprising MNT1 deletion, under-production, under-expression, inactivation, or negative attenuation. In some embodiments, the overexpression, overproduction, activation, or positive attenuation of a phosphoketolase (XPK) encoding gene, overexpression, overproduction, activation, or positive attenuation of a Malic Enzyme (ME2) encoding gene, or overexpression, overproduction, activation, or positive attenuation of a VEL4 encoding gene modifications increase production of lipids by the microorganism. In some embodiments, the overexpression, overproduction, activation, or positive attenuation of a phosphoketolase (XPK) encoding gene, overexpression, overproduction, activation, or positive attenuation of a Malic Enzyme (ME2) encoding gene, or overexpression, overproduction, activation, or positive attenuation of a VEL4 encoding gene modifications increase production of lipids by the microorganism by at least 8, 9, 10, 11, 12, 13, or 14%. In some embodiments, the TAGs comprise less than 6% polyunsaturated fatty acids.

In some embodiments, one or more of the genetic modifications is a targeted deletion of genomic DNA comprising a heterozygous deletion of a native gene. In some embodiments, one or more of the genetic modifications is the replacement of genomic DNA comprising a homozygous deletion of a native gene. In some embodiments, the microorganism further comprises addition of exogenous DNA. In some embodiments, the exogenous DNA is a selectable marker and is flanked by recombination sites. In some embodiments, the exogenous DNA comprises a single recombination site and does not comprise a selectable marker.

In some embodiments, the TAGs comprise less than 5% linoleic acid. In some embodiments, the TAGs comprise less than 5% PUFA and less than 4% linoleic acid. In some embodiments, the TAGs comprise less than 1% PUFA and less than 0.5% linoleic acid. In some embodiments, the TAGs comprise less than 1% PUFA and less than 0.1% linoleic acid.

In some embodiments, one or more of the genetic modifications is the replacement of genomic DNA, wherein the replacement reduces or eliminates expression of a native gene. In some embodiments, one or more of the genetic modifications is the replacement of genomic DNA, wherein the replacement increases expression of a native gene. In some embodiments, the genomic DNA encodes at least a portion of a fatty acid metabolism protein.

In some embodiments, the fatty acid metabolism protein is selected from a fatty acid desaturase, TGL3, MFE1, NADPH dependent GDH dehydrogenase, PEX10; Triol|246656 (unknown protein that is potentially homologous to enzyme from Kim et al 2019), TFDH1, TOR1, OLE1, or FAD2A. In some embodiments, the genomic DNA encodes at least a portion of a protein selected from A12 desaturase FAD2A; TGL3-370130 (lipase); MFE1-385434 (B-oxidation); TOR1 (372351) (Target of rapamycin-signaling); GSY1; Glycogen synthase 365053 EC: 2.4.1.11; Trehalose synthase 371254 (TPS1) EC: 2.4.1.15; TFDH1 (C1-tetrahydrofolate synthase) 395209; PEX10 (lipase) 367175 E3 ubiquitin ligase; OLE1 348634 EC: 1.14.19.1; UDP-Glc pyrophosphorylase (UGP) 347999 EC: 2.7.7.9; UDP-Glc decarboxylase (UXS1) 370161 EC: 4.1.1.35; UDP-glucose 4-epimerase (GALE1) 367812 EC: 5.1.3.2; UDP-galactose transporter (UGT) 334886; Mannose pyrophosphorylase (MPP2) 370163 EC: 2.7.7.13; B-glucan synthesis (SKN1A) 306218; B-glucan synthesis (SKN1B) 333973; CAS1 (o-acetyltransferase) 177902; Chitin synthase (CHS2) 215086; Chitin synthase (CHS4) 348500; Chitin synthase (CHS1) 390231 EC: 2.4.1.16; Glycosylphosphatidylinositol mannosyltransferase (PIGB) 385852; Glycosylphosphatidylinositol mannosyltransferase (PIGS) 413175; UDP-glucose 4-epimerase 365740 (GALE2); PIGH 394303; Mannose pyrophosphorylase (MPP1) 367061 EC: 2.7.7.13; B-glucan synthesis (SKN1C) 370117. In some embodiments, the exogenous DNA encodes a fatty acid metabolism protein. In some embodiments, the fatty acid metabolism protein is selected from GAPN, DGAT, PTA, ME, XPK, POS5, ZWF, CTP1, CAT2, ACC1, TOR, ACL, OLE1, FAT5, ELO1, ELO2, EUTE, MPHF, ALD6, ADH2, EDD, EDA, CBBM, PRK.

In some embodiments, the microorganism comprises three copies of a gene, wherein two of the copies are endogenous to the microorganism. In some embodiments, the gene encodes a fatty acid metabolism protein selected from GAPN, DGAT, PTA, ME, XPK, POS5, ZWF, CTP1, CAT2, ACC1, TOR, ACL, OLE1, FAT5, ELO1, ELO2, EUTE, MPHF, ALD6, ADH2, EDD, EDA, CBBM, PRK.

In some embodiments, the microorganism is Cutaneotrichosporon oleaginosus.

In some aspects, provided herein are liquid cultures comprising the microorganism. In some embodiments, the liquid culture comprises a viscosity of up to 15, 10, or 5 cP. In some embodiments, the liquid culture is configured to increase a transfer of dissolved oxygen within the culture. In some embodiments, the microorganism comprises at least one a MNT1 deletion, under-production, under-expression, inactivation, or negative attenuation, an AGS1 deletion, under-production, under-expression, inactivation, or negative attenuation, a VEL1 overexpression, overproduction, activation, or positive attenuation, or a VEL4 overexpression, overproduction, activation, or positive attenuation modifications reduces the fermentation broth viscosity. In some embodiments, the microorganism comprises at least one of a modification comprising overexpression, overproduction, activation, or positive attenuation of a phosphoketolase (XPK) encoding gene, an overexpression, overproduction, activation, or positive attenuation of a Malic Enzyme ME2 (ME2), or an overexpression, overproduction, activation, or positive attenuation of a VEL4, the modification increasing production of lipids by the microorganism, and wherein the microorganism further comprises at least one second modification comprising a MNT1 deletion, under-production, under-expression, inactivation, or negative attenuation, an AGS1 deletion, under-production, under-expression, inactivation, or negative attenuation, a VEL1 overexpression, overproduction, activation, or positive attenuation, or a VEL4 overexpression, overproduction, activation, or positive attenuation, the MNT1 deletion, under-production, under-expression, inactivation, or negative attenuation, the AGS1 deletion, under-production, under-expression, inactivation, or negative attenuation, the VEL1 overexpression, overproduction, activation, or positive attenuation, or the VEL4 overexpression, overproduction, activation, or positive attenuation, the second modification reducing the fermentation broth viscosity. In some embodiments, the phosphoketolase is from Clostridium acetylbutilicum (XPK_Ca). In some embodiments, the Malic Enzyme is from C. oleaginosus (ME2_Co). In some embodiments, the microorganism comprises a VEL4 overexpression, overproduction, activation, or positive attenuation modification which increases production of lipids by the microorganism and reduces the fermentation broth viscosity. In some embodiments, the microorganism comprises at least one of a MNT1 deletion, under-production, under-expression, inactivation, or negative attenuation or a AGS1 deletion, under-production, under-expression, inactivation, or negative attenuation, wherein the MNT1 deletion, under-production, under-expression, inactivation, or negative attenuation or the AGS1 deletion, under-production, under-expression, inactivation, or negative attenuation reduces a viscosity of the culture comprising the oleaginous microorganism. In some embodiments, the microorganism comprises at least one of a VEL1 overexpression, overproduction, activation, or positive attenuation or a VEL4 overexpression, overproduction, activation, or positive attenuation, wherein the VEL1 overexpression, overproduction, activation, or positive attenuation or the VEL4 overexpression, overproduction, activation, or positive attenuation reduces a viscosity of the culture comprising the oleaginous microorganism.

In some embodiments, the average TAG content of the oleaginous microorganisms in the culture is greater than 50%. In some embodiments, the concentration of TAG is 50 to 160 grams per liter of culture.

In some aspects, provided herein is a method of altering a fatty acid profile of a diploid oleaginous microorganism, the method comprising modifying target native genomic DNA of the microorganism by homologous recombination, wherein the modifying modulates expression of a fatty acid metabolism protein in the microorganism, thereby altering the fatty acid profile of the microorganism. In some embodiments, the target native genomic DNA comprises a gene encoding the fatty acid metabolism protein. In some embodiments, the modifying comprises making a heterozygous deletion, under-production, under-expression, inactivation, or negative attenuation of the gene encoding the fatty acid metabolism protein. In some embodiments, wherein the modifying comprises making a homozygous deletion of the gene encoding the fatty acid metabolism protein. In some embodiments, the modifying comprises inserting a gene encoding the fatty acid metabolism protein. In some embodiments, the fatty acid metabolism protein is coupled to an exogenous promoter. In some embodiments, the microorganism's genome further comprises two native copies of the fatty acid metabolism protein. In some embodiments, the modifying comprises introducing into the microorganism a linear DNA cassette comprising a selectable marker flanked by DNA homologous to genomic DNA flanking the target native genomic DNA, thereby causing the linear DNA cassette to recombine with genomic DNA by homologous recombination and modifying the target native genomic DNA. In some embodiments, the selectable marker on the cassette is flanked by recognition sequences for a recombinase. In some embodiments, the method further comprises removing the selectable marker from the genome of the microorganism using the recombinase. In some embodiments, the cassette further comprises a gene encoding a fatty acid metabolism protein.

In some embodiments, the altered fatty acid profile of the microorganism comprises less than 6% polyunsaturated fatty acids (PUFA) and less than 5% linoleic acid. In some embodiments, the altered fatty acid profile of the microorganism comprises less than 5% PUFA and less than 4% linoleic acid. In some embodiments, the altered fatty acid profile of the microorganism comprises less than 1% PUFA and less than 0.5% linoleic acid.

In some aspects, provided herein is an oleaginous microorganism comprising a genetic modification and having greater than 50% by weight of triacylglycerides (TAGs), wherein the TAGs comprise less than 6% polyunsaturated fatty acids, wherein the microorganism is genetically diploid, and wherein the genetic modification causes underexpression of a Δ12 desaturase. In some embodiments, the microbe produces triacylglycerides (TAGs); wherein when cultured, the microorganism yields at least 55 grams of TAGs per liter of culture; wherein the TAGs comprise less than 5% linoleic acid. In some embodiments, the genetic modification is an a deletion, under-production, under-expression, inactivation, or negative attenuation in the gene encoding FAD2A in C.o. to eliminate linoleic acid production In some embodiments, the TAGs comprise less than 6% polyunsaturated fatty acids (PUFA). In some embodiments, the TAGs comprise less than 5% polyunsaturated fatty acids (PUFA). In some embodiments, the TAGs comprise less than 1% polyunsaturated fatty acids (PUFA). In some embodiments, the TAGs comprise less than 4% linoleic acid. In some embodiments, the TAGs comprise less than 1% linoleic acid. In some embodiments, the TAGs comprise less than 0.5% linoleic acid.

In some embodiments, the microorganism is a diploid oleaginous microorganism. In some embodiments, the microorganism is genetically modified to comprise at least a heterozygous deletion of a native gene. In some embodiments, the microorganism is genetically modified to comprise a homozygous deletion of a native gene.

In some embodiments, the native gene encodes a Δ12 desaturase. In some embodiments, the microorganism when cultured produces at least 1% more C18:1 fatty acid content as compared to a microorganism which has not been genetically modified to reduce expression of a Δ12 desaturase. In some embodiments, the microorganism when cultured produces at least 5% more C18:1 fatty acid content as compared to a microorganism which has not been genetically modified to reduce expression of a Δ12 desaturase. In some embodiments, the microorganism when cultured produces at least 15% more C18:1 fatty acid content as compared to a microorganism which has not been genetically modified to reduce expression of a Δ12 desaturase. In some embodiments, the microorganism when cultured produces at least 50% more C18:1 fatty acid content as compared to a microorganism which has not been genetically modified to reduce expression of a Δ12 desaturase. In some embodiments, the microorganism when cultured produces at least 1% less C18:2 fatty acid content as compared a microorganism which has not been genetically modified to reduce expression of a Δ12 desaturase. In some embodiments, the microorganism when cultured produces at least 5% less C18:2 fatty acid content as compared a microorganism which has not been genetically modified to reduce expression of a Δ12 desaturase. In some embodiments, the microorganism when cultured produces at least 15% less C18:2 fatty acid content as compared a microorganism which has not been genetically modified to reduce expression of a Δ12 desaturase. In some embodiments, the microorganism when cultured produces at least 50% less C18:2 fatty acid content as compared a microorganism which has not been genetically modified to reduce expression of a Δ12 desaturase.

In some embodiments, the microorganism is genetically modified to express or overexpress one or more desaturases. In some embodiments, the expression or overexpression of the desaturase is caused by a heterozygous genetic modification. In some embodiments, the expression or overexpression of the desaturase is caused by a homozygous genetic modification. In some embodiments, the expression or overexpression of the desaturase is caused by an ectopic gene integration. In some embodiments, the desaturase is a stearoyl-CoA desaturase. In some embodiments, the desaturase is a 49 desaturase. In some embodiments, the microorganism when cultured produces at least 10% more C18:1 fatty acid content as compared a microorganism which has not been genetically modified to reduce expression of a Δ12 desaturase and increase expression of a Δ9 fatty acid desaturase. In some embodiments, an amino acid sequence of the Δ9 desaturase is selected from a group consisting of: OLE1, FAT-5, OLE1 Y lipolytica, OLE1 Scheffersomyces, Pg SCD, T66d9des XYZ.

In some aspects, provided herein is an oleaginous microorganism that has been genetically modified to underproduce one or more of the proteins set forth in Table 1 or to overproduce one or more of the proteins set forth in Table 2. In some embodiments, the microorganism has been genetically modified to underproduce a combination of 2, 3, 4, 5, 6, 7, 8, 9, or 10 of the proteins set forth in Table 1. In some embodiments, the microorganism has been genetically modified to overproduce a combination of 2, 3, 4, 5, 6, 7, 8, 9, or 10 of the proteins set forth in Table 2. In some embodiments, the microorganism has been genetically modified to underproduce a combination of 2, 3, 4, 5, 6, 7, 8, 9, or 10 of the proteins set forth in Table 1 and to overproduce a combination of 2, 3, 4, 5, 6, 7, 8, 9, or 10. In some embodiments, the microorganism is genetically diploid. In some embodiments, the microorganism is C. oleaginosus.

In some embodiments, the microorganism has an increase in oil productivity, yield, or titer as compared to an oleaginous microorganism that has not had the genetic modification. In some embodiments, the microorganism has an increased susceptibility to lysis as compared to an oleaginous microorganism that has not had the modification. In some embodiments, the microorganism when cultured produces at least 1% more C18:1 fatty acid content as compared to a microorganism that has not had the genetic modification. In some embodiments, the microorganism when cultured produces at least 5% more C18:1 fatty acid content as compared to a microorganism that has not had the genetic modification. In some embodiments, the microorganism when cultured produces at least 15% more C18:1 fatty acid content as compared to a microorganism that has not had the genetic modification. In some embodiments, the microorganism when cultured produces at least 50% more C18:1 fatty acid content as compared to a microorganism that has not had the genetic modification. In some embodiments, the microorganism when cultured produces at least 1% less C18:2 fatty acid content as compared a microorganism that has not had the genetic modification. In some embodiments, the microorganism when cultured produces at least 5% less C18:2 fatty acid content as compared a microorganism that has not had the genetic modification. In some embodiments, the microorganism when cultured produces at least 15% less C18:2 fatty acid content as compared a microorganism that has not had the genetic modification. In some embodiments, the microorganism when cultured produces at least 50% less C18:2 fatty acid content as compared a microorganism that has not had the genetic modification.

In some aspects, provided herein is a method of producing a lipid with an oleaginous microorganism comprising genetic modifications, the method comprising: culturing an oleaginous microorganism comprising genetic modifications, the genetic modifications: decreasing a viscosity of a fermentation culture comprising the microorganism and decreasing a productivity of linoleic acid by the oleaginous microorganism. In some embodiments, the microorganism comprises a genetic modification that increases a productivity of lipids by the oleaginous microorganism. In some embodiments, the genetic modification comprises a targeted deletion or a replacement of genomic DNA as compared to a wild-type oleaginous microorganism. In some embodiments, the genetic modification comprises at least one of following genetic modifications: a deletion, under-production, under-expression, inactivation, or negative attenuation in a gene encoding MNT1; a deletion, under-production, under-expression, inactivation, or negative attenuation in a gene encoding AGS1; a overexpression, overproduction, activation, or positive attenuation in a gene encoding VEL1; a overexpression, overproduction, activation, or positive attenuation in a gene encoding VEL4. In some embodiments, the genetic modification comprises at least one of following genetic modifications: a deletion, under-production, under-expression, inactivation, or negative attenuation in the gene encoding FAD2A; a overexpression, overproduction, activation, or positive attenuation in the gene encoding VEL4; an overexpression, overproduction, activation, or positive attenuation of a XPK gene encoding a phosphoketolase; or an overexpression, overproduction, activation, or positive attenuation of a ME2 gene encoding a Malic Enzyme. In some embodiments, the phosphoketolase is from Clostridium acetylbutilicum. In some embodiments, the Malic Enzyme is from C. oleaginosus (ME2_Co). In some embodiments, wherein the genetic modification comprises a deletion, under-production, under-expression, inactivation, or negative attenuation in the gene encoding FAD2A. In some embodiments, the microorganism comprises at least 2, 3, 4, or 5 of the genetic modifications. In some embodiments, the microorganism comprises each of the genetic modifications. In some embodiments, the microorganism comprises genetic modifications comprising a deletion, under-production, under-expression, inactivation, or negative attenuation in the gene encoding FAD2A, overexpression, overproduction, activation, or positive attenuation of a phosphoketolase (XPK) encoding gene, and overexpression, overproduction, activation, or positive attenuation of a Malic Enzyme (ME2). In some embodiments, the microorganism comprises an additional genetic modification comprising MNT1 deletion, under-production, under-expression, inactivation, or negative attenuation. In some embodiments, the method further comprises increasing production of lipids by the microorganism by inducing an overexpression, overproduction, activation, or positive attenuation of a phosphoketolase (XPK) encoding gene, an overexpression, overproduction, activation, or positive attenuation of a Malic Enzyme (ME2), an overexpression, overproduction, activation, or positive attenuation of a VEL4, or any combination thereof.

In some embodiments, the increasing production of lipids by the microorganism increase the production of lipids by at least 8, 9, 10, 11, 12, 13, or 14% as a result of the overexpression, overproduction, activation, or positive attenuation. In some embodiments, the culturing the microorganism occurs in a liquid culture. In some embodiments, the method further comprises reducing a viscosity of the liquid culture as a result of a MNT1 deletion, under-production, under-expression, inactivation, or negative attenuation, an AGS1 deletion, under-production, under-expression, inactivation, or negative attenuation, a VEL1 overexpression, overproduction, activation, or positive attenuation, or a VEL4 overexpression, overproduction, activation, or positive attenuation. In some embodiments, the method further comprises decreasing a viscosity of the liquid culture and increasing a transfer rate of dissolved oxygen within the liquid culture. In some embodiments, the method further comprises decreasing a viscosity of the liquid culture and reducing the power of an agitator which agitates the liquid culture. In some embodiments, the microorganism comprises at least one the MNT1 deletion, under-production, under-expression, inactivation, or negative attenuation, AGS1 deletion, under-production, under-expression, inactivation, or negative attenuation, VEL1 overexpression, overproduction, activation, or positive attenuation, or VEL4 overexpression, overproduction, activation, or positive attenuation modifications reduces the fermentation broth viscosity. In some embodiments, the microorganism comprises at least one overexpression of a phosphoketolase (XPK) encoding gene, an overexpression, overproduction, activation, or positive attenuation of a Malic Enzyme (ME2), or a VEL4 overexpression, overproduction, activation, or positive attenuation modification which increase production of lipids by the microorganism, and wherein the microorganism comprises at least one a MNT1 deletion, under-production, under-expression, inactivation, or negative attenuation, an AGS1 deletion, under-production, under-expression, inactivation, or negative attenuation, an VEL1 overexpression, overproduction, activation, or positive attenuation, or a VEL4 overexpression, overproduction, activation, or positive attenuation modification which reduces the liquid culture viscosity. In some embodiments, the method further comprises reducing a liquid culture viscosity and increasing the production of lipids by the microorganism by culturing the microorganism, the microorganism comprising a VEL4 overexpression, overproduction, activation, or positive attenuation modification which increases production of lipids by the microorganism and reduces the liquid culture viscosity.

In some embodiments, the reduction in liquid culture viscosity or fermentation broth viscosity is at least 20, 30, 40, 50, 60, 70, 75, 80, 85, 90, 95, 96, 97, 98, or 99% as compared to a viscosity of a liquid culture viscosity or a fermentation broth comprising a same microorganism which lacks MNT1 deletion, under-production, under-expression, inactivation, or negative attenuation, an AGS1 deletion, under-production, under-expression, inactivation, or negative attenuation, a VEL1 overexpression, overproduction, activation, or positive attenuation, or a VEL4 overexpression, overproduction, activation, or positive attenuation. In some embodiments, the native genomic DNA of the microorganism comprises a modification, wherein the modification modulates expression of a fatty acid metabolism protein in the microorganism, thereby altering the fatty acid profile of the microorganism. In some embodiments, the modification modulates a target native genomic DNA which comprises a gene encoding the fatty acid metabolism protein. In some embodiments, the modification comprises a heterozygous deletion of the gene encoding the fatty acid metabolism protein. In some embodiments, the modification comprises a homozygous deletion of the gene encoding the fatty acid metabolism protein. In some embodiments, the modification comprises inserting a gene encoding the fatty acid metabolism protein. In some embodiments, the fatty acid metabolism protein is coupled to an exogenous promoter. In some embodiments, the microorganism's genome further comprises two native copies of the fatty acid metabolism protein.

In some embodiments, the modification a linear DNA cassette comprising a selectable marker flanked by DNA homologous to genomic DNA flanking the target native genomic DNA, thereby causing the linear DNA cassette to recombine with genomic DNA by homologous recombination and modifying the target native genomic DNA. In some embodiments, the modification wherein the selectable marker on the cassette is flanked by recognition sequences for a recombinase. In some embodiments, the selectable marker from the genome of the microorganism using the recombinase. In some embodiments, the cassette further comprises a gene encoding a fatty acid metabolism protein. In some embodiments, the culturing the microorganism produces lipids with a fatty acid profile comprising less than 6% polyunsaturated fatty acids (PUFA) and less than 5% linoleic acid. In some embodiments, the culturing the microorganism produces lipids with a fatty acid profile comprising less than 5% PUFA and less than 4% linoleic acid. In some embodiments, the culturing the microorganism produces lipids with a fatty acid profile comprising less than 1% PUFA and less than 0.5% linoleic acid. In some embodiments, the culturing the microorganism produces lipids with a fatty acid profile comprising at least 1% more C18:1 fatty acid content as compared to a microorganism that has not had the genetic modification. In some embodiments, the culturing the microorganism produces lipids with a fatty acid profile comprising at least 5% more C18:1 fatty acid content as compared to a microorganism that has not had the genetic modification. In some embodiments, the culturing the microorganism produces lipids with a fatty acid profile comprising at least 15% more C18:1 fatty acid content as compared to a microorganism that has not had the genetic modification. In some embodiments, the culturing the microorganism produces lipids with a fatty acid profile comprising at least 50% more C18:1 fatty acid content as compared to a microorganism that has not had the genetic modification. In some embodiments, the culturing the microorganism produces lipids with a fatty acid profile comprising at least 1% less C18:2 fatty acid content as compared a microorganism that has not had the genetic modification. In some embodiments, the culturing the microorganism produces lipids with a fatty acid profile comprising at least 5% less C18:2 fatty acid content as compared a microorganism that has not had the genetic modification. In some embodiments, the culturing the microorganism produces lipids with a fatty acid profile comprising at least 15% less C18:2 fatty acid content as compared a microorganism that has not had the genetic modification. In some embodiments, the culturing the microorganism produces lipids with a fatty acid profile comprising at least 50% less C18:2 fatty acid content as compared a microorganism that has not had the genetic modification. In some embodiments, the microorganism has been genetically modified to underproduce one or more of the proteins set forth in Table 1 or to overproduce one or more of the proteins set forth in Table 2. In some embodiments, the microorganism has been genetically modified to underproduce a combination of 2, 3, 4, 5, 6, 7, 8, 9, or 10 of the proteins set forth in Table 1. In some embodiments, the microorganism has been genetically modified to overproduce a combination of 2, 3, 4, 5, 6, 7, 8, 9, or 10 of the proteins set forth in Table 2. In some embodiments, the microorganism has been genetically modified to underproduce a combination of 2, 3, 4, 5, 6, 7, 8, 9, or 10 of the proteins set forth in Table 1 and to overproduce a combination of 2, 3, 4, 5, 6, 7, 8, 9, or 10.

In some aspects, provided herein is a method for collecting cells comprising: a) providing a suspension comprising cells of a microorganism comprising one or more lipids; b) modulating the density of the suspension by adding a density modulating agent to the suspension; and c) removing the microorganism from the suspension.

In some aspects, provided herein is a method for collecting cells comprising: a) providing a suspension comprising cells of a genetically modified microorganism comprising one or more lipids; and b) filtering the suspension, thereby removing the microorganism from the suspension.

In some aspects, provided herein is a method for collecting an oil from an oil-producing microorganism comprising: a) providing a suspension comprising cells of a genetically modified microorganism comprising one or more lipids; b) removing the microorganism from the suspension; and c) lysing the cells of the microorganism to release the one or more lipids.

In some aspects, provided herein is a method for collecting an oil from an oil-producing microorganism comprising: a) providing a suspension comprising cells of a microorganism comprising one or more lipids; b) modulating the density of the suspension by adding a density modulating agent to the suspension; c) removing the microorganism from the suspension; and d) lysing the cells of the microorganism to release the one or more lipids.

In some embodiments, the suspension further comprises water. In some embodiments, the microorganism is an oleaginous microorganism. In some embodiments, the microorganism comprises yeast, bacterial, fungal, or algal cells. In some embodiments, the microorganism is yeast. In some embodiments, the microorganism comprises greater than 50 weight percent (%) of lipids. In some embodiments, the yeast is selected from a group consisting of Lipomyces tetrasporus, Lypomyces starkeyi, Cutanetrichosporon oleaginosus, Rhodotorula toluroides, and Yarrowia Lypolytica. In some embodiments, at least 90% of the cells have intact cell walls. In some embodiments, the cells have a cell wall that is substantially intact. In some embodiments, the one or more lipids comprise fatty acids. In some embodiments, the fatty acids comprise monounsaturated fatty acids. In some embodiments, the fatty acids comprise at least 40% by weight of oleic acid. In some embodiments, the monounsaturated saturated fatty acids is at least 40% by weight of a total weight of fatty acids. In some embodiments, the one or more lipids further comprise saturated fatty acids. In some embodiments, the one or more lipids further comprise polyunsaturated fatty acids. In some embodiments, the fatty acids comprise less than 10% by weight of linoleic acid.

In some embodiments, the density modulating agent increases the density of the suspension. In some embodiments, the density modulating agent is a salt. In some embodiments, the salt is selected from a group consisting of sodium chloride and calcium chloride. In some embodiments, the salt is aluminum sulfate, ferric chloride, calcium chloride, sodium chloride, or combinations thereof. In some embodiments, the density modulating agent is a solute. In some embodiments, the solute is selected a from a group consisting of hydrochloric acid, glycerol, and sulfuric acid. In some embodiments, the pH of the culture broth is adjusted. In some embodiments, the pH is lowered. In some embodiments, the pH is lowered by at least 2 units. In some embodiments, the pH is lowered to about 2 units. In some embodiments, the pH is lowered by sulfuric acid. In some embodiments, the pH is adjusted at least 8 hours before the removing. In some embodiments, the pH is adjusted at least 24 hours before the removing. In some embodiments, the density modulating agent decreases the density of the suspension.

In some embodiments, the density modulating agent is a solute. In some embodiments, the solute is selected a from group consisting of methanol, ethanol, propanol, isopropanol, and butanol. In some embodiments, adding the density modulating agent to the suspension directs the microbial cells to a top portion of the suspension. In some embodiments, aluminum sulfate directs the microbial cells to a top portion of the suspension. In some embodiments, ferric chloride directs the microbial cells to a top portion of the suspension. In some embodiments, sodium chloride directs the microbial cells to a top portion of the suspension. In some embodiments, adding the density modulating agent to the suspension directs the microbial cells to a bottom portion of the suspension. In some embodiments, calcium chloride directs the microbial cells to a bottom portion of the suspension. In some embodiments, adding the density modulating agent to the suspension demulsifies the suspension.

In some embodiments, removing comprises performing one or more of pipetting, decanting, filtering, or centrifugating. In some embodiments, removing comprises centrifugating. In some embodiments, the suspension is maintained within a given temperature range before centrifugation. In some embodiments, the temperature range is 10-100° C. In some embodiments, the temperature range is 60-80° C. In some embodiments, the temperature range is about 70° C. In some embodiments, the temperature range is maintained for at least 8 hours before centrifugation. In some embodiments, the temperature range is maintained for at least 24 hours before centrifugation. In some embodiments, the centrifugation speed is 1,000 to 15,000 rcf. In some embodiments, the centrifugation speed is about 4350 rcf. In some embodiments, the suspension is centrifugated for 1 to 60 minutes. In some embodiments, the suspension is centrifugated about 7 minutes. In some embodiments, the suspension is centrifugated about 30 minutes. In some embodiments, the microorganism is the microorganism of as previously described. In some embodiments, the filtering comprises microfiltration or ultrafiltration. In some embodiments, the suspension comprises the liquid culture as previously described. In some embodiments, the filtering the suspension comprises removing at least 50, 60, 70, 75, 80, 83% wt. of the water or a liquid component of the suspension. In some embodiments, the filtering the suspension comprises passing at least 50, 60, 70, 75, 80, 83% wt. of the water or a liquid component of the suspension through a filter membrane. In some embodiments, the filtering comprises passing of the water or a liquid component of the suspension through a filter membrane with a flux of at least 5, 10, 15, 25, 50, 75, 100, 125, 150, 170, 175, 200, or 250 kg/hr*m\hat{ }2. In some embodiments, the filter membrane comprises a pore size of up to 0.75, 0.8, 0.9, 1, 1.1, 1.2, 1.3, 1.4, or 1.5 μm. In some embodiments, the filtering is performed under a pressure of at least 500, 600, 700, 800, 830, 900, or 1000 mbar. In some embodiments, the filtering is performed under a pressure of up to 500, 600, 700, 800, 830, 900, or 1000 mbar. In some embodiments, a cell density of the cells in the suspension is at least 3, 3.5, 4, 4.5, 4.8, or 5 g/L. In some embodiments, a cell density of the cells in the suspension is up to 3, 3.5, 4, 4.5, 4.8, or 5 g/L. In some embodiments, a viscosity of the suspension is up to 5, 10, 15, 25, 50, 75, 100 cP. In some embodiments, the filtering is performed at a temperature of at least 80° C. In some embodiments, the method further comprises, e) collecting the one or more lipids.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIGS. 1A-D show various cassette construction strategies used in the engineering of C. oleaginosus. A vector with a selective marker driven by a unique promoter and individual terminator sequence is shown in (FIG. 1A) that is flanked by LoxP sites to allow for marker recycling when exposed to a Cre Recombinase. The cassettes for expression or overexpression of a gene (FIG. 1B) or pathway (FIG. 1C) along with a selective marker. A homology donor cassette is shown in FIG. 1D with the solid regions representing homologous regions flanking a selective marker. The cassettes can optionally be flanked by a left border (LB) and right border (RB) representing T-DNA border sequences necessary for use in Agrobacterium tumefaciens mediated transformation (AtMT).

FIG. 2 illustrates the diploid nature of the C. oleaginosus strain when compared to a haploid and diploid S. cerevisiae.

FIG. 3 illustrates % composition of major fatty acid constituents in WT, FAD2a/fad2aΔ (heterozygous), and fad2aΔ/fad2aΔ (homozygous) strains.

FIG. 4 illustrates small scale fermentation screening data that reveal a range of C18:1 concentrations resulting from an ectopically integrated library of OLE1 overexpression. The lead lines indicate the control. All other bars are CoOLE1 integrants.

FIG. 5 illustrates fermentation broth viscosity in WT, Δmnt1 homozygous, Δags1 homozygous, VEL1 overexpression (OE), and VEL4 OE strains.

FIG. 6 illustrates viscosities of fermentation broths of unmodified and mnt1 deletion strains.

FIG. 7 illustrates specific performance of WT, phosphoketolase OE (XPK_Ca), malic enzyme OE (ME2), and VEL4 OE strains.

FIG. 8 illustrates the total solids analyzed before (TS, fresh) and after 1-kDA cutoff dialysis (TS, dialyzed) for C. oleaginous WT and MNT1 KO strains after 50 hours of fermentation in nitrogen limited media containing 4.8 g/L total nitrogen.

FIG. 9 illustrates the cell suspension upon the addition of water;

FIG. 10 illustrates the cell suspension upon the addition of >99.5% methanol;

FIG. 11 illustrates the cell suspension upon the addition of acetone;

FIG. 12 illustrates the cell suspension upon the addition of 10M H₂SO₄;

FIG. 13 illustrates the cell suspension upon the addition of water;

FIG. 14 illustrates the cell suspension upon the addition of >99.5% methanol;

FIG. 15 illustrates the cell suspension upon the addition of >99.5% ethanol;

FIG. 16 illustrates the cell suspension upon the addition of >99% allyl alcohol;

FIG. 17 illustrates the cell suspension upon the addition of glycerol;

FIG. 18 illustrates the cell suspension upon the addition of xylitol;

FIG. 19 illustrates the cell suspension after centrifugation of the culture broth;

FIG. 20 illustrates the cell suspension after centrifugation of the culture broth after the addition of sulfuric acid or ethanol;

FIG. 21 illustrates the cell concentration, size and granularity of culture samples submitted to different ethanol concentrations as measured by flow cytometry;

FIG. 22 illustrates cell morphology and granularity of the suspensions submitted to different ethanol concentrations as measured by flow cytometry;

FIG. 23 illustrates the cell suspension after the addition of various amounts of ethanol;

FIG. 24 illustrates flow cytometry data reporting cell distribution between pellet and supernatant when centrifuged at different ethanol concentrations; and

FIG. 25 illustrates the fluorescence of cells in the supernatant and pellet when centrifuged at different ethanol concentrations.

FIG. 26 illustrates fermentation broth after centrifugation at 4350 rcf for 7 minutes.

FIG. 27 illustrates fermentation broth mixed with liquid dish detergent after centrifugation at 4350 rcf for 7 minutes.

FIG. 28 at panels A-E illustrate pH-adjusted fermentation broth with (A) no salts added, (B) 100 g/L aluminum sulfate added, (C) 100 g/L ferric chloride added, (D) 100 g/L calcium chloride added, and (E) 100 g/L sodium chloride added after centrifugation at 4350 rcf for 30 minutes.

FIG. 29 at panels A-D illustrate fermentation broth mixed at 20° C. (A), mixed at 70° C. (B), with sulfuric acid mixed at 20° C. (C), and with sulfuric acid mixed at 70° C. (D) after centrifugation at 4350 rcf for 7 minutes.

FIG. 30 at panels A-B illustrate fermentation broth with no sulfuric acid mixed at 70° C. for (A) 8 hours and (B) 24 hours after centrifugation at 4350 rcf for 7 minutes.

FIG. 31 at panels A-B illustrate fermentation broth with 0.5 g of sulfuric acid added per 100 g of broth mixed at 70° C. for (A) 8 hours and (B) 24 hours after centrifugation at 4350 rcf for 7 minutes.

FIG. 32 at panels A-B illustrate fermentation broth with 1 g of sulfuric acid added per 100 g of broth mixed at 70° C. for (A) 8 hours and (B) 24 hours after centrifugation at 4350 rcf for 7 minutes.

FIG. 33 at panels A-B illustrate fermentation broth with 2 g of sulfuric acid added per 100 g of broth mixed at 70° C. for (A) 8 hours and (B) 24 hours after centrifugation at 4350 rcf for 7 minutes.

FIG. 34 illustrates fermentation broth of unmodified strain having high broth viscosity after centrifugation at 4350 rcf for 7 minutes.

FIG. 35 illustrates fermentation broth of an MNT1 KO strain having lowbroth viscosity after centrifugation at 4350 rcf for 7 minutes.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Linoleic acid, an omega-6 polyunsaturated fatty acid (PUFA), is believed to be an essential fatty acid. Like the essential omega-3 PUFAs, linoleic acid is required in only very small quantities in the diet, less than 2% of daily calories. As a result of the change in the consumption of fats and oils over the last century from animal and fats and lipids to those derived from plants, primarily seed or “vegetable” oils, there has been a dramatic increase in the level of linoleic acid being consumed, such that today greater than 7% of daily calories are derived solely from linoleic acid. This level of linoleic acid would be extremely difficult to consume through eating whole foods. The transition to vegetable oils and the high daily intake of linoleic acid is associated with and believed to have caused a variety of health and environmental problems. To remedy these, alternative sources of fats and oils that are sustainable, cost effective, high performing, and contain little to no linoleic acid are needed. Embodiments described here result in high performing oils and fats with extremely low PUFA, that have high cooking performance, low levels of the inflammatory omega-6 PUFAs, and are produced at very high levels from novel engineered yeast strains that enable cost effective scalable industrial fermentations.

Attempts to commercialize single cell oils have focused on either native or classically improved strains of high oil producing microorganisms or on the engineering of lower oil producing strains for which there are more genetic tools. Described herein is the development of genetic tools and the specific genetic manipulation of the highly oleaginous basidiomycete yeast, Cutaneotrichosporon oleaginosus. C. oleaginosus naturally grows on a diversity of common and waste feedstocks, commonly produces greater than 40% of cell weight as lipid, and naturally produces low levels of linoleic acid. The natural fat composition of C. oleaginosus resembles that of beef tallow. Described here is the application of robust genetic tool sets developed to engineer this strain, and to develop it as a commercial host for the production of high performing fats and oils.

While linoleic acid is an essential fatty acid, it is only necessary in small quantities. Linoleic acid (C18:2) currently represents 7.21% of current consumed calories with another 0.72% coming from the further unsaturated linolenic acid molecule (Blasbalg et al 2011). This level of C18:2 consumption has sharply risen from 2% of consumed calories at the turn of the 20th century and is a direct result of the increased consumption of vegetable oils such as soybean and sunflower oil which are rich in the omega-6 polyunsaturates, C18:2 and C18:3. These values also far exceed the essential need of C18:2 which even in rapidly developing infants is only 1-2% (Hansen et al 1958). The amounts present in commonly consumed foods, especially protein sources, tend to easily meet the essential needs for C18:2, thus C18:2 deficiency is typically not a concern. Described herein are methods to produce fats and oils which are extremely low (<1%) in polyunsaturated fats (PUFA).

In the production of fats and oils using oleaginous yeast, specific productivity and viscosity of fermentation broth play critical roles in the efficiency and cost of production. Fermentation productivity is the amount of product made over time and specific productivity is the amount of product made per unit biomass per unit time. High specific productivity rates translate to significant cost savings for fermentation as more product may be made faster, from less biomass, for the same size bioreactor, or a smaller bioreactor may be used to achieve similar amounts of cumulative product as a larger more costly unit. Improved specific productivity can also result in an increase in the overall process yield, since less biomass is required to produce more product. Yield is a measure of the mass of product derived from a defined mass of raw material, and is a key driver of process costs.

Viscosity is a rheological property that in the context of a complex fermentation broth will generally follow a non-Newtonian behavior. In wild-type C. oleaginosus fermentations; as well as in other fungi, the culture broth may become viscous. Increased viscosity of culture broth may result from the interactions of extracellular metabolites such as polysaccharides; the interaction of physical cell properties, such as filamentation, may also result in increased viscosity. A high viscosity broth may have limitations on mass transfer; for example, high broth viscosity may result in limitation of oxygen transfer or result in less productive fermentations for aerobic processes. Reducing broth viscosity may decrease the power needed to mix and aerate the fermentation broth; the decreased power may result in cost savings as a result of less power consumption and the need to install lower cost motors for mixing. Further, lower broth viscosity may also result in efficiency in downstream processing. For example, lower broth viscosity may allow water to be more efficiently removed by settling, centrifugation, or filtration. Reducing broth viscosity may also allow cells to be more efficiently lysed, for example by the use of chemical, biochemical, physical, or mechanical methods, to liberate intracellular products such as TAGs.

As such, described herein is the application of robust genetic tool sets and selection of one or more genetic modifications developed to engineer engineered microorganisms to produce desired fatty acid profiles, reduce the production of linoleic acid, to increase specific productivity, and decrease viscosity in the production of fats and oils from oleaginous yeast. The engineered microorganisms disclosed herein efficiently produce lipids with improved fatty acid profile at commercially feasible rates. Described here are novel microbial strains that produce novel compositions of high value fats and oils, in cultures with reduced viscosity, low linoleic acid content, and/or increased lipid production rates; and methods developed to create these strains, and the processes for their competitive commercial production.

### Microorganisms

In an aspect, the present disclosure provides for a method of manufacturing an oil comprising a triacylglyceride (TAG). The method may comprise: (a) providing an oleaginous microorganism; (b) genetically modifying the oleaginous microorganism to modify the concentration of deleterious fatty acids and increase the concentrations of beneficial fatty acids; (c) culturing the oleaginous microorganism in a medium comprising a carbon source; and (d) harvesting the oil from the oleaginous microorganism.

In the systems and methods described herein, the oil is generated by a microorganism.

The microorganism may be, for example, a yeast. The yeast can be used to synthesize TAGs. The utilization of yeast for oil may be advantageous over other sources such as microalgae or vegetable oils. In some cases, the trace compounds or fatty acid profile of oil from yeast may be different than the trace compounds generated from microalgae and higher plants leading to an oil fingerprint. In some cases, the oil may be generated from a non-photosynthetic microorganism. The non-photosynthetic microorganism may generate an oil (e.g., a non-photosynthetic microbial oil).

The oil may be generated from yeast that may come from Agaricomycotina, Ascomycota, Basidiomycota, Candida, Chlorellales, Chlorellaceae, Cryptococcus, Cuniculitremaceae, Debaryomycetaceae, Filobasidiales, Incertae sedis, Lipomyces, Metschnikowiaceae, Pichiaceae, Rhodosporidium, Rhodotorula, Rhizpus, Saccharaomycotina, Saccharomyceses, Saccharomycesles, Tremellomycetes, Trichomonoascaceae, Trichosporon, Trichosporonales, Viridiplantae, or Yarrowia, etc. The yeast may be, for example, Cutaneotrichosporon oleaginous, Rhodotorula toluroides, Rhodosporidium toruloides, Lipomyces starkeyi, Lipomyces lipofer, Lipomyces arxii, Lipomyces doorenjongii, Lipomyces oligophage, Lipomyces spencer-mar tinsiae, Lipomyces knonenkode, Lypomyces tetrasporus, Lipomyces anomalus, Lipomyces japonicus, Lipomyces kockii, Lipomyces kononenkode, Lipomyces mesembrius, Rhodosporidium sp., Rhodotorula sp., Yarrowia sp., Yarrowia lipolytica, Cryptococcus sp., Cryptococcus aerius, Lipomyces sp., Candida curvata, Candida aff. insectorum, Candia aff. sagamina, Candida aff. kazuoi, Rhodotorula glutinis, Rhodotorula graminis, Rhodotula 110, Rhodotorula aurantiaca, Leucosporidiella creatinivora, Rhodotorula colostri, Rhodotorula dairenensis, Rhodotorula mucilaginosa, Rhodosporidium babjevae, Rhodosporidium diobovatum, Rhodosporidium fluviale, Rhodosporidium paludigenum, Rhodosporidium sphaerocarpum, Rhodosporidium toruloides, Cryptococcus podzolicus, Trichosporon porosum, Trichosporon guehoae, Pichia segobiensis, Trichosporonoides spathulata, Kodamaea ohmeri, Cryptococcus sp., Cryptococcus music, Lipomyces tetrasporus, Lipomyces sp, Myxozyma geophila, Myxozyma lipomycoides, Myxozyma mucilagina, Myxozyma udenii, Myxozyma vanderwaltii, Myxozyma cf. melibiosi, Myxozyma melibiosis, Torulaspora delbrueckii, Trigonopsis varaibilis, Cutaneotrichosporon oleaginosus, Cutaneosporon oleaginosus, Scheffer somyces stipitis, Kurtzmaniella cleridarum, Pichia manshurica, Cuniculitrema polymorpha, Filobasidium floriforme, Filobasidium globisporum, Filobasidium aff. globisporum, Filobasidium inconspicuum, Cryptococcus albidus, Cryptococcus gastricus, Cryptococcus magnus, Cryptococcus oeirensis, Cryptococcus terreus, Cryptococcus aff. taibaiensis, Cryptococcus flavescens, Cryptococcus aff. laurentii, Cryptococcus luteolus, Cryptococcus victoriae, Cryptococcus cf. curvatus, Cryptococcus humicola, Cryptococcus ramirezgomezianus, Cryptococcus wieringae, Filobasidium cf. uniguttulatus, Hannaella aff. zeae, Tremella aurantia, Tremella enchepala, Prototheca aff. zopfii, Prototheca stagnora, Prototheca aff. zopfii var. hydrocarbonea, Prototheca zopfii var. zopfii, ATC (20509, or Metschnikowia pulcherrim, etc.

In some embodiments, the microorganism may be a haploid microorganism. In some cases, the microorganism may be a haploid yeast. In some embodiments, the microorganism may be a diploid microorganism. In some cases, the microorganism may be a diploid yeast (2N). In some embodiments the microorganism may be a multiploid yeast (>2N) such as triploid, tetraploid, etc.

In some embodiments, the microorganism may be an oleaginous microorganism. The oleaginous microorganism may produce a high concentration of lipids such as TAGs before any modification. For instance, when cultured the oleaginous microorganism may produce more than 30% TAGs by weight of microorganism biomass.

In some cases, the microorganism may be a chemically or physically induced mutant of a natural or recombinant microorganism. In some cases, the microorganism may be a recombinant microorganism. In some cases, the microorganism may be a recombinant of a chemically or physically induced mutant of a wildtype microorganism.

In some cases, an oleaginous microorganism may comprise one or more genetic modifications. The one or more genetic modifications may alter the lipid profile generated by the microorganism. In some embodiments, the genetic modifications may be the underexpression of one or more native genes in the microorganism. In some embodiments, the genetic modifications may be the replacement of genomic DNA with exogenous DNA. In some embodiments, the genetic modifications may be the overexpression of one or more native genes in the microorganism or expression of one or more heterologous genes in the microorganism.

### Gene Knock-Outs (KO)

In some cases, lipids and oils produced by microorganisms comprise polyunsaturated fats (PUFA) such as linoleic acids which may have detrimental effects upon consumption. In some embodiments, a microorganism may be genetically modified to underexpress one or more native genes responsible for the production of deleterious PUFAs such as linoleic acid (C18:2). In some embodiments, a microorganism may be genetically modified to underexpress one or more native genes involved in polysaccharide synthesis and contribute to decreasing fermentation broth viscosity. Underexpression of genes may be caused by genetic knock-down, such as by RNA interference, or by genetic knock-out of endogenous genes, such as by homologous recombination to delete one or more genomic copies of the gene. It may also be caused by the overexpression of a regulatory protein that results in the repression or underexpression of one or more genes involved in polysaccharide synthesis and contribute to decreasing fermentation broth viscosity. Underexpression may also be accomplished by CRISPR interference. In some embodiments, gene knock out can include a genetic modification which leads to under-production, under-expression, inactivation, or negative attenuation of a gene or a protein encoded by the gene.

In some embodiments, the microorganism is engineered to express at least 10%, 15%, 20%, 30%, 40%, 50%, 60%, 70%, 80%, 90%, or 95% less of a given protein relative to a microorganism which has not been engineered to underproduce said protein. In some embodiments, the microorganism is engineered to knock out the production of a given protein, wherein the knockout leads to no activity of the protein in the microorganism.

In some embodiments, the microorganism is engineered to produce at most 10%, 15%, 20%, 30%, 40%, 50%, 60%, 70%, 80%, 90%, or 95% of a given protein relative to a microorganism which has not been engineered to underproduce said protein. In some embodiments, this results in the incapability or reduction of the protein's ability to exert its known function. Means of underproduction may include gene silencing (e.g. RNAi genes antisense), knocking-out (e.g., deleting), altering gene expression level, altering expression pattern, by mutagenizing the gene sequence, disrupting the sequence, insertions, additions, mutations, modifying expression control sequences, and the like.

In some embodiments, a genetic knockout may be accomplished by introducing into a cell a linear DNA cassette configured to alter target genomic DNA by homologous recombination. Such introduction may be accomplished, for example, by electroporation. In addition, other DNA transformation methods resulting in DNA integration can be used, such as Agrobacterium tumefaciens mediated transformation (AtMT) or biolistic transformation. In addition, DNA may be introduced into targeted regions by generating a cut in the genome via a nuclease such as CRISPR/CAS9-like systems and then either integrated via NHEJ at the cut site or targeted via HR (homologous recombination) using a DNA cassette with homology on either side of a cut site. Further improvements in HR targeted DNA may be made through knocking out the main genes involved in NHEJ (KU70/KU80 & LIG4) which have been successfully utilized in a number of oleaginous yeasts including Y. lipolytica, Rhodosporidium toruloides, and Lipomyces starkeyi to enhance the efficiency of homologous recombination for more efficient targeted gene knockouts. Use of small molecules for cell cycle synchronization and inhibition of NHEJ can also be utilized in promoting homologous recombination with varying degrees of success.

As mentioned herein, a microorganism of the present disclosure is preferably engineered to underexpress a polynucleotide encoding a protein having an amino acid as defined herein. This includes that, if a microorganism may have more than one copy of such a polynucleotide, also the other copies of such a polynucleotide are underexpressed. For example, a microorganism of the present invention may not only be haploid, but it may be diploid, tetraploid or even multi-ploid. Accordingly, in a preferred embodiment all copies of such a polynucleotide are underexpressed, such as two, three, four, five, six or even more copies.

The terms “underproduce,” “underproducing,” “underproduced” and “underproduction” in the present invention refer to the production of a gene product or a polypeptide at a level less than the production of the same gene product or polypeptide prior to a genetic alteration of the microorganism or in a comparable microorganism which has not been genetically altered. “Less than” includes, e.g., 10%, 20%, 30%, 40%, 50%, 60%, 70%, 80, 90% or more. In some embodiments, no production of the gene product or a polypeptide is also encompassed by the term “underexpression.”

In some embodiments, the protein that is underproduced may be an enzyme, and that enzyme may be one that catalyzes conversion of oleic acid (C18:1) to linoleic acid (C18:2). For instance, the underproduced protein may be a delta-12 desaturase enzyme. The underproduced protein may have an amino acid sequence that may be selected from Table 1.

Underexpression of a gene may result in the attenuated production of a protein, which may be defined as underproduction of a protein. Underproduction of a protein may be caused by a heterozygous deletion of the gene encoding the protein, such as delta-12 desaturase, in a diploid microorganism. Underproduction of a protein may be caused by a homozygous deletion of the gene encoding the protein, such as delta-12 desaturase, in a diploid microorganism. Underproduction of the proteins may comprise replacing genomic DNA with exogenous DNA. Underproduction also encompasses activity wherein a mutation may inactivate the function of a protein. Underproduction also encompasses activity wherein a mutation may silence the gene or prevent transcription or translation of the gene or gene product. Underproduction may also result from the change in the regulation of gene expression of a gene encoding a particular protein, such as delta-12 desaturase. Such change in regulation may result from the overexpression or underexpression of a regulatory protein that may induce or repress a particular protein, such as a delta-12 desaturase (or any of the proteins described in Tables 1 and 2).

In some embodiments a protein may be underproduced that is part of fatty acid metabolism. Fatty acid metabolism proteins are understood to be those proteins that are directly acting on and/or regulating the conversion of an initial substrate, for example a carbohydrate, to a lipid.

In some embodiments a protein may be underproduced that is described as important for cell wall or other byproducts. In some examples cell wall may be described as a byproduct; a byproduct is any product that uses carbon that is not converted to desired triglycerides. In some examples a byproduct may be trehalose; glycogen; polysaccharide; glycoprotein; glycolipid; phospholipids or other products that are not triglycerides. In some examples reduction in protein important for cell wall or other byproducts will result in enhanced fermentation characteristics that result in yield; titer; or specific productivity greater than a strain that has not been modified. Yield is the amount of product produced in a process relative to the amount of raw material put into the process i.e. (product (kg)/raw material (kg)). Titer is the concentration or amount per volume of product.

In some embodiments, more than one protein may be underproduced in the microorganism. In some examples, two proteins may be underproduced in the microorganism. In some examples, three proteins may be underproduced in the microorganism. In some examples, four proteins may be underproduced in the microorganism. In some cases, more than four proteins may be underproduced in the microorganism.

In some embodiments the protein underproduced is a fatty acid metabolism protein including SEQ ID NOs: 1-3, 8, 9.

In some embodiments, the protein that is underproduced may be important for cell wall or other byproducts, for instance, polysaccharides. In some examples the underproduced protein is encoded by trehalose biosynthetic genes such as the trehalose synthase TPS1 and glycogen synthesis genes such as glycogen synthase GSY1. In some examples the protein underproduced is selected from [SEQ ID NOs: 10-28] UDP-Glc pyrophosphorylase (UGP), UDP-Glc decarboxylase (UXS1), UDP-glucose 4-epimerase (GALE1), UDP-galactose transporter (UGT), Mannose pyrophosphorylase (MPP2), B-glucan synthesis (SKN1A) 306218, B-glucan synthesis (SKN1B) 333973, o-acetyltransferase (CAS1), Chitin synthase (CHS2), Chitin synthase (CHS4), Chitin synthase (CHS1), Glycosylphosphatidylinositol mannosyltransferase (PIGB), Glycosylphosphatidylinositol mannosyltransferase (PIGS), UDP-glucose 4-epimerase 365740 (GALE2), PIGH 394303, Mannose pyrophosphorylase (MPP1) 367061 EC: 2.7.7.13, B-glucan synthesis protein (SKN1C) 370117, alpha 1,3 mannosyltransferase (MNT1), alpha 1, 3 glucan synthase (AGS1).

In some embodiments, the genetic modification leads to lower fermentation broth viscosity when compared to an unmodified control. For example, SEQ ID NOs: 27 and 28, predicted alpha 1,3 mannosyltransferase (MNT1) and alpha 1, 3 glucan synthase (AGS1) could be the protein targeted for underproduction to cause lower fermentation broth viscosity. There is no guidance within the art as to selection of these particular modifications or their effect on the fermentation broth. Alpha 1,3 mannosyltransferase (MNT1) and alpha 1, 3 glucan synthase (AGS1) are both involved in polysaccharide biosynthesis. Alpha 1,3 mannosyltransferase is a Golgi mannosyltransferase and acts upon mannose to promote formulation of polysaccharides from mannose. It is unexpected that the deletion of a Golgi transferase such as alpha 1,3 mannosyltransferase would not negatively impact cell metabolism, function, or production of lipids due to its important role in secretion and localization of many proteins. Alpha 1,3 glucan synthase is glucosyltransferase involved in the production of alpha glucans which are polysaccharides found in the cell wall. It is also unexpected that the deletion of a glucosyltransferase such as alpha 1,3 glucan synthase would not negatively impact cell structure, function, or proliferation or negatively impact specific productivity due to its role in cell wall construction and septum deposition during division. Indeed, changes in the expression of these proteins that result in a decrease in fermentation broth viscosity or that do not negatively impact cell structure and fermentation performance would be surprising.

The non-functional protein of interest is written in lower case letters, optionally preceded or followed by a Δ symbol.

### Production of Proteins of Interest

One or more proteins of interest (POIs) may be produced or overproduced in the microorganism. Microorganisms described herein may be genetically modified to produce one or more heterologous proteins (or proteins of interest). Microorganisms described herein may be genetically modified to overproduce one or more native proteins in the microorganism. Proteins of interest produced or overproduced in the microorganism may include enzymes that lead to the modification of a fatty acid profile in the microorganism. For instance, enzymes that produce fatty acids with beneficial effects. Proteins of interest produced or overproduced in the microorganism may include proteins that lead to decreased fermentation broth viscosity, increased specific productivity, or both decreased fermentation broth viscosity and increased specific productivity.

A POI may be an enzyme that catalyzes the production of C18:1 TAGs. In one example, the POI may be a stearoyl-CoA desaturase (SCD) such as OLE1. The SCD enzyme amino acid sequence produced or overproduced may be native to the microorganism. The SCD amino acid sequence produced or overproduced may be heterologous to the microorganism. The POI may have an amino acid sequence selected from Table 2.

A POI may be an enzyme involved in the production of cytosolic NADPH for lipid biosynthesis. For instance, malic enzyme, such as ME2, may be a POI. The enzyme amino acid sequence produced or overproduced may be native to the microorganism. The enzyme amino acid sequence produced or overproduced may be heterologous to the microorganism.

A POI may be a protein that acts on the synthesis of or the downstream metabolism of UDP-glucose, UDP-mannose, UDP-galactose or other nucleotide sugars. A POI may be a velvet-like protein. For example, VEL1 or VEL4. Velvet-like proteins contain a distinct protein domain consisting of around 150 amino acids; these regulatory proteins are known to play a role in fungal development and have been implicated in regulation of glucan synthesis. The protein amino acid sequence produced or overproduced may be native to the microorganism. The protein amino acid sequence produced or overproduced may be heterologous to the microorganism. The protein amino acid sequence produced or overproduced may result in the overexpression or underexpression of one or more genes that encode proteins that affect fatty acid composition, specific productivity, fermentation broth viscosity, or susceptibility to lysis.

A POI may be an enzyme that catalyzes the production of sugar phosphates. A POI may be an enzyme that produces a sugar phosphate that contains two carbons less than the substrate. A POI may produce Acetyl-P, glyceraldehyde 3-phosphate or both. Acetyl-P may be converted to cytosolic acetyl-coA either through the activity of a phosphotransacetylase or the conversion of Acetyl-P to acetate and then activation of the acetate into acetyl-CoA by acetyl COA synthetase. A POI may be an enzyme that results in increased cytosolic acetyl-CoA. For instance, phosphoketolase may be a POI. Phosphoketolase may increase cytosolic acetyl-CoA. Phosphoketolase may also reduce oxygen consumption as compared to a process that strictly adheres to sugar metabolism via glycolysis and the pentose phosphate pathway. A POI may be an enzyme that uses Xylulose 5-phosphate as a substrate. The enzyme amino acid sequence produced or overproduced may be native to the microorganism. The enzyme amino acid sequence produced or overproduced may be heterologous to the microorganism.

The terms “overproduce,” “overproducing,” “overproduced” and “overproduction” in the present disclosure refer to a production of a gene product or a polypeptide at a level more than the production of the same gene product or polypeptide prior to a genetic alteration of the microorganism or in a comparable microorganism which has not been genetically altered. “More than” includes, e.g., an increase of 10%, 20%, 30%, 40%, 50%, 60%, 70%, 80%, 90%, 100%, 150%, 200%, 250%, 300%, 350%, 400%, 450%, or 500% or more. The terms produce, producing, produced, and production may be generally understood to apply to proteins and in context equivalent to the terms express, expressing, expressed, and expression as generally understood to apply to genes.

Production or overproduction of the POI may comprise a heterozygous addition of the POI in the microorganism. Production or overproduction of the POI may comprise a homozygous addition of the POI in the microorganism. Production or overproduction of the POI may comprise an ectopic addition of the POI in the microorganism.

The POI may be produced in the microorganism by using random integration of an expression cassette possessing the gene of interest (GOI), where the GOI encodes the POI. The GOI may be integrated into the microorganism using targeted integration of an expression cassette, for example, by homologous recombination. The targeted integration of the expression cassette producing the POI may be performed using homologous recombination (HR).

In some examples the specific productivity, the rate at which lipid is produced by a cell, may be improved by the introduction of expression cassettes that result in production of the POI, such as the ones presented in SEQ ID NOs: 29-58. In some examples the specific productivity, the rate at which lipid is produced by a cell, may be improved by the introduction of expression cassettes that result in production of the POIs, including but not limited to GAPN_Sm, GAPN_Bc, GAPN_Ca, DGAT1_Co, DGAT2_Co, DGAT_Ce, PTA_Ec, ME1_Co, ME2_Co, ME_Rt, XPK_Co, XPK_Ca, POS5_Sc, ZWF_Co, CTP1_Co, CAT2_Sc, ACC1_Co, TOR1_Co, ACL_Co, OLE1_Y1, OLE1_Ss, DGAT Ss, DGAT_Y1 and VEL4. There is no guidance within the art as to selection of these particular modifications or their effect on lipid specific productivity. Velvet-like proteins (VEL4) are regulatory proteins that play a role in fungal development and have been implicated in regulation of glucan synthesis. It is unexpected that the overexpression of a protein involved in regulation of glucan synthesis such as VEL4 would not negatively impact cell structure, function, or proliferation or negatively impact specific productivity due to the importance of glucans in cell wall construction and septum deposition during division.

In some examples the composition of lipid may be altered by the introduction of expression cassettes that result in production of the POI, such as the ones presented in SEQ ID No: 32-34, 48-53, 60-65. In some examples the specific productivity, the rate at which lipid is produced per cell, may be improved by the introduction of expression cassettes that result in production of the POIs, including but not limited to ELO2_Ma, OLE1_Pg, EDA_Ec, EDD_Ec, CBBM_Td, PRK_So, OLE1_Co, FAT5 Ce, SCD_T66, ELO1_T66, ELO1_Co, ELO2_Y1 as is exemplified in SEQ ID NOs: 29-58. In some examples the substrate utilization of the microorganism may be altered. For example, the utilization of ETOH may be improved via introduction of expression cassettes that result in production of the POIs, such as the ones presented in SEQ ID NOs: 66-69. In some examples the substrate utilization of the microorganism may be altered. For example, the utilization of ETOH may be improved via introduction of expression cassettes that result in production of the POIs, including but not limited to ADH2_Sc, ALD6_Sc, EUTE_Ec, MPHF_Ec.

In some examples additional protein folding machinery may be necessary to improve activity of the POI for specific productivity (such as the ones presented in SEQ ID No: 29-58), composition (such as the ones presented in SEQ ID No: 60-65), or substrate utilization (such as the ones presented in SEQ ID No: 66-69). Examples of protein folding machinery include POIs including but not limited to GROEL_Ec and GROES_Ec. Introduction of expression cassettes that result in production of the POIs, such as the ones described in SEQ ID Nos: 70-71 may enable improved specific productivity, composition, or substrate utilization as the consequence of the strain. In some embodiments, the strains may also contain an expression cassette that results in production of the POI from SEQ ID NOs: 29-69.

In some examples the fermentation broth viscosity may be lowered by the introduction of expression cassettes that result in production of the POI, such as the ones presented in SEQ ID NOs: 58-59. In some examples the fermentation broth viscosity may be lowered by the introduction of expression cassettes that result in production of the POIs, including but not limited to VEL1 and VEL4. Velvet-like proteins (VEL1 or VEL4) are regulatory proteins that play a role in fungal development and have been implicated in regulation of glucan synthesis. There is no guidance within the art as to selection of these particular modifications or their effect on the fermentation broth. It is unexpected that the overexpression of a protein involved in regulation of glucan synthesis such as VEL1 or VEL4 would not negatively impact cell structure, function, or proliferation or negatively impact specific productivity due to the importance of glucans in cell wall construction and septum deposition during division.

In some examples the POIs, such as the ones presented in SEQ ID No: 29-71 designated as affecting characteristics for specific productivity, composition, substrate, protein folding, fermentation broth viscosity, or susceptibility to lysis will affect more than one characteristic. In some embodiments the improvement in specific productivity resulting from production of POIs, such as the ones presented in SEQ ID No: 29-58 may only be observed when the composition has been altered by a POIs, such as the ones presented in SEQ ID No: 60-65.

### Oil Compositions

The microorganisms described herein may produce oils comprising TAGs. The oils (and TAGs) produced by the microorganism may be modified by the one or more genetic modifications described herein.

In some embodiments, the oil may have a particular percentage of a particular fatty acid. The oil may have a C16:0 fatty acid percentage of at least about 20%, 25%, 27%, 30%, 32%, 35%, 40%, 50%, or more. The oil may have a C16:0 fatty acid percentage of at most about 50%, 40%, 35%, 32%, 30%, 27%, 25%, 20%, or less. The oil may have a C16:0 fatty acid percentage from about 20% to 50%, 20% to 40%, 20% to 30%, 25% to 40%, 30% to 40%, 35% to 40%, or 28% to 35%.

The oil may have a C16:1 fatty acid percentage of at least about 0%, 1%, 2%, 3%, 4%, 5%, 6%, 7%, 8%, 9%, 10%, 20%, 40% or more. The oil may have a C16:1 fatty acid percentage of at most about 40%, 20%, 10%, 9%, 8%, 7%, 6%, 5%, 4%, 3%, 2%, 1%, or less. The oil may have a negligible percentage of a C16:1 fatty acid. The oil may have a C16:1 fatty acid percentage from about 0% to 40%, 0% to 20%, 0% to 10%, 0% to 9%, 0% to 8%, 0% to 7%, 0% to 6%, or 0% to 5%.

In some embodiments, the oil may have a C18:0 fatty acid percentage of at least about 5%, 6%, 7%, 8%, 9%, 10%, 11%, 12%, 13%, 14%, 15%, 16%, 17%, 18%, 19%, 20%, 25%, 30%, or more. The oil may have a C18:0 fatty acid percentage of at most about 20%, 19%, 18%, 17%, 16%, 15%, 14%, 13%, 12%, 11%, 10%, 9%, or less. The oil may have a negligible percentage of a C18:0 fatty acid. The oil may have a C18:0 fatty acid percentage from about 7% to 25%, 8% to 22%, 9% to 21%, or 8% to 12%. In some cases, the oil may have at least about 10% C18:0 fatty acid.

In some embodiments, the oil may have a C18:1 fatty acid percentage of at least about 35%, 40%, 42%, 45%, 50%, 55%, 58%, 60%, 75%, 90% or more. The oil may have a C18:1 fatty acid percentage from 35% to 40%, 35% to 45%, 35% to 50%, 35% to 55%, 35% to 58%, 35% to 60%, 35% to 65%, 40% to 45%, 40% to 50%, 40% to 55%, 40% to 58%, 40% to 60%, 40% to 65%, 45% to 50%, 45% to 55%, 45% to 58%, 45% to 60%, 45% to 65%, 50% to 55%, 50% to 58%, 50% to 60%, 50% to 65%, 55% to 58%, 55% to 60%, 55% to 65%, 58% to 60%, 58% to 65%, 60% to 65%, 65% to 75%, or 75% to 90%. The oil may have a C18:1 fatty acid percentage about 35%, 40%, 45%, 50%, 55%, 58%, 60%, 65%, 75%, or 90%. The oil may have a C18:1 fatty acid percentage at least 35%, 40%, 45%, 50%, 55%, 58%, 60%, 65%, 75%, or 90%.

The oil may have a C18:2 fatty acid percentage of at least about 0%, 1%, 2%, 3%, 4%, 5%, 6%, 7%, 8%, 9%, 10%, or more. The oil may have a C18:2 fatty acid percentage of at most about 10%, 9%, 8%, 7%, 6%, 5%, 4%, 3%, 2%, 1%, 0.5%, 0.1%, 0.01% or less. The oil may have a C18:2 fatty acid percentage from about 0% to 10%, 0% to 9%, 0% to 8%, 0% to 7%, 0% to 6%, 0% to 5%, 0 to 4%, 0 to 3%, 0 to 2%, 0 to 1%, or 0 to 0.5%. In some cases, the oil may be at most about 1% C18:2 fatty acid. In some cases, the oil may be at most about 5% C18:2 fatty acid. In some cases, the oil may be at most about 0.1% C18:2 fatty acid or less.

The oil may have a C18:3 fatty acid percentage of at least about 0%, 1%, 2%, 3%, 4%, 5%, or more. The oil may have a C18:3 fatty acid percentage of at most about 5%, 4%, 3%, 2%, 1%, or less. The oil may have a negligible percentage of a C18:3 fatty acid. The oil may have a C18:3 fatty acid percentage from about 0% to 1%, 0% to 2%, 0% to 3%, 0% to 4%, or 0% to 5%.

The oil may have a C20:0 fatty acid percentage of at least about 0%, 1%, 2%, 3%, 4%, 5%, or more. The oil may have a C20:0 fatty acid percentage of at most about 5%, 4%, 3%, 2%, 1%, or less. The oil may have a negligible percentage of a C20:0 fatty acid. The oil may have a C20:0 fatty acid percentage from about 0% to 1%, 0% to 2%, 0% to 3%, 0% to 4%, or 0% to 5%.

The oil may have a C20:1 fatty acid percentage of at least about 0%, 1%, 2%, 3%, 4%, 5%, or more. The oil may have a C20:1 fatty acid percentage of at most about 5%, 4%, 3%, 2%, 1%, or less. The oil may have a negligible percentage of a C20:1 fatty acid. The oil may have a C20:1 fatty acid percentage from about 0% to 1%, 0% to 2%, 0% to 3%, 0% to 4%, or 0% to 5%.

The oil may have a C22:0 fatty acid percentage of at least about 0%, 1%, 2%, 3%, 4%, 5%, or more. The oil may have a C22:0 fatty acid percentage of at most about 5%, 4%, 3%, 2%, 1%, or less. The oil may have a negligible percentage of a C22:0 fatty acid. The oil may have a C22:0 fatty acid percentage from about 0% to 1%, 0% to 2%, 0% to 3%, 0% to 4%, or 0% to 5%.

The oil may have a C22:0 fatty acid percentage of at least about 0%, 1%, 2%, 3%, 4%, 5%, or more. The oil may have a C22:0 fatty acid percentage of at most about 5%, 4%, 3%, 2%, 1%, or less. The oil may have a negligible percentage of a C22:0 fatty acid. The oil may have a C22:0 fatty acid percentage from about 0% to 1%, 0% to 2%, 0% to 3%, 0% to 4%, or 0% to 5%.

In some embodiments, the underproduction of a protein such as a delta 12 desaturase and/or production/overproduction of a POI such as OLE1 may lead to a reduction in the C18:0 production by the microorganism. In some embodiments, the reduction in C18:0 production is at least 0.1% to 6%. In some embodiments, the reduction in C18:0 production is at least 0.1%. In some embodiments, the reduction in C18:0 production is at most 6%. In some embodiments, the reduction in C18:0 production is from 0.1% to 0.5%, 0.1% to 1%, 0.1% to 1.5%, 0.1% to 1.8%, 0.1% to 2%, 0.1% to 3%, 0.1% to 4%, 0.1% to 5%, 0.1% to 6%, 0.5% to 1%, 0.5% to 1.5%, 0.5% to 1.8%, 0.5% to 2%, 0.5% to 3%, 0.5% to 4%, 0.5% to 5%, 0.5% to 6%, 1% to 1.5%, 1% to 1.8%, 1% to 2%, 1% to 3%, 1% to 4%, 1% to 5%, 1% to 6%, 1.5% to 1.8%, 1.5% to 2%, 1.5% to 3%, 1.5% to 4%, 1.5% to 5%, 1.5% to 6%, 1.8% to 2%, 1.8% to 3%, 1.8% to 4%, 1.8% to 5%, 1.8% to 6%, 2% to 3%, 2% to 4%, 2% to 5%, 2% to 6%, 3% to 4%, 3% to 5%, 3% to 6%, 4% to 5%, 4% to 6%, or 5% to 6%. In some embodiments, the reduction in C18:0 production is about 0.1%, 0.5%, 1%, 1.5%, 1.8%, 2%, 3%, 4%, 5%, or 6%. In some embodiments, the % change is a weight % relative to the total weight (e.g., wt. %).

In some embodiments, the underproduction of a protein such as a delta 12 desaturase and/or production/overproduction of a POI such as OLE1 may lead to an increase in the C18:1 production by the microorganism. In some embodiments, C18:1 may refer to a mono-unsaturation at the delta-9 or delta-11 position. The amount of C18:Δ9 may represent 100%, 75%, 50%, 25% or less of the overall C18:1 composition. The amount of C18:Δ11 may represent 0%, 25%, 50%, 75% or greater of the overall C18:1 composition. In some embodiments, the increase in C18:1 production is about 0.1% to 10%. In some embodiments, the increase in C18:1 production is more than 0.1%. In some embodiments, the increase in C18:1 production is more than at most 10%. In some embodiments, the increase in C18:1 production is from 0.1% to 0.5%, 0.1% to 1%, 0.1% to 2%, 0.1% to 5%, 0.1% to 7%, 0.1% to 8%, 0.1% to 10%, 0.5% to 1%, 0.5% to 2%, 0.5% to 5%, 0.5% to 7%, 0.5% to 8%, 0.5% to 10%, 1% to 2%, 1% to 5%, 1% to 7%, 1% to 8%, 1% to 10%, 2% to 5%, 2% to 7%, 2% to 8%, 2% to 10%, 5% to 7%, 5% to 8%, 5% to 10%, 7% to 8%, 7% to 10%, or 8% to 10%. In some embodiments, the increase in C18:1 production is about 0.1%, 0.5%, 1%, 2%, 5%, 7%, 8%, or 10%.

In some embodiments, the underproduction of a protein such as a delta 12 desaturase and/or production/overproduction of a POI such as OLE1 may lead to a reduction in the C18:2 production by the microorganism. In some embodiments, the reduction in C18:2 production is at least 0.1%. In some embodiments, the reduction in C18:2 production is at most 6%. In some embodiments, the reduction in C18:2 production is from 0.1% to 0.5%, 0.1% to 1%, 0.1% to 1.5%, 0.1% to 1.8%, 0.1% to 2%, 0.1% to 3%, 0.1% to 4%, 0.1% to 5%, 0.1% to 6%, 0.5% to 1%, 0.5% to 1.5%, 0.5% to 1.8%, 0.5% to 2%, 0.5% to 3%, 0.5% to 4%, 0.5% to 5%, 0.5% to 6%, 1% to 1.5%, 1% to 1.8%, 1% to 2%, 1% to 3%, 1% to 4%, 1% to 5%, 1% to 6%, 1.5% to 1.8%, 1.5% to 2%, 1.5% to 3%, 1.5% to 4%, 1.5% to 5%, 1.5% to 6%, 1.8% to 2%, 1.8% to 3%, 1.8% to 4%, 1.8% to 5%, 1.8% to 6%, 2% to 3%, 2% to 4%, 2% to 5%, 2% to 6%, 3% to 4%, 3% to 5%, 3% to 6%, 4% to 5%, 4% to 6%, or 5% to 6%. In some embodiments, the reduction in C18:2 production is about 0.1%, 0.5%, 1%, 1.5%, 1.8%, 2%, 3%, 4%, 5%, or 6%.

In some embodiments, the underproduction of a protein such as a delta 12 desaturase and/or production/overproduction of a POI such as OLE1 may lead to a reduction in the C18:3 production by the microorganism. In some embodiments, the reduction in C18:3 production is at least 0.1% to 1%. In some embodiments, the reduction in C18:3 production is at least 0.1%. In some embodiments, the reduction in C18:3 production is at most 1%. In some embodiments, the reduction in C18:3 production is from 0.1% to 0.2%, 0.1% to 0.3%, 0.1% to 0.4%, 0.1% to 0.5%, 0.1% to 1%, 0.2% to 0.3%, 0.2% to 0.4%, 0.2% to 0.5%, 0.2% to 1%, 0.3% to 0.4%, 0.3% to 0.5%, 0.3% to 1%, 0.4% to 0.5%, 0.4% to 1%, or 0.5% to 1%. In some embodiments, the reduction in C18:3 production is about 0.1%, 0.2%, 0.3%, 0.4%, 0.5%, or 1%.

In some embodiments, the underproduction of a protein such as a delta 12 desaturase and/or production/overproduction of a POI such as OLE1 may lead to a reduction in the C20:1 production by the microorganism. In some embodiments, the reduction in C20:1 production is at least 0.1% to 1%. In some embodiments, the reduction in C20:1 production is at least 0.1%. In some embodiments, the reduction in C20:1 production is at most 1%. In some embodiments, the reduction in C20:1 production is from 0.1% to 0.2%, 0.1% to 0.3%, 0.1% to 0.4%, 0.1% to 0.5%, 0.1% to 1%, 0.2% to 0.3%, 0.2% to 0.4%, 0.2% to 0.5%, 0.2% to 1%, 0.3% to 0.4%, 0.3% to 0.5%, 0.3% to 1%, 0.4% to 0.5%, 0.4% to 1%, or 0.5% to 1%. In some embodiments, the reduction in C20:1 production is about 0.1%, 0.2%, 0.3%, 0.4%, 0.5%, or 1%.

In some embodiments, the underproduction of a protein such as a delta 12 desaturase and/or production/overproduction of a POI such as OLE1 may lead to a reduction in the C22:0 production by the microorganism. In some embodiments, the reduction in C22:0 production is at least 0.1% to 1%. In some embodiments, the reduction in C22:0 production is at least 0.1%. In some embodiments, the reduction in C22:0 production is at most 1%. In some embodiments, the reduction in C22:0 production is from 0.1% to 0.2%, 0.1% to 0.3%, 0.1% to 0.4%, 0.1% to 0.5%, 0.1% to 1%, 0.2% to 0.3%, 0.2% to 0.4%, 0.2% to 0.5%, 0.2% to 1%, 0.3% to 0.4%, 0.3% to 0.5%, 0.3% to 1%, 0.4% to 0.5%, 0.4% to 1%, or 0.5% to 1%. In some embodiments, the reduction in C22:0 production is about 0.1%, 0.2%, 0.3%, 0.4%, 0.5%, or 1%.

In some embodiments, the underproduction of a protein such as a delta 12 desaturase and/or production/overproduction of a POI such as OLE1 may lead to an increase in C16:0 production by the microorganism. In some embodiments, the increase in C16:0 production is about 0.1% to 2.5%. In some embodiments, the increase in C16:0 production is more than 0.1%. In some embodiments, the increase in C16:0 production is more than 2.5%. In some embodiments, the increase in C16:0 production is from 0.1% to 0.5%, 0.1% to 1%, 0.1% to 1.5%, 0.1% to 2%, 0.1% to 2.5%, 0.5% to 1%, 0.5% to 1.5%, 0.5% to 2%, 0.5% to 2.5%, 1% to 1.5%, 1% to 2%, 1% to 2.5%, 1.5% to 2%, 1.5% to 2.5%, or 2% to 2.5%. In some embodiments, the increase in C16:0 production is about 0.1%, 0.5%, 1%, 1.5%, 2%, or 2.5%.

In some embodiments, the overproduction of a protein such as the C16:0 desaturases FAT5 (exemplified in SEQ ID NO: 61), SCD_T66 (exemplified in SEQ ID NO: 62), or any stearoyl-CoA desaturases such as the SCD from Y. lipolytica and S. segobiogensis (exemplified in SEQ ID NO: 48 and 49) which may use C16:0 as substrate may be used to increase the C16:1 content.

In some embodiments strains that overproduce (exemplified in SEQ ID NO: 61), SCD_T66 (exemplified in SEQ ID NO: 62), or any stearoyl-CoA desaturases such as the OLE1 from Y. lipolytica and S. segobiogensis (exemplified in SEQ ID NO: 48 and 49) which will use C16:0 as a substrate may be combined with an additional overproduction of a C16:1 elongase such as the ELO1_T66 (exemplified in SEQ ID NO: 63) which converts C16:1 to C18:1 where C18:1 is C18:1411.

### Oil Productivity

In some embodiments, specific productivity of the host cell, for instance the rate at which oils comprising TAGs are produced may be higher than a control microorganism which has not been genetically modified as described herein. In some cases, the microorganisms with higher oil specific productivity may be microorganisms modified to underexpress one or more proteins from Table 1. In some cases, the microorganisms with higher oil specific productivity may be microorganisms modified to overexpress one or more proteins from Table 2. In some cases, oil specific productivity for a microorganism being cultured may be 0.5 g/L/h to 3 g/L/h. In some cases, oil specific productivity for a microorganism being cultured may be at least 0.5 g/L/h. In some cases, oil specific productivity for a microorganism being cultured may be at most 3 g/L/h. In some cases, oil specific productivity for a microorganism being cultured may be 0.5 g/L/h to 1 g/L/h, 0.5 g/L/h to 1.25 g/L/h, 0.5 g/L/h to 1.5 g/L/h, 0.5 g/L/h to 1.75 g/L/h, 0.5 g/L/h to 2 g/L/h, 0.5 g/L/h to 2.5 g/L/h, 0.5 g/L/h to 3 g/L/h, 1 g/L/h to 1.25 g/L/h, 1 g/L/h to 1.5 g/L/h, 1 g/L/h to 1.75 g/L/h, 1 g/L/h to 2 g/L/h, 1 g/L/h to 2.5 g/L/h, 1 g/L/h to 3 g/L/h, 1.25 g/L/h to 1.5 g/L/h, 1.25 g/L/h to 1.75 g/L/h, 1.25 g/L/h to 2 g/L/h, 1.25 g/L/h to 2.5 g/L/h, 1.25 g/L/h to 3 g/L/h, 1.5 g/L/h to 1.75 g/L/h, 1.5 g/L/h to 2 g/L/h, 1.5 g/L/h to 2.5 g/L/h, 1.5 g/L/h to 3 g/L/h, 1.75 g/L/h to 2 g/L/h, 1.75 g/L/h to 2.5 g/L/h, 1.75 g/L/h to 3 g/L/h, 2 g/L/h to 2.5 g/L/h, 2 g/L/h to 3 g/L/h, or 2.5 g/L/h to 3 g/L/h. In some cases, oil specific productivity for a microorganism being cultured may be about 0.5 g/L/h, 1 g/L/h, 1.25 g/L/h, 1.5 g/L/h, 1.75 g/L/h, 2 g/L/h, 2.5 g/L/h, or 3 g/L/h. In some cases, oil specific productivity for a microorganism being cultured may be at least 0.5 g/L/h, 1 g/L/h, 1.25 g/L/h, 1.5 g/L/h, 1.75 g/L/h, 2 g/L/h, or 2.5 g/L/h. In some cases, oil specific productivity for a microorganism being cultured may be at most 1 g/L/h, 1.25 g/L/h, 1.5 g/L/h, 1.75 g/L/h, 2 g/L/h, 2.5 g/L/h, or 3 g/L/h.

In some cases, oil specific productivity for a microorganism may be higher than a control microorganism which has not been genetically modified as described herein. In some cases, oil specific productivity for a microorganism may be 5% to 300% higher than a control microorganism. In some cases, oil specific productivity for a microorganism may be at least 5% higher than a control microorganism. In some cases, oil specific productivity for a microorganism may be at most 300% higher than a control microorganism. In some cases, oil specific productivity for a microorganism may be 5% to 10%, 5% to 25%, 5% to 50%, 5% to 75%, 5% to 100%, 5% to 150%, 5% to 200%, 5% to 300%, 10% to 25%, 10% to 50%, 10% to 75%, 10% to 100%, 10% to 150%, 10% to 200%, 10% to 300%, 25% to 50%, 25% to 75%, 25% to 100%, 25% to 150%, 25% to 200%, 25% to 300%, 50% to 75%, 50% to 100%, 50% to 150%, 50% to 200%, 50% to 300%, 75% to 100%, 75% to 150%, 75% to 200%, 75% to 300%, 100% to 150%, 100% to 200%, 100% to 300%, 150% to 200%, 150% to 300%, or 200% to 300% higher than a control microorganism. In some cases, oil specific productivity for a microorganism may be about 5%, 10%, 25%, 50%, 75%, 100%, 150%, 200%, or 300% higher than a control microorganism. In some cases, oil specific productivity for a microorganism may be at least 5%, 10%, 25%, 50%, 75%, 100%, 150%, or 200% higher than a control microorganism. In some cases, oil specific productivity for a microorganism may be at most 5%, 10%, 25%, 50%, 75%, 100%, 150%, 200%, or 300% higher than a control microorganism.

In some cases, oil specific productivity for a microorganism can be increased by overexpression of either phosphoketolase, malic enzyme, or VEL4 (respectively SEQ ID NOs: 39-40, 36-38, 58). For example, overexpressing phosphoketolase can increase specific productivity of lipids 14.1% as compared to WT, overexpressing malic enzyme ME2 can increase specific productivity of lipids of as compared to WT 12.7%, and overexpressing VEL4 exhibited an increase in specific productivity of lipids of 9.2% as compared to WT.

### Cell Wall Enhancements and Byproduct Elimination

In some embodiments, cell walls from the microorganisms described herein may be by products in the oils produced during fermentation. The microorganisms described herein may be modulated to reduce the level of byproducts produced as a result of fermentation. In some cases, one or more proteins may be underexpressed to reduce cell wall byproducts. In some cases, one or more proteins selected from any one of SEQ ID NOs: 10-28 may be underexpressed to alter the microorganism's cell walls or cell wall related processes therefore decreasing fermentation broth viscosity and increasing ease of purification. In some cases, one or more proteins may be overexpressed to reduce cell wall byproducts. In some cases, one or more proteins selected from any one of SEQ ID NOs: 58-59 may be overexpressed to alter the microorganism's cell walls or cell wall related processes decreasing fermentation broth viscosity, increasing ease of purification, and/or increasing specific productivity.

In some cases, underexpression or overexpression of one or more proteins described in SEQ ID NOs: 27-28, 58-59 may lead to a reduction in cell wall related processes and in some cases may also improve oil specific productivity. In some cases, this underexpression or overexpression may alter one or more fermentation characteristics such as oxygen transfer rate, broth viscosity and cell shear. In some cases, oxygen transfer rate may be improved by underexpressing or overexpressing one or more cell wall related proteins. In some cases, broth viscosity may be improved by underexpressing or overexpressing one or more cell wall related proteins. In some cases, cell shear may be improved by underexpressing or overexpressing one or more cell wall related proteins.

In some cases, underexpressing or overexpressing one or more cell wall related proteins may modify the microorganism and make it more susceptible to lysis. In some cases, these modified cell wall related proteins may also reduce the fermentation broth viscosity. A microorganism is more susceptible to lysis if it is more likely to lyse than unmodified microorganisms. This increased susceptibility may lead to more economical and less intensive cell disruption process with higher oil recovery yield. In some cases, the modifications to cell wall related proteins may make the microorganism more susceptible to mechanical lysis. In some cases, the modifications to cell wall related proteins may make the microorganism more susceptible to physical lysis. In some cases, the modifications to cell wall related proteins may make the microorganism more susceptible to chemical lysis. In some cases, the modifications to cell wall related proteins may make the microorganism more susceptible to biochemical lysis.

In some cases, modifications to cell wall proteins may alter cell size which would increase oil yield and titer of the microorganism. Not wishing to be bound by a theory, this improvement in yield or titer may be based on the geometry of a sphere, whereby larger cells have a larger volume relative to surface area and where surface area represents the outer periphery of the cell and volume the inside of the cell where lipid is accumulated.

### Methods and Tools for Genetic Manipulation

Production of a recombinant protein such as the POI can be provided by an expression vector, a plasmid, a nucleic acid integrated into the host genome or other means. For example, a vector for expression can include: (a) a promoter element, (b) a signal peptide, (c) a heterologous protein sequence, and (d) a terminator element. In some embodiments, a promoter element can comprise additional genetic regulatory elements.

Expression vectors that can be used for production of a POI include those containing an expression cassette with elements (a), (b), (c) and (d). In some embodiments, the signal peptide (c) need not be included in the vector. In general, the expression cassette is designed to mediate the transcription of the transgene when integrated into the genome of a cognate host microorganism.

To aid in the amplification of the vector, for example in another host microorganism, prior to transformation into the target production host microorganism, a replication origin (e) may be contained in the vector (such as pMB1, pBR322, ColE1, R6K, p15A, pSC101, etc). To aid in the selection of microorganisms stably transformed with the expression vector, the vector may also include a selection marker (f) such as G418 gene and Neomycin resistance gene (NeoR). The expression vector may also contain a restriction enzyme site (g) that allows for linearization of the expression vector prior to transformation into the host microorganism to facilitate the expression vectors stable integration into the host genome. In some embodiments the expression vector may contain any subset of the elements (b), (e), (f), and (g), including none of elements (b), (e), (f), and (g). Other expression elements and vector elements known to one of skill in the art can be used in combination or substituted for the elements described herein.

Exemplary promoter elements (a) may include, but are not limited to, a constitutive promoter, inducible promoter, and hybrid promoter. Promoters include, but are not limited to promoters for the native C. oleaginosus genes AAC2; GAPDH; TEF1; L21E.

A signal peptide (b), also known as a signal sequence, targeting signal, localization signal, localization sequence, signal peptide, transit peptide, leader sequence, or leader peptide, may support secretion of a protein or polynucleotide or target that protein or polypeptide to a membrane or subcellular organelle. Extracellular secretion of a recombinant or heterologously produced protein from a microorganism may facilitate protein purification. Intracellular targeting of a protein or polypeptide may facilitate placing the activity of that protein or polypeptide selectively into an area of the cell where it can have greatest benefit. For example, the targeting of a regulatory protein to the nucleus, where it would be most able to interact with the genome. A signal peptide may be derived from a precursor (e.g., prepropeptide, preprotein) of a protein. Signal peptides can be derived from a precursor of a protein other than the signal peptides in native a recombinant POI.

Any nucleic acid sequence that encodes a recombinant POI can be used as (c). Preferably such sequence is codon optimized for the species/genus/kingdom of the microorganism.

Exemplary transcriptional terminator elements include, but are not limited to terminators from the native C. oleaginosus genes AAC2; GAPDH; TEF1; OLE1.

Exemplary selectable markers (f) may include but are not limited to: an antibiotic resistance gene (e.g. neomycin, zeocin, ampicillin, blasticidin, kanamycin, geneticin, nourseothricin, chloramphenicol, tetracycline, triclosan, ganciclovir, and any combination thereof).

A microorganism may be transformed to include one or more expression cassettes. As examples, a microorganism may be transformed to express one expression cassette, two expression cassettes, three expression cassettes or more expression cassettes. In one example, a microorganism is transformed to express a first expression cassette where a first GOI is translated to a first POI and to express a second expression cassette where a second GOI is translated to a second POI.

Table 1 provides genetic modifications including: Knock Out Proteins. Proteins representing specific enzyme classes whose activity attenuation are designed to improve oil yield, specific productivity, titer, composition, fermentation viscosity, and/or susceptibility to lysis.

TABLE 1
Genetic Modifications

| SEQ ID | Sequence | |
|---|---|---|
| NO. | Info | Amino acid sequence |
| 1 | FAD2A | MSAATLRQRNVDKPGAADKAELLREAEDLELTEGQKFVVPNFTVKQ |
| | EC | LLDAIPAHCYKRSAFKSSLYVLQDFVLLAALVYGAYHIDSFLSRFNLG |
| | 1.14.19.6 | SVAHTAAKIGLWFTYQVLAGMVGTGIWVIAHECGHQAYSESKTINN |
| | | AVGWVLHSILLVPYHSWRISHGRHHAATGHLTRDEVFVPRTREQLGI |
| | | QAPKTEEEKKGINVPAWRQAELREALEESPIGALYGAILHQLFGWPM |
| | | YLIRNASGQLWYPKMTNHFQPSSIIFKPSHFWQIIASDIGVVLTAAALG |
| | | VFVYYRGFAEMARIYLIPYLWVNHWLVFITFLQHTDPVLPHYSEKTW |
| | | TFARGALATIDRNCLGPVGPYLFHGITETHVAHHTSSRIPHYNAWEAT |
| | | EALKKFLGPHYHYNPENMFVSFWKAHRYCKFIEAGEDVAFYRNAAG |
| | | VAQKVGIIEENGAVSDSGVEHK |
| 2 | TGL3- | MTAIDDIPEGVDPTLWTYNHINEEHLAAFEHALSLQDVAPIEGDMSPA |
| | 370130 | MLKSPQLTPHDAPEWSKGVEKLTATSDFAPIHSRVRKRRSAGQQSLG |
| | (lipase) | LTYHLIRWPLLAFFFLIIWIEFSAYVLTRQVVNVFEWLFAWRGVQRVL |
| | EC | KRKLRNAKSYEEWRATAKELDDELCFDDWKETDSDGYFDANLVRR |
| | 3.1.1.3 | VRATLYKLRLAKDTRALMDALSICVRPNFAGTESSRMYSETFYGTKT |
| | | LVEAHIKEVAASLDYVRTASNVTLEEKRAFFRRLNKNYGTSALCLSG |
| | | GASFGYYHFGVARALLDHGLLPRVITGTSAGGLIAALVCTYTDSELK |
| | | QLIRPELADKITALEDPFSVWIRRWMQTGARFSALEWARKAMYFTRG |
| | | SLTFKEAYERTGRALNVSVVPADRHSPTILLNHLTAPNCVIWSAIIASA |
| | | AVPGILNPVVLMAKDRDGSVRPHNLGGSRFKDGSLREDIPLSSLHTQF |
| | | NCNFSIVSQTNPHIHLFFFAPRGAVGRPVAHRKGRGWRGGFILSALES |
| | | YIKLDLSKHFKVIRDLDLLPQLLQSDWSGVFLQRFFGDLTLIPKSRPLD |
| | | WLHILDDPDRKGLARMILVGQRVTWPTIHMVHNRAKIEDAIHRGRAE |
| | | VRMALARERPSAAPTGTMPLESDADSAIALRARRADGQGSGSSRRRR |
| | | PDRLQIPRTQNITDVQAVTSPNELPRSNTAPSFGDTFRQMRSSPLAAFG |
| | | FKNRTRSRRSISAWLGGGSDSSLSDDEVDLGGPEWRADAAATANGLD |
| | | LNDDVFLDSGEVSAALPDSTSDPTQDGSASDP |
| 3 | MFE1- | MASLDFKDLVVLITGAGGGIGAGYSHFYGARGAKVVVNDVSAQSAQ |
| | 385434 | KVVDEIKAAGGQAAIAVGSVTEGQKIVDQAVQAFGTVHVLINNAGIL |
| | (B- | RDKSFKKMDDAQWDAVIAVHLKGAYSMTKAVWPLFRQQKFGRVIN |
| | oxidation) | TSSPAGIYGNMGQANYSAAKMALVAFSKSLSIEGVKYDIRANTIVPLA |
| | | ASAMTQTVMPPEVLKGLNPDFIAPIIGALTARNGPDVRGRIFEVGGGF |
| | | YSELRWERSKGHLFKTDDTFTPTAIRHEWKKITDFSKDVEYPGDLKA |
| | | AGRNLMAVIEKQPGLKPNEQGPPPPVDFKGQTVIITGAGAGLGRAYA |
| | | HLYSKLGANVVVNDVSAQNAEAVVKECEQLGAKAAAAVCSAEEGE |
| | | KIVKVALDKFGGVQVLVCNAGVLRDKAFVNMTEEQWDTVMAVHLR |
| | | GTFRCCKAVWPIFQEQKYGRILVTSSPNGIIGAHGQGNYASAKAGLIG |
| | | FGRSLAIEGKKNNILTNILAPSAGTAMTATVWTEEMVKTFSPDFVAPV |
| | | VAFLTSKDQKGTGQVIETYGGQVAALRWQRTHGAVFPNNRVPTPEQ |
| | | VVAKWDEITTFDDKATHPRNTQESGMQIFENFSNLAPEEEQKDYVDP |
| | | EDPEAIVKAKRAEPKGIEYEYTERDVMLYNLGIGAHATELQWTYEND |
| | | QDFQPLPTFGVIPQFTAGLGDYDFLPNYNPGKLLHGEQYLSIKGKVPT |
| | | SGKLVNKARLFEVLDKGKAAAVTTVIESRDAQTNELIYENHSTVFIRD |
| | | SGGFGGKRAAKDRGAATAPNAPPKRKPDAVVEEKTSPDQAALYRLS |
| | | GDYNPLHIDPQFSIIGGFKKPILHGLCSMGFAGRHVLKTFGPYKDIKVR |
| | | FAGTVIPGETLVTEMWKEGDKVIFQTKVKERDAPALSAAAVTLINSG |
| | | APKSKM* |
| 4 | TOR1 | MSSQPEALENIFSRLGSKSEDVRVAAAEDLKAQINSANIEFPGDDSKK |
| | (372351) | NVWKEVFHRTFELTRSNTTSERLGSIAAIDAMLEETNNDSPDRALQNN |
| | (Target of | IRLYGYLRPLLTYPDSTVMVAAAHVVGNMVKLAGANLGESFFNKEV |
| | rapamycin- | GQAIQMIDDSRQEVGRFSAVLLLHQFAVYAPSQFYPFISRVLEKIWIPL |
| | signaling) | RDSRTMVRERASMLLSSCLDIMKARERSSSEIYQVIFEEAKSGLLKASS |
| | | VDSILGSLLAFSAMLQNQQISMGEYYRQICEITLKYRDSKETVIRKSVI |
| | | TLIPSMATYDSDEFEVHYLHRSMAYLLQALNKPTDRDIAYVALGHLSI |
| | | QLASKMRPFIDDIVKIIKEHLRMRGKKGAPFEAPIFQCLAMLTTAVGP |
| | | MLTRQMHEILDLMFPWGLSEALYHALEVISSHIPPLLRTIQDRLLETLS |
| | | VILTGHSFRPLGAPVRRAKGGEITRDVVIQASGGGQSPETIALALKVLG |
| | | NFDFGGHTLNEFVQEAALPYLEHDSPEVRKEAVLASTQLFVNDPICNQ |
| | | TSSHAIEIVNDVLEKLLTVGITDQVPGIRQAVLESLDEKFDRHLAQAED |
| | | IRCLFVALNDEEFKIRELAIGIIGRLAHHNPAYVMPSLRKSLINLITELE |
| | | YSTANKQKEESAKLLCLLIGASSSLVKSYASTILSVILRIARSPSTPATV |
| | | TASCVNCIGELARVAGEELVSNAKTIMDLLLELLNDPASTVKRNAAL |
| | | KALGQVVSNTGEVITPYSDYPQLLGILFRILRLETSSQIRLEAIRTLGML |
| | | GALDPFKHKQLLGGDNDNTTESTGPRVTDITLLMNHNGGSNDDYYQ |
| | | MVVINSLVNVLNDANMKEHHYDAINSVMLIFRTQRLRCVGFLPQILP |
| | | AFLGVIRMGSRDSQDVFLKQLAQLISIVKQHIRNYLSEVFALVHDYW |
| | | NPNSMMQITIISLVEAIARAVEGEFKAYLPQLLQQILRTFDGDLSARNL |
| | | TDQRENTLLHILKAFIVFGSSIEDYLHLVLPVIIRSFENPAAPTKLRKAA |
| | | LTTTAQLCRKVNFSDHASQIIHPLVRTLSTQDPELRNAAMETLCVLVL |
| | | QFGPDYAIFIPMVNKALLENKIVWPMYDQMVTKLLNRERLPPDLGPV |
| | | EQYAADSMNEVSVSAEQHKLPVNQQQLKSAWDCSHVATKKEWLEW |
| | | LKRLGVEFMRESPSQAIRACTKLADQHGAFARELFNVAFVSCWTELY |
| | | ESYQEDLVHHLEQALTNDGVPSEVVNTVLNLAEFMEHDDKPLALEPR |
| | | LLGDYATAFHAYAKALHYKELEFFVDASPTVVEDLISVNQKLQQSDA |
| | | AWGTLEYAQNNLQMTHDVLWYEKLGRWEEALEVWNERSADPDSQF |
| | | DEQALAMGKFQALHALGEWEELADVIQVRWPNAAGEEKKTIAPLGA |
| | | AASWALYQWDMMDDYISAMKTDSADRNFFKAILAVHRNQFTSAKR |
| | | HITKARERLDGELTTLTGESYGRAYDVIVRVQMLSELEEIISYKDHAD |
| | | QPERQATQRQTWHKRLAGCQRDVEVWQRILQVRSLVLTPSEDMETW |
| | | ISFADLCRVSDRLNLAEKTLTSLVGSSCSNLDAESRSRAPPPIVFAYYR |
| | | LKWAQAVAVDSRDERVETLGYLHDFTQTLSDDMGLGPRNNQGRLIL |
| | | PDVKLYGEYTKLLARCHVELGKWEASMLEQGFTAAPASVLSNYALA |
| | | TELDPDWYQAWHTWALANFEVIGSLEVSQSGLQPAHFATYIIPAVEG |
| | | FLRSIALSPGNSLQDTLRLLTLWFKYGYQSGVNQAIQQGMASVNIDV |
| | | WLEVIPQIIARIQTPRPTIRELIVRLLHSIGRAHPQALIYPLTVASKSNVA |
| | | TRKQVAQDIMDKMREHSATIVDQADLVSTELIRAAILWHEQWHEGLE |
| | | EASKHYFADHNEEAMFEVLEPLHDMIEKGPETVRETSFVQSFGHDLR |
| | | LARDLCRRYRQQGDLNELNQAWDIYCATFQRLARQIKLLNVIELQYV |
| | | SPKLMNVRDLDLAVPGTYQSGKPVIGIAYVLPTFTVINSKQRPRRFTM |
| | | RGRDGKEYTFVLKGHEDLRQDERVMQLFGLVNTLLSADQESSKRHL |
| | | SIRQFSITPLSPGAGLLGWVPHSDTLHALIKHYRDSRKILIDIETRLMNQ |
| | | MADDNYDSLPLLHKVEVFQYALDNTTGQDLYRILWLRSRNSESWLE |
| | | RRTTYTRSLALTSMVGYILGLGDRHPSNLMLDQITGEIVHIDFGDCFE |
| | | VAMQRDKYPEKVPFRLTRMLIHAMEVCGITGTFSRSCEVSMEVLRAN |
| | | KESLMAVLEAFVYDPLINWRLTAGQGGQRQGAHDVEERGGQPYAV |
| | | QRGGRADERDILVEADRPEVMNDKALQVVERVRRKLTGRDFKPNVT |
| | | LDIKEQVEELVTQATSVENLCVAFVGWCSFW |
| 5 | GSY1 | MAVQRNVHQPLLFECAWEVANKVGGIYTVIKTKVPVTVKEYGDRYT |
| | Glycogen | LIGPLSYKTAHIEVESEDPEPGSPMEGTLNAMRDHGVKYIYGRWLIEG |
| | synthase | APRVLLFDTGSCYNRMDEWKGDLWNVAGIPTPPNDHETNETLVFGY |
| | 365053 | MVAWFLGEFSGRCLDTAIIAHFHEWQAGLAIPLARKRKIDVTTIFTTH |
| | EC:2.4.1.11 | ATLLGRYLCAGSVDFYNNLQYFDVDHEAGKRGIYHRYCIERSAAHCA |
| | | DVFTTVSHITAYESEHLLKRKPDGVLPNGLNVVKFAAMHEFQNLHVK |
| | | AKEKINDFIRGHFYGHYDFDLDNTIYMFTAGRYEFRNKGVDMFIESLA |
| | | RLNHRLKAMNSKMTVVAFIIMPAATNSYTIEALKGQAVTGQLRDVV |
| | | KQITDRIGNRLFEHAARYDGTHGTEVPKPEELMSAEDLVLLKRRVFA |
| | | LKRNSLPPVVTHNMQDDSNDPILNQIRRVQLFNRTSDRVKVIFHPEFL |
| | | NSNNPILGLDYEEFVRGCHLGVFPSYYEPFGYTPAECTVMGIPSVTTN |
| | | LSGFGCFMEDLLESPEDYGCYIVDRRGQGVDESVEQLTQVLTEFTTKS |
| | | RRQRINQRNRTERLSELLDWKSLGLEYAKARQLALRRAYPDSFNDDE |
| | | PEFTGVQRVGAPLSAPGSPRYRSGMMTPGDYATLTEEMEHLNTSDY |
| | | MGAKHPWNWNKEDSDEEEGYTFPIAGLKPRGRSDSLASAISGTMTPS |
| | | GKHRLNESDLAQADKALESHSSAANANGNGNK* |
| 6 | Trehalose | MNFDASYWLAYREANIRFADVVASFVRTGDVVWVQDYHLMLLPML |
| | synthase | LRSMITGESNQGEMTRREMGRVKEGVDEAVVNDINIKMPPPNRGNG |
| | 371254 | GGADEGVEMLEDVEEESNPKSPTHRQSSSTPGMTPFQKSEIQARRRGK |
| | (TPS1) | GGVRIGFFLHTPFPSSEIYRILPVRREILLGVLQCDLIGFHTYDYARHFL |
| | EC:2.4.1.15 | SACTRILGLETQPNGIEFEGRWVQVGTFPIGIDPWQFVEGVKLPVVQE |
| | | RLDRLEKRFEDCKVIIGVDRLDYIKGIPQKLHAFEVFLTQHPEWIEKVI |
| | | LIQLAIPSRQDVEEYMNLRSCVNELVGRINGQFSTPTWTPIVFMHRSVP |
| | | FEELTAMYALADVCLVTSTRDGMNLVAYEYISSQAEKHGSMILSEFA |
| | | GASQSLAGSLIINPWDVDSTASAIHQALTLAPEIRAQNWTKLFAYVSN |
| | | FTAQSWGLSFLNELNRSTGRRPTGPLLGGRRKSSSNLSRASSRASIKRR |
| | | FTVNSSTPVAPVVAS* |
| 7 | TFDH1 | MSAAKSYEGSAQLIDGNAIAKEIRQNIHAAITELQAKERSFKTPVLNIY |
| | (C1- | QLGSDPASSTYIRMKLKAAEESGMKVEHIQVPADSEVEGAAKGTGVR |
| | tetrahydro | KLLDLVHKANEDEKVAGILVQLPLPGASKEEEKKVVESISVGKDVDG |
| | folate | FHPENIGLLSGRLTEPHFTPCTPAGIVRLIDSTGFDLKGANVVVLGRSDI |
| | synthase) | VGTPTCALLRKRDATVTQCHSKTVGLPDIVRQADVVVAAIGQGQFVK |
| | 395209 | GDWIRPGAIVIDVGTNFIPDASKKSGQRMVGDVDFPAASNVAGFITPV |
| | EC | PGGVGPMTVAMLMNNTFRAARRQWESSRQRKLTPLPLNILPQVPSDI |
| | 6.3.2.17 | EIAVAQTPKPVADIAREIGVHDDEIESYGKYKAKIELSVLERLQERKD |
| | | GKYIVVAGITPTPLGEGKSTTTIGLAQALGAHLQKTAIACVRQPSQGP |
| | | TFGVKGGAAGGGYSQVIPMTEFNLHLTGDIHAVTAANNLLAAAIDAR |
| | | MFHESTQTDKALFNRLCPPKKGVRSFAKPMLKRLEKLGINKTNPADL |
| | | TEEEAARFARLDIDPATITWNRVLDVNDRYLRKITVGQAPTEKGFTRE |
| | | TAFDIAVASEVMAVLALATDVADMRERLGRMVVASSKKGEPITAEDI |
| | | GCAGAMAVLMKDAIKPTLMQTLEGTPVFVHAGPFANIAHGNSSIIAD |
| | | RIALKLAGIEEGDAEDRNGYVITEAGFGADIGMEKFCNIKTRVSGLRP |
| | | NAVVLVATIRALKMHGGGPTVSPGKPLDPVYSTENLELLQKGCANLG |
| | | KHIENAKKFGLKVVVAINKFVFDTEAEMKLVQDFALQAGADYAVPA |
| | | NHWAKGGEGAVELAQAVVEACKDESQFKFLYDVNLPLEEKMSIIAR |
| | | EMYGADGVELSPEAKAEVERYERQGYAHLPICMAKTALSLSDDPSRK |
| | | GVPTGFTLPIRNVRLSAGASFVYPLVGDMSTMPGLTTRPGFYDIDLDP |
| | | ETGDILGLF |
| 8 | PEX10 | MDSTRSVSHTIQGQSVAQPLTASAAVAGTVTPPDAFDRLQPEPASQA |
| | (lipase) | HILRSYQRDTAQVGRLTELVSELLRSMAGTRWLTHKQTIVDLAVRAV |
| | 367175 | YLMLTFGRGRQTLGEEYTDILPTANKLAPTRRRRLLTVALLLLPTALT |
| | E3 | SPSMLHYLRGDPYDHSRWAAAKRRVARFIESPLGQSLPEIHLVMFMF |
| | ubiquitin | GGRFYEFARRATGMVYVSDMPPLPAEHKPASYEPLGLIMALPLLYRL |
| | ligase | FPRSTGGASGASATRAVAGSMEEKVKENDYSGTATPIRNAPEDSVVV |
| | | TSEEQYDLPNTYLDEEALDLPERQCTLCLESRGTGEGSGGTTAVTECG |
| | | HIFCWGCLGGLDKMECPLCRQALRMERLIAAYNL |
| 9 | OLE1 | MSASTKQAATTVAQPSGKPVTNVIDPERDDFIVPDNYVTRTVENMKM |
| | 348634 | LPPVTWRNLHKNIQWISFLALTIPPAMAIYGLCTVPVQTKTFIWSVVY |
| | EC:1.14.19.1 | YFITGLGITAGYHRLWAHRSYNASKPLQYFLALAGAGSVQGSIRWWS |
| | | RGHRAHHRYTDTKLDPYSAHEGFWHAHMGWMLIKPRGKIGVADISD |
| | | LSKNPVVKWQHNNYVALLFFMGLAFPTLVAGLGWGDWWGGLFFAG |
| | | AARLVFVHHSTFCVNSLAHWLGETPFDNKHTPKDHFITALVTVGEGY |
| | | HNFHHQFPMDFRNAIKWYQYDPTKWFIWTMAQLGLASHLKKFPDNE |
| | | IKKGQYTMKLMQLQEQSEKLEWPKHSNDLPVISWEDFQAESKTRALI |
| | | AVHGFIHDCSSFIEDHPGGAHLIKRAIGTDSTTAFFGGVYDHSNAAHN |
| | | LLAMMRVGVLDGGMEVEHLKVGRLTRSNSADSLVSDAPSSASSTVS |
| | | ASSILSEVEDMDKPERKPVYQPVAQVSNPWTLAVPPSEKYRIIRTSPEI |
| | | RPGLISRIQSAASPSLTRAASFDAPVAP* |
| 10 | UDP-Glc | MRNELNRMVQGVTDPAKKKMFNAEMESFFLLFNRYLTEKAKGEKID |
| | pyrophosphorylase | WEKIQPPKPEQVRPYKVLDKVDPSILNKLAVLKLNGGLGTTMGCVGP |
| | (UGP) | KSVIEVREGMTFLDLSVRQIEHLNEKYNVNVPFILMNSFNTDDDTARII |
| | 347999 | QKYQNHNINILTFNQSRYPRVNKESLLPCPADATSDKANWYPPGHGD |
| | EC:2.7.7.9 | IYDAINNSGLLDQLIAAGKEYIFISNVDNLGAVVDLNIFQTMIDAQAEY |
| | | VMEVTDKTKADIKGGTIIDYEGKPRLLEVAQVPKDHLDEFCSTRKFKI |
| | | FNTNNIWCSLKAIKRVVDNDELNLEIIVNNKVTDKGEAVIQLETAIGA |
| | | AIKHFDSSIGINVPRSRFLPVKSCSDLLLIKSGLYNLQHGVLTMDKSRE |
| | | FGGTPVVKLGDQFKKVANFEKRFKSIPNITELDHLTVSGDVWFGKRV |
| | | RLAGTCIIVATEGNKIMIPDGTDLENKLITGNLSIIDH* |
| 11 | UDP-Glc | MSPKTADEIAAAADATIHEVGYDRNGFDSRIDTKTLRSLYLAPAFIKA |
| | decarboxylase | GAETTNVGSASKDYGTESVYDKDSNKIQYTTFAKFPPVRLLPDAQRK |
| | (UXS1) | RILVTGGAGFVGSHLVDRLMLLGHEVTVLDNFFTGSRTTVSHWVGHP |
| | 370161 | NFEMVRHDVVEPFMIEVDQIYHLACPASPPHYQYNAVKTIKTSFMGT |
| | EC:4.1.1.35 | LNMLGLAKRTKARFLITSTSEVYGDPEEHPQREDYWGHVNCIGPRAC |
| | | YDEGKRVAETLTYGYMRQDGVDVRVARIFNTFGPRMNPYDGRVVSN |
| | | FIIQALKGEDMTVYGDGQQTRSFQYIHDLVDGLILLMNGEETRPVNIG |
| | | SSHEFTIMEFAEAVRDIVEEVQKAEGVPNPKRVGIIYNDIPTDDPQRRR |
| | | ADTTRAKETLDWQPRWMVRDGVREMVLYYKQMIDQGKL* |
| 12 | UDP- | MSRDNSQLKRVLVTGGLGYIGSHVVLSLLLSGRYLPIVIDNCHNAFPE |
| | glucose 4- | AVNRCAEIARDEMGADAPQPIFHQVDLRNPADIDAVFEKYQADGGIW |
| | epimerase | GVVHLAALKAVGESSEIPLAYYQVNVGGSVSLLQSMAKYGCNSFVFS |
| | (GALE1) | SSATVYGTPEIIPIPETSRVQAESAYGRTKAMVENVIQDVCRSGTMEAP |
| | 367812 | RKEPLKAVSVRYFNPAGAHPSGKLGEEPRGKPGNLLPLLAQMAVGRE |
| | EC:5.1.3.2 | KSELKVFGNDYPGSPDGTCVRDYLHIMDLAQGHVLALDAIAVPADQP |
| | | NIFDKCDPRADGFFRAFNLGRGKGQSVLTMIEAMRKATGFDYKYEIV |
| | | GRRMGDVPDLTADPALAEKELGFLAKRDLPEMCRDLWNFQTKNPNG |
| | | YEPSANAA* |
| 13 | UDP- | MYSNNVERRSDADEKVLMANLGGAQRPPSPSLAAKDTPAAPSSREQI |
| | galactose | LPIVNYCAASIMMTVVNKYVVSGAKFNMNFLLLAIQSSVCVLAVYSV |
| | transporter | KRAGVISFRDFDLKDAKQWYPVSCLLVAVIYTGSKSLQFLSIPVYTIFK |
| | (UGT) | NLTIILIAYGEVFMFSGIVTGLTLVSFALMVGSSVIAAWADISSAWSTT |
| | 334886 | LDQTTGRELTAMPTIGGVSVGYFWMALNCACSAAYVLYMRKRIKVT |
| | | GFKDWDSMFYNNLLSIPVLVVASLVFEDWGSKSLSDNFPAENRTILLS |
| | | AIVFSGAAAVFISYSTAWCVRVCGSTTYSMVGALNKLPVAASGMLFF |
| | | GDPVSFGNVSAIGVGGIAGIVYAIAKTNQARVEAAAKARQSGGP* |
| 14 | Mannose | MANTVTKGVILVGGPSKGTRMRPLTLDCPKPLLPIGGKPMVWHPLNA |
| | pyrophosphorylase | LAQVPGLTDIIMIGFYEDSVMAPFVRESQREFPNIRISYMREYRSLGTA |
| | (MPP2) | GGLYHFRDLILRTPLPQHIFICNIDICSSFPFTELLAKHSAHRGVGTILGV |
| | 370163 | NVPKDTATRYGCIVSDPETKQMLHYVEKPESWISNTVNGGVYLFDKS |
| | EC:2.7.7.13 | VFDEIRVAMDKKTECAAHNPLIELDETLQLEQDVIAPLAGTGKMYVC |
| | | ETRDFWRQIKTAASAVTASGLYLKQFQRSRPELLARAGGAIIEPVYID |
| | | GSAHIDGSAKIGPNATIGPNVVVGEGARIANAIVLDNSVIDKHAVVLN |
| | | SIVGSNSRLGQWARVDGEPEPVSDVKGQISVSVLASDVHVAPETHIRS |
| | | CIVLPNKNLPKNAYNQVLL* |
| 15 | B-glucan | MSLRRHSPQFGGAGAGGSGTSPLLASAQTPAGQYARLSETSLAGNDS |
| | synthase | PSSSMRYGPSAGGGARGYGPQATVSSFSSGTRYGPTADSPSSSSHGHY |
| | (SKN1A) | AAQVEPMSLRAAGAGAGTGPVAALDEADLDDQLHTFTAAEKKDLTA |
| | 306218 | PFTLRSWRGWANALTLLVMLSGFVVLFAGYPIISWYYSDKNALGAN |
| | EC | QPGFNLGGVNATGQYPEIPGFPQMIDPDTPESAKKHISFEGDTWDLVF |
| | 2.4.1.34 | SDEFNKDGRTFFEGDDPFFTAMDIHYWPTNDFEWYDPSVPTTRDGHL |
| | | VLTITQEPINGLMFKSGMIQSWNKLCFNRNAYFEVSASLPGTPRIGGF |
| | | WPGVWTMGNLGRAGYGGTNDGMWPYSYSSCDIGILPNQSADGVTPE |
| | | VAFTTGDPNYNMTLSYLPGQRLSSCNCPGYDHPGPEGVGRGAVEIDM |
| | | IEAQMDLKVQRGMVSQSAQVAPYDDYYQFNNESDAAIIYDSEVTRY |
| | | NTYLGSVYQQAVSGLSYTDTRIYSNETSLHVNESSKFGVFGVEYLAD |
| | | YDDVSKGYITWINMGKRAWTMWAKAVGPNPRTGVGQRLISQEPMY |
| | | MIFNLGMSNNFEYVDFANLMLPNEMRIDYIRVYQKPGEGTIGCDPPD |
| | | RPTASYIAKHADIYNNPNITIFPDADKGRVFPKNRMMDPNC* |
| 16 | B-glucan | MGPEEDDDLHNPTVRQSYKTGFFRSLLTVRGFTNVGCLLIITLILVVLF |
| | synthase | MGYPIITEFTERHQTTNGAFGLGGTNASGQVPLVRQPIDPDTPPEAYQ |
| | (SKN1B) | KVSLETGETWDLIFSDEFNHPGRSFYPGDDPYWEAEDLHYWPTNNLE |
| | 333973 | WYDPKRLTTRDGSLVITLTNEQYNRRNYTGGMMNTWNKFCFTGGYI |
| | EC | EAAVSLPGKSNVQGLWPAVWTMGNLGRAGYGGTTDGMWPYSYDS |
| | 2.4.1.34 | CDVGTLKNQSLNGVPEVAQTQGDPSYNMTLSYLSGQRLSACTCPGD |
| | | KSHPGPIRPDGTYVGRAAPEIDMIEAAVSDSKVGEVSLSGQWAPFNP |
| | | WYGWINQTDLVTVYNVSNSHFNQYRGSIWQQATSLLAKTDQACYTH |
| | | EQGCFQVFGVEWQPSKDNDNDGYISWLSDGKKAWTLREGAMGPNS |
| | | DAGIGQRIVSREPMYMIVNLGISPNFGYIDFEGLDELWPVEMHVDYIR |
| | | VYQDPKKRNIGCDPPDYPTADFIDKHRVAFTNPNITTFEQLQDAMPGL |
| | | EWPKNRFLGEC* |
| 17 | CAS1 (o- | MPPAQKQAQAARLNPVWYHYACATVAAALVLGNLLRWAYLDAPD |
| | acetyltransferase) | NYHCGAMLSTGKWLDEGTYKNWQPEGCYQAAIKAPALAKCLTSPSA |
| | 177902 | VTPVTDGLSNRRVLFVGDSTVRQLYFAAVRQVDKGTDYPKSWESDG |
| | EC | EKHSDRKAVITTKGGQQLTIEFWWDPYLNTTQTHDYLASRPETPAAM |
| | 2.3.1.84 | LVMGSGLWYLRNPSSGGATAFTKTIEDTFETLRAKQGSPLAPLLNPW |
| | | DAMTTTGSNWLPGLMPDSTGAGVSAGGHDFALADAVFFVPVANPVV |
| | | EKLSAERAETIVPADVEMMNAEVATRLAQSNPPPIIVPSVFNELLVDE |
| | | ETSDGLHFSDRIMDKQAELLYSWRCNDVLRKEVSEGACCRRYTTLRP |
| | | LQALLLLYLGVWAPISCLIAPRLRPGHPLLAVIPADKVAKAISSFGLAI |
| | | AYLYLADRTTIFLKEQKDWDPKIFSALMIAALIAGLATVKNKGKDLG |
| | | FLNRDITDEWKGWMQIAILIYHFFGASKISGIYNPIRVMVAAYLFMTG |
| | | YGHFFFYFKKADYGFNRIAQVLVRLNLLSVVLPYTMNTDYAFYYFAP |
| | | LVSWWYMIIYGTMFLGSKYNDKPAFLLTKLVVCAGAITAFMHQTWM |
| | | MEGLFSFLNTVFRIQWSAKEWSFRVTLDLYIVWGGMLAAYAFIKFNE |
| | | LRIAERPFFPALRTGAIVASVLGLIWYMWFQLSRKDKFEYNGYHAAV |
| | | SCIPILSFVVLRNASALLRTCTSQLFCFVGQISLETFILQFHGWLAADTK |
| | | AVLLVLPATRWRPVNLVVSSIAFGWLSYKVSGATGDITSWAVGKKKP |
| | | ALPPPVTQGEPIFDAGQENIPLMEPAKDENGNAVNGSANASAAYAAP |
| | | EKSWRDSTVMSVLGNIGVLAQTHDSVKLGLVLLVLWVANWLY* |
| 18 | Chitin | MLYLTADKFPVFVPFGVIGFYRYLWYCIRLIARYVYRPIPLPETPTYRA |
| | synthase | DEDVTIIVPTIDAGEEFREAALSWLVGNPKEIIIVTEEKMLAPLQELANS |
| | (CHS2) | VDPTRIRVLTVPFANKRLQMAHGIRNTTTDIIVFADDDAIWPPQLLPY |
| | 215086 | VLACFEDQQVGGVGTSQRVKSVGKKMTVWEVLAGFRLTIRNIEIASA |
| | | THIDGGIPCLSGRTAAYRTVILKDPDFLHGFTNDLWLNKYHLNSGDD |
| | | KFLTRWMVSHGWNTYVQVCKEAELLSTMKPNWRFLKQVLRWTRNT |
| | | WRSDFRSVFTERYVWTKHPYVCYTMIDKFINPLTLLVGPALVCYLLY |
| | | KSTKPIDQGGYHLPAWDIVVSYLVWLLCTRTLKVLPHLWDRPKDIIY |
| | | VPVFIIFGYYFAIMKIYALFTLHKTGWGTRSGIGDPATATTAAENVNE |
| | | KVPRPVELGGPAPSHSRPYDVEMAQNVNNPYRA* |
| 19 | Chitin | MSSQYSHDDINGNGGSARSSQYRLPGPPPPLMDQSHLRPGKQAELLS |
| | synthase | HDRTLELYRENAKKTNDPELIFEFAVFMIDAAKSMVPPPNPDDPNDPA |
| | (CHS4) | NRPNAAALEKRDELIKEATSLLKRLADRGYPDAQYFLADCYANGIGT |
| | 348500 | TRGKQDFDKAFPLFALAAKHGHPDACYRAGTCCEHGWGTRRESAKA |
| | | IQFYRKAAVGLHPGAMYRLGTAELNGSLGLSKRPKEGVKWLKRSAE |
| | | HATEEFPHALHELALLHERGIENIVFVDLDYSAELLAQAAELGYAPSA |
| | | FKLGECYEYGKMGCPVDAALSIHYYNIAAQQGHRDACFALTAWYLV |
| | | GAEGVLPQSDTEAYLWAKKAADKGLAKAQYALGYFTETGVGVPPNI |
| | | QQAMKFYRQAADGGDKRAAKRLAGGVSGGGAALNRQREMDAMKE |
| | | AGGMNGKDKDKDCIIM* |
| 20 | Chitin | MDIGDVYRAPPQGQVPRRRDPYDAGHPAPPPPVPPNLRMDRNNVTPS |
| | synthase | PGPYPNYPPSSSAHENPFQHPSDHGNDWGQAPPVPPPPQPYAQLGRTD |
| | (CHS1) | SSQSYQTPYMADNRLTSPPPLFPAPSASPGFSQSGPYGHPLDDDTNDA |
| | 390231 | QPLLHHAQPHPAFNIQPMPPRPQAPYDPPIQGSAGGRVHFDDPYQTQG |
| | EC:2.4.1.16 | VNDEQERNNVHYGAPPARVLRRNRTQKRVQLFRGHLVLDVDVPSML |
| | | MDQSAIKEGNEFTKMRYTAVTCNPDEFVLDKYTLRQRLYEQPRKTEL |
| | | FIVVTMYNEDDVLFCRTMRGVMQNVAHLCSRSRSKTWGKDGWKKV |
| | | VVCIVSDGRMKINKRTQSVLAALGVYQDGVAKNVVNNKPVTAHLYE |
| | | YTTQLWIDSKGRVGPGGDNAVPVQLIFCLKEKNQKKINSHRWFFNAF |
| | | GDCLRPNVCVLLDVGTQPGPDSIYHLWKAFEVNQSIGGACGEIVALK |
| | | GTMGKNLLNPLVAAQNFEYKMSNILDKPLESAFGYITVLPGAFSAYR |
| | | YIALQNDDNGEGPLEKYFYGEKMHDANAGIFKANMYLAEDRILCWE |
| | | LVSKRSSRWTLHYVKSAYAITDVPDTVPELVSQRRRWLNGSFFAAVH |
| | | SIAHFHYLYRSSHSVMRMFWLHVELIYQIISNVFAWFSLGNFYIAFIVL |
| | | SKSMEYAVDLAPNEPAFDEFGNRKTNKVFEYINIPLQYAYLALLLLCF |
| | | LLSLGNRPAGSKWGYTISMIGFALITVYMIFAAVWLTVAGVNQIKED |
| | | GATVGELVQNPTFRNIVLSLLATYGLYIVSSLMALDPWHLVTSFVQYL |
| | | LLAPSYINVLNVYAFSNVHDVSWGTKGSDKVSEDLGVVKSSGGDGE |
| | | VTVDMFSEQKDLNAIYAAELQVLATKPPKEEAKVNTEQQHEDYYKN |
| | | FRTNVLLSWTMTNAALAIVILRVGVDGQFVIAQTYMAVMLYTVAGL |
| | | AFFRFLGAIVYLVVRLFMGE* |
| 21 | Glycosylphosphatidylinositol | MACGLPRALQVALAVRALSLALPHTFFQPDEFYQAFEPAYAVVFGQG |
| | mannosyltransferase | HLTWEWRDLPLPEGVAHTWWTRVVTGGRLRGWIWPGVFVLAYKLV |
| | (PIGB) | QVLALDSTDLIVFVPRVVGVILAAVTDYYTSRLAGKVLGEGYAAGAL |
| | 385852 | FLSLTSLFNAHLLPRALSTSPETLLTTIGLYYYPLPAFRPASLGATCVPS |
| | | QDGTAEHHEAQRTCGAMDRAEGGKTRPSSDNMLFAAAVAALSLCIR |
| | | PTMLVFWAYLHLETSIRHWVTHGATATFCLTADTLLGGLTVLGLSTL |
| | | LDWHMMGRLTIPTLSFFHQNVVRNVALFYGGTGLLYHLVQSLPLLL |
| | | MPIWFWWGEGFLAAILPQSMQPPALKKLDIPLALSTLGRTIVVTIATLS |
| | | LSPHSEWRFLHPLLPPLMLFALPPLQASYKPTPMGYHSFLTSVRQYCR |
| | | LPSRPCYLILLCPIIPYLYLNSFHGAAQVAVINGLRHGDFGAASFVVAL |
| | | MPCHSTPWSSHLGNIPGWFLTCEPPLVPQSADVHWTQQDLFYASPVT |
| | | YIQHVFPSPPAKLSDVPTMAGEERMPSHVLVFGEVLARQDTLGNSSLS |
| | | VEQALAERGYHEKARLWNGFDFAQDEEKRRGGVRVWTHGESYDLV |
| | | GEPAA* |
| 22 | Glycosylphosphatidylinositol | MAETDPPLDAAPLLAAVNPPRSRRWAIMLCFPLFLALASPWWWTTTS |
| | mannosyltransferase | IVRLPLPVERIGALESQSDPVITTLVSFTGEDAAFPKAPPGRADFPTATK |
| | (PIGS) | LDALAAEVVKGVDGMLARDRPNVRGTRRWDVVVNGPKSVRHRVHI |
| | 413175 | GVSETAKPDWPLEPYVQTSYGTADVPPGTVVIPIHPSMVSDLNLKRH |
| | | YKIALVNALHNLLPAHLPPVANRALAYSPNITLSFVVVNEDAAEGAF |
| | | VNDWDIDGALRDHIFPHLEPLSPVFNFSIESQVLYHAPLAFEPAQQDD |
| | | GWVVDEDGMKMFVSERWTPDSLSTNNPVLKFLLFVPSAQHAPLRLA |
| | | ANATSFLIPQFGSVVLLNPPAEGGAYRLSLDALDAPFKQFTEHLYGLL |
| | | SLPALPEKLHHCPEPASEGKTRAQRGLRDPLSLWQLDTIFRVRMAEN |
| | | AAEARKTLGGIVRLVDKIKEMQLGPGVRDKVLGAVRRLEELPETAQD |
| | | MSPLTAFVLSRDAVGLANEAFFDPSMMGLLYFPDQHKFAVYTPLFAP |
| | | IGVSIVVGLLREIANLIKKRKAARKAAQAKKDDERLDNGTQVVSEAM |
| | | TTAREAPAAARLRATTTS* |
| 23 | UDP- | MHVLLTGALGRCGMRTLSQLLDEGHTVLATDRTSPSSPLGDGAEFMA |
| | glucose 4- | ADLTLLHDIDALFKHRFDGVIHLGAIPNPVPEIDDRELHANNVVSSYN |
| | epimerase | VMRTAVDHGVRRIVQASSVNAHGLSFAPPGHTAFDRFPITEAVERRD |
| | 365740 | EDAYATSKHECEVQASALVRYAPGTRIASLRPHMVRDDYASAYPDTE |
| | (GALE2) | ARDIFSWVSYAAVARAAVLALTSEGWGGHEVFNIVAPEICWEGSVEE |
| | | QYIDKDGEPPAHKVGTLELVRKYWPNVEVDEEYWRDRPRRAVWDSS |
| | | KAERLLGWRHE* |
| 24 | PIGH | MRSLTSPLPSHPQYSVLVVTTTAGARDAAGVVTEHRVAPRRNNLGPR |
| | 394303 | TYLLPAAIVLLCWWRFPHPLVLASAVAVALALRWRVLYQSVTPLPGL |
| | | GVQLATVRGFGVGPKSTARQFVPLDDISTVVIHEGLRRWSVRYYLGV |
| | | VRKRGNVVVAFDAVHPHPEVLREVYHGVREALYEEYAGDASETGE* |
| 25 | Mannose | MKALILVGGFGTRLRPLTLSWPKPLVEFCNKAMILHQIEALVKAGVK |
| | pyrophosphorylase | DIVLAVNYRPEVMVSVLKKTEEEYGITINFSVETEPLGTAGPLALARDI |
| | (MPP1) | LGKDDSPFFVLNSDVTCTYPFEAFRDFHLKHGCEGSIMVTKVSEPSAY |
| | 367061 | GVVVTKPGSTIIDRFVEKPVEFVGNRINAGIYMFNPSVLDRIELRPTSIE |
| | EC:2.7.7.13 | KEIFPAIAADQQLHAYDLQGFWMDVGQPKDFISGTCLYLSHLTSQGSP |
| | | DLADPAQNKWVHGGNVLVDPTAEIDPTALVGPNVVIGPGAKIGKGA |
| | | RLQRCVIMSNAEVRDHAWIASSIIGWNSTVGRWCRVENITVLGDDVSI |
| | | KDELYVNGASVLPHKSISTSITEPRIVM* |
| 26 | B-glucan | MSASGDHSALLPPGTGTRSPNLDYSQRSSRRYGPAASTDPVPSTASLL |
| | synthesis | GGSAGSRLSSGSRLSAPRSTSTHGTRAGATSSASDGWGWNDPEADDY |
| | (SKN1C) | LHNPDPKRDRKNDRGGSIFTLRGIVNIGCLVLLLLCLITLFAGYPIITTY |
| | 370117 | TERRQSNNGAFNLGGTNATGQVPLIAGFPSLIDPDTPMDVRSRVGYD |
| | EC | GKAYELVFSDEFNKDGRTFWPGDDPFWTAVDIHYWPTVDLEWYHPS |
| | 2.4.1.34 | AITTENGHLKITMTQEPINDLQFKSGMLQSWNQMCFQWSYYFEISVSL |
| | | PGNNRIGGFWPGVWTLGNLGRAGYGGTTDGLWPYTYTACDLGTLA |
| | | NQTWPAGSGEVTPPGAITTSDGDPLSYLPGQRLSSCTCKGEDHPGPNP |
| | | SVGRGAPEVDVIEGQIDTRMRMGEMSQSAQVAPFDADYQFDNSSDA |
| | | VTIWATDITKWNTYLGGVYQQAVSGLVYVDNGVYTGTSGLFNIYGV |
| | | EVFADKNDESKGYVTWVSGNEKSWTMEAKATGPNPLSGVSQRPITQ |
| | | EPMYLIMNFGMSNGFQQVDLQNLQWPAHMLIDYVRVYQIPDYGYLG |
| | | CDPEDHPTAEYINSHPEPYNNANITLWSQAGYSWPKNRLVNTC* |
| 27 | predicted | MRPLTSSTLWKRAPSAGSLLIGVLVGLLVASLLRRGEPAPPPWQGSGG |
| | alpha-1,3- | GLYPNAMTGNRVGKITSLPNMDQRLRILKLLGTISAKHTRECTRNPQK |
| | mannosyltransferase | LYVDQAKERYEPLIGHKTGWRSSGGLSGLFGGGASSRDREQLHLADT |
| | (MNT1) | RFSRRDVLTRNLMSLQGPDNHKYFFAINLYESFDVIPDLFSTLFKTASI |
| | 387385 | LGYHNVMVSIYENGSKDQTKALLRLFDALCRSVGMRVIIRSSARTRG |
| | EC 2.4.1 | QFNHRIEYLAEVRNSAMVPLHELRDQGDYYDSIVFMNDILPCVDDLL |
| | | ELIWQSRRQNAGITCAADYMYHDDIGSPVFYDNWVARDLNGTALEN |
| | | APFEQVFHHAESSQRFQRHLPLQVQSCWNGVAVLDPAPFYETFPVKF |
| | | RMAKLTENECSASECSLICNDYWAMGYGRIIMVPRVKLAYDKKVYDI |
| | | IHPERRNLTAIRGYKRLGGMPDDPRSDPQDRSWYGPHDRLFTPEEDLE |
| | | LDFKPGPKSVWCWGWDGAGDLDGPDVDPIWEDQAPQSSQPDAVKIK |
| | | HHRNLRGY |
| 28 | alpha-1,3- | MSAGSRTWSAGPLRQATHLLVALLASALCVTAIRYNETLDPWNINKA |
| | glucan | QSAEDVLSYSTPLRSDPYFPSPTNWRTLPTYTLLLDKWIDGDPDNNDF |
| | synthase | FRLIYEYDWRETQLRAGGDVIGLMDDRGLDYLQSMGYEAIYIAGTNY |
| | (AGS1) | LNMPWQADGYSAIDFTLLDPHFGNLTDWKLFIDRLHARGMYIIFDLT |
| | 298029 | VGTMGDMIGFEGHLNSSTPFSESEYNAVWKRPNYGPWGINYYPDFNF |
| | EC | TNEYNESCVYPTFFDSAGNIIDPGLTGCMNSPFDQYGDVEAFGTHPD |
| | 2.4.1.183 | WKRQLAKFASVQDRLREWNPHVLDRLKHFSCLCVQALDIDGIRIDKA |
| | | TQVTVDALAEWGRYTRQCAKELGKDNFFIPGEVTGGDTFGSIYYGRG |
| | | RTPGMRTTYMEALNLTNADTSKFLREEGHAPLDSAAFHFSVYRALAR |
| | | YLGLDGYLTVVPDVDVNFVNSWNQMLVNNDFINPQTGEFDPRHQYG |
| | | TTNQDVFRWPMMSNGTQRQLAGMFVTHLLMPGIPISWYGEEQEMYI |
| | | LDSSANNYLYGRQAMVASQAWKRHGCYRLGAQQYYNVSYDKVTT |
| | | GCYDPQVALDHFDPTAPLRRSSRNMNWMRRTFPAVQDAFMLSALAN |
| | | FTRYDKYANSDQRTEIGLWAAERTPSEKQRADGGPGGPWASDGQISQ |
| | | VAYYIYTNENVSVSYDFDCSNPLRSMNTSFPGGTTIKNLIYPYDEILLS |
| | | SSSVSFYNDGQPPYRGCIPRLALQPYDFRVYVPIERWRSPPPALSGFSP |
| | | GHDARVEYTGDSVEIELQYDQPMDCAGLQQAIRLTYVGQPGATPRIT |
| | | WPSDACLSITGEASDYVSVTPSAWRMKGVITGAGEGMYRISIGNASA |
| | | QAAGSGNGTLGGGVTGYTDHLHFRYGKADNTFVFVDSDYDNSIFSAS |
| | | EGVYTLTHKAWGADRYRYSANYGKTWTAWTPMEASTTLNASLFAQ |
| | | ETMFKGADEEKLWDGDHLMVQYWSQLAGSAGHVVHADANWNGG |
| | | QRRYPQMLLRGGFNNWGYDLGMPNKFQHTGDSVWEMPIMSAWPAE |
| | | YQLNIWAFDDYFYGDADGNGVIDRLPPSSLTPNFLNVSLPPRPHLAW |
| | | QLQINDKTGRWGVVPIGHEASSIIAFVLLMIIPVMTALVAAGVFRYSF |
| | | YAIKINKWGVKPTKEGSNYFPIVGGAHNGEKKDGSHLGVSEKKVTHH |
| | | EKIKPLERVIGWPEDSNKRRKVLIATLEYEIMDWKLKVKIGGLGVMSS |
| | | LMGKAMNDVDLIWVVPKVQDIDYPQGDFAEPIEVIIFGEPYLIEVETH |
| | | CVDNITYVILDSPVFRAQTKSDPYPQRMDDLSSAIFYSTWNQAIAETL |
| | | RRFEDVDIYHINDYHGALAPLYLLPKIMPVCLSLHNAEFQGLWPLRTK |
| | | DEMKEVCAAFNISKDVCSKYVQFGNTFNLLHAAASFISQHQKSVGVA |
| | | GVSDKYGKRSWARYPALWTLRNIDSLPNPDPTDIAALDEQPVAMDKI |
| | | QVDQDAEAKRPEHKRQAQEWAHLKQDPKADLFVFVGRWSKQKGV |
| | | DLIADVMPSLLEKRQNIQIICVGPVIDLYGRFAAEKLARLMEMYPDRV |
| | | FSKPEFTQLPPFLFSGADFALIPSRDEPFGLVAVEFGRKGALGVGSRLG |
| | | GLGLMPGWWFPVESPAAEHMLSQLTKTIKLALKSTQEERAILRARSA |
| | | VQRFPVVEWRQRLEDFQKRSITMSRSLAGEHAWGWEYADPSGNGPV |
| | | RSSMLPSRDSMVSLQQYTRPGSPDSQLSVPSMDPRRMSDNGEPLPAG |
| | | AAPAGDRASLGVDYHRRFEKHQSADSIYDEDPPEHRGRPKFFTGYDD |
| | | DSESHISSERDTTIVAPHAMADGGTGHVYDNFLAAANRQFAKTQGNK |
| | | NVPDPFMERRMSSLSIDSSNFAPPRPFSIHSRVSSFDSIGSLADEKHSST |
| | | LNKAMDTFTDADGEVAQAFVQKLRNLSSGNSKGDLCIEKFLMKSEK |
| | | QFFSDMKKEKLSAMSIRSRDSIMAGRTPSTLDGFRAESPMQYSMSGHS |
| | | HPEDHFHESMMFDNGDDEKPMTRIQVLMERQIAGWPLYAIVISLGQL |
| | | LSATSFQLSLLGGANTQTATDMYIICAIFVVATLLWYTLFRMRPSVYV |
| | | LSIPWAIFGVAFLLIGFPSLYGPFTAPRKWIVRVATWCYAVASGAGFL |
| | | FFGLNFGEEAGAAAEVWIMRACIVQGLQQIWVSALWYWGFTLNGTN |
| | | PLTFVVPRAIIYVVWPLAVMSMAFAVLMYFGLPEYYHQIPPYVPNFFK |
| | | TLVRRKLVIWFMISEVLRNYWLSTVYGRNWQFLWRDARVPTWSVV |
| | | VMIIIFFIGIWGLIMGIIMKYAKIHAWLLPVFAVGLGAPRWCQMFWGIS |
| | | GLGLYVSWGGPAGPYIGTCLWLWLGVLDAVQGVGLGMILLQTLSRL |
| | | HVCATLAAAQFVGSIVVMIARATAPNKEGPGNVFPNAAFYDPATGAN |
| | | NPVKHWEFWLCLCCQLVIPFGYFFFFRKEQLTKP |

Table 2 provides proteins of interest including: proteins representing specific enzyme classes whose activity increase are designed to improve oil yield, specific productivity, titer, composition, fermentation viscosity, and/or susceptibility to lysis.

TABLE 2
Proteins of Interest

| SEQ ID | | |
|---|---|---|
| NO. | Sequence Info | Amino acid sequence |
| 29 | GAPN_Sm | MTKQYKNYVNGEWKLSENEIKIYEPASGAELGSVPAMSTE |
| | [EC: 1.2.1.9] | EVDYVYASAKKAQPAWRSLSYIERAAYLHKVADILMRDKE |
| | | KIGAVLSKEVAKGYKSAVSEVVRTAEIINYAAEEGLRMEG |
| | | EVLEGGSFEAASKKKIAVVRREPVGLVLAISPFNYPVNLA |
| | | GSKIAPALIAGNVIAFKPPTQGSISGLLLAEAFAEAGLPA |
| | | GVFNTITGRGSEIGDYIVEHQAVNFINFTGSTGIGERIGK |
| | | MAGMRPIMLELGGKDSAIVLEDADLELTAKNIIAGAFGYS |
| | | GQRCTAVKRVLVMESVADELVEKIREKVLALTIGNPEDDA |
| | | DITPLIDTKSADYVEGLINDANDKGAAALTEIKREGNLIC |
| | | PILFDKVTTDMRLAWEEPFGPVLPIIRVTSVEEAIEISNK |
| | | SEYGLQASIFTNDFPRAFGIAEQLEVGTVHINNKTQRGTD |
| | | NFPFLGAKKSGAGIQGVKYSIEAMTTVKSVVFDIK |
| 30 | GAPN_Bc | MTTSNTYKFYLNGEWRESSSGETIEIPSPYLHEVIGQVQA |
| | [EC: 1.2.1.9] | ITRGEVDEAIASAKEAQKSWAEASLQDRAKYLYKWADELV |
| | | NMQDEIADIIMKEVGKGYKDAKKEVVRTADFIRYTIEEAL |
| | | HMHGESMMGDSFPGGTKSKLAIIQRAPLGVVLAIAPFNYP |
| | | VNLSAAKLAPALIMGNAVIFKPATQGAISGIKMVEALHKA |
| | | GLPKGLVNVATGRGSVIGDYLVEHEGINMVSFTGGTNTGK |
| | | HLAKKASMIPLVLELGGKDPGIVREDADLQDAANHIVSGA |
| | | FSYSGQRCTAIKRVLVHENVADELVSLVKEQVAKLSVGSP |
| | | EQDSTIVPLIDDKSADFVQGLVDDAVEKGATIVIGNKRER |
| | | NLIYPTLIDHVTEEMKVAWEEPFGPILPIIRVSSDEQAIE |
| | | IANKSEFGLQASVFTKDINKAFAIANKIETGSVQINGRTE |
| | | RGPDHFPFIGVKGSGMGAQGIRKSLESMTREKVTVLNLV |
| 31 | GAPN_Ca | MFENISSNGVYKNLFDGKWVESKTNKTIETHSPYDGSLIG |
| | [EC: 1.2.1.9] | KVQALSKEEVDEIFKSSRTAQKKWGETPINERARIMRKAA |
| | | DILDDNAEYIAKILSNEIAKDLKSSLSEVKRTADFIRFTA |
| | | NEGTHMEGEAINSDNFPGSKKDKLSLVERVPLGIVLAISP |
| | | FNYPVNLSGSKVAPALIAGNSVVLKPSTTGAISALHLAEI |
| | | FNAAGLPAGVLNTVTGKGSEIGDYLITHEEVNFINFTGSS |
| | | AVGKHISKIAGMIPMVLELGGKDAAIVLEDANLETTAKSI |
| | | VSGAYGYSGQRCTAVKRVLVMDKVADELVELVTKKVKELK |
| | | VGNPFDDVTITPLIDNKAADYVQTLIDDAIEKGATLIVGN |
| | | KRKENLMYPTLFDNVTADMRIAWEEPFGPVLPIIRVKSMD |
| | | EAIELANRSEYGLQSAVFTENMHDAFYIANKLDVGTVQVN |
| | | NKPERGPDHFPFLGTKSSGMGTQGIRYSIEAMTRHKSIVL |
| | | NL |
| 32 | DGAT1_Co | MSTPLVPVVPLSPAEDGAKSPVDHAPAPTPSPESSSPRSP |
| | [EC: 2.3.1.20 | LVALKHDKPLSRSSSLLELLHLKDVHIPTPPPVKFAPLVV |
| | 2.3.1.75 | PRHRRLQTAVVATWSVMIPICLTVFYGSLFFSKWLRPLAV |
| | 2.3.1.76] | IYAVWFIVVDRAWRHKGGRRKDWVRRSAFWRYFADYYPIT |
| | | TVKEADLPADRKYVFAYHPHGIISMGAACTFATEATGFSS |
| | | LFPGVTCHLLTLDANFWIPLYRDILMGMGLASVSKRSCRS |
| | | ILKMGRSICIVIGGASESLYAYPGTNNLTLKKRLGFIKIA |
| | | IREGANLVPVYGFGENDIYELLPNEKGTMTYKFQKWFQGT |
| | | FGFTVPFFHGRGVFTYNYGLMPHRRPVTVVVGAPIPVKQI |
| | | EKPTDEEVQAVHNQYIEALQALWDKHKDEYAKDRKSELKL |
| | | VA |
| 33 | DGAT2_Co | MSTSASSASSSSSSPGTSPVTTHSALDSEPGTPSGLGLNV |
| | [EC: 2.3.1.0 | KLDTFGGRIRSVDGPSPAPSLITLHSSLPASPDPTTPSTE |
| | 20 2.3.1.75 | EGELEFSPDPAEDAPQMRQRKVPSPNMAMTTLANGAATQT |
| | 2.3.1.76] | LKKPQKKLSRHKSPLLDALKLPPLSEISIPRPVQIKFAPL |
| | | HIPPHRRLQTFAILTWAILMPLCLMLYFYFLSIPGLWPVL |
| | | VPYTIWALAIDKAPFKGGRPKRWARRFPLWKYFCQYYPCS |
| | | MIKEADLPADRTYVFGYHPHGIISMGAVATFATEYTGFSE |
| | | NYPGIKSHLLTLDSNFGIPFYRELLMFQGICSVSKRSCRN |
| | | ILRKGPGNAITIVVGGAAESLAAHPGTQDLTLKKRFGFIK |
| | | VAIREGAQLVPVFSFGENDIYEQLSNEKGTTVWKLQKGFQ |
| | | KIFGFTLPLFHGRGIFNYSLGLMPYRHPIVSVIGRPIDVV |
| | | KSHNPSDEYVQQVHAQYIEELMRIWDKYKDIYAKTRTREL |
| | | RVVD |
| 34 | DGAT_Ce | MGNKETMETPKTNTRPPLLKSIVALFLWMGIIQLNVVLLV |
| | [EC: 2.3.1.20 | TALFVPLRIGTMIIGFLVLMRLLPVNPKSKFGSKVAKFID |
| | 2.3.1.75 | KNAYGYFPITVHLEDEKAFDPNQAYVFGYEPHTIYALGAW |
| | 2.3.1.76] | ALTDRSSLAPVPKIKFTAASIAFNIPILRHIWTWLGLVPV |
| | | TRKSFIKQLSAGNSCIVVPGGVQEMLHLEQDSEVAFLKSR |
| | | KGFVKLAIEMGSPLVPVFSFGQSQAYKWWRPQGKIFHKIY |
| | | KIVKQPIVLSWGRFGSPIPFRVPMHIVIGKPIQLKKNAQP |
| | | TNDEVNEVHAQFVAAMQELFEKHKAQYGSKDLQLRVL |
| 35 | PTA_Ec | MSRIIMLIPTGTSVGLTSVSLGVIRAMERKGVRLSVFKPI |
| | [EC: 2.3.1.8] | AQPRTGGDAPDQTTTIVRANSSTTTAAEPLKMSYVEGLLS |
| | | SNQKDVLMEEIVANYHANTKDAEVVLVEGLVPTRKHQFAQ |
| | | SLNYEIAKTLNAEIVFVMSQGTDTPEQLKERIELTRNSFG |
| | | GAKNTNITGVIVNKLNAPVDEQGRTRPDLSEIFDDSSKAK |
| | | VNNVDPAKLQESSPLPVLGAVPWSFDLIATRAIDMARHLN |
| | | ATIINEGDINTRRVKSVTFCARSIPHMLEHFRAGSLLVTS |
| | | ADRPDVLVAACLAAMNGVEIGALLLTGGYEMDARISKLCE |
| | | RAFATGLPVFMVNTNTWQTSLSLQSFNLEVPVDDHERIEK |
| | | VQEYVANYINADWIESLTATSERSRRLSPPAFRYQLTELA |
| | | RKAGKRIVLPEGDEPRTVKAAAICAERGIATCVLLGNPAE |
| | | INRVAASQGVELGAGIEIVDPEVVRESYVGRLVELRKNKG |
| | | MTETVAREQLEDNVVLGTLMLEQDEVDGLVSGAVHTTANT |
| | | IRPPLQLIKTAPGSSLVSSVFFMLLPEQVYVYGDCAINPD |
| | | PTAEQLAEIAIQSADSAAAFGIEPRVAMLSYSTGTSGAGS |
| | | DVEKVREATRLAQEKRPDLMIDGPLQYDAAVMADVAKSKA |
| | | PNSPVAGRATVFIFPDLNTGNTTYKAVQRSADLISIGPML |
| | | QGMRKPVNDLSRGALVDDIVYTIALTAIQSAQQQ |
| 36 | ME1_Co | MSNSTARKMTATSHQLSAHVRPNGPTPPLTPKSNSLQLPL |
| | [EC: 1.1.1.40] | GGGLGRSRPNINREQSLLRLQHQLDQQPTSLMKHSLLSRT |
| | | RREDPELFFAALQADLVNLAPIVYTPTVGEACQKYSQVYS |
| | | GPEGLYLSIDDKDQLPQILAEYAATLKHQPQIIVVTDGSR |
| | | ILGLGDLGLGGMGISVGKLNLYVAGGGLDPSGTLPVVLDM |
| | | GTNNESIREDPLYLGLKRPRASFDEAVSFMDAFMAAASSQ |
| | | FPKTVIQHEDFYSEAAFEFLERYQNKYRMFNDDIQGTGSV |
| | | ILGGFVAAAKAACGVSGRDPKDHKVVFLGGGSAAIGVAKE |
| | | MMNYFRMLGLSEQEAKERFWLIDTKGLITETRQDVVDGRL |
| | | ADHKKYFIRRDTQGQEYKSLLEVIEYVKPTTLVGLSTTFG |
| | | AFPEPVVRRMAELNQAPIIFPLSNPTSKCELSFEDALNWT |
| | | DGRVLYASGSPYDPVVYDGQLREPGQGNNFLIFPGLGSGA |
| | | LLSGATHISDDMVTASAIALSEALTPAEKEASLLYPRLTR |
| | | IRDVSAVVATGVIRAAQKAGLDTNVQLRDLDDASLLEHVK |
| | | LAQWSPYHPQEAAAPIKGASL |
| 37 | ME2_Co | MVTYDFKEKTPMPIRTNLRGSALLNTPSLNKGAGFTREER |
| | [EC: 1.1.1.40] | SIFGLEGFLPYDVHTLEKQCDRAYNQLKRQPTPLLQHVFL |
| | | ASLRDQNTVLFYRLLQDHLKELLGIIYTPTAGEAVAQYSN |
| | | LFRRPVGCYLSFPNRDGMRAQLEAHLQNINIVADVARHDE |
| | | TTAESIDLIVVTDGEAILGIGDYGVGGITISTSKSALYTL |
| | | GAGINPNRILPVVLDVGTNNPILFASDLYMGWKRTRMRGK |
| | | NYDDFVETFINHARDLFPNAVIHFEDFGLNNAYRLLEKYR |
| | | NKFAMFNDDIQGTGAVTLAAIIAALKVSGGGALQDQRIVI |
| | | YGAGSAGMGVADAIREGMEVQDKLGTADASQRFWAVDRNG |
| | | LLLESMAPALRGNQVRYARPDDEIKTWALENPELPSMAQL |
| | | LDVVRNVKPTILIGTSTATGAFTEEVVREMAKHVERPIIM |
| | | PLSNPTSLCEVDPQDAITWINGKALVATGSPFSPVKLPDG |
| | | HEYHVAQTNNAIVYPSLAAGAILARANTISPRMLMAGVDG |
| | | LSELSPALDDPSQALLPSLDNIRAVSVHVTAAVIRAAVKE |
| | | ENAKNELVIKIAEDKGEMSLEDYIKARMWDPVYRPLELVD |
| 38 | ME_Rt | MPSTFAPSQPLQGGPSPSQLGPKELLIERALTRLRSIPSD |
| | [EC: 1.1.1.40] | LEKYTFLAGLRCRNPDVFYGLVGGNMKECCPIIYTPVIGL |
| | | ACQNWSLIHPPPPESDPTIEALYLSYSDLPNLPSLIKGLK |
| | | TRLPHNQMQISVVTDGSRVLGLGDLGVGGMGISQGKLSLY |
| | | VAAGGVNPKATLPIAIDFGTDNEKLLADPLYVGQRMRRLS |
| | | EEKCLEFMDVFMRCMHETFPNMVIQHEDWQTPLAFPLLHK |
| | | NRDLYPCFNDDIQGTGAVVLAGAIRAFHLNGVALKDQKIL |
| | | FFGAGSSGVGVAETICKYFELQGMSEQEAKSKFWLVDSKG |
| | | LVAHNRGDTLPSHKKYLARSEPDAPKLRSLKEVVEHVQPT |
| | | ALLGLSTVGGTFTKEILESMATYNKRPIVFALSNPVAQAE |
| | | CTFEEAIEGTDGRVLYASGSPFDPVEYKEKRYEPGQGNNM |
| | | YIFPGLGIGAILARVSKIPEELVHASAQGLADSLTPEETA |
| | | RHLLYPDIERIREVSIKIAVTVIQAAQKLGVDRNEELRGK |
| | | SSAEIEAYVRKGMYHPLLEAEQQAQ* |
| 39 | XPK_Co | KDRWRVQGYIEEGTTTSPWSMLRVNHCSRYDLADIAVELV |
| | [EC:4.1.2.9 | CKLTPNMAPGKYDATTPPKASQLDPKLIGKLLVDLRSEID |
| | 4.1.2.22] | TLKSSYDVQAVMDFQRAANFLSAAQIFLRSNALMKRPLEQ |
| | | RDVKRRLLGHWGTCPGLNFAYAHATQLISELEEKGEDPDW |
| | | LFITGPGHGAPAVLSTLYLEGSFTKFYPEYDFSEDGFEKF |
| | | VRAFSFPGGFPSHVNAETPGAIHEGGELGYALAVAYGAVM |
| | | DRPDAISVVVVGDGEAETGPTAAAWHAHKYLDPATNGAVI |
| | | PILHINGYKIGERTIAGCMDDLELAALFVGYGFLPRIVEY |
| | | GKVGDREPDGSTSHEHDVSVNYDMAASMRWALGEIRKIQK |
| | | AARGGKPITKPRWPVILMRTPKGWTGPREVGGNPVEGSWR |
| | | SHQVPVNACATDKGHFEQLVHWLKSYRPEELFHTGGSGHL |
| | | IDPRVTKIVPKKEKLRMGQNTATYDAFEPLKVPDWRNFLH |
| | | NKDDDVSNMKAIGAYVKEIVALNPKSFRIFSPDEITSNKL |
| | | DACLEITHRDFQWDPETAHDGGRVTEMLSEHTLQGFLQGY |
| | | TLTGRHGMFPSYEAFLGIVATMIDQYAKYIKMAEETPWRR |
| | | PIAGLLYIMTSTLWRQEHNGYSHQNPGLVGSFISLPRNLA |
| | | RIYFPADANTSVSTADHCLRSKNFINLIVGSKNPTRVLLT |
| | | AEEAEEHCVAGASVWKAYSTDGGVNPDVVLVGCGVEVTHE |
| | | VIAASAILRNHGIRVRVVNVNDLLVLGPAEGLRAHPHALS |
| | | DSAFYGLFTVDKPVVVNFHGYPKDIEGLLFGRAQHPISAD |
| | | ANRLQMNWRHELRNHQKYVEDTGEDPAWCNEIPEVKERK |
| 40 | XPK_Ca | MQSIIGKHKDEGKITPEYLKKIDAYWRAANFISVGQLYLL |
| | [EC: 4.1.2.9 | DNPLLREPLKPEHLKRKVVGHWGTIPGQNFIYAHLNRVIK |
| | 4.1.2.22] | KYDLDMIYVSGPGHGGQVMVSNSYLDGTYSEVYPNVSRDL |
| | | NGLKKLCKQFSFPGGISSHMAPETPGSINEGGELGYSLAH |
| | | SFGAVFDNPDLITACVVGDGEAETGPLATSWQANKFLNPV |
| | | TDGAVLPILHLNGYKISNPTVLSRIPKDELEKFFEGNGWK |
| | | PYFVEGEDPETMHKLMAETLDIVTEEILNIQKNARENNDC |
| | | SRPKWPMIVLRTPKGWTGPKFVDGVPNEGSFRAHQVPLAV |
| | | DRYHTENLDQLEEWLKSYKPEELFDENYRLIPELEELTPK |
| | | GNKRMAANLHANGGLLLRELRTPDFRDYAVDVPTPGSTVK |
| | | QDMIELGKYVRDVVKLNEDTRNFRIFGPDETMSNRLWAVF |
| | | EGTKRQWLSEIKEPNDEFLSNDGRIVDSMLSEHLCEGWLE |
| | | GYLLTGRHGFFASYEAFLRIVDSMITQHGKWLKVTSQLPW |
| | | RKDIASLNLIATSNVWQQDHNGYTHQDPGLLGHIVDKKPE |
| | | IVRAYLPADANTLLAVFDKCLHTKHKINLLVTSKHPRQQW |
| | | LTMDQAVKHVEQGISIWDWASNDKGQEPDVVIASCGDTPT |
| | | LEALAAVTILHEHLPELKVRFVNVVDMMKLLPENEHPHGL |
| | | SDKDYNALFTTDKPVIFAFHGFAHLINQLTYHRENRNLHV |
| | | HGYMEEGTITTPFDMRVQNKLDRFNLVKDVVENLPQLGNR |
| | | GAHLVQLMNDKLVEHNQYIREVGEDLPEITNWQWHV |
| 41 | POS5_Sc | MQSIIGKHKDEGKITPEYLKKIDAYWRAANFISVGQLYLL |
| | [EC: 2.7.1.86] | DNPLLREPLKPEHLKRKVVGHWGTIPGQNFIYAHLNRVIK |
| | | KYDLDMIYVSGPGHGGQVMVSNSYLDGTYSEVYPNVSRDL |
| | | NGLKKLCKQFSFPGGISSHMAPETPGSINEGGELGYSLAH |
| | | SFGAVFDNPDLITACVVGDGEAETGPLATSWQANKFLNPV |
| | | TDGAVLPILHLNGYKISNPTVLSRIPKDELEKFFEGNGWK |
| | | PYFVEGEDPETMHKLMAETLDIVTEEILNIQKNARENNDC |
| | | SRPKWPMIVLRTPKGWTGPKFVDGVPNEGSFRAHQVPLAV |
| | | DRYHTENLDQLEEWLKSYKPEELFDENYRLIPELEELTPK |
| | | GNKRMAANLHANGGLLLRELRTPDFRDYAVDVPTPGSTVK |
| | | QDMIELGKYVRDVVKLNEDTRNFRIFGPDETMSNRLWAVF |
| | | EGTKRQWLSEIKEPNDEFLSNDGRIVDSMLSEHLCEGWLE |
| | | GYLLTGRHGFFASYEAFLRIVDSMITQHGKWLKVTSQLPW |
| | | RKDIASLNLIATSNVWQQDHNGYTHQDPGLLGHIVDKKPE |
| | | IVRAYLPADANTLLAVFDKCLHTKHKINLLVTSKHPRQQW |
| | | LTMDQAVKHVEQGISIWDWASNDKGQEPDVVIASCGDTPT |
| | | LEALAAVTILHEHLPELKVRFVNVVDMMKLLPENEHPHGL |
| | | SDKDYNALFTTDKPVIFAFHGFAHLINQLTYHRENRNLHV |
| | | HGYMEEGTITTPFDMRVQNKLDRFNLVKDVVENLPQLGNR |
| | | GAHLVQLMNDKLVEHNQYIREVGEDLPEITNWQWHV |
| 42 | ZWF_Co | MAASNSPRPSRAKRGSIPSLEALGEAFKNETIVVVFGASG |
| | [EC: 1.1.1.49 | DLAKKKTFPALFTLYRQGHLPKDVHIVGYARTKMSEEEFH |
| | 1.1.1.363] | KRVTGYIKKDDSVDDFDERLEGFKKALTYISGPYDKDEGY |
| | | QELEKHLVEMESKRGPSEKANRVFYMALPPSAFTDVAAGV |
| | | RKNNYSKNGGNRIIVEKPFGKDLESCREMMSALKAEWNED |
| | | ETYRIDHYLGKEMVKNLLVLRFGNIVLDAVLNKYSVSNVQ |
| | | ITFKESFGTQGRGGYFDEFGIIRDVCQNHLMQVLSILAME |
| | | RPVSFAAEDLRDEKVKVLRSIPPIKKEDMYLGQYVAGNGE |
| | | PGYLDDETVPKGSTCPTYAALCLWINNPRWEGVPFIMKAG |
| | | KALNESKVEVRVQFKDVTSGIFTDIARNELVMRIQPDEAV |
| | | YIKLNSKLPGLITKAIPTELDLTYKDRFTDVEVPQAYEAL |
| | | ILDAFKGDRSNFVRDDELEQSWKIFTPILHYIDQEKPKPE |
| | | PYPFGARGPKDSLAFSEKYGYKRTDEGTYTWPVTSVSANL |
| 43 | CTP1_Co | MSPKGKEKEKPVASLIAGATAGGFEAFVTYPLESLKTQLQ |
| | | FQADAKTTPWSLLKDTVQKRGIKGLYAGVPAVVIGNAAKA |
| | | GVRFTTYDQFKSALKDEEGHLSAPRSMLAGLGAGMMEAII |
| | | AVTPSETIKTKMIEDSKRDVPRYNGMVDGVRKIVAEEGVG |
| | | GLYRGLGPVMLRQGANSAVRFSSYSTIKQLAQGSLPPGSV |
| | | LPGWMTFGIGSMAGIITVYTTMPFDVIKTRMQTIEAKKQY |
| | | RNALHCAYRIVTEEGVLKLWKGTVPRLGRLVLSGGIVFSV |
| | | YEKIYPIAASFIS |
| 44 | CAT2_Sc | MRICHSRTLSNLKDLPITSRRAMHSAIVNYSTQKAQFPVE |
| | [EC: 2.3.1.7] | TNNGEHYWAEKPNKFYQNKRPNFQGITFAKQQDLPSLPVP |
| | | ELKSTLDKYLQTIRPFCNDVETFERQQLLCKDFSEHMGPI |
| | | LQDRLKEYANDKRNWMAKFWDEQSYLQYNDPIVPYVSYFY |
| | | SHMPLPNHLSKIDNDPLIKATAIISTVVKFIEAIKDESLP |
| | | VEIIKGMPFCMNSFSLMFNTSRLPGKPEDNQDTNIFYSVY |
| | | ENNFVTIAYKGKFYKLMTHDGNDKPLSENEIWRQLYSVVF |
| | | QGSQSDPKLGGIGSLTSLPRDQWREVHLELMKDPISQDSL |
| | | ETIHKSSFMLCLDLDQSPVTLEEKSRNCWHGDGINRFYDK |
| | | SLQFLVTGNGSSGFLAEHSKMDGTPTLFLNNYVCQQLNKL |
| | | DVDDFMRKVITPSSTVAMKPMELPFIITPKIHKAIESAQL |
| | | QFKETIGEHDLRVWHYNKYGKTFIKRHGMSPDAFIQQVIQ |
| | | LAVFKYLKRQLPTYEAASTRKYFKGRTETGRSVSTASLEF |
| | | VSKWQNGDVPIAEKIQALKHSAKEHSTYLKNAANGNGVDR |
| | | HFFGLKNMLKSNDDQIPPLFKDPLFNYSSTWLISTSQLSS |
| | | EYFDGYGWSQVNDNGFGLAYMLNNEWLHINIVNKPAKSGA |
| | | SVNRLHYYLSQAADEIFDALENENKRKAKL |
| 45 | ACC1_Co | MAAVAPSGVNGGAAGGQYDHSRVSQFIGGNSIEVAPSSEV |
| | [EC: 6.4.1.2 | TDFVKKQGGHTVISKVLIANNGIAAVKEIRSVRKWSYETF |
| | 6.3.4.14 | GSDRMIEFTVMATPEDLRINAEYIRMADRYVEVPGGTNNN |
| | 2.1.3.15] | NYANVDLIVDVAERAGVHAVWAGWGHASENPRLPEMLRKA |
| | | KIVFIGPPASAMHSLGDKISSTIVAQSADVSCMAWSGTGI |
| | | TDTVLSPSGFVTVPDKAYDDACVHDWEEGLKRSETIGWPV |
| | | MIKASEGGGGKGIRMVDSGEKFKQAFQAVASEVPGSPIFI |
| | | MKLASAARHLEVQLMADQYGNAISLFGRDCSVQRRHQKII |
| | | EEAPVTIARPETFEKMEKAAVRLAKLVGYVSAGTVEYLYS |
| | | HHDDSYYFLELNPRLQVEHPTTEMVSGCNIPAIQLQVAMG |
| | | IPLHRIRDIRTLYGMDPHGSTEIDFDGDKPQTATTQRKPK |
| | | PKGHVIACRITGENPDAGFKPSSGALTELNFRSNSNVWGY |
| | | FSVSSAGGLHEYADSQFGHIFAYGMERSEARKSMVVALKE |
| | | LSIRGEFRTTVEYLIKLLEKPEFEDNTITTQWLDGLIAEG |
| | | MTSERPDTTLAVICGATVKAHIAYEASVAKYKSVLDRGQI |
| | | PTKDTLQTFFKTEFIYDDVRYSFAMAKSAPLQFTLYLNGG |
| | | RIYVGCRSLADGGLLISLDGASHTVYYREEVGAMVVSVDH |
| | | KTTMIEDERDPTQLRSPSPGKLVRYLIDNGDHVDAGQAYA |
| | | EIEVMKMIMPVTATESGIAQFMKQPGAQLAQGELIGILTL |
| | | DDPTKVKFARPFTGMLPSFELKNGRYGTKPHQRLREHLEV |
| | | LYDNLAGYDNTSAVTASLKVVQSTLRDQDLPFSNAHDVLS |
| | | TLSGRMPQKLEEQVRAAIETCRKDKLEFPSDKLKRIIHSF |
| | | IDENVDFKDRQTVTNAIAPLQTLIDAFAYGLKVHEWQIYA |
| | | DLLNYFCEIEEPFADSTRTQEEIILKLREENKDLEQVVKL |
| | | VLSHSKVASKTKLILQLLDIVKSESPKAESTTESGVAKAL |
| | | TRLSELDSRPTAKAALKAKEVLIVGSLPSYQERLDQMERT |
| | | LNSAVTTNYYGEAGGGHRLPSGESLKEITDSRYTVFDVLS |
| | | TFFDYKDPWVVLAALEVYIRRAYRVYNLMHLDYEPSTETG |
| | | GPNIVTWKFKFGGAGLEAGTPRVDSSRDITRIAAMSDLTY |
| | | LVHAKSEPMRFGLMTSYNSLEELKGGFASLLTKYPSFDRE |
| | | EFREKYGNEARDPNVLNVALRLFHDEQVQDAELLTEFLAV |
| | | VNNNLKAVAKTGSRRITFVVCRKDQYPSYITLREGADGYW |
| | | KEQEEIRNIEPALAYQLELGRLSNFKITPKMSTNRQIHVY |
| | | HAVGRENSSDVRFFVRALLRPGRFTGTMKQTEYLISETDR |
| | | LVGDVLDTLEIAQQEYRQADCNHISVNCVYSLNVSFDDVQ |
| | | EALAGFIERHGKRLWRLRVTQAEIRVVIEEEDGNQIPIRA |
| | | FIENVSGFVVKYEAYQEVTNERGKTILKSIGDQGQFHLQP |
| | | VNFPYPTKESLQPKRYQAHVIGTTYVYDFPDLFRQAIDKT |
| | | WQQVQQLLPNVKIPSDLLQATELVLDEHGELVEVNRPPGM |
| | | NSCGMVAWIYTMKTPEYPKGRRVVVISNDITYQIGSFGPL |
| | | EDEYFYKATQYARKHGLPRVYLSANSGARIGLADEVMALF |
| | | DVAWKEAGKPEKGFDYLYLTPASLEKLEAMGEGSVQTEEI |
| | | EVAGERRHKITAIIGLKDGLGVECLKGSGLIAGETSRAYD |
| | | DIFTISMVTARSVGIGAYLVRLGQRVVQVEGQPIILTGAQ |
| | | AINKVLGKEVYTSNIQLGGPQIMYRNGISHLTAGSDLDGA |
| | | LQIVNYLTFIPAKRGSAIPILPTGDSWDRDVAWKPPKGPY |
| | | DPRNFLAGAVEEDGFRPGILDNGSFFETMGGWAQTIVTGR |
| | | GRLNGIPVAVIAAETRTIERIDPADPANENSTESRVALAG |
| | | TVWFPDSSRKTATAIEDANREGLPLIIFANFRGFSGGMSD |
| | | MAQAILKEGAKIVDGLSSYKHPIIVYLVPNGELRGGAWVV |
| | | LDPAINPEYMTMYVDNESRGGVLEPEGIVEVKYRRPRIQA |
| | | TMARLDPEYAKLKAAAESDAAGPEEKAKAALALEAREKHL |
| | | APAYHNVATEFADLHDRAGRMKAKANCIPCDWENSRRAIY |
| | | WALRRKLSEVRIMKKIAAANPDLTYPERKELFAQFLPSEV |
| | | GNDSEVAAFLEKSSDAVEDFVQQIRDDYCANNIVSWAGSN |
| | | QRGVMEGFQRILDSLPANERAQFIASLSQQ* |
| 46 | TOR1_Co | MSSQPEALENIFSRLGSKSEDVRVAAAEDLKAQINSANIE |
| | | FPGDDSKKNVWKEVFHRTFELTRSNTTSERLGSIAAIDAM |
| | | LEETNNDSPDRALQNNIRLYGYLRPLLTYPDSTVMVAAAH |
| | | VVGNMVKLAGANLGESFFNKEVGQAIQMIDDSRQEVGRFS |
| | | AVLLLHQFAVYAPSQFYPFISRVLEKIWIPLRDSRTMVRE |
| | | RASMLLSSCLDIMKARERSSSEIYQVIFEEAKSGLLKASS |
| | | VDSILGSLLAFSAMLQNQQISMGEYYRQICEITLKYRDSK |
| | | ETVIRKSVITLIPSMATYDSDEFEVHYLHRSMAYLLQALN |
| | | KPTDRDIAYVALGHLSIQLASKMRPFIDDIVKIIKEHLRM |
| | | RGKKGAPFEAPIFQCLAMLTTAVGPMLTRQMHEILDLMFP |
| | | WGLSEALYHALEVISSHIPPLLRTIQDRLLETLSVILTGH |
| | | SFRPLGAPVRRAKGGEITRDVVIQASGGGQSPETIALALK |
| | | VLGNFDFGGHTLNEFVQEAALPYLEHDSPEVRKEAVLAST |
| | | QLFVNDPICNQTSSHAIEIVNDVLEKLLTVGITDQVPGIR |
| | | QAVLESLDEKFDRHLAQAEDIRCLFVALNDEEFKIRELAI |
| | | GIIGRLAHHNPAYVMPSLRKSLINLITELEYSTANKQKEE |
| | | SAKLLCLLIGASSSLVKSYASTILSVILRIARSPSTPATV |
| | | TASCVNCIGELARVAGEELVSNAKTIMDLLLELLNDPAST |
| | | VKRNAALKALGQVVSNTGEVITPYSDYPQLLGILFRILRL |
| | | ETSSQIRLEAIRTLGMLGALDPFKHKQLLGGDNDNTTEST |
| | | GPRVTDITLLMNHNGGSNDDYYQMVVINSLVNVLNDANMK |
| | | EHHYDAINSVMLIFRTQRLRCVGFLPQILPAFLGVIRMGS |
| | | RDSQDVFLKQLAQLISIVKQHIRNYLSEVFALVHDYWNPN |
| | | SMMQITIISLVEAIARAVEGEFKAYLPQLLQQILRTFDGD |
| | | LSARNLTDQRENTLLHILKAFIVFGSSIEDYLHLVLPVII |
| | | RSFENPAAPTKLRKAALTTTAQLCRKVNFSDHASQIIHPL |
| | | VRTLSTQDPELRNAAMETLCVLVLQFGPDYAIFIPMVNKA |
| | | LLENKIVWPMYDQMVTKLLNRERLPPDLGPVEQYAADSMN |
| | | EVSVSAEQHKLPVNQQQLKSAWDCSHVATKKEWLEWLKRL |
| | | GVEFMRESPSQAIRACTKLADQHGAFARELFNVAFVSCWT |
| | | ELYESYQEDLVHHLEQALTNDGVPSEVVNTVLNLAEFMEH |
| | | DDKPLALEPRLLGDYATAFHAYAKALHYKELEFFVDASPT |
| | | VVEDLISVNQKLQQSDAAWGTLEYAQNNLQMTHDVLWYEK |
| | | LGRWEEALEVWNERSADPDSQFDEQALAMGKFQALHALGE |
| | | WEELADVIQVRWPNAAGEEKKTIAPLGAAASWALYQWDMM |
| | | DDYISAMKTDSADRNFFKAILAVHRNQFTSAKRHITKARE |
| | | RLDGELTTLTGESYGRAYDVIVRVQMLSELEEIISYKDHA |
| | | DQPERQATQRQTWHKRLAGCQRDVEVWQRILQVRSLVLTP |
| | | SEDMETWISFADLCRVSDRLNLAEKTLTSLVGSSCSNLDA |
| | | ESRSRAPPPIVFAYYRLKWAQAVAVDSRDERVETLGYLHD |
| | | FTQTLSDDMGLGPRNNQGRLILPDVKLYGEYTKLLARCHV |
| | | ELGKWEASMLEQGFTAAPASVLSNYALATELDPDWYQAWH |
| | | TWALANFEVIGSLEVSQSGLQPAHFATYIIPAVEGFLRSI |
| | | ALSPGNSLQDTLRLLTLWFKYGYQSGVNQAIQQGMASVNI |
| | | DVWLEVIPQIIARIQTPRPTIRELIVRLLHSIGRAHPQAL |
| | | IYPLTVASKSNVATRKQVAQDIMDKMREHSATIVDQADLV |
| | | STELIRAAILWHEQWHEGLEEASKHYFADHNEEAMFEVLE |
| | | PLHDMIEKGPETVRETSFVQSFGHDLRLARDLCRRYRQQG |
| | | DLNELNQAWDIYCATFQRLARQIKLLNVIELQYVSPKLMN |
| | | VRDLDLAVPGTYQSGKPVIGIAYVLPTFTVINSKQRPRRF |
| | | TMRGRDGKEYTFVLKGHEDLRQDERVMQLFGLVNTLLSAD |
| | | QESSKRHLSIRQFSITPLSPGAGLLGWVPHSDTLHALIKH |
| | | YRDSRKILIDIETRLMNQMADDNYDSLPLLHKVEVFQYAL |
| | | DNTTGQDLYRILWLRSRNSESWLERRTTYTRSLALTSMVG |
| | | YILGLGDRHPSNLMLDQITGEIVHIDFGDCFEVAMQRDKY |
| | | PEKVPFRLTRMLIHAMEVCGITGTFSRSCEVSMEVLRANK |
| | | ESLMAVLEAFVYDPLINWRLTAGQGGQRQGAHDVEERGGQ |
| | | PYAVQRGGRADERDILVEADRPEVMNDKALQVVERVRRKL |
| | | TGRDFKPNVTLDIKEQVEELVTQATSVENLCVAFVGWCSF |
| | | W |
| 47 | ACL_Co | MSTKAIREYDAKRLVNYWLTRSPTPIPTVTESTAAPVRVA |
| | [EC: 2.3.3. 8] | QVEWDDESSSLQPPIKPGAGLPEFVFNEAKLVGKPDQLIK |
| | | RRGKAGLLSIGKDWQTTGDWIAQRAGKPVKVESTTGTLKT |
| | | FIIEPFLPHTQDTEYYVCINSLRDGDWIYFTHEGGVDVGD |
| | | VDAKALKLFLPVNKPFPSREEVIKTLLPNVPEKKKDVLCD |
| | | FIIRLYGVYVDLHFAYLEINPLVCLDPVDGKPAEIHYLDM |
| | | AAKLDQTADFLCGPKWAIARDDSTPAAAGKGPIKADRGPP |
| | | MVWPAPFGRDLTKEEAYIQKLDASTGASLKLTVLNQHGSV |
| | | WTMVAGGGASVVYSDAIAAAGYAHELANYGEYSGAPTEGQ |
| | | TYEYAKTILDLMTRGEPLKDGKVLIIGGGAANFSDVAATF |
| | | KGIIRALKEHKEGLIRHNVKIWVRRAGPNYQEGLKAMRLC |
| | | GESLGVPIKVYGPEAPITAIVPMALGVNKDKDISRTTSSA |
| | | SIAAAVENAANGSAKKRSTKQEERPVIGEIKSDGSREQPG |
| | | DQIVHFETEKISQRPWYRPFDASTRSFVFGLQPRAIQGML |
| | | DFDYSCGRKTPSVAAMIYPFGGHHIQKFYWGTKETLLPVY |
| | | TTVGEAIKKHPDVDVVVNFASSRSVYSSTLDILSYPQIKA |
| | | IGIIAEGVPERHAREILHLAQEKNIIIIGPATVGGIKPGC |
| | | FRIGNTGGMMDNLLACKLYRPGSVGYVSKSGGMSNELNNI |
| | | LSQTTNGVYEGIAIGGDRYPGTSFIDHILRYEADPECKLI |
| | | LLLGEVGGTEEYRVIEAVKSGKVTKPIVAWAIGTCAKMFT |
| | | SEVQFGHAGSMANSDMETADAKNRAMREAGFLVPDTFEDL |
| | | PDLLKNVYDKMVSTGAIKPAPEQEPPAIPMDYQWASKLGL |
| | | IRKPAAFISTISDERGQELMYAGMRISDVFKEDIGIGGVI |
| | | SLLWFKRRLPTYACKFIEMVLQLTADHGPAVSGAMNTIIT |
| | | ARAGKDLISSLVAGLLTIGDRFGGALDGAASEFSRGVASG |
| | | HTPREFVDSMRKANKLIPGIGHKIKSKTNPDLRVVLVVDY |
| | | VKKHFPSHKTLDFALAVEDVTTQKSNTLILNVDGAIAASF |
| | | CDLLNGCGAFTEEEATEYLKSGTLNGLFVLGRSMGFIGHY |
| | | LDQRMLKQPLYRHPADDIFINMESRVLAGP |
| 48 | OLE1_Y1 | MVKNVDQVDLSQVDTIASGRDVNYKVKYTSGVKMSQGAYD |
| | EC: 1.14.1 9.1 | DKGRHISEQPFTWANWHQHINWLNFILVIALPLSSFAAAP |
| | | FVSFNWKTAAFAVGYYMCTGLGITAGYHRMWAHRAYKAAL |
| | | PVRIILALFGGGAVEGSIRWWASSHRVHHRWTDSNKDPYD |
| | | ARKGFWFSHFGWMLLVPNPKNKGRTDISDLNNDWVVRLQH |
| | | KYYVYVLVFMAIVLPTLVCGFGWGDWKGGLVYAGIMRYTF |
| | | VQQVTFCVNSLAHWIGEQPFDDRRTPRDHALTALVTFGEG |
| | | YHNFHHEFPSDYRNALIWYQYDPTKWLIWTLKQVGLAWDL |
| | | QTFSQNAIEQGLVQQRQKKLDKWRNNLNWGIPIEQLPVIE |
| | | FEEFQEQAKTRDLVLISGIVHDVSAFVEHHPGGKALIMSA |
| | | VGKDGTAVFNGGVYRHSNAGHNLLATMRVSVIRGGMEVEV |
| | | WKTAQNEKKDQNIVSDESGNRIHRAGLQATRVENPGMSGM |
| | | AA |
| 49 | OLE1_Ss | MTEQLDTADITALNALAAGTNKKTPRIVAAGLGGRLMGTA |
| | EC: 1.14.1 9.1 | QMVKVTAEQITEDSLESLADKDAREKAMYAKTKHISELPW |
| | | TFGNFFEKINWLNMILVVALPAFGTYQAIFYPPPLKTLVL |
| | | AFVLYLSCGLSITAGYHRLFSHRSYDAVTPIRLFFAFFGA |
| | | GAIEGSIKWWGHSHRIHHRYTDTDRDPYDARQGFWYSHMG |
| | | WMLTKANPKNRARADISDLSTDWVVTFQHRHYLLIMLVSA |
| | | FVVPTLTAGLLWGDYWGGFIYGGILKSFFIQQVTFCINSL |
| | | AHWIGVQPFDDRRTPRDHALTAVFTFGEGYHNFHHEFPSD |
| | | YRNALKWYQYDPTKIAIWILSKLGLAWNLKKFSQNAIEQG |
| | | LVQQQQKKLDRMKSKLNWGLSVDELPVWDREEFNAKAKTD |
| | | GLIIISGIIHNVKNFIKEHPGGQALVRASLGKDATKAFNG |
| | | AVYAHSNAAHNLLASMRVAVIRDGNINANTFDLQQQFLDK |
| | | EKLN |
| 50 | DGAT_Ss | MDGIDTRLHLDDPTAISTGVEPPIATKRRTTRTSSALSKD |
| | | SNTSMNSVGSAGSTSSSSGRSRRPLLNVAPLNTPLTNRLQ |
| | | TVGVIWHTISIPSFACLFLLFVSLGWILWLTVVLPYFIWW |
| | | YGFDLHTPTNGKVVYRVRDWMRNFIIWEWFVNYFPIVVHK |
| | | TVDLEPTFSEVEVIEDDETDDEEDLISEDSRTLIDKIFKG |
| | | LGLSKRLNDTESRSPPPQNEVDSSENSSSSQSSTSLSSSK |
| | | LKNVKYRRVSTGPRYIFGYHPHGVISMGVMGTFATNAVRN |
| | | EPYEPPLKILKPLFHDPSKGERMFAGIGNVFPLTLTTQFT |
| | | LPLYRDYLLGLGLTSASAKNIKSLINNGDNSVCIVVGGAQ |
| | | ESLLNNMVSGINKVGKGYKGNVHDDEPQEPIVEETIVGSD |
| | | SVTSQSPQSITKENKRQIRLVLHKRKGFVKLAIELGNVSL |
| | | VPIFAFGEADIYKLTIPKPGSIGYAFQQWMKKTFLFTVPF |
| | | FSARGVFIYDFGFLPFRNPINVCVGRPIHIPQGVLAEYKL |
| | | KHPDPEEIAPPPPLLGDKKISRKDSFSNLFKLGGQPKPKR |
| | | PPTKTRMPPELLDHYHKLYVDELKRVYEENKCKYGYEDVE |
| | | LNIIE |
| 51 | DGAT_Yl | MTIDSQYYKSRDKNDTAPKIAGIRYAPLSTPLLNRCETFS |
| | | LVWHIFSIPTFLTIFMLCCAIPLLWPFVIAYVVYAVKDDS |
| | | PSNGGVVKRYSPISRNFFIWKLFGRYFPITLHKTVDLEPT |
| | | HTYYPLDVQEYHLIAERYWPQNKYLRAIISTIEYFLPAFM |
| | | KRSLSINEQEQPAERDPLLSPVSPSSPGSQPDKWINHDSR |
| | | YSRGESSGSNGHASGSELNGNGNNGTTNRRPLSSASAGST |
| | | ASDSTLLNGSLNSYANQIIGENDPQLSPTKLKPTGRKYIF |
| | | GYHPHGIIGMGAFGGIATEGAGWSKLFPGIPVSLMTLTNN |
| | | FRVPLYREYLMSLGVASVSKKSCKALLKRNQSICIVVGGA |
| | | QESLLARPGVMDLVLLKRKGFVRLGMEVGNVALVPIMAFG |
| | | ENDLYDQVSNDKSSKLYRFQQFVKNFLGFTLPLMHARGVF |
| | | NYDVGLVPYRRPVNIVVGSPIDLPYLPHPTDEEVSEYHDR |
| | | YIAELQRIYNEHKDEYFIDWTEEGKGAPEFRMIE |
| 52 | ELO2_Ma | MESGPMPAGIPFPEYYDFFMDWKTPLAIAATYTVAVGLFN |
| | | PKVGKVSRVVAKSANAKPAERTQSGAAMTAFVFVHNLILC |
| | | VYSGITFYHMFPAMVKNFRTHTLHEAYCDTDQSLWNNALG |
| | | YWGYLFYLSKFYEVIDTIIIILKGRRSSLLQTYHHAGAMI |
| | | TMWSGINYQATPIWIFVVFNSFIHTIMYCYYAFTSIGFHP |
| | | PGKKYLTSMQITQFLVGITIAVSYLFVPGCIRTPGAQMAV |
| | | WINVGYLFPLTYLFVDFAKRTYSKRTAIAAQKKAQ |
| 53 | OLE1_Pg | MSKPSPSTPATAPHLRQRQRKNLPDYDPDSDLSESEGLGG |
| | EC: 1.14.19.1 | LRSQVGNTWEDDEETAVDDDSYVQRTLRKEKPLPPITWCN |
| | | FYREINMISTLALTVVPILAIYGAFTTPLYRSTLAWSILY |
| | | YYFTGLGITAGYHRLWAHRSYNASLPLQYFLALGGSGAVE |
| | | GSIRWWARGHRAHHRYTDTDLDPYSAHKGLLWSHVGWMIV |
| | | KPRRKPGVADVSDLSRNQVVRWQHRWYLPLIFGMGFFFPT |
| | | LVAGLGWGDWRGGFFYAGAARLLFVHHSTFCVNSLAHWLG |
| | | EAPFDDKHTPRDHIITAFVTIGEGYHNFHHEFPQDFRNAI |
| | | RWYQYDPTKWFIAVAAFLGLASELKTFPDNEVRKGQYSMK |
| | | LKELQRDFRDVKWPKSSNDLPIVTWEQFVEEADKKNGRDL |
| | | IVVGGFIHDVTEFIDEHPGGRALIKTRLGKDATTAFHGGV |
| | | YDHSNAAHNLLAMLRVGVIEGGYEVEHLKKKVGVFRKEQQ |
| | | IPICGPKSLGTISTPESPVVEVKPIYT |
| 54 | EDA_Ec | MKNWKTSAESILTTGPVVPVIVVKKLEHAVPMAKALVAGG |
| | [EC: 4.1.2.14 | VRVLEVTLRTECAVDAIRAIAKEVPEAIVGAGTVLNPQQL |
| | 4.1.3.42] | AEVTEAGAQFAISPGLTEPLLKAATEGTIPLIPGISTVSE |
| | | LMLGMDYGLKEFKFFPAEANGGVKALQAIAGPFSQVRFCP |
| | | TGGISPANYRDYLALKSVLCIGGSWLVPADALEAGDYDRI |
| | | TKLAREAVEGAKL |
| 55 | EDD_Ec | MNPQLLRVTNRIIERSRETRSAYLARIEQAKTSTVHRSQL |
| | EC: 4.2.1.12 | ACGNLAHGFAACQPEDKASLKSMLRNNIAIITSYNDMLSA |
| | | HQPYEHYPEIIRKALHEANAVGQVAGGVPAMCDGVTQGQD |
| | | GMELSLLSREVIAMSAAVGLSHNMFDGALFLGVCDKIVPG |
| | | LTMAALSFGHLPAVFVPSGPMASGLPNKEKVRIRQLYAEG |
| | | KVDRMALLESEAASYHAPGTCTFYGTANTNQMVVEFMGMQ |
| | | LPGSSFVHPDSPLRDALTAAAARQVTRMTGNGNEWMPIGK |
| | | MIDEKVVVNGIVALLATGGSTNHTMHLVAMARAAGIQINW |
| | | DDFSDLSDVVPLMARLYPNGPADINHFQAAGGVPVLVREL |
| | | LKAGLLHEDVNTVAGFGLSRYTLEPWLNNGELDWREGAEK |
| | | SLDSNVIASFEQPFSHHGGTKVLSGNLGRAVMKTSAVPVE |
| | | NQVIEAPAVVFESQHDVMPAFEAGLLDRDCVVVVRHQGPK |
| | | ANGMPELHKLMPPLGVLLDRCFKIALVTDGRLSGASGKVP |
| | | SAIHVTPEAYDGGLLAKVRDGDIIRVNGQTGELTLLVDEA |
| | | ELAAREPHIPDLSASRVGTGRELFSALREKLSGAEQGATC |
| | | ITF |
| 56 | CBBM_Td | MDQSARYADLSLKEEDLIKGGRHILVAYKMKPKSGYGYLE |
| | EC: 4.1.1.39 | AAAHFAAESSTGTNVEVSTTDDFTKGVDALVYYIDEASED |
| | | MRIAYPLELFDRNVTDGRFMLVSFLTLAIGNNQGMGDIEH |
| | | AKMIDFYVPERCIQMFDGPATDISNLWRILGRPVVNGGYI |
| | | AGTIIKPKLGLRPEPFAKAAYQFWLGGDFIKNDEPQGNQV |
| | | FCPLKKVLPLVYDAMKRAQDDTGQAKLFSMNITADDHYEM |
| | | CARADYALEVFGPDADKLAFLVDGYVGGPGMVTTARRQYP |
| | | GQYLHYHRAGHGAVTSPSAKRGYTAFVLAKMSRLQGASGI |
| | | HVGTMGYGKMEGEGDDKIIAYMIERDECQGPVYFQKWYGM |
| | | KPTTPIISGGMNALRLPGFFENLGHGNVINTAGGGSYGHI |
| | | DSPAAGAISLRQSYECWKQGADPIEFAKEHKEFARAFESF |
| | | PKDADKLFPGWREKLGVHS* |
| 57 | PRK_So | MAVCTVYTIPTTTHLGSSFNQNNKQVFFNYKRSSSSNNTL |
| | EC: 2.7.1.19 | FTTRPSYVITCSQQQTIVIGLAADSGCGKSTFMRRLTSVF |
| | | GGAAEPPKGGNPDSNTLISDTTTVICLDDFHSLDRNGRKV |
| | | EKVTALDPKANDFDLMYEQVKALKEGKAVDKPIYNHVSGL |
| | | LDPPELIQPPKILVIEGLHPMYDARVRELLDFSIYLDISN |
| | | EVKFAWKIQRDMKERGHSLESIKASIESRKPDFDAYIDPQ |
| | | KQHADVVIEVLPTELIPDDDEGKVLRVRMIQKEGVKFFNP |
| | | VYLFDEGSTISWIPCGRKLTCSYPGIKFSYGPDTFYGNEV |
| | | TVVEMDGMFDRLDELIYVESHLSNLSTKFYGEVTQQMLKH |
| | | QNFPGSNNGTGFFQTIIGLKIRDLFEQLVASRSTATATAA |
| | | KA* |
| 58 | VEL4_Co | MPSGSLSAPTATGSSRRERTASASSATSSQQDARALSTPV |
| | (322563) | TPRHPSAATSQSHAKPRRESRESWGNPPRVASASRSASAG |
| | | GSMAATPDPASERARSTELDEGARKRWEARRQAYSQLALV |
| | | ICQQPQRARLCSFKEENETIDRRPVDPPPVLELISRDGQY |
| | | PDLLDSTQFFIRASLVSKDPVSYDEGKPCYQPQVTQSGAE |
| | | ATAGDVIQTPERLRHMDGTPGALCIFAKLSVRLPGPFRLK |
| | | FTLYETSPDGSSEICDTVSDIFEVYSPKQFKGMHESTPLT |
| | | RHLATQGLKVKIRQGNDSQGKAGNKRRASSNSQFSRQQQR |
| | | SPASDALSPVPPSSAIQPQPPHLRYSQGPAGPPGGPITAP |
| | | SYHSQRGLVWPGHFPYPGYHDEMGGHSATRRSPGAVDADR |
| | | PRKVRRSMGHEDVYSAHDIPSRLDMTLPSPSSDLSAFSLG |
| | | SGRPASASSAPPSSRHDWVRREYPPGDDGLPHLVLSPPPP |
| | | QYPSSSRAAPPPSHHAAREFAYPRLPRMDSASGHPRYPPA |
| | | HHRHHAIPHPHELQHHASSHYSHPHSHSLQHSHPHPHPHP |
| | | HPQRHPDHSQHRPPSSPSSYSHHPRAMEYHHGYRAGPPPP |
| | | ASHSQYTPGSPPPVLPPIRHSRSRSPSLPRHYDRDPRA* |
| 59 | VEL1_Co | MSHSRSSPSLALDDEERLPASPPPSTTDNERPSGDSTRIA |
| | (388151) | PRPSSSSSASKGGFAQWATSSSQVQPSDVPPAKSRIRPQR |
| | | SRNATGTTTATASTEDSVGSSDEDSDELDDDESRHERGMS |
| | | TSSVTIPPDQQLSRSDLADVRSVYVLEVVQQPQRARACGF |
| | | GDKDRRPLSPPPIIQLRIFDKLGNAIKPHAIDVHRFILMV |
| | | DLWNGDRTQQRSIVMHPGARPDQYALFTHTASRTASTHRY |
| | | DAPHSTFNRAPHRSMTGPAPGPYAWPSYGRPSLSPSPSYH |
| | | SQPLPSYPPTPGSGSVPGWPPPGHPEHPMASPAWVGAYSL |
| | | PPEEYDHHRSPPPPRRRPMPGEMRPPPIHDPSAWSPESTM |
| | | GGPDYSSERHASSSWSSYRPATSSADSGWGREPSYREPRP |
| | | HTSGFGEWDRGSVGPPGARPWSSTASDPHRPWSSHGSLYT |
| | | HAPSPVTQQSSLPLTASPVSTPSQLYPETPYPERLSTTSY |
| | | YSRVLVGSMGAVCQRLKDLNGDPGLFFFAHDLGIRTEGTF |
| | | SLRFTLVDLTSSLAGPEVHKDDSTEILARIFSDAFTVYSA |
| | | KRFPGVLPTTELTQCFADQSVRLPTRQKRAIAGIGSSSKR |
| | | KK* |
| 60 | OLE1_Co | MSASTKQAATTVAQPSGKPVTNVIDPERDDFIVPDNYVTR |
| | [EC: 1.14.19.1] | TVENMKMLPPVTWRNLHKNIQWISFLALTIPPAMAIYGLC |
| | | TVPVQTKTFIWSVVYYFITGLGITAGYHRLWAHRSYNASK |
| | | PLQYFLALAGAGSVQGSIRWWSRGHRAHHRYTDTKLDPYS |
| | | AHEGFWHAHMGWMLIKPRGKIGVADISDLSKNPVVKWQHN |
| | | NYVALLFFMGLAFPTLVAGLGWGDWWGGLFFAGAARLVFV |
| | | HHSTFCVNSLAHWLGETPFDNKHTPKDHFITALVTVGEGY |
| | | HNFHHQFPMDFRNAIKWYQYDPTKWFIWTMAQLGLASHLK |
| | | KFPDNEIKKGQYTMKLMQLQEQSEKLEWPKHSNDLPVISW |
| | | EDFQAESKTRALIAVHGFIHDCSSFIEDHPGGAHLIKRAI |
| | | GTDSTTAFFGGVYDHSASSTVSASSILSEVEDMDKPERKP |
| | | VYQPVAQVSNPWTLAVPPSEKYSNAAHNLLAMMRVGVLDG |
| | | GMEVEHLKVGRLTRSNSADSLVSDAPSRIIRTSPEIRPGL |
| | | ISRIQSAASPSLTRAASFDAPVAP* |
| 61 | FAT5_Ce | MTQIKVDAIISKQFLAADLNEIRQMQEESKKQVIKMEIVW |
| | EC: 1.14.19.1 | KNVALFVALHIGALVGLYQLVFQAKWATVGWVFLLHTLGS |
| | | MGVTGGAHRLWAHRAYKATLSWRVFLMLINSIAFQNDIID |
| | | WARDHRCHHKWTDTDADPHSTNRGMFFAHMGWLLVKKHDQ |
| | | LKIQGGKLDLSDLYEDPVLMFQRKNYLPLVGIFCFALPTF |
| | | IPVVLWGESAFIAFYTAALFRYCFTLHATWCINSVSHWVG |
| | | WQPYDHQASSVDNLWTSIAAVGEGGHNYHHTFPQDYRTSE |
| | | HAEFLNWTRVLIDFGASIGMVYDRKTTPEEVIQRQCKKFG |
| | | CETEREKMLHKLG |
| 62 | SCD_T66 | MCKVEPTRHEQTATAKPEALAPAEPQEQQQQPIIHGKHNP |
| | EC: 1.14.19.1 | DLPTLGEIRAVVPKHCFERSLVTSSLYLGRDLVMAACLLF |
| | | LARQYLPVYDMGLSGALGWTAYVIVQGTVCAGLWVLGHEC |
| | | GHQAFSNYRIVNDGVGYLVHTSLLVPYFSWAYTHGLHHAR |
| | | VNHMLDGESHTPNLQKKVAASFQKFCDMMGDEAFAVLHVF |
| | | IYLLLAWPLYIINGSGASKRNHEGKRWSKDFWKRPNHFMP |
| | | TSELFPDKMRVKAAISTIGVLTVIAGLCYWGAVEGGRTVL |
| | | LQYFLPYLVVNAYLIGFTWMQHTHPDVPHLGEDEWSWVAG |
| | | TVLTVDRPYPPFIDVLTHRIGSTHVAHHLFSKMPWYHARE |
| | | ATTHIRALLEPKGVYNYDPMPFYKALFHTAKHCHYMEGVE |
| | | GIQFFKHATAQPKAKTL* |
| 63 | ELO1_T66 | MMEPLDRYRALAELAARYASSAAFKWQVTYDAKDSFVGPL |
| | | GIREPLGLLVGSVVLYLSLLAVVYALRNYLGGLMALRSVH |
| | | NLGLCLFSGAVWIYTSYLMIQDGHFRSLEAATCEPLKHPH |
| | | FQLISLLFALSKIWEWFDTVLLIVKGNKLRFLHVLHHATT |
| | | FWLYAIDHIFLSSIKYGVAVNAFIHTVMYAHYFRPFPKGL |
| | | RPLITQLQIVQFIFSIGIHTAIYWHYDCEPLVHTHFWEYV |
| | | TPYLFVVPFLILFLNFYLQQYVLAPAKTKKA |
| 64 | ELO1_Co | MVAIWKQGQGPLITTLKALWSSANLPYPPKEYVEWIPGKS |
| | | PLSLQKEVIIALITYLVVIFGGRELMRNREPFKLRTLFRI |
| | | HNAFLSGGSLILLVLLMEEVAAFYFSRGFYYSICHPGAFT |
| | | PTAVTYYMINYYFKYIELIDTVFLFLKKKPLAFLHVFHHS |
| | | ATALLCFTQLEGATSVQWVVISINLLVHVIMYYYYWATAG |
| | | GAKIWWKKYLTTMQITQFVIDLFITYYATTNHFFYKYNIN |
| | | LPWVKDCTGGEGAALFGCGLLTSYLFLFIAFYKATYKKKG |
| | | ARAAAAAAAANGNGVKKTN* |
| 65 | ELO2_Y1 | MLSSISPDLYSSFSFKNSLAEAMPSVPHELINSKTLSWMY |
| | | NASLDIRVPLTIGTIYAVSVHLTNSSERIKKRQPIAFAKT |
| | | ALFKWLCVLHNAGLCLYSAWTFVGILNAVKHAYQITGDSS |
| | | APFSFNTLWGSFCSRDSLWVTGLNYYGYWFYLSKFYEVVD |
| | | TMIILAKGKPSSMLQTYHHTGAMFSMWAGIRFASPPIWIF |
| | | VVFNSLIHTIMYFYYTLTTLKIKVPKILKASLTTAQITQI |
| | | VGGGILAASHAFIYYKDHQTETVCSCLTTQGQFFALAVNV |
| | | IYLSPLAYLFIAFWIRSYLKAKSN* |
| 66 | EUTE_Ec | MNQQDIEQVVKAVLLKMQSSDTPSAAVHEMGVFASLDDAV |
| | [EC: 1.2.1.10] | AAAKVAQQGLKSVAMRQLAIAAIREAGEKHARDLAELAVS |
| | | ETGMGRVEDKFAKNVAQARGTPGVECLSPQVLTGDNGLTL |
| | | IENAPWGVVASVTPSTNPAATVINNAISLIAAGNSVIFAP |
| | | HPAAKKVSQRAITLLNQAIVAAGGPENLLVTVANPDIETA |
| | | QRLFKFPGIGLLVVTGGEAVVEAARKHTNKRLIAAGAGNP |
| | | PVVVDETADLARAAQSIVKGASFDNNIICADEKVLIVVDS |
| | | VADELMRLMEGQHAVKLTAEQAQQLQPVLLKNIDERGKGT |
| | | VSRDWVGRDAGKIAAAIGLKVPQETRLLFVETTAEHPFAV |
| | | TELMMPVLPVVRVANVADAIALAVKLEGGCHHTAAMHSRN |
| | | IENMNQMANAIDTSIFVKNGPCIAGLGLGGEGWTTMTITT |
| | | PTGEGVTSARTFVRLRRCVLVDAFRIV* |
| 67 | MPHF_ | MSKRKVAIIGSGNIGTDLMIKILRHGQHLEMAVMVGIDPQ |
| | Ec | SDGLARARRMGVATTHEGVIGLMNMPEFADIDIVFDATSA |
| | | GAHVKNDAALREAKPDIRLIDLTPAAIGPYCVPVVNLEAN |
| | | VDQLNVNMVTCGGQATIPMVAAVSRVARVHYAEIIASIAS |
| | | KSAGPGTRANIDEFTETTSRAIEVVGGAAKGKAIIVLNPA |
| | | EPPLMMRDTVYVLSDEASQDDIEASINEMAEAVQAYVPGY |
| | | RLKQRVQFEVIPQDKPVNLPGVGQFSGLKTAVWLEVEGAA |
| | | HYLPAYAGNLDIMTSSALATAEKMAQSLARKAGEAA* |
| 68 | ALD6_Sc | MTKLHFDTAEPVKITLPNGLTYEQPTGLFINNKFMKAQDG |
| | [EC: 1.2.1. | KTYPVEDPSTENTVCEVSSATTEDVEYAIECADRAFHDTE |
| | 4] | WATQDPRERGRLLSKLADELESQIDLVSSIEALDNGKTLA |
| | | LARGDVTIAINCLRDAAAYADKVNGRTINTGDGYMNFTTL |
| | | EPIGVCGQIIPWNFPIMMLAWKIAPALAMGNVCILKPAAV |
| | | TPLNALYFASLCKKVGIPAGVVNIVPGPGRTVGAALTNDP |
| | | RIRKLAFTGSTEVGKSVAVDSSESNLKKITLELGGKSAHL |
| | | VFDDANIKKTLPNLVNGIFKNAGQICSSGSRIYVQEGIYD |
| | | ELLAAFKAYLETEIKVGNPFDKANFQGAITNRQQFDTIMN |
| | | YIDIGKKEGAKILTGGEKVGDKGYFIRPTVFYDVNEDMRI |
| | | VKEEIFGPVVTVAKFKTLEEGVEMANSSEFGLGSGIETES |
| | | LSTGLKVAKMLKAGTVWINTYNDFDSRVPFGGVKQSGYGR |
| | | EMGEEVYHAYTEVKAVRI |
| 69 | ADH2_Sc | DVFNHVVKSISIVGSYVGNRADTREALDFFARGLVKSPIK |
| | [EC: 1.1.1.1] | VVGLSSLPMSIPETQKAIIFYESNGKLEHKDIPVPKPKPN |
| | | ELLINVKYSGVCHTDLHAWHGDWPLPTKLPLVGGHEGAGV |
| | | VVGMGENVKGWKIGDYAGIKWLNGSCMACEYCELGNESNC |
| | | PHADLSGYTHDGSFQEYATADAVQAAHIPQGTDLAEVAPI |
| | | LCAGITVYKALKSANLRAGHWAAISGAAGGLGSLAVQYAK |
| | | AMGYRVLGIDGGPGKEELFTSLGGEVFIDFTKEKDIVSAV |
| | | VKATNGGAHGIINVSVSEAAIEASTRYCRANGTVVLVGLP |
| | | AGAKCSSEIYEKMEKGQIAGRYVVDTSK* |
| 70 | GROES_Ec | ILENGEVKPLDVKVGDIVIFNDGYGVKSEKIDNEEVLIMS |
| | | ESDILAIVEMNIRPLHDRVIVKRKEVETKSAGGIVLTGSA |
| | | AAKSTRGEVLAVGNGRA* |
| 71 | GROEL_Ec | MAAKDVKFGNDARVKMLRGVNVLADAVKVTLGPKGRNVVL |
| | | DKSFGAPTITKDGVSVAREIELEDKFENMGAQMVKEVASK |
| | | ANDAAGDGTTTATVLAQAIITEGLKAVAAGMNPMDLKRGI |
| | | DKAVTAAVEELKALSVPCSDSKAIAQVGTISANSDETVGK |
| | | LIAEAMDKVGKEGVITVEDGTGLQDELDVVEGMQFDRGYL |
| | | SPYFINKPETGAVELESPFILLADKKISNIREMLPVLEAV |
| | | AKAGKPLLIIAEDVEGEALATLVVNTMRGIVKVAAVKAPG |
| | | FGDRRKAMLQDIATLTGGTVISEEIGMELEKATLEDLGQA |
| | | KRVVINKDTTTIIDGVGEEAAIQGRVAQIRQQIEEATSDY |
| | | DREKLQERVAKLAGGVAVIKVGAATEVEMKEKKARVEDAL |
| | | HATRAAVEEGVVAGGGVALIRVASKLADLRGQNEDQNVGI |
| | | KVALRAMEAPLRQIVLNCGEEPSVVANTVKGGDGNYGYNA |
| | | ATEEYGNMIDMGILDPTKVTRSALQYAASVAGLMITTECM |
| | | VTDLPKNDAADLGAAGGMGGMGGMGGMM* |

Table 3 contains relevant homologues to AGS1, MNT1, VEL1, and VEL4. Deletion or inactivation of MNT1 was motivated by the relationship of MNT1 to the Cryptococcus neoformans homologue which is important in the formation of the capsule; which is composed of polysaccharide. Deletion or inactivation of AGS1 was motivated by the relationship of our AGS1 to the AGS of A. nidulans where mutants showed loss of cell wall polysaccharide containing alpha 1,3 glucans. The overexpression of VEL1 and VEL4 was also motivated by their relationship to the A. nidulans homologues VELB and VOSA which have been found to regulate the synthesis of beta-glucans in connection to fungal cell growth and development.

TABLE 2A
Relevant Homologues to AGS1, MNT1, VEL1, VEL4

| SEQ ID | Homologous | |
|---|---|---|
| NO. | to: | Amino acid sequence |
| 72 | MNT1 | >AFR92851.2 alpha-1,3-mannosyltransferase |
| | | [Cryptococcus neoformans var. |
| | | grubii H99] |
| | | MKPVGSLSLGRKLPSAGSLLVGILVGLLVASLLRRGDSNTETWR |
| | | GSGVNKLRPNPVAGASRLQRSQIMLPSIEQRLHILELISTLSAHH |
| | | TKECLRNPPLYVEQVKERYAPLVGYKKPWSNSGWLGKLFGGKSDS |
| | | HLTMASTTGNDRMDSIKRDLQARQHKYFFAINLYNSFDVIPDIFA |
| | | TLFRAAAILGYHNVFVSIYENGSNDQTKALLKIFDALARTVGLRI |
| | | IIRTSMRTRGLFNHRIEYLAEVRNAAMLPLHELRDNDGEVFDSVV |
| | | FMNDILPCVDDLLELIWQSRRQNAGITCAADYMYHDDIGAPVFYD |
| | | NWVARDINGTALENAPFEQIFHHTESNHRFQRHLPIQVQSCWNGI |
| | | AVLDPTPFYEPPHVKFRMAHLDQGECSASECSLICNDYFNAGYGR |
| | | IIMVPRVKLAYDRKVWDIIHPERRNLTAIRGYKRIGGLPDDPHSD |
| | | PQDRSWYGPHDRLFTPEETEELEFVPGPEYVWCWGWDGAGDLDGP |
| | | DVDPIWEHMQPRSYSEEAIQIKHYRNMPGW |
| 73 | MNT1 | >CDR40896.1 RHTO0S05e08482g1_1 |
| | | [Rhodotorula toruloides] |
| | | MQHYTSPYSAPTAARLRQRGRRQRSSISLLVDREAPRTLLSFARR |
| | | KAGSLASLGIGVLVGLLIASLLGGSQPAAVPWKGSAVVETWTQRQ |
| | | GTHRNLVSLPTLEQRERVFKILSTLTGYHTRYCLRNAQKAYRRQI |
| | | YERYEPLVGYEQHGKRKGLRGHLKAIVPKVARRGFFGGADDEKTE |
| | | VEQIFSHHRDGGSGGGNSLSRSYEGHKYFFAINLYNSFDIIPDLF |
| | | SNMFKVSAILGFQNVFVSVYENGSKDQTKALLRLFDALARSIGLR |
| | | VVIRTSLRTRGAFNHRIEYLAEVRNAALAPLWELRDAEGEVFDSI |
| | | IFMNDVLPCVDDLLELVWQSRRQNAGITCGSDYIFHDEVGQPVFY |
| | | DNWVARDMNGTALENAPFEAVFRDMESSHRFQRHLPVQVQSCWNG |
| | | IAVLDPAPFYAHPHVKFRMAKLSAGECSASECSLICNDYWQAGYG |
| | | RIIMIPRVKLAYDRQVWDIIHPERRNLTYIRGYTRLGGNPDDPHS |
| | | DPQDRSWYGPHDRLFRQEESEAINFRQPPKYVWCWGWDGAGDLEG |
| | | PDVEPVWEQMPNQTIDPIVVRHDRSFTAT |
| 74 | AGS1 | >XP_663489.1 protein agsA |
| | | [Aspergillus nidulans FGSC A4] |
| | | MRWRPLNPLLPLLAATAAGWPYEESLVDYNLNVNKNAATPADYYA |
| | | PEWRNHTYMPSPENWRFPFYTLFLDRFVNGDPTNDNINGTVYEHD |
| | | LNSNQMRHGGDAQGLVDTLDYLQGMGIKGIYLAGTILMNQPWGAD |
| | | GYSILDTTLLDQHFGTIQTWRNAITEIHKRGMYVLFDNTIATMGD |
| | | LIGFKGYLNVSAPFSVKEHEAVWKSDRRYVDFDFGNTYNQTCEYP |
| | | RFWNETGWPVDKDVRDELQGCYSSDFDQYGDREAFGVYPDWQRQL |
| | | AKFASVQDRLREWNPSVRERLIRHSCMIIKALDIDGFRYDKATQA |
| | | TVDALGDMSSAYREITGGNNFGSIYLGRGRQPNQYPDSAMDSMAM |
| | | NNESDHQYFLREDGLQALDSAAFHYSIYRSLTRFLGLDGNLAAGY |
| | | DTPIDWTDAWNVMVMTNDMINANTGKFDPRHMFGATNQDVFRWPA |
| | | IKQGIERQLLAMFITTLHLPGIPILLWGEEQGFYILDATADNYVY |
| | | GRQAMSPATAWKTHGCFQLTADQYHNWPISKGREGCHDETVTYDH |
| | | RDPSHPLRNIIKHMYQLRQDYQVLNDGYSVQKLSNQTRQIFYPGS |
| | | NGTATETGMWSVLRDSVYKIQELHNEQPVWLVYQNDNKTVEYNFD |
| | | CSDNDTALISPFATKTTVVNLFYPHDEYDLKDGPKKLHLNGSAEF |
| | | NGCLDSMTLKPFEFKAFVPKERFVKPRPMITKITPGHDQPIISKV |
| | | VASEAEDLDLSIYFSAEMDCDSVTKAIKVQSTTEVNKTALIDKDS |
| | | VKCRRIDPNETRWTAQLPSVWAWSSKLTGVYNGIHRLTVTNATSE |
| | | VGGSTQAVDHFLIRIGQIDNPMVFTTANYSTDLLHQHENGTLYIR |
| | | HKAAGADKYRYSTNWGSSFSNWREYKGGDEFIEEQPWSGTKKQKW |
| | | NGKHVRVEYWSKLTGSSSYVQEGDYDTKHQRRFPHLFFNGPYNQY |
| | | GYDAGLDNEVKQDSDGYWKYRLRAEFPAQGQFNVWGMNPDGKPDQ |
| | | SFVFGDLDSDGVLDRMPPSSLNTLSINVTDRPPSSYLSWNIWVDD |
| | | GTMSIQFQPTGSRTIQMVVYFLLWFVPLVTAIGCVYAFMKSFYQV |
| | | KFNQIGISQKRSLFGFSVGRKPSLNPLTRLANKSGFLQSTPVFGT |
| | | GSSRRRSVLIATMEYDIEDWGIRIKIGGLGVMAQLMGKNLGHQDL |
| | | IWVVPCAGDVDYPEDQPAEPMFVTVLGNIYEVKVQYHVLNNITYV |
| | | LLDAPVFRQQSKAEPYPARMDDLDSAIYYSAWNQCIAETIKRFPI |
| | | DLYHINDYHGSIAPLYLLPQTIPVCLSLHNAEFQGLWPMRTQKER |
| | | DEVCSVENIDVDVARRYVQFGEVENMLHAGASYLRVHQQGFGAVG |
| | | VSRKYGKRSYARYPIFWGLKKVGNLPNPDPSDTAEWNKELPKESE |
| | | IQVDQNYEASRAELKRQAQEWAGLEQNPNADLMVFVGRWSMQKGI |
| | | DLIADVMPAVLEAHPNVQLICVGPVIDLYGKFAALKLDRMMQLYP |
| | | GRVFSKPEFTALPPYIFSGAEFALIPSRDEPFGLVAVEFGRKGAL |
| | | GIGARVGGLGQMPGWWYNVESTTTAHLLHQFKLAIGCALNSKPQV |
| | | RARMRARSAKQRFPVAQWVEDLEILQSTAMRIHSKGLAKASVQPY |
| | | NSGSNTPLGMMTPPIASTGTVTPTGIQTPPLAHSRSGSYSNINRL |
| | | SAYGPQQRNTIIYSRDPSPGGEDQPRSGIRQLSLGVRAGPGHLMR |
| | | RGRRRLRRNSHAGTDENASVSMTEESSDDDIIPSFYGEEEYTLTP |
| | | EQAEEVRRADMTPQQEQNHGSVRDFFTRRHSSQSSILSRSVLSPA |
| | | SSTTFDGDETFVPPAPPFAEPGNRLSSASVLSVDSVVGEKKDYKL |
| | | QKVDPTFTDSTGEFYKVFERKLEKLNGSNSISQLCIEEYLEKSEK |
| | | KWFDRFRDARLGRKQSPSSSIFRTKFEGSSPMALVSNDEVGSRAS |
| | | GSEPRMRPDEFCLGNDYVPPSGLKKWMQVRIFDWPIYSFILGLGQ |
| | | IIAANSYQITLLTGEVGQRPEKLYGIATVYLVSSIVWWFLFRFCK |
| | | SVVVLSLPWLFYGFAFVLIGVAHYEGDSFARAWIQNVGAGVYAAA |
| | | SASGSLFFALNFGDENGAPVKNWVWRACIIQGTQQAYIIGLWYWG |
| | | TSISQAVTRGVPDVQAHITETWRMTTICMPIAVFLWVLGILVFFG |
| | | LPNYYRQTPGKVPSFYQSVCRRKIILWNFVVVILQNFFLSAPYGR |
| | | NWSFLWSSVHAEPWHIGLLVVAFFGVAWVLILCIFARLSKSHSWI |
| | | LPVFACGLGAPRWAQIWWGVSGMGLFLPWAGSYTTGALVSRSLWL |
| | | WLGILDSLQGLGFGMILLQTLTRMHICFTLLASQVLGSIATICAR |
| | | AFAPNNIGPGPISPDITDGAGAVANAWFWIALFFQLLICSFPIDV |
| | | MS |
| 75 | AGS1 | >XP_660911.1 protein agsB |
| | | [Aspergillus nidulans FGSC A4] |
| | | MGRLQLSSGLKAIALLTFAATATCWPYDESLVDYNVNTNKSATNP |
| | | ADYWGEWSDHKYHPSPENWRFPFYTLFMDRFVNGDPTNDNINGTT |
| | | FEHDLNSNQMRHGGDVAGLVDTLDYLQGMGIKGLYLAGTPLMNQP |
| | | WGSDGYSALDTTLLDQHFGTIQVWRDAITEIHKRGMYVLFDNTIA |
| | | TLGDLIGFEGHLNDTTPFSEKEHKALWKSNRRYVDFDIGNTYNAT |
| | | CDYPRFWYEDGMPVNESLTAGLVGCYDSDFDQYGDIEAFGVWPDW |
| | | KRQLAKFASVQDRLREWYPPVRERLIRHTCMIIASFDIDGIRYDK |
| | | ATQATVDALGDMSKAYRECARAVGKENFFIAGEITGGNTFGSIYL |
| | | GRGRQSNQVDSVGNIYDAMKLTNESDPQLFLREVGHEAIDAGAFH |
| | | YSTYRALTRFLGMDGQLEAGYDVPLDWVQAWGNMTVINDLINANT |
| | | GKFDPRHMYGVTNQDVFRWPAIEWGVERQMLGSFITTLMLPGIPL |
| | | LLWGEEQAFYVLDATASNYIYGRQAMSSATAWKTHGCFSLESSQY |
| | | YQWPLVAALDGCNDETVTYDHRDPSHPVRNIIKHMYQMREQYPML |
| | | NDGFIIETLSNQTEPVYYPGSNGTETETGMWSVRRDRNEETQDFG |
| | | SSDDNEPIWLVYSNMNRTHDYTFDCSDNETALIAAFPSGTKVRNL |
| | | FHPYDTLTLGDGPKEMVYGNSTELVGCLPNLTLSRYEFRAYVKNE |
| | | LWKKPRPMITKFQPGDDEANGHDSPLRSTVAPDASETVRLTLQFS |
| | | EAMGCDSVTDSISFNSSTETGKIPSIDASTVQCGNITEVANSNAT |
| | | GHIPGKWQWAADLRGVYNGIHRVTVNNASNADGDDSTHAVDHFLF |
| | | RIGQIDNPMVFTSANYSSSLLHEKEDGTFYIQHHAAGADKYRYST |
| | | NWGTTFSDWKTYKGGNDTITMLPWNGTKAQEWEGHHIRVEYWSRW |
| | | TGSSSHVQEGDSGWKYKTPRRFPHAFFNGPYNQYGYDGGLDNQIK |
| | | LDAGAGGDGYWKYHFTSEWPAVGQVNVWGINPDGEPDQSWVMGDV |
| | | DGDKVLDRMPPSALSATLINITDHPTHPYISWKLYINDATMRYYL |
| | | IPAGHQSGQIAMFVLFWIIPLLSGSACVYIFMKSFYKVKFNEIGA |
| | | AGASTEMKSLVPLALRRRMKQLASGNGKNGPSFNPLMRLAEKSGF |
| | | MQSTTALAGAASGKRRMVLIATMEYDIEDWGIKIKIGGLGVMAQL |
| | | MGKTLGHQDLIWVVPCVGGVDYPVDTPAEPMTVTILGQAYQVNVQ |
| | | YHVLKNITYVLLDAPVFRQQTKSEPYPARMDDLDSAVYYSAWNQC |
| | | IAEAIKRFPVDLYHINDYHGSVAPLYLLPGTIPACLSLHNAEFQG |
| | | LWPMRTQKEKEEVCSVFNLDVEVVRNYVQFGEVENLLHAGASYLR |
| | | VHQQGFGAVGVSKKYGKRSYARYPIFWGLRKIGNLPNPDPSDVGE |
| | | WTKEDSLIKDEDIKVDPEFEAGRAELKRQAQEWAGLDQNPDADLL |
| | | VFVGRWSMQKGVDLIADVMPAVLEARPNVQLICVGPVIDLYGRFA |
| | | ALKLDRMMKVYPGRVFSRPEFTALPPYIFSGAEFALIPSRDEPFG |
| | | LVAVEFGRKGALGIGARVGGLGQMPGWWYNVESVSTSHLLMQFKL |
| | | AIEAALSSKTETRAMMRARSAKQRFPVAQWVEDLEILQSTAIQVH |
| | | EKEVSRGHAGGRPMTPMTPSGATTPSGMMTPTTGSRGLKPLSQGV |
| | | GMGLSVPHSRESSYSNLNRLSEYVAQKTPGESQPRESSGLQRSLS |
| | | LGVRSGPGHRGRARKQKPGADNIPEGNEDGSSSDTESIPDYYDDE |
| | | YTLTPAQIEESRRAQATRSISFSPETLQPPRSPLPAPPMSPGTPP |
| | | SVEQTLLPPPKPFAAADAGNRLSSASVLSLDSVVGGKKDFKLQKV |
| | | DPFFTDSNGEYARNFEQQLENLNGSNSESQLCIEEFLVKSERRWF |
| | | NKFRDARLGRLRSPTPSVFRDNHSHGRGSPDGSMYVDEAGHRNSG |
| | | DAVHDNGSDDTDDEFLLGKDYVPPTGLKKWMQIKIGDWPVYTLFL |
| | | ALGQIIAANSYQITLLTGEVGQTAEKLYGIATTYAITSALWWLVF |
| | | RYFKSIVCLSTPWFLYGIAFLFIGSAHFESDSFTRGWIQNVGSGF |
| | | YAAASSSGSIFFALNFGDEGGAPVSKWIFRACVIQGIQQVYVIVL |
| | | WYWGSTMAHQSSQGLLTADNTISNTWKMTAICYPIAMLLWAIGLL |
| | | LIFGLPNYYRQKPGKVPSFYKSLFRRKIVLWNFVAVILQNFFLSA |
| | | PYGRNWSFLWTSSHTKPWQIVILCVIFFGLLWCAFLYIVAVLSKQ |
| | | HSWFLPVFACGLGAPRFLQIWWGVSGIGHYLPWVAGGYTGGALVS |
| | | RSIWLWLGVLDSIQGLGFGIILLQTLTRMHMCFTLIVSQVLGSIA |
| | | TIVARACAPNNVGPGPVSPDITKGAGELANAWFWVALFCQLLVCA |
| | | GFLLFFRKEQLAKP |
| 76 | AGS1 | >CDR40896.1 RHTO0S05e08482g1_1 |
| | | [Rhodotorula toruloides] |
| | | MQHYTSPYSAPTAARLRQRGRRQRSSISLLVDREAPRTLLSFARR |
| | | KAGSLASLGIGVLVGLLIASLLGGSQPAAVPWKGSAVVETWTQRQ |
| | | GTHRNLVSLPTLEQRERVFKILSTLTGYHTRYCLRNAQKAYRRQI |
| | | YERYEPLVGYEQHGKRKGLRGHLKAIVPKVARRGFFGGADDEKTE |
| | | VEQIFSHHRDGGSGGGNSLSRSYEGHKYFFAINLYNSFDIIPDLF |
| | | SNMFKVSAILGFQNVFVSVYENGSKDQTKALLRLFDALARSIGLR |
| | | VVIRTSLRTRGAFNHRIEYLAEVRNAALAPLWELRDAEGEVFDSI |
| | | IFMNDVLPCVDDLLELVWQSRRQNAGITCGSDYIFHDEVGQPVFY |
| | | DNWVARDMNGTALENAPFEAVFRDMESSHRFQRHLPVQVQSCWNG |
| | | IAVLDPAPFYAHPHVKFRMAKLSAGECSASECSLICNDYWQAGYG |
| | | RIIMIPRVKLAYDRQVWDIIHPERRNLTYIRGYTRLGGNPDDPHS |
| | | DPQDRSWYGPHDRLFRQEESEAINFRQPPKYVWCWGWDGAGDLEG |
| | | PDVEPVWEQMPNQTIDPIVVRHDRSFTAT |
| 77 | VEL1 | >XP_657967.2 protein velB |
| | | [Aspergillus nidulans FGSC A4] |
| | | MYAVEDRAHSGHHPPPLSMDRIPPPSTMYPSSAGPSAMVSPAGQP |
| | | EPESLSTVHDGRIWSLQVVQQPIRARMCGFGDKDRRPITPPPCIR |
| | | LIVKDAQTQKEVDINSLDSSFYVVMADLWNADGTHEVNLVKHSAT |
| | | SPSISTAMSSSYPPPPHPTSSDYPASYQTNPYGQPVGQPVGQPVG |
| | | YAGVGNYYGGSTQLQYQNAYPNPQAQYYQPMYGGMAQPQMPAAQP |
| | | VTPGPGGMFTRNLIGCLSASAYRLYDTEDKIGVWFVLQDLSVRTE |
| | | GIFRLKFSFVNVGKSVSDLPQSDIAEVINKGTAPILASTFSEPFQ |
| | | VFSAKKFPGVIESTPLSKVFANQGIKIPIRKDGVKGQGSRGRHSD |
| | | EDDGLDNEY |
| 78 | VEL 1 | >ODQ74421.1 hypothetical protein LIPSTDRAFT_ |
| | | 62442 [Lipomyces starkeyi NRRL Y-11557] |
| | | MSSTLSPTFSFPIYPGQQQPPAPVPVPQPQIPSHHAPQQQQQQQL |
| | | QQQHQQQQQQQLPPNMATQLLGQQHTSTAQYGYLHPPPPPPLHHI |
| | | SAPQLYAGQLTPRSSPRLLFDDPSEPHIYQLLVVQQPIRARMCGF |
| | | GDKDRRPLTPPPCIQLLIKDKATGQDADISKLDVSFYALTVELWS |
| | | VDETQNLSLVSTLPLSAASTTQPVHSQFMQQSYHGQVPQPHALQT |
| | | PTFVAPPIKNLIGSVVATAFKLHDTQGKLGVWFILQDLSVRTEGE |
| | | FKLKVSFVNLGSPLSPPSASTQSSSTPAPSTHGNGPHSHAGDNPN |
| | | AQQQPVMVVNTGTARVQATCFSNPFRVYIAKRFPGVIESTELSRC |
| | | FAMQGIKIPIRKDQKKNTEDPQQPQL |
| 79 | VEL1 | >XP_056046225.1 velvet factor-domain- |
| | | containing protein [Lipomyces tetrasporus] |
| | | MSSTLSPTFNFPIYPGQQQQQQQQQPAAAPSSQSQITSHLPQQQQ |
| | | QSPPNMVTQLLGQQHALTAQYGHLPPPPPPPPPLHHISVPQLYAG |
| | | QLTPRSSPGLGVDDPSEPYIYQLRVVQQPIRARMCGFGDKDRRPL |
| | | TPPPCIQLIIKDKVTGQDADISKLDVSFYALTVELWSGDETQNLS |
| | | LVSTLPLSAGSATQSVHSQVMQQSYYGQMPQHHAHQTPTFVAPPI |
| | | KNLIGSVVATAFKLHDTQGKLGVWFILQDLSVRTEGEFKLKASFV |
| | | NLGSPLSPPPASSSSSAPSMHGNGPHSHAGDSATGQQQLPVMVVN |
| | | TGTARVQATCFSAPFRVYIAKRFPGVIESTELSRCFAMQGIKIPI |
| | | RKDQKKNAEEQPQQPQ |
| 80 | VEL1 | >XP_016269978.1 Velvet factor family protein |
| | | [+Rhodotorula toruloides NP11] |
| | | MDEPVSPPTAGPSSRRIYSLTVVQQPVRARMCGFGDKDRRPISPP |
| | | IIVKLSSTDAATGREVEVEGIDPTFLVLAADLRGPNGWDDANLVQ |
| | | QHRSTASRPDRTPSLRASSTTSSRSPSQHSHADTPSPSPTTREAV |
| | | LPNAEHKMSVSRLLSLAEESARPIEADSPAQLAAGLPVLRLSPSQ |
| | | PPPARPVPPQNKRSSPTSSSSSSSKRHRPSPPPSAAFIPYARFNR |
| | | PAYSADSARTLINGSSSEDGETEAALVPNLIGTLHTNAYKLRGLD |
| | | GEKGVYFVLPDLSVRTEGAFRLRLRLVDLRQSMSGANSLSSAAVL |
| | | TDKFQVFTAKRFTGMLDPTSLSQCFAKQGVRIPTRRAAKAKNAVE |
| | | QGDEVKQEEE |
| 81 | VEL4 | >sp\|Q5BBX1.2\|VOSA_EMENI RecName: |
| | | Full = Spore development regulator vosA; |
| | | AltName: Full-Viability of spores protein A |
| | | MSAANYPDPSLPRPSTSDDFELIVRQNPNRARVAGGKEKERKPVD |
| | | PPPIVQIRVREEGTYLAQHYLQSPYFFMSCSLYDAQEDAPASIPP |
| | | STALTGTLVSSLHRLKDVDNTDGGFFVWGDLSIKVEGDFRLKFSL |
| | | FEMRKTDVVFLKSIVSERFTVSPPKSFPGMAESTFLSRSFADQGV |
| | | KLRIRKEPRTLIKRTAPRPEEYPQAAIPRSPSDRTAMQIPGSSYP |
| | | APPYQPTSRDYSYYAPVKRQRTSVDYGARGMYDADGRMRQMETYP |
| | | QTATLYGQPGGYPTPMMGYPSGHGGVPDYAMSYGLPPSAQVPQMQ |
| | | DPAAQSRSSQQATMQSLGMVNPPGTPTPDSARAMMQQAYPRPQYS |
| | | ASTAVLPPLQQSRNYPQGTNGATRGYYEQSPQATPILPSQPLGTS |
| | | EAERYGVPPGHTGYDHTGSANGTPR |
| 82 | VEL4 | >ODQ73683.1 hypothetical protein LIPSTDRAFT_ |
| | | 267564 [Lipomyces starkeyi NRRL Y-11557] |
| | | MTSAAAPDSNATAYPIRPDHVKSDIVTSLPGNASSLYLPASLASV |
| | | AYDRQQRSPILMTSTQQAPSRRLLATSNPNSIGGAMPSMQPQTAT |
| | | PHVCTFKLEVAQHPVQARASGWGSRSDTRRPVDPPPVVNLYVYLD |
| | | GVDVYVMTTYAIGICHRLISHRTSCCKNTFILQATLHAAHPVAPS |
| | | LTSPTTPTTPPQTSNLIGTTITSLNFMKQPPPGRAYFLFPDLSVR |
| | | QEGTYRLVFSVFEICEDSGAVWRAEAVSDVLTVYSPKKFPGLDVS |
| | | SALTREIASQGGKVRIRREARLRGRRPSSGVTAVSFVSVPTRPVT |
| | | ADSKDSLRAPLGSESMISKELLPDGYVLADGKRKLVDPSRIVESP |
| | | MISPLSAPTQQPQHEYSYQSATYAPYTSTTTVTSTAMYYPYHTPS |
| | | AYNIFPPPMNTQQTYLPPPPSSTWAVPRNFSQHVILPHVSQTQGQ |
| | | VPQYSYTSAPQYSMANSIDHAAGQPRFTYVDAMMIPSQQRPPSDY |
| | | STSTLPSSSGSVTSTRSGSTAGTSHHPSMSWPSRATSASAQQLPL |
| | | SMIPGQQAGVPMMQPLPVSMPMQVSVGTQMTMMQQQQQQQPQQQE |
| | | QQQQQQHQHHHQQQHQQQHQQQPQPQPQAQQPQRQQQQHQQPVPP |
| | | MPQHQTGQLGHNIGIVGGQYLYDEGFDE |
| 83 | VEL4 | >XP_056043301.1 velvet factor-domain- |
| | | containing protein [Lipomyces tetrasporus] |
| | | MASAAAPGSSATAEYHVGPDPDISPPSPDIVSSRPVSASLVSAAY |
| | | DRPQLSPILSTSTQSASSKRLLASRNSSSGVMTSIQSQIATPHAC |
| | | TFKLEVAQHPVQARASGWGSRSDTRRPVDPPPVVNLYVYLDGVDV |
| | | TSCCKNTFILQATLHAAHPVAPSLTSPTTPTTPPQTSNLIGTTIT |
| | | SLNFMKQPPPGRAYFLFPDLSVRQEGAYRLVFSVFEICEDVGAVW |
| | | RAEAVSDVLTVYSPKKFPGLDVSSALTREIASQGGKVRIRREARL |
| | | RGRRPSSSVTAVSFGSVPSRPVTADSKDSRLPPFGSESVMSRELL |
| | | SDEHLIADGKRKLVDPSRTVESPMISPLSAPTQQQPQHEYSYQSA |
| | | TYTPYTSTSTTTVASTATYYPYHSSSAYNIFPQPIATQQTYLPPP |
| | | PLSTWAVPRNFSQPQVVLPQVSQTQGQLPQYAYGSTPQYTMASTI |
| | | DQTTGQPRFAYIDGMILPSQQRPPSEYSSSALPSSSSASVSSSIS |
| | | SGSRAGTSQHPSISWQSPATSTSAQQLPMSMIPGQQVAGVPVMQQ |
| | | MPPVVSMPMQVSVGPQITMVQQQPLHPPPHQQQHQQQHQQQHQQQ |
| | | HQHQQPAQPVPQPQTGQLGLNIGSVSGPFLYDEGFDE |
| 84 | VEL4 | >XP_016276764.1 velvet factor family protein |
| | | [Rhodotorula toruloides NP11] |
| | | MAAQAHHAMPPPPDRPPAAAQASPRASMYALDVRQQPRQARMAGS |
| | | GEKADRRPIDPPPIIRLRIRRPSARKKPAHLLTDDDLTTPTLTHT |
| | | LFMFASLVSEDGEEEADEAAECKNALVAGTVVSSLFHLRDESCFV |
| | | FPDLSIRTEGRWRFKMSLYELTEDGVRFCSSVLTDVFQVYSSKRF |
| | | PGMSKSTELSKSFAQQGIKLRVRRPGSRLDDAAPEEPPVGPRREP |
| | | PLIAKAPARRVSATMAAPAPRRPSAPASSAAANEALFDSSARKRP |
| | | WSSFDAANDTRMVRPAPPPPHPHGLHGAAERWRYVPPNPNARRVH |
| | | SESFHPHDYRAHGPPPPAYPHPGVLTAPQGLAFTAPDPRLPPNTH |
| | | PRPFAFTTPAPPRPLDPTRTNAYPAHPHAHAHAHAHVHMYPPPLR |
| | | PVSPPPVLPPLRNVPHPSTRPRALSSSPPKSASRQQQPSPDPSPE |
| | | SDFDAAEALSFMRSGRVESVVRPVPVKPGAGGPPEPEKAEREGSR |
| | | PSTATGGSLAKLLGEGSSTAKGAGEVFFM |

In some embodiments, the microorganisms disclosed herein (e.g., an oleaginous microorganism) may comprise one or more genetic modifications. In some embodiments, the microorganisms disclosed herein (e.g., an oleaginous microorganism) may comprise one or more genetic modifications from Table 1, Table 2, or Table 2A. In some embodiments, the microorganisms disclosed herein (e.g., an oleaginous microorganism) may comprise one or more genetic modifications which are homologues to a protein disclosed in Table 1, Table 2, or Table 2A. In some embodiments, the microorganisms disclosed herein (e.g., an oleaginous microorganism) may comprise one or more genetic modifications which target or catalyze a same reaction as a protein disclosed in Table 1, Table 2, or Table 2A, as defined the Enzyme Commission (EC) number.

Some embodiments provided herein include an oleaginous microorganism (e.g., a diploid microorganism; e.g., C. oleaginosus) that has been genetically modified to underproduce a combination of 2, 3, 4, 5, 6, 7, 8, 9, 10, 11, 12, 13, 14, 15, 16, 17, 18, 19, 20, 21, 22, 23, 24, 25, or 26, 27, 28 of the proteins set forth in Table 1. In some embodiments, the oleaginous microorganism has been genetically modified to overproduce a combination of 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 of the proteins set forth in Table 2. In some embodiments, the oleaginous microorganism has been genetically modified to underproduce a combination of 2, 3, 4, 5, 6, 7, 8, 9, 10, 11, 12, 13, 14, 15, 16, 17, 18, 19, 20, 21, 22, 23, 24, 25, 26, 27, or 28 of the proteins set forth in Table 1 and to overproduce a combination of 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 of the proteins set forth in Table 2.

### Separation

The large-scale commercial success of single cell oils (oils made by fermentation as intracellular triglycerides) may require a significant improvement in their cost of manufacture. In some cases, a primary limitation of current manufacturing is the recovery of the oil (e.g., lipids) from the fermentations. In some cases, at the end of fermentation, reactors contain approximately 80-85% water and 15-20% biomass, and this biomass contains on average 70-90% intracellular oil. In some cases, the recovery of this oil requires several steps, the separation of the cells from the broth, the disruption of the cells to release the oil, and the separation and purification of the oil from the cell debris. One of the major steps is water removal. In some cases, this water removal occurs as a first step in the recovery process. Flocculation, or the process of separating cells by adding coagulants to make the cells form clumps, is often used for water removal. However, flocculation is costly as it requires large volumes of inorganic flocculants and creates unwanted byproducts.

An alternative to flocculation is the use of gravity for cell separation. In some cases, however, since the product of the fermentation are cells filled with oil, the use of gravity for this process may not be possible. In some case, this is because the density of the cells containing high levels of oil may be very close to the density of the broth and do neither settle nor float quantitatively. In some cases, there can be a distribution of cells of different oil content that have a range of density that range from below to above the density of water. In some cases, fermentations can result in extracellular materials that increase the viscosity of the broth. This increase in broth viscosity can further impede cell separations. Accordingly, such samples when exposed to a centrifugal force can result in cells partitioning to the bottom as a pellet, throughout the “water column” and to the top as a floating raft, or simply not separate well at all, and form what appears as a stable colloid. In some cases, by adding different density modulators to the broth (e.g., suspension), the density of the broth can be changed, either above or below that of the oil containing cells, such that it can then be separated from the cells using gravity in the form of settling, sedimentation, decantation, or centrifugation.

As such, described here is the application of different density modulators in the separation of cells from broth. Additionally, described here are methods for altering the characteristics of cell broths (e.g. pH, temperature) to increase cell separation. Also described herein are methods of filtering of the separation of cells from broth. In some cases, the filtering of the separation of cells from broth is enhanced by one or more genetic modification to the microorganism described herein.

### Suspension Contents

In an aspect, the present disclosure provides a method for collecting cells. The method may include providing a suspension. The cells may contain one or more lipids and may be within the suspension. The suspension may include cells of a microorganism. The suspension may further include water. The suspension may also include peptone, yeast extract, one or more sugars, agar, one or more vitamins, one or more nitrogen sources, one or more salts, one or more alkali metals, one or more trace elements, one or more amino acids, and/or one or more carbon sources. The suspension may include one or more alcohols (e.g., ethanol).

The suspension may include one or more nutrients that may remain at the end of fermentation. The suspension may include one or more components of the media that may remain at the end of fermentation. The suspension may include one or more extracellular polymeric substances. The suspension may include exopolysacharides. The suspension may include extracellular polymers.

The suspension may include a carbon source, such as sugars, alcohols, or mixtures thereof. In some cases, the carbon source comprises ethanol and glucose. In some embodiments, the carbon source may also include, for example, starch, ethanol, industrial ethanol, acetic acid, glucose, fructose, sucrose, raffinose, molasses, bagasse, xylose, glycerol, methanol, synthesis gas, carbon dioxide, carbon monoxide, formic acid, cellulose hydrolysates, and industrial, agricultural, food, and municipal organic wastes. In some cases, the carbon source concentration in the suspension may be reduced due to consumption of the carbon source by the oleaginous microorganism.

The suspension may include one or more sugars. The one or more sugars may be a monosaccharide and/or a disaccharide. The sugar may be a 5-carbon sugar. The sugar may be a 6-carbon sugar. The sugar may be a monosaccharide. The sugar may be a disaccharide. The sugar may be a mixture of different sugars. The one or more sugars may be selected from glucose, fructose, dextrose, galactose, lactose, maltose, and sucrose. The sugar may be selected from, for example, glucose, fructose, galactose, sucrose, lactose, maltose, dextrose, maltodextrin, etc. The carbon source may include one or more non-sugar carbon sources. The non-sugar carbon sources may be selected from starch, glycerol, cellulosic biomass, hydrolyzed cellulosic biomass, methane, methanol, syngas, or other suitable carbon sources known in the art, etc. In some cases, the sugar concentration in the suspension may be reduced due to consumption of the sugar by the oleaginous microorganism.

The one or more alcohols may be selected from ethanol and methanol. In some cases, the carbon source comprises acetic acid. In some cases, the carbon source may be a blend of carbon sources. In some cases, the source of the carbon source is from a fossil source and/or a renewable bio-based source. In some embodiments, the alcohol concentration in the suspension may be reduced due to consumption of the alcohol by the oleaginous microorganism.

The suspension may include a nitrogen source, such as yeast extract, ammonium salt, amino acids, or mixtures thereof. The suspension may contain 1, 2, 3, 4, 5, or more different compounds that serve as nitrogen sources. The amino acids may be selected from alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In some embodiments, the nitrogen source in the suspension may be reduced due to consumption of the nitrogen source by the oleaginous microorganism.

The suspension may include one or more salts. The suspension may include one or more metal salts. The one or more salts may be selected from NH₄Cl, KH₂PO₄, Na₂HPO₄ 12H₂O, MgSO4 7H₂O, FeCl₃ 6H₂O, ZnSO₄ 7H₂O, CaCl₂ 2H₂O, MnSO₄ 5H₂O, CuSO₄ 5H₂O, and Co(NO₃)₂ 6H₂O. In some embodiments, the one or more salts in the suspension may be reduced due to consumption of the one or more salts by the oleaginous microorganism.

The suspension may include one or more organic acids. The one or more organic acids may include, for example, carboxylic acids and/or sulfonic acids. The one or more organic acids may be selected from lactic acid, acetic acid, formic acid, citric acid, oxalic acid, uric acid, malic acid, and tartaric acid, etc.

The cells of the microorganism may include one or more lipids. The major lipid components of the microorganism may be triacylglycerols. The triacylglycerols may be composed of, for example, C16 to C18 series long chain fatty acids. The triacylglycerols may be also composed of, for example, C1 to C6 series short chain fatty acids. The triacylglycerols may be also composed of, for example, C8 to C14 series mid chain fatty acids. The triacylglycerols may be also composed of, for example, C18 to C24 series very long chain fatty acids. The triacylglycerols may be as described elsewhere herein. In some embodiments, the lipids may have a particular percentage of a particular fatty acid. The lipid may have a C16:0 fatty acid percentage of at least about 10%, 15%, 20%, 25%, 27%, 30%, 32%, 35%, 40%, 50%, 60%, or more. The lipid may have a C16:0 fatty acid percentage of at most about 60%, 50%, 40%, 35%, 32%, 30%, 27%, 25%, 20%, 15%, 10%, or less. The lipid may have a C16:0 fatty acid percentage from about 10% to 50%, 20% to 50%, 20% to 40%, 20% to 30%, 25% to 40%, 30% to 40%, 35% to 40%, or 28% to 35%.

The lipids may have a C16:1 fatty acid percentage of at least about 0%, 1%, 2%, 3%, 4%, 5%, 6%, 7%, 8%, 9%, 10%, or more. The lipids may have a C16:1 fatty acid percentage of at most about 10%, 9%, 8%, 7%, 6%, 5%, 4%, 3%, 2%, 1%, or less. The lipids may have a negligible percentage of a C16:1 fatty acid. The lipids may have a C16:1 fatty acid percentage from about 0% to 10%, 0% to 9%, 0% to 8%, 0% to 7%, 0% to 6%, or 0% to 5%.

In some embodiments, the lipids may have a C18:0 fatty acid percentage of at least about 5%, 6%, 7%, 8%, 9%, 10%, 11%, 12%, 13%, 14%, 15%, 16%, 17%, 18%, 19%, 20%, 25%, 30%, or more. The lipids may have a C18:0 fatty acid percentage of at most about 30%, 25%, 20%, 19%, 18%, 17%, 16%, 15%, 14%, 13%, 12%, 11%, 10%, 9%, or less. The lipids may have a negligible percentage of a C18:0 fatty acid. The lipids may have a C18:0 fatty acid percentage from about 7% to 25%, 5% to 15%, 5% to 20%, 10% to 20%, 15% to 20%, 8% to 22%, 9% to 21%, or 8% to 12%. In some cases, the lipids may have at least about 15% C18:0 fatty acid.

In some embodiments, the lipids may have a C18:1 fatty acid percentage of at least about 35%, 40%, 42%, 45%, 50%, 55%, 60%, 70%, 80%, 90%, 91%, 92%, 93%, 94%, 95%, or more. The lipids may have a C18:1 fatty acid percentage of at most about 95%, 94%, 93%, 92%, 91%, 90%, 80%, 70%, 60%, 55%, 50%, 45%, 42%, 40%, 35% or less. The lipids may have a C18:1 fatty acid percentage from about 35% to 95%, 35% to 90%, 40% to 80%, 45% to 70%, 35% to 50%, 40% to 45%, 40% to 50%, 37% to 45%, 40% to 48%, or 45% to 50%. In some cases, the lipids may be at least about 35% C18:1 fatty acid. In some cases, the lipids may be at least about 40% C18:1. In some cases, the lipids may be at least about 50%, 60%, 70%, 80%, or 90% C18:1 fatty acid.

The lipids may have a C18:2 fatty acid percentage of at least about 0%, 1%, 2%, 3%, 4%, 5%, 6%, 7%, 8%, 9%, 10%, or more. The lipids may have a C18:2 fatty acid percentage of at most about 10%, 9%, 8%, 7%, 6%, 5%, 4%, 3%, 2%, 1%, or less. The lipids may have a C18:2 fatty acid percentage from about 0% to 10%, 0% to 9%, 0% to 8%, 0% to 7%, 0% to 6%, or 0% to 5%. In some cases, the lipids may be at least about 2% C18:2 fatty acid. In some cases, the lipids may be at least about 5% C18:2 fatty acid.

The lipids may have a C18:3 fatty acid percentage of at least about 0%, 1%, 2%, 3%, 4%, 5%, 6%, 7%, 8%, 9%, 10%, or more. The lipids may have a C18:3 fatty acid percentage of at most about 10%, 9%, 8%, 7%, 6%, 5%, 4%, 3%, 2%, 1%, or less. The lipids may have a negligible percentage of a C18:3 fatty acid. The lipids may have a C18:3 fatty acid percentage from about 0% to 10%, 0% to 9%, 0% to 8%, 0% to 7%, 0% to 6%, or 0% to 5%.

### Density Modulating Agents

The method may include modulating the density of the suspension. The density of the suspension may be modulated by adding a density modulating agent to the suspension. The density modulating agent may increase the density of the suspension. The density of the suspension may become more dense than the density of the cells of the microorganism containing the one or more lipids. The density of the suspension may be increased by adding mass to the suspension.

The density modulating agent may increase the density of the suspension. The density modulating agent may increase the density of the suspension by at least about 5%, 10%, 15%, 20%, 25%, 50%, or more. The density modulating agent may increase the density of the suspension by at most about 50%, 25%, 20%, 15%, 10%, 5%, or less. The density modulating agent may increase the density of the suspension from about 5% to 50%, 5% to 25%, 5% to 20%, 5% to 15%, 5% to 10%, 15% to 50%, 15% to 25%, or 15% to 20%.

The density modulating agent may decrease the density of the suspension. The density modulating agent may decrease the density of the suspension by at least about 5%, 10%, 15%, 20%, 25%, 50%, or more. The density modulating agent may increase the density of the suspension by at most about 50%, 25%, 20%, 15%, 10%, 5%, or less. The density modulating agent may decrease the density of the suspension from about 5% to 50%, 5% to 25%, 5% to 20%, 5% to 15%, 5% to 10%, 15% to 50%, 15% to 25%, or 15% to 20%.

The addition of the density modulating agent to the suspension may decrease the time for gravity to separate the lipid containing cells from the suspension. The addition of the density modulating agent may decrease the time for gravity to separate the lipid containing cells from the suspension by at least about 5%, 10%, 20%, 40%, 50%, 60%, 70%, 80%, or more. The addition of the density modulating agent may decrease the time for gravity to separate the lipid containing cells from the suspension by at most about 80%, 70%, 60%, 50%, 40%, 30%, 20%, 10%, 5%, or less. The addition of the density modulating agent may decrease the time for gravity to separate the lipid containing cells from the suspension by about 5% to 80%, 5% to 50%, 5% to 20%, 10% to 80%, 10% to 70%, 10% to 60%, 10% to 50%, 10%, to 30%, or 10% to 20%.

The addition of the density modulating agent may decrease the time for settling, sedimentation, decantation, or centrifugation to separate the lipid containing cells from the suspension by at least about 5%, 10%, 20%, 40%, 50%, 60%, 70%, 80%, or more. The addition of the density modulating agent may decrease the time for settling, sedimentation, decantation, or centrifugation to separate the lipid containing cells from the suspension by at most about 80%, 70%, 60%, 50%, 40%, 30%, 20%, 10%, 5%, or less. The addition of the density modulating agent may decrease the time for settling, sedimentation, decantation, or centrifugation to separate the lipid containing cells from the suspension by about 5% to 80%, 5% to 50%, 5% to 20%, 10% to 80%, 10% to 70%, 10% to 60%, 10% to 50%, 10%, to 30%, or 10% to 20%.

In some embodiments, the density modulating agent adjusts the density of the solution. The density modulating agent may be a liquid. The density modulating agent may be a solvent. The density modulating agent may be a polar solvent. The density modulating agent may be a solute. The solute may be an acid. The solute may be an alcohol. The solute may be a salt as described elsewhere herein. The solute may be a dissolvable solid. The solute may be selected from formic acid, acetic acid, nitric acid, hydrochloric acid, glycerol, and sulfuric acid. In some cases, the solute consisting of methanol, ethanol, propanol, isopropanol, and butanol.

In some embodiments, the density modulating agent increases the density of the suspension. The density modulating agent may be a liquid. The density modulating agent may be a solvent. The density modulating agent may be a solute. The solute may be an acid. The solute may be an alcohol. The solute may be a salt. The solute may be a dissolvable solid. The solute may be selected from formic acid, acetic acid, nitric acid, hydrochloric acid, glycerol, and sulfuric acid.

In some embodiments, the solute may be a heavy solute. A heavy solute may be a liquid solute that has a greater density than water. The heavy solute may increase the volume of the suspension while increasing the overall density of the solution. The heavy solute may be selected from a group consisting of formic acid, acetic acid, nitric acid, hydrochloric acid, glycerol, and sulfuric acid. The heavy solute may be selected a group consisting of hydrochloric acid, glycerol, and sulfuric acid. The heavy solute may be an acid. The heavy solute may be an alcohol. The heavy solute may be hydrochloric acid. The heavy solute may be glycerol. The heavy solute may be sulfuric acid. In some cases, the sulfuric acid may be about 1 molar (M), 6M, 10M, 12M, or 18M. In some cases, the sulfuric acid may be from about 1 M to 10M, 6M to 10M, 1M to 18M, or 12M to 18M. In some cases, the hydrochloric acid may be about 1M, 6M, or 12M. In some cases, the hydrochloric acid may be from about 1M to 12M, 1M to 6M, or 0.1M to 1M.

In some embodiments, the density modulating agent is a salt. In some cases, the salt increases the density of the suspension. The salt may be selected from a group consisting of nitrate salts, ammonium salts, chloride salts, sulphate salts, carbonate salts, acetate salts, sodium salts, potassium salts, phosphate salts, and calcium salts. The salt may be selected from a group consisting of sodium chloride, calcium chloride, potassium nitrate, sodium carbonate, sodium acetate, potassium cyanide, zinc chloride hydroxide, sodium hydroxide, potassium chlorate, calcium phosphate, sodium bisulfate, copper sulfate, magnesium sulfate, and potassium permanganate. The salt may be selected from a group consisting of aluminum sulfate, ferric chloride, calcium chloride, and sodium chloride. The salt may be selected from a group consisting of sodium chloride and calcium chloride. In some embodiments, one, two, three, or more salts may eb used, and the salt may be selected from a group consisting of nitrate salts, ammonium salts, chloride salts, sulphate salts, carbonate salts, acetate salts, sodium salts, potassium salts, phosphate salts, sodium chloride, calcium chloride, potassium nitrate, sodium carbonate, sodium acetate, potassium cyanide, zinc chloride hydroxide, sodium hydroxide, potassium chlorate, calcium phosphate, sodium bisulfate, copper sulfate, magnesium sulfate, and potassium permanganate, aluminum sulfate, ferric chloride, or combinations thereof.

In some embodiments, the density modulating agent decreases the density of the suspension. The density modulating agent may be a liquid. The density modulating agent may be a solvent. The density modulating agent may be a solute. The density modulating agent may be an acid. The density modulating agent may be an alcohol.

In some embodiments, the solute may be a light solute. A light solute may be a liquid solute that has a density less than water. The light solute may increase the volume of the suspension while decreasing the overall density of the solution. The density modulating agent may be a light solute. The light solute may be selected a group consisting of methanol, ethanol, propanol, isopropanol, and butanol. The light solute may be methanol. The light solute may be ethanol. The light solute may be propanol. The light solute may be isopropanol. The light solute may be butanol.

In some embodiments, the density modulating agent increases the total volume of the suspension. The density modulating agent may increase the total volume of the suspension by at least about 5%, 10%, 15%, 20%, 25%, 50%, or more. The density modulating agent may increase the total volume of the suspension by at most about 50%, 25%, 20%, 15%, 10%, 5%, or less. The density modulating agent may increase the total volume of the suspension from about 5% to 50%, 5% to 25%, 5% to 20%, 5% to 15%, 5% to 10%, 15% to 50%, 15% to 25%, or 15% to 20%.

In some embodiments, the density modulating agent increases the total mass of the suspension. The density modulating agent may increase the total mass of the suspension by at least about 5%, 10%, 15%, 20%, 25%, 50%, or more. The density modulating agent may increase the total mass of the suspension by at most about 50%, 25%, 20%, 15%, 10%, 5%, or less. The density modulating agent may increase the total mass of the suspension from about 5% to 50%, 5% to 25%, 5% to 20%, 5% to 15%, 5% to 10%, 15% to 50%, 15% to 25%, or 15% to 20%.

In some embodiments, the adding of the density modulating agent to the suspension directs the microbial cells to a top portion of the suspension. In some cases, the top portion may be a distinct separate layer from the suspension. In some cases, adding the density modulating agent to the suspension directs the microbial cells to a bottom portion of the suspension. In some cases, the bottom portion may be a distinct separate layer from the suspension.

In some embodiments, adding the density modulating agent to the suspension demulsifies the suspension. In some cases, the adding of the density modulating agent to the suspension may generate one or more distinct layers (e.g., two total layers, three total layers).

### Oleaginous Microorganisms

The microorganism may be an oleaginous microorganism. The oleaginous microorganism may generate one or more lipids. In some cases, the average weight percentage (%) of lipids to dry cell weight of the oleaginous microorganism culture may be at least about 20%, 30%, 40%, 50%, 60%, 70%, 80%, 90%, or more. In some cases, the average weight percentage (%) of lipids to dry cell weight of the oleaginous microorganism culture may be at most about 90%, 80%, 70%, 60%, 50%, 40%, 30%, 20%, or less. In some cases, average weight percentage (%) of lipids to dry cell weight of the oleaginous microorganism culture may be from about 20% to 90%, 40% to 90%, 50% to 90%, 60% to 90%, 70% to 90%, 80% to 90%, 40% to 80%, 50% to 80%, 60% to 80%, 70% to 80%, 40% to 70%, 50% to 70%, or 60% to 70%.

The oleaginous microorganism culture may be a mixture of cells having different percent lipid content. The oleaginous microorganism culture may contain a mixture of cells that have an average lipid content of at least about 30%, 40%, 50%, 60%, 70%, 80%, or 90% of dry cell weight and contain a distribution of cells having different lipid contents. The oleaginous microorganism culture may contain a mixture of cells that have an average lipid content of at most about 90%, 80%, 70%, 60%, 50%, 40%, or 30% of dry cell weight and contain a distribution of cells having different lipid contents. The oleaginous microorganism culture may contain a mixture of cells that have an average lipid content from about 30% to 90%, 30% to 80%, 30% to 70%, 30% to 60%, 30% to 50%, 40% to 90%, 40% to 80%, 40% to 70%, 50% to 90%, 50% to 80%, 50% to 70%, or 50% to 60% of dry cell weight and contain a distribution of cells having different lipid contents.

The distribution of cells may contain at least about 30%, 40%, 50%, 60%, 70%, 80%, 90%, or more lipid content. The distribution of cells may contain at most about 30%, 40%, 50%, 60%, 70%, 80%, 90%, or more lipid content. The distribution of cells may contain from about 30% to 90%, 30% to 80%, 30% to 70%, 30% to 60%, 30% to 50%, 40% to 90%, 40% to 80%, 40% to 70%, 50% to 90%, 50% to 80%, 50% to 70%, or 50% to 60%.

The distribution of lipid content of the mixture of cells may have a difference of at least about 1%, 2%, 3%, 4%, 5%, 6%, 7%, 8%, 9%, 10%, 20%, 30%, 40%, 50%, 60%, 70%, 80%, 90%, or more. The distribution of lipid content of the mixture of cells may have a difference of at most about 90%, 80%, 70%, 60%, 50%, 40%, 30%, 20%, 10%, 9%, 85, 7%, 6%, 5%, 4%, 3%, 2%, 1%, or less. The distribution of lipid content of the mixture of cells may have a difference from about 1% to 90%, 1% to 50%, 1% to 30%, 1% to 20%, 1% to 10%, 1% to 5%, 5% to 90%, 5% to 50%, 5% to 20%, or 5% to 10%. In some cases, the distribution of lipid content of the mixture of cells may be substantially negligible.

In some embodiments, the oleaginous microorganism comprises greater than 50 weight percent (%) of lipids to oleaginous microorganism. In some cases, the oleaginous microorganism comprises greater than about 20%, 30%, 40%, 50%, 60%, 70%, 80%, or 90% of lipids to oleaginous microorganism. In some cases, the oleaginous microorganism comprises less than about 90%, 80%, 70%, 60%, 50%, 40%, 30%, or 20% of lipids to oleaginous microorganism. In some cases, the oleaginous microorganism comprises from about 20% to 90%, 30% to 90%, 40% to 90%, 50% to 90%, 60% to 90%, 70% to 90%, 80% to 90%, 20% to 70%, 30% to 70%, 40% to 70%, 50% to 70%, or 60% to 70%. In some cases, the lipid content may be determined using the Bligh-Dyer method.

In some embodiments, cells of the microorganism may be intact cell walls. In some cases, the cell walls may be substantially intact. In some cases, at least about 60%, 70%, 80%, 90%, 99% of the cells have intact cell walls. In some cases, at most about 99%, 90%, 80%, 70%, 60% or less of the cells have intact cell walls. In some cases, from about 60% to 99% 60% to 90%, 60% to 80%, 60% to 70%, 70% to 99% 70% to 90%, 70% to 80%, 80% to 99%, or 80% to 90% of the cells have intact cell walls.

In some embodiments, the microorganism may be for example, yeast. In some cases, the microorganism is an oleaginous microorganism. The oleaginous microorganism may be yeast. In some cases, the microorganism comprises yeast, bacterial, fungal, or algal cells. The yeast can be used to synthesize lipids.

The lipid may be generated from an oleaginous microorganism that may come from Agaricomycotina, Ascomycota, Basidiomycota, Candida, Chlorellales, Chlorellaceae, Cryptococcus, Cuniculitremaceae, Debaryomycetaceae, Filobasidiales, Incertae sedis, Lipomyces, Metschnikowiaceae, Pichiaceae, Rhodosporidium, Rhodotorula, Rhizpus, Saccharaomycotina, Saccharomyceses, Saccharomycesales, Tremellomycetes, Trichomonoascaceae, Trichosporon, Trichosporonales, Viridiplantae, or Yarrowia, etc. The yeast may be, for example, Cutaneotrichosporon oleaginous, Lypomyces starkeyi, Rhodotorula toluroides, Rhodosporidium toruloides, Lipomyces starkeyi, Lipomyces lipofer, Lipomyces arxii, Lipomyces doorenjongii, Lipomyces oligophage, Lipomyces spencer-martinsiae, Lipomyces knonenkoae, Lypomyces tetrasporus, Lipomyces anomalus, Lipomyces japonicus, Lipomyces kockii, Lipomyces kononenkode, Lipomyces mesembrius, Rhodosporidium sp., Rhodotorula sp., Yarrowia sp., Yarrowia lipolytica, Cryptococcus sp., Cryptococcus aerius, Lipomyces sp., Candida curvata, Candida aff. insectorum, Candia aff. sagamina, Candida aff. kazuoi, Rhodotorula glutinis, Rhodotorula graminis, Rhodotula 110, Rhodotorula aurantiaca, Leucosporidiella creatinivora, Rhodotorula colostri, Rhodotorula dairenensis, Rhodotorula mucilaginosa, Rhodosporidium babjevae, Rhodosporidium diobovatum, Rhodosporidium fluviale, Rhodosporidium paludigenum, Rhodosporidium sphaerocarpum, Rhodosporidium toruloides, Cryptococcus podzolicus, Trichosporon porosum, Trichosporon guehoae, Pichia segobiensis, Trichosporonoides spathulata, Kodamaea ohmeri, Cryptococcus sp., Cryptococcus music, Lipomyces tetrasporus, Lipomyces sp, Myxozyma geophila, Myxozyma lipomycoides, Myxozyma mucilagina, Myxozyma udenii, Myxozyma vanderwaltii, Myxozyma cf. melibiosi, Myxozyma melibiosis, Torulaspora delbrueckii, Trigonopsis varaibilis, Cutaneotrichosporon oleaginosus, Cutaneosporon oleaginosus, Scheffersomyces stipitis, Kurtzmaniella cleridarum, Pichia manshurica, Cuniculitrema polymorpha, Filobasidium floriforme, Filobasidium globisporum, Filobasidium aff. globisporum, Filobasidium inconspicuum, Cryptococcus albidus, Cryptococcus gastricus, Cryptococcus magnus, Cryptococcus oeirensis, Cryptococcus terreus, Cryptococcus aff. taibaiensis, Cryptococcus flavescens, Cryptococcus aff. laurentii, Cryptococcus luteolus, Cryptococcus victoriae, Cryptococcus cf. curvatus, Cryptococcus humicola, Cryptococcus ramirezgomezianus, Cryptococcus wieringae, Filobasidium cf. uniguttulatus, Hannaella aff. zeae, Tremella aurantia, Tremella enchepala, Prototheca aff. zopfii, Prototheca stagnora, Prototheca aff. zopfii var. hydrocarbonea, Prototheca zopfii var. zopfii, ATCC 20509, or Metschnikowia pulcherrim, etc. In some cases, the yeast is selected from a group consisting of Cutaneosporon oleaginosus, Lypomyces starkeyi, Lipomyces tetrasporus, Rhodotorula toluroides, and Yarrowia Lypolytica. In some cases, the yeast may be a recombinant yeast. In other cases, the yeast may be a chemically or physically induced mutant of a natural or recombinant yeast. The lipid may be extracted from the yeast upon production.

### Lipid Compositions

The lipids harvested from the systems and methods described herein may include one or more fatty acids. The one or more fatty acids may include a polyunsaturated fatty acid content. An unsaturated fatty acid is a fatty acid that contains a carbon-double bond, and that double bond can be in the cis configuration or the trans configuration. A polyunsaturated fatty acid is a fatty acid containing more than one carbon-carbon double bonds. The polyunsaturated fatty acid content may be a percentage relative to the total TAG content of the lipids. The polyunsaturated fatty acids (PUFA) content may be less than about 1.0%, 2.0%, 3.0%, 4.0%, 5.0%, 6.0%, or 10.0%. The PUFA content may be more than about 0.1%, 0.5%, 1.0%, 1.5%, 2.0%, 2.5%, or 3.0%. The PUFA content may be from about 0.1% to 10.0%, 0.1% to 5.0%, 0.5% to 3.0%, or 1.0% to 2.0%. The PUFA content may be less than 2.0%. The PUFA content may be less than 5.0%. The PUFA content may be less than 3.0%. In some embodiments, the PUFA content may be below a detection limit or considered negligible. In some cases, the percentage may be relative to a total weight percent. In some cases, there may be no PUFA.

The one or more fatty acids may include a monounsaturated fatty acids (MUFA) content. The MUFA content may be a percentage relative to the total TAG content of the lipids. The MUFA content may be more than about 40%, 50%, 60%, 70%, 80%, 90% or 95%. The MUFA content may be less than about 95%, 90%, 80%, 70%, 60%, or 55%. The MUFA content may be from about 40% to 95%, 50% to 95%, 50% to 75%, 50% to 55%, 40% to 95%, 50% to 95%, 50% to 75%, 50% to 55%. The MUFA content may be more than about 50%. The MUFA content may be from about 50% to 95%. In some embodiments, the MUFA content may be below a detection limit or considered negligible. In some cases, the percentage may be relative to a total weight percent.

The one or more fatty acids may include an omega-6 fatty acids content. The omega-6 fatty acids content may be a percentage relative to the total TAG content of the lipids. The omega-6 fatty acids content may be present at up to 10% by weight. The lipids may comprise omega-6 fatty acids at less than about 9.5%, 9.0%, 8.5%, 8.0%, 7.5%, 7.0%, 6.5%, 6.0%, 5.5%, 5.0%, 4.5%, 4.0%, 3.5%, 3.0%, 2.5%, 2.0%, 1.5%, 1.0%, 0.9%, 0.8%, 0.7%, 0.6%, 0.5%, 0.4%, 0.3%, 0.2%, or about 0.1% by weight. The lipids may comprise omega-6 fatty acids at up to 0.1%, 0.2%, 0.3%, 0.4%, 0.5%, 0.6%, 0.7%, 0.8%, 0.9%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0% 8.5%, 9.0%, 9.5% or at most about 10.0% by weight. The omega-6 fatty acid content may be from about 0.2% to 2.0%, 0.2% to 1.0%, 0.2% to 0.5%, or 0.5% to 1.5%. In some embodiments, the omega-6 fatty acids content may be below a detection limit or considered negligible. In some cases, there may be no omega-6 fatty acids content.

The one or more fatty acids may include an oleic acid content. The oleic acid content may be a percentage relative to the total TAG content of the lipids. The oleic acid content may be more than about 40%, 50%, 60%, 70%, 80%, 90% or 95%. The oleic acid content may be less than about 95%, 90%, 80%, 70%, 60%, or 55%. The oleic acid content may be from about 40% to 95%, 50% to 95%, 50% to 75%, 50% to 55%, 40% to 95%, 50% to 95%, 50% to 75%, or 50% to 55%. The oleic acid content may be more than about 50%. The oleic acid content may be from about 50% to 95%. The oleic acid content may be at least about 30%, 40%, 50%, 60%, 70%, 80%, 90%, or more of the total TAG content lipids. The oleic acid content may be at most about 90%, 80%, 70%, 60%, 50%, 40%, 30%, or less of the total TAG content of lipids. In some embodiments, the oleic acid content may be below a detection limit or considered negligible. In some cases, the percentage may be relative to a total weight percent.

The one or more fatty acids may include a linoleic acid content. The linoleic acid content may be a percentage relative to the total TAG content of the lipids. The linoleic acid content may be at least about 1%, 5%, 10%, or more of the total TAG content. The linoleic acid content may be at most about 10%, 5%, 1%, or less of the total TAG content. The linoleic acid content may be from about 1% to 10%, 1% to 5%, or 5% to 10% of the total TAG content.

The one or more lipids may also comprise an oxidative stability index (OSI) which may be used to determine the relative oxidative stability of the fatty materials within the lipids. The oxidative stability of the lipids may be determined using the American Lipids Chemical Society (AOCS) standard method CD 12B-92 and/or the Rancimat method. In some embodiments, the oxidative stability of the lipids may be measured isothermally at elevated temperatures to accelerate oxidation. In some cases, the OSI is the point of maximum change of the rate of oxidation. In some cases, the OSI is a method that may determine the relative resistance of fats or lipids to oxidation from the lipids.

The lipids may have an OSI value. The lipids OSI value may be determined without the use of external antioxidants in the lipids. The lipids may have an OSI value of greater than about 30 hrs, 40 hrs, 50 hrs, 60 hrs, 70 hrs, 80 hrs, 90 hrs, 100 hrs, 150 hrs or more when measured at 110 degrees Celsius. The lipids may have an OSI value of less than about 150 hrs, 100 hrs, 90 hrs, 80 hrs, 70 hrs, 60 hrs, 50 hrs, or less when measure at 110 degrees Celsius. The lipids may have an OSI value from about 30 hrs to 200 hrs, 50 hrs to 200 hrs, 50 hrs to 150 hrs, or 50 hrs to 100 hrs when measure at 110 degrees Celsius.

### Microorganism Removal

The method may also include removing the microorganism from the suspension. Removing may include performing one or more of pipetting, decanting, filtering, or centrifugating.

Removing may include pipetting of the cells from the suspension. Removing may include decanting of the cells from the suspension. Removing may include filtering of the cells from the suspension. Removing may include centrifugating of the cells from the suspension. The centrifugation of the cells from the suspension may employ a bucket centrifuge, a discstack centrifuge, a decantor, or other unit operation that use gravity and density differences to affect separation.

In another aspect, the present disclosure provides a method for collecting an oil from an oil-producing microorganism. The method may include providing a suspension. The suspension may include cells of a microorganism comprising one or more lipids. The suspension may be as described elsewhere herein. In some cases, the method may also include modulating the density of the suspension by adding a density modulating agent to the suspension. In some cases, the method may also include removing the microorganism from the suspension. In some cases, the method may also include the separating of the microorganism from the suspension using gravity, a centrifuge, or a decanter. In some cases, the method may include lysing the cells of the microorganism to release the one or more lipids. In some cases, the method may also include collecting the one or more lipids.

In some cases, the culture broth may be centrifuged. In some cases, the culture broth may be centrifuged at about 1,000 rcf to about 15,000 rcf. In some cases, the culture broth may be centrifuged at about 1,000 rcf to about 3,000 rcf, about 1,000 rcf to about 4,000 rcf, about 1,000 rcf to about 4,350 rcf, about 1,000 rcf to about 5,000 rcf, about 1,000 rcf to about 7,000 rcf, about 1,000 rcf to about 10,000 rcf, about 1,000 rcf to about 12,000 rcf, about 1,000 rcf to about 15,000 rcf, about 3,000 rcf to about 4,000 rcf, about 3,000 rcf to about 4,350 rcf, about 3,000 rcf to about 5,000 rcf, about 3,000 rcf to about 7,000 rcf, about 3,000 rcf to about 10,000 rcf, about 3,000 rcf to about 12,000 rcf, about 3,000 rcf to about 15,000 rcf, about 4,000 rcf to about 4,350 rcf, about 4,000 rcf to about 5,000 rcf, about 4,000 rcf to about 7,000 rcf, about 4,000 rcf to about 10,000 rcf, about 4,000 rcf to about 12,000 rcf, about 4,000 rcf to about 15,000 rcf, about 4,350 rcf to about 5,000 rcf, about 4,350 rcf to about 7,000 rcf, about 4,350 rcf to about 10,000 rcf, about 4,350 rcf to about 12,000 rcf, about 4,350 rcf to about 15,000 rcf, about 5,000 rcf to about 7,000 rcf, about 5,000 rcf to about 10,000 rcf, about 5,000 rcf to about 12,000 rcf, about 5,000 rcf to about 15,000 rcf, about 7,000 rcf to about 10,000 rcf, about 7,000 rcf to about 12,000 rcf, about 7,000 rcf to about 15,000 rcf, about 10,000 rcf to about 12,000 rcf, about 10,000 rcf to about 15,000 rcf, or about 12,000 rcf to about 15,000 rcf. In some cases, the culture broth may be centrifuged at about 1,000 rcf, about 3,000 rcf, about 4,000 rcf, about 4,350 rcf, about 5,000 rcf, about 7,000 rcf, about 10,000 rcf, about 12,000 rcf, or about 15,000 rcf. In some cases, the culture broth may be centrifuged at at least about 1,000 rcf, about 3,000 rcf, about 4,000 rcf, about 4,350 rcf, about 5,000 rcf, about 7,000 rcf, about 10,000 rcf, or about 12,000 rcf. In some cases, the culture broth may be centrifuged at at most about 3,000 rcf, about 4,000 rcf, about 4,350 rcf, about 5,000 rcf, about 7,000 rcf, about 10,000 rcf, about 12,000 rcf, or about 15,000 rcf.

In some cases, the culture broth may be centrifuged for about 1 minute to about 60 minutes. In some cases, the culture broth may be centrifuged for about 1 minute to about 5 minutes, about 1 minute to about 7 minutes, about 1 minute to about 10 minutes, about 1 minute to about 20 minutes, about 1 minute to about 30 minutes, about 1 minute to about 60 minutes, about 5 minutes to about 7 minutes, about 5 minutes to about 10 minutes, about 5 minutes to about 20 minutes, about 5 minutes to about 30 minutes, about 5 minutes to about 60 minutes, about 7 minutes to about 10 minutes, about 7 minutes to about 20 minutes, about 7 minutes to about 30 minutes, about 7 minutes to about 60 minutes, about 10 minutes to about 20 minutes, about 10 minutes to about 30 minutes, about 10 minutes to about 60 minutes, about 20 minutes to about 30 minutes, about 20 minutes to about 60 minutes, or about 30 minutes to about 60 minutes. In some cases, the culture broth may be centrifuged for about 1 minute, about 5 minutes, about 7 minutes, about 10 minutes, about 20 minutes, about 30 minutes, or about 60 minutes. In some cases, the culture broth may be centrifuged for at least about 1 minute, about 5 minutes, about 7 minutes, about 10 minutes, about 20 minutes, or about 30 minutes. In some cases, the culture broth may be centrifuged for at most about 5 minutes, about 7 minutes, about 10 minutes, about 20 minutes, about 30 minutes, or about 60 minutes.

In some cases, the pH of the culture broth may be adjusted before centrifugation. In some cases, the pH of the culture broth may be about 1 to about 8. In some cases, the pH of the culture broth may be about 1 to about 1.5, about 1 to about 1.9, about 1 to about 2, about 1 to about 2.1, about 1 to about 2.5, about 1 to about 3, about 1 to about 4, about 1 to about 5, about 1 to about 6, about 1 to about 7, about 1 to about 8, about 1.5 to about 1.9, about 1.5 to about 2, about 1.5 to about 2.1, about 1.5 to about 2.5, about 1.5 to about 3, about 1.5 to about 4, about 1.5 to about 5, about 1.5 to about 6, about 1.5 to about 7, about 1.5 to about 8, about 1.9 to about 2, about 1.9 to about 2.1, about 1.9 to about 2.5, about 1.9 to about 3, about 1.9 to about 4, about 1.9 to about 5, about 1.9 to about 6, about 1.9 to about 7, about 1.9 to about 8, about 2 to about 2.1, about 2 to about 2.5, about 2 to about 3, about 2 to about 4, about 2 to about 5, about 2 to about 6, about 2 to about 7, about 2 to about 8, about 2.1 to about 2.5, about 2.1 to about 3, about 2.1 to about 4, about 2.1 to about 5, about 2.1 to about 6, about 2.1 to about 7, about 2.1 to about 8, about 2.5 to about 3, about 2.5 to about 4, about 2.5 to about 5, about 2.5 to about 6, about 2.5 to about 7, about 2.5 to about 8, about 3 to about 4, about 3 to about 5, about 3 to about 6, about 3 to about 7, about 3 to about 8, about 4 to about 5, about 4 to about 6, about 4 to about 7, about 4 to about 8, about 5 to about 6, about 5 to about 7, about 5 to about 8, about 6 to about 7, about 6 to about 8, or about 7 to about 8. In some cases, the pH of the culture broth may be about 1, about 1.5, about 1.9, about 2, about 2.1, about 2.5, about 3, about 4, about 5, about 6, about 7, or about 8. In some cases, the pH of the culture broth may be at least about 1, about 1.5, about 1.9, about 2, about 2.1, about 2.5, about 3, about 4, about 5, about 6, or about 7. In some cases, the pH of the culture broth may be at most about 1.5, about 1.9, about 2, about 2.1, about 2.5, about 3, about 4, about 5, about 6, about 7, or about 8.

In some cases, the temperature of the culture broth may be adjusted before centrifugation. In some cases, the temperature of the culture broth may be about 10° C. to about 80° C. In some cases, the temperature of the culture broth may be about 10° C. to about 20° C., about 10° C. to about 30° C., about 10° C. to about 40° C., about 10° C. to about 50° C., about 10° C. to about 60° C., about 10° C. to about 70° C., about 10° C. to about 80° C., about 20° C. to about 30° C., about 20° C. to about 40° C., about 20° C. to about 50° C., about 20° C. to about 60° C., about 20° C. to about 70° C., about 20° C. to about 80° C., about 30° C. to about 40° C., about 30° C. to about 50° C., about 30° C. to about 60° C., about 30° C. to about 70° C., about 30° C. to about 80° C., about 40° C. to about 50° C., about 40° C. to about 60° C., about 40° C. to about 70° C., about 40° C. to about 80° C., about 50° C. to about 60° C., about 50° C. to about 70° C., about 50° C. to about 80° C., about 60° C. to about 70° C., about 60° C. to about 80° C., or about 70° C. to about 80° C. In some cases, the temperature of the culture broth may be about 10° C., about 20° C., about 30° C., about 40° C., about 50° C., about 60° C., about 70° C., or about 80° C. In some cases, the temperature of the culture broth may be at least about 10° C., about 20° C., about 30° C., about 40° C., about 50° C., about 60° C., or about 70° C. In some cases, the temperature of the culture broth may be at most about 20° C., about 30° C., about 40° C., about 50° C., about 60° C., about 70° C., or about 80° C.

In some cases, the length of time between addition of acid or change in temperature and centrifugation may be varied. In some cases, the length of time may be about 0.1 minutes to about 60 minutes. In some cases, the length of time may be about 0.1 minutes to about 0.5 minutes, about 0.1 minutes to about 1 minute, about 0.1 minutes to about 5 minutes, about 0.1 minutes to about 15 minutes, about 0.1 minutes to about 60 minutes, about 0.5 minutes to about 1 minute, about 0.5 minutes to about 5 minutes, about 0.5 minutes to about 15 minutes, about 0.5 minutes to about 60 minutes, about 1 minute to about 5 minutes, about 1 minute to about 15 minutes, about 1 minute to about 60 minutes, about 5 minutes to about 15 minutes, about 5 minutes to about 60 minutes, or about 15 minutes to about 60 minutes. In some cases, the length of time may be about 0.1 minutes, about 0.5 minutes, about 1 minute, about 5 minutes, about 15 minutes, or about 60 minutes. In some cases, the length of time may be at least about 0.1 minutes, about 0.5 minutes, about 1 minute, about 5 minutes, or about 15 minutes. In some cases, the length of time may be at most about 0.5 minutes, about 1 minute, about 5 minutes, about 15 minutes, or about 60 minutes. In some cases, the length of time may be about 1 hour to about 72 hours. In some cases, the length of time may be about 1 hour to about 2 hours, about 1 hour to about 4 hours, about 1 hour to about 8 hours, about 1 hour to about 16 hours, about 1 hour to about 24 hours, about 1 hour to about 48 hours, about 1 hour to about 72 hours, about 2 hours to about 4 hours, about 2 hours to about 8 hours, about 2 hours to about 16 hours, about 2 hours to about 24 hours, about 2 hours to about 48 hours, about 2 hours to about 72 hours, about 4 hours to about 8 hours, about 4 hours to about 16 hours, about 4 hours to about 24 hours, about 4 hours to about 48 hours, about 4 hours to about 72 hours, about 8 hours to about 16 hours, about 8 hours to about 24 hours, about 8 hours to about 48 hours, about 8 hours to about 72 hours, about 16 hours to about 24 hours, about 16 hours to about 48 hours, about 16 hours to about 72 hours, about 24 hours to about 48 hours, about 24 hours to about 72 hours, or about 48 hours to about 72 hours. In some cases, the length of time may be about 1 hour, about 2 hours, about 4 hours, about 8 hours, about 16 hours, about 24 hours, about 48 hours, or about 72 hours. In some cases, the length of time may be at least about 1 hour, about 2 hours, about 4 hours, about 8 hours, about 16 hours, about 24 hours, or about 48 hours. In some cases, the length of time may be at most about 2 hours, about 4 hours, about 8 hours, about 16 hours, about 24 hours, about 48 hours, or about 72 hours.

### EXAMPLES

### Example 1: Genetic Tools for Stable Integration of Transformed DNA

In general, oleaginous yeasts are recalcitrant to genetic modification. Tools have been described related to the transformation and overexpression (OE) of heterologous genes in C. oleaginosus. Methods described for introducing exogenous DNA include Agrobacterium tumefaciens mediated transformation (AtMT) and electroporation. While not described; DNA may be introduced via other common methods such as biolistics. Genome sequences have been published for two isolates of C. oleaginosus. The existence of these resources facilitates metabolic understanding and enables the further development of genetic tools.

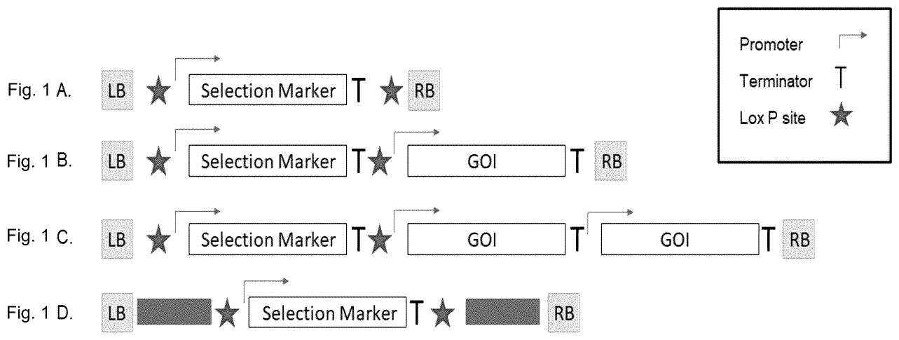

DNA introduced into C. oleaginosus may be linear or circular (plasmids); the introduced linear DNA may be referred to as a cassette and may include configurations such as those described in FIG. 1.

While C. oleaginosus is not known to contain naturally occurring plasmids, and CEN/ARS (centromere/autonomously replicating sequence) are not described, it does appear to transiently replicate both circular and linear DNA. This phenomenon of transient extrachromosomal DNA maintenance has also been described in other organisms such as the basidiomycete yeast, Cryptococcus neoformans (Lin et al 2020). In C. neoformans researchers also found that biolistic transformation resulted in a higher percentage of DNA integrating into the genome (Toffaletti et al 1993). To select for presence of the transformed DNA, the cassette must include a gene encoding for a selectable marker or a means of interfering with production or activity of a counterselectable protein already produced by the organism. Linear DNA may be made through direct linearization of a circular DNA such as may be done with a restriction enzyme, or by amplifying DNA using PCR, or other methods known in the art.

In some examples it may be beneficial to remove the selectable marker by various means known in the art. Examples of methods used for selectable marker removal, often referred to as marker recycling, include but are not limited to the Cre-LoxP system; Piggy-Bac Sytem; FLP-recombinase; Short Repeat Sequences; and targeting of selectable markers for mutation via CRISPR.

Plasmids were constructed containing a selectable marker cassette conferring resistance to the aminoglycoside antibiotics G418 or Neomycin (G418R or NEOR) (FIG. 1A). DNA containing these selectable marker cassettes were either transformed via AtMT or electroporated into C. oleaginosus as a) a circular plasmid, b) a plasmid linearized via restriction endonuclease cutting interior to the RB and LB (FIG. 1A), or c) a DNA cassette containing the selectable marker amplified from the plasmid via PCR interior to the RB and LB (FIG. 1A). Stably maintained transformation events could be readily obtained from all of the tested conditions, presumably as a consequence of the transformed DNA having ectopically integrated into the genome.

### Example 2: Determination of the Diploid Nature of C. oleaginosus

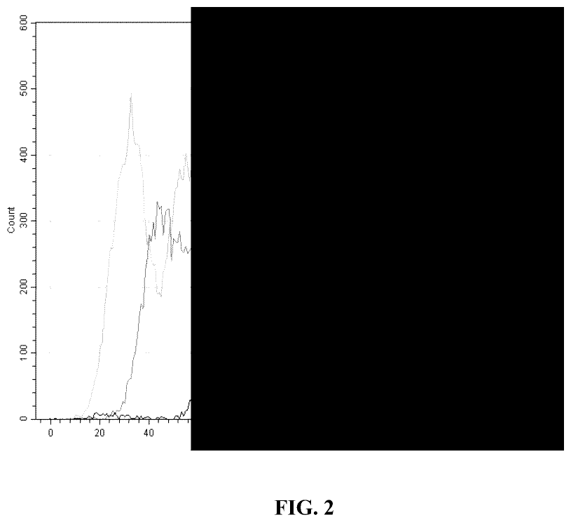

It is expected that DNA transformed into C. oleaginosus is randomly integrated into the genome, typically at a double stranded break; this is common amongst fungi and most other organisms possessing non-homologous end joining (NHEJ) machinery. Hi-Tail PCR (Liu and Chen 2007) was used to characterize the genomic location of ectopically integrated DNA cassettes, such as those described in Example 1. Using this method, the genome sequence flanking either side of the randomly integrated DNA cassette could be amplified and sequenced, and its location in the genome determined. However, this method surprisingly identified that the resulting strains had two copies of the sequence into which the vector inserted, one with the insertion and one wild-type sequence that did not contain the insertion. This suggested that the strain of C. oleaginous in use could be diploid or have multiple nuclei. This was especially surprising as C. oleaginosus is described in the literature as haploid (Kourist et al 2015). To test these possibilities, the strain of C. oleaginosus used at Zero Acre was stained with propidium iodide, a fluorescent DNA dye that is commonly used to stain nuclei, and observed using fluorescent microscopy to evaluate the number of nuclei in each cell. Under these growth conditions, only mono-nucleated cells were observed. The same C. oleaginosus cells were evaluated by flow cytometry, which is commonly used to characterize genome size and predict ploidy (Stovicek et al 2015). To this end, the genomes of the isolate of C. oleaginosus used here (published genome size of 19 Mb), a stable haploid of Saccharomyces cerevisiae (genome 12 Mb), and a stable diploid of S. cerevisiae (genome 24 Mb) were stained with propidium iodide and evaluated by flow cytometry. As shown in FIG. 2 (black=C.o. (19 Mb); Gray=S. cerevisiae haploid (12 Mb); Dark Gray=S. cerevisiae diploid) the graypeaks show propidium iodide fluorescence for haploid S. cerevisiae and should correspond to a 12 Mb and 24 Mb genome; where 24 Mb is the result of actively dividing cells; the dark gray diploid S. cerevisiae should correspond to a 24 Mb diploid and 48 Mb tetraploid (actively dividing) and the black corresponds to C.o. As the smallest blackpeak for C.o. is larger than the 24 Mb S. cerevisiae peak we conclude that it corresponds to a diploid of size ˜38 Mb and that the larger peak being larger than the 48 Mb S. cerevisiae peak corresponds to ˜76 Mb. The flow cytometry results revealed the average fluorescence of C. oleaginosus exceeds the fluorescence of both S. cerevisiae haploid (12 Mb) and diploid (2× 12 mb) strain populations, suggesting that the predicted 19 Mb (oleaginosus is stably maintained as a diploid containing 2 copies of the 19 Mb genome.

### Example 3: Identification of Potential Delta-12 Desaturases in C. oleaginosus

C. oleaginosus produces approximately 5-10% linoleic acid in its TAG when grown under high lipid producing conditions. Since linoleic acid is linked to chronic disease, oil instability, and poor cooking performance, removing linoleic acid from the TAG produced by C. oleaginosus was desirable. The enzyme delta-12 desaturase has been shown to catalyze the conversion of oleic acid (C18:1) to linoleic acid (C18:2) and is believed to provide the key activity needed to produce linoleic acid in many oleaginous yeast. Low linoleic acid producing oleaginous yeasts have been engineered through creation of a delta-12 desaturase mutation in Yarrowia lipolytica (Tezaki et al 2017; cited and followed up on by Tsakraklides et al 2018; Konzock et al 2022) and in Rhodosporidium (Liu et al 2021; Cui et al 2016). In neither case, did these yeasts produce high levels of TAG, and in both cases these yeasts are known to be haploids. A bioinformatic search of the C. oleaginosus published genomes identified a few candidate genes having homology to previously annotated delta-12 desaturases, two of which were most similar, and are referred to here as FAD2a (Crycu|339079) and FAD2b (Crycu|377415). These genes had never been characterized, and it was unclear if either, neither, or both of the genes were functional or linked to linoleic acid production in C. oleaginosus.

### Example 4: Methods for Producing and Screening Lipids Produced by C. oleaginosus

Small scale fermentations are performed in 96-well plates where cultures are initially seeded in a pre-seed media consisting of 0.5 ml YPD prior to transfer to a nitrogen-limited production media in a volume of 0.5 ml, typically for 48 h. Fermentations are performed at 30° C. with 1000 rpm on a 3 mm throw orbital shaker. The small-scale fermentation endpoint samples are used for lipid determination.

3 L bioreactors are inoculated from a YPD seed culture grown at 28-31° C. The Bioreactor is charged with a defined minimal medium comprising trace elements, vitamins, carbon, phosphate, and sulfate typical for published defined media for oil production in C. oleaginosus that has a limiting concentration of nitrogen and the fermentation is executed in fed-batch mode with glucose as a carbon source. The reactor is maintained at a temperature between 28-34C, and at a pH between 4 and 6. The glucose feed is triggered by a spike in dissolved oxygen which is maintained at 20% when glucose is present. The fermentation is continued for sufficient time to fill the bioreactor and/or until no additional glucose is being consumed. Samples are collected at various time points for lipid determination.

To determine lipid composition TAG/FFA are liberated from whole cells using 6 M hydrochloric acid (HCl) prior to extraction with butyl acetate and methanol. TAG/FFA extracted into the butyl acetate layer is transesterified into FAME with acidified methanol prior to analysis on GC-FID/MS. The most abundant FAME species observed in Cutaneotrichosporon oleaginosus are methyl palmitate (C16:0), methyl stearate (C18:0), methyl oleate (C18:1), and methyl linoleate (C18:2). Additional species that are analyzed are methyl myristate (C14:0), methyl pentadecanotate (C15:0), methyl palmitoleate (C16:1), and methyl linolenate (C18:2), methyl arachidate (C20:0), methyl gadoleate (C20:1), methyl behenate (C22:0), and methyl lignocerate (C24:0). Methyl pentadecanoate is not expected to appear in the samples. Methyl heptadecanoate (C17:0) is used as an internal standard for the transesterification reaction. Methyl undecanoate (C11:0) is used as the internal standard for injection. Quantification of these compounds is carried out using a seven point calibration curve based on FAME standards prepared at concentrations ranging from 7.5 mg/L to 3000 mg/L. TAG may also be quantified directly using direct extraction with MTBE prior to analysis of specified fragment ions on a triple-Quadrupole LCMS.

### Example 5: Generation of a KO Mutation of a Delta-12 Desaturase in C. oleaginosus

The genetic manipulation of strains for study or specific commercial improvement requires the ability to both increase the expression of specific genes as well as to decrease or eliminate the expression of specific genes. To prevent the biosynthesis of linoleic acid and other PUFAs, the elimination of the putative delta-12 desaturase activity was believed to be required, which could be accomplished by genetically knocking out (KO) gene(s) that encode this activity. However, there has been no description in the literature of the successful targeted KO of a gene from the oleaginous microorganism C. oleaginosus.

In many organisms, including oleaginous yeasts, DNA repair occurs via the endogenous non-homologous end joining (NHEJ) pathway, and homology directed repair (homologous recombination HR) occurs at a very low frequency if at all. As such, integration of DNA sequences will often occur in a non-targeted manner. This makes targeted gene KO and other site-specific integrations difficult. Conveyed here is a method providing the ability to KO genes in C. oleaginosus. Some common examples of KO could include full gene replacement; insertion/deletion (INDEL) that prevents a functional genetic element; and the introduction of inactivating nucleotide polymorphisms.

In an attempt to integrate DNA via homologous recombination in C. oleaginosus an antibiotic selectable marker, for example G418R or NEOR, is flanked with homologous sequence (0.1-2 kb) (FIG. 1D). The homologous sequence was designed such that it would integrate at a desired target region. To generate a KO for FAD2a or FAD2b DNA cassettes were constructed with G418R or NEOR with 0.5-1 kb of homology targeting FAD2a or FAD2b. In this example, the homology regions were immediately upstream and downstream of the gene of interest such that successful homologous recombination would result in a gene replacement. These cassettes were amplified by PCR and transformed into C. oleaginosus via electroporation, as this method was shown to reliably produce transformants with DNA integrated into the genome (see Example 1). The resultant colonies were screened by PCR for detection of the successful KO i.e. producing a PCR generated band of a molecular weight consistent with the successful integration of the KO cassette at the FAD2A locus. Approximately 5% of colonies screened produced a PCR band consistent with the KO, and expectedly, given the determination of C. oleaginosus as a diploid, these strains also produced a PCR band consistent with a functional copy of the FAD2A gene. This suggested that the FAD2A KO cassette had successfully replaced one of the two copies of FAD2A in the diploid genome. This is the first demonstration of targeting the genome of C. oleaginosus via homologous recombination and the only known example of a heterozygous deletion in an oleaginous microbe. This was confirmed by the sequencing of the KO and WT PCR bands. This approach was then successfully repeated with a cassette targeting the FAD2b delta-12-desaturase candidate gene. This resulted in a heterozygous FAD2b KO, which had G418R replacing a single copy of the FAD2B gene. This further demonstrated the effectiveness of this first of a kind homologous recombination directed KO of genes in C. oleaginosus and can be expected to be broadly applicable to other genetic loci and diploid oleaginous microorganisms.

### Example 6. Evaluation of the Oil Composition of Heterozygous Genetic Knockouts of FAD2a and FAD2b

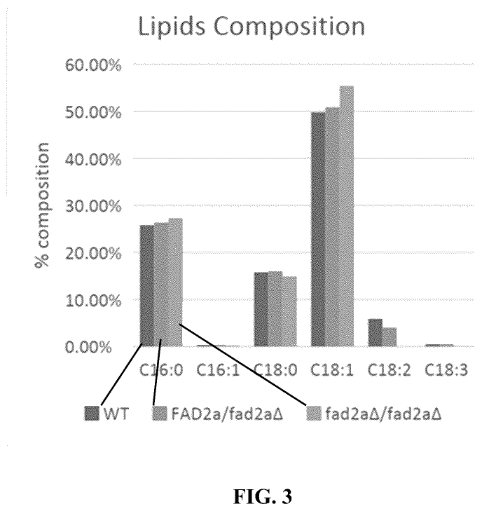

To evaluate the link between the putative delta 12-desaturase genes FAD2a and FAD2b on the production of linoleic acid, the heterozygous KO strains in each of these genes were grown and evaluated for their production of TAG and its content of linoleic acid and other PUFAs. This evaluation was carried out as described in Example 4. As shown in FIG. 3, the heterozygous FAD2a KO strain produced approximately 4% C18:2 down from 5.9% in the WT, whereas the heterozygous FAD2b KO strain was not changed in composition from the WT control (5.9% C18:2). This result suggested that the FAD2a gene did indeed encode a delta-12 desaturase and that it was responsible for the generation of at least some of the linoleic acid produced by WT C. oleaginosus. If so, a homozygous deletion of FAD2a should further decrease the amount of linoleic acid and PUFA relative to the WT and heterozygous FAD2a KO strains.

### Example 7: Generation of a Homozygous Knock Out of FAD2A

To generate a homozygous FAD2a KO strain the fad2aΔ::loxP/FAD2a strain was transformed with a PCR amplified cassette targeting FAD2a and processed using methods similar to those described in examples 5 and 6. This resulted in identified and sequence verified strains having the genotype fad2aΔ::loxP/fad2a::loxP. Newly generated homozygous FAD2a KO were evaluated for TAG and lipid composition, as described in example 4. As shown in FIG. 3, the homozygous FAD2a deletion produced approximately 0.01% C18:2, down from 5.9% C18:2 in a WT and 4% C18:2 in the heterozygous mutant. This is the first example of gene targeting in C. oleaginosus to create a tailored lipid composition; using heterozygous and homozygous gene KO. The remaining 0.01% LA is believed to result from other desaturases present in the cell; these possibilities include the FAD2b and the putative delta-9 desaturase (Crycu|348634).

The method of successful targeted gene manipulation in C. oleaginosus has been made possible as a consequence of combining functional genetic parts (promoters, terminators, selectable markers) with the knowledge that PCR amplified parts reliably integrate into the genome; and the determination that the C. oleaginosus strain used here is a diploid and thus must have both copies of any gene inactivated to be fully depleted of activity.

Table 3 provides the oil compositions in strains with FAD2A knockouts (heterozygous and homozygous) as compared to the wild-type strains. Table 4 provides the relative TAG titer as compared to WT. TAG titer in the WT strain was about 85.73 g/L, 79.4 g/L in the heterozygous KO and 81.60 g/L in the homozygous KO strains.

TABLE 3
TAG compositions (% of composition)

| | | Heterozygous | Homozygous |
|---|---|---|---|
| | WT | FAD2a KO | FAD2a KO |
| TAG_C14:0 | 0.44 | 0.61 | 0.44 |
| TAG_C16:0 | 24.74 | 26.31 | 26.92 |
| TAG_C16:1 | 0.40 | 0.34 | 0.78 |
| TAG_C18:0 | 15.85 | 15.99 | 14.65 |
| TAG_C18:1 | 50.31 | 50.83 | 55.71 |
| TAG_C18:2 | 5.84 | 4.02 | 0.04 |
| TAG_C18:3 | 0.33 | 0.47 | 0.04 |
| TAG_C20:0 | 0.61 | 0.62 | 0.53 |
| TAG_C20:1 | 0.44 | 0.01 | 0.38 |
| TAG_C22:0 | 0.32 | 0.04 | 0.25 |
| TAG_C24:0 | 0.73 | 0.75 | 0.25 |

TABLE 4
Relative TAG compositions

| | Heterozygous FAD2a | Homozygous |
|---|---|---|
| | KO (% relative to | FAD2aKO (% |
| | WT) | relative to WT) |
| TAG_C14:0 | 138.6 | 100.0 |
| TAG_C16:0 | 106.3 | 108.8 |
| TAG_C16:1 | 85.0 | 195.0 |
| TAG_C18:0 | 100.9 | 92.4 |
| TAG_C18:1 | 101.0 | 110.7 |
| TAG_C18:2 | 68.8 | 0.7 |
| TAG_C18:3 | 142.4 | 12.1 |
| TAG_C20:0 | 101.6 | 86.9 |
| TAG_C20:1 | 2.3 | 86.4 |
| TAG_C22:0 | 12.5 | 78.1 |
| TAG_C24:0 | 102.7 | 34.2 |

### Example 8: Increasing C18:1 Content in C. oleaginosus

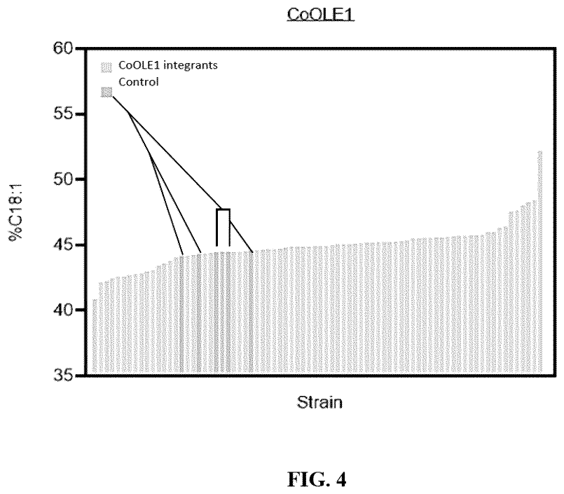

Increased expression of a stearoyl-CoA desaturase (SCD) such as OLE1 from C. oleaginosus is expected to increase C18:1 content. SCD overexpression (Tsakraklides et al 2018, Stephanopoulos et al 2018) for high oleic content in Yarrowia has been demonstrated. A DNA cassette based on that depicted in FIG. 1B where OLE1 is the GOI and G418R is the selectable marker was constructed. This cassette was transformed into C. oleaginosus via electroporation and strains containing stable ectopically integrated copies of the OLE1 overexpression cassette were isolated and sequence verified by High Tail PCR. In some cases, the cassette was concatenated (usually end to end) with one or more additional copies at the site of ectopic integration. Strains containing the ectopic integration were evaluated in a small-scale fermentation as described in Example 4 and as shown in FIG. 4 produced a range of C18:1 levels, suggesting the OLE1 overexpression cassette was functional and was being differentially expressed based on where in the genome the cassette ectopically integrated. The C18:1 content of a WT C. oleaginosus is typically around 50% when grown in controlled bioreactors. However, several of the isolated strains having the OLE1 overexpression cassette ectopically integrated produced almost 60% C18:1 under the same conditions. This is the first example of the specific alteration of the TAG composition of C. oleaginosus by the overexpression of a heterologous gene.

Table 5 provides the oil compositions in strains with ectopic OLE1 overexpression as compared to the wild-type strains. Table 6 provides the relative TAG titers as compared to WT. TAG titer in the WT strain was about 85.73 g/L and about 74.9 g/L in the OLE1 overexpressing strain.

TABLE 5
TAG compositions with OLE1 overexpression
(OE) (% composition)

| | WT | OLE1 OE |
|---|---|---|
| TAG_C14:0 | 0.44 | 0.00 |
| TAG_C16:0 | 24.74 | 23.00 |
| TAG_C16:1 | 0.40 | 1.00 |
| TAG_C18:0 | 15.85 | 11.00 |
| TAG_C18:1 | 50.31 | 58.00 |
| TAG_C18:2 | 5.84 | 5.00 |
| TAG_C18:3 | 0.33 | 0.00 |
| TAG_C20:0 | 0.61 | 1.00 |
| TAG_C20:1 | 0.44 | 0.00 |
| TAG_C22:0 | 0.32 | 0.00 |
| TAG_C24:0 | 0.73 | 1.00 |

TABLE 6
Relative TAG compositions

| | OLE1 OE |
|---|---|
| | (% relative |
| | to WT) |
| TAG_C14:0 | 0.0 |
| TAG_C16:0 | 93.0 |
| TAG_C16:1 | 250.0 |
| TAG_C18:0 | 69.4 |
| TAG_C18:1 | 115.3 |
| TAG_C18:2 | 85.6 |
| TAG_C18:3 | 0.0 |
| TAG_C20:0 | 163.9 |
| TAG_C20:1 | 0.0 |
| TAG_C22:0 | 0.0 |
| TAG_C24:0 | 137.0 |

### Example 9. Further Improvements in Oil Yield, Specific Productivity, Titer, Composition, Fermentation Viscosity, and/or Susceptibility to Lysis

Applying the genetic tools described in the previous examples, further improvements in the oil yield, specific productivity, titer, composition, fermentation viscosity and/or susceptibility to lysis are achieved by manipulating activity of the proteins described in tables 1 and 2. In this example, genes encoding proteins listed in tables 1 and 2 are individually or in combination selectively attenuated or overexpressed, and the resulting genetically modified strains when grown under oil producing conditions demonstrate significant improvement(s) in oil yield, specific productivity, titer, composition, fermentation viscosity and/or susceptibility to lysis cell lysis. As a non-limiting example, a C. oleaginosus strain is genetically modified to increase the expression XPK, PTA, and/or ME. The resulting genetically modified cells are grown in 3 L bioreactors as described in the Examples above, and analyzed for oil production as described in the Examples above. In comparison to cultures without these genetic modifications, the genetically modified cultures show a significant improvement in the oil yield, specific productivity, titer, composition, fermentation viscosity, and/or susceptibility to lysis.

### Example 10: Generation of a Homozygous Knock Out of MNT1

A predicted alpha 1,3 mannosyl transferase, MNT1 (Seq ID NO: 27), was targeted for knockout in C. oleaginosus. Alpha 1,3 mannosyl transferase is a Golgi mannosyltransferase, and is a type of glycosyltransferase that acts upon mannose to promote the formulation of polysaccharides from mannose. C. oleaginousis fermentation broths are highly viscous, such that viscosity impeded commercially viable fermentation and downstream oil recovery. We hypothesized that this viscosity could be a result of extracellular polysaccharide, and that these may arise from surprisingly unexpected sugar transferases encoded in the genome. One of these was MNT1. MNT1 (Seq ID 27), was accordingly targeted for knockout in C. oleaginosus to determine whether its deletion might reduce fermentation broth viscosity and improve production conditions.

A strain with a heterozygous KO mutation of MNT1 (Seq ID NO: 27) was created using methods similar to those described in Example 5. In an attempt to integrate DNA via homologous recombination in C. oleaginosus, an antibiotic selectable marker, for example G418R or NEOR, is flanked with a homologous sequence (0.1-2 kb) (FIG. 1D). The homologous sequence was designed such that it would integrate at a desired target region. To generate a KO for MNT1, a DNA cassette was constructed with G418R or NEOR with 0.5-1 kb of homology targeting MNT1. In this example, the homology regions were immediately upstream and downstream of the gene of interest such that successful homologous recombination would result in a gene replacement. These cassettes were amplified by PCR and transformed into C. oleaginosus via electroporation, as this method was shown to reliably produce transformants with DNA integrated into the genome (see Example 1). The resultant colonies were screened by PCR for detection of the successful KO, i.e. producing a PCR generated band of a molecular weight consistent with the successful integration of the KO cassette at the MNT1 locus. More than 5% of colonies screened produced a PCR band consistent with the KO, and expectedly, given the determination of C. oleaginosus as a diploid, these strains also produced a PCR band consistent with a functional copy of the MNT1 gene. This suggested that the MNT1 KO cassette had successfully replaced one of the two copies of MNT1 in the diploid genome. This was confirmed by the sequencing of the KO and WT PCR bands. This further demonstrated that homologous recombination directed KO of genes in C. oleaginosus is broadly applicable to other genetic loci and diploid oleaginous microorganisms.

A homozygous MNT1 KO strain was created using methods similar to those described in Example 7. To generate a homozygous MNT1 KO strain, the mnt1Δ::loxP/MNT1 containing strain was transformed with a PCR amplified cassette targeting MNT1 and processed using methods similar to those described in Examples 5 and 6. This resulted in identified and sequence verified strains having the genotype mnt1Δ::loxP/mnt1::loxP. The method of successful targeted gene manipulation in C. oleaginosus has been made possible as a consequence of combining functional genetic parts (promoters, terminators, selectable markers) with the knowledge that PCR amplified parts reliably integrate into the genome; and the determination that the C. oleaginosus strain used here is a diploid and thus must have both copies of any gene inactivated to be fully depleted of activity.

### Example 11: Generation of a Homozygous Knock Out of MNT1 and FAD2a

Strains that are optimized for a combination of parameters including specific productivity, composition and viscosity can be generated by sequentially targeting a combination of multiple genes that affect each parameter. For example, a strain with a FAD2a KO and a MNT1 KO could have both eliminated linoleic acid production and decreased viscosity. Accordingly, a strain with a homozygous MNT1 KO and a homozygous FAD2a KO was also created by transforming a homozygous FAD2a KO strain with PCR amplified cassettes targeting MNT1. This resulted in a sequence verified strains having the genotype fad2aΔ mnt1Δ::loxP/fad2aΔ mnt1::loxP. The resultant homozygous MNT1 KO and FAD2a KO strain was evaluated in fed-batch bioreactors with a limiting nitrogen content of 3.2 g/L after 43.3 hours. The resulting fermentation broth viscosity was 8 cP, a 94% reduction relative to WT, indicating that MNT1 can be targeted to reduce fermentation broth viscosity. As expected deletion of FAD2a alone does not alter fermentation broth viscosity nor would deletion of MNT1 be expected to alter linoleic acid production.

### Example 12: Generation of a Homozygous Knock Out of AGS1

An alpha 1, 3 glucan synthase, ags1 (Seq ID 28), was targeted for knockout in C. oleaginosus. Alpha 1, 3 glucan synthase is a glucosyltransferase involved in the production of alpha glucans which are polysaccharides found in the cell wall. C. oleaginousis fermentation broths are highly viscous, such that viscosity impeded commercially viable fermentation and downstream oil recovery. We hypothesized that this viscosity could be a result of extracellular polysaccharide, and that these may arise from surprisingly unexpected sugar transferases encoded in the genome. One of these was AGS1. C. oleaginousis fermentation broths are highly viscous, such that viscosity impeded commercially viable fermentation and downstream oil recovery. We hypothesized that this viscosity could be a result of extracellular polysaccharide, and that these may arise from surprisingly unexpected sugar transferases encoded in the genome. One of these was MNT1. AGS1 (Seq ID 28) was targeted for knockout in C. oleaginosus to evaluate whether its reduced activity would reduce fermentation broth viscosity and improve production conditions.

A strain with a heterozygous KO mutation of AGS1 was created using methods similar to those described in Example 5. In an attempt to integrate DNA via homologous recombination in C. oleaginosus an antibiotic selectable marker, for example G418R or NEOR, is flanked with a homologous sequence (0.1-2 kb) (FIG. 1D). The homologous sequence was designed such that it would integrate at a desired target region. To generate a KO for AGS1, a DNA cassette was constructed with G418R or NEOR with 0.5-1 kb of homology targeting AGS1. In this example, the homology regions were immediately upstream and downstream of the gene of interest such that successful homologous recombination would result in a gene replacement. These cassettes were amplified by PCR and transformed into C. oleaginosus via electroporation, as this method was shown to reliably produce transformants with DNA integrated into the genome (see Example 1). The resultant colonies were screened by PCR for detection of the successful KO i.e. producing a PCR generated band of a molecular weight consistent with the successful integration of the KO cassette at the AGS1 locus. More than 5% of colonies screened produced a PCR band consistent with the KO, and expectedly, given the determination of C. oleaginosus as a diploid, these strains also produced a PCR band consistent with a functional copy of the AGS1 gene. This suggested that the AGS1 KO cassette had successfully replaced one of the two copies of AGS1 in the diploid genome. This was confirmed by the sequencing of the KO and WT PCR bands. This further demonstrated that homologous recombination directed KO of genes in C. oleaginosus is broadly applicable to other genetic loci and diploid oleaginous microorganisms. AGS1 (Seq ID 28), was targeted for knockout in C. oleaginosus to reduce fermentation broth viscosity and improve production conditions.

A homozygous AGS1 KO strain was created using methods similar to those described in example 7. To generate a homozygous AGS1 KO strain, the ags14: loxP/AGS1 containing strain was transformed with a PCR amplified cassette targeting AGS1 and processed using methods similar to those described in examples 5 and 6. This resulted in identified and sequence verified strains having the genotype ags14: loxP/ags14: loxP. The method of successful targeted gene manipulation in C. oleaginosus has been made possible as a consequence of combining functional genetic parts (promoters, terminators, selectable markers) with the knowledge that PCR amplified parts reliably integrate into the genome; and the determination that the C. oleaginosus strain used here is a diploid and thus must have both copies of any gene inactivated to be fully depleted of activity.

### Example 13: Generation of C. oleaginosus Strain with Velvet Domain Protein Overexpression

Two different velvet domain containing proteins, VEL1 and VEL4 were tested for their impact on C. oleaginosus strain performance when overexpressed. Velvet-like proteins contain a distinct protein domain consisting of around 150 amino acids; these regulatory proteins are known to play a role in fungal development and have been implicated in regulation of glucan synthesis. The modification of genes, as demonstrated by overexpression of two different velvet proteins, acting on the synthesis of and the downstream metabolites of UDP-glucose, UDP-mannose, UDP-galactose, as well as other nucleotide sugars we believed could alter culture broth viscosity. This notion is consistent and in agreement with the viscosity reduction observed with knockout of MNT1 or AGS1. VEL1 (Seq ID 59) and VEL4 (Seq ID 58) were targeted for overexpression in C. oleaginosus and were evaluated for their impact on fermentation broth viscosity and ability to improve production conditions.

A strain that overexpresses VEL1 (Seq ID 59) which encodes a velvet domain containing protein was created using methods similar to those described in example 8. A DNA cassette based on that depicted in FIG. 1B where VEL1 is the GOI and G418R is the selectable marker was constructed. This cassette was transformed into C. oleaginosus via electroporation and strains containing stable integrated copies of the VEL1 overexpression cassette were isolated and sequence verified. In some cases, the cassette was concatenated (usually end to end) with one or more additional copies at the site of integration. A strain that overexpresses VEL4 (Seq ID 58) which encodes a velvet domain containing protein was also created using the same method as the strain that overexpresses VEL1.

The VEL1 and VEL4 overexpressing strains were evaluated in fed-batch bioreactors with a limiting nitrogen content of 3.2 g/L after 42 hours. The VEL1 overexpressing strain resulted in a fermentation broth of 43 cP, a 68% reduction in fermentation broth viscosity. The reduction in fermentation broth viscosity suggests that the VEL1 overexpression cassette was functional. The VEL4 overexpressing strain resulted in a fermentation broth of 44 cP, a 68% reduction relative to the unmodified control strain. A VEL4 overexpressing strain was also evaluated for its specific productivity relative to the unmodified strain and the VEL4 overexpressing strain had a specific productivity of 66.2 mg/g/hr after 65 hours, a 9.2% improvement relative to WT. The reduction in fermentation broth and increase in specific productivity suggest that the VEL4 overexpression cassette was functional and provided a significant improvement to fermentation performance. This further indicates that VEL1 and VEL4 overexpression can be targeted for reducing fermentation broth viscosity and improving specific performance.

### Example 14: Generation of C. oleaginosus Strain with Overexpression of a Phosphoketolase from Clostridium acetylbutylicum

A heterologous phosphoketolase from Clostridium acetylbutylicum, XPK_Ca (Seq ID No: 40) was selected for overexpression in C. oleaginosus, which already has a predicted native phosphoketolase. Phosphoketolase is a unique enzyme that may produce Acetyl-P and glyceraldehyde 3-phosphate when using Xylulose 5-phosphate as a substrate; it may also act on other sugar phosphates and in all cases produces an Acetyl-P and another sugar phosphate that is two carbons less than the substrate. Acetyl-P may be converted to cytosolic acetyl-CoA either through the activity of a phosphotransacetylase or the conversion of Acetyl-P to acetate and then activation of the acetate into acetyl-CoA by acetyl COA synthetase. In addition to increased cytosolic acetyl-CoA, phosphoketolase may also reduce oxygen consumption as less NADH may be produced as compared to a process that strictly adheres to sugar metabolism via glycolysis and the pentose phosphate pathway. XPK_Ca (Seq ID No: 40) which encodes phosphoketolase was targeted for overexpression in C. oleaginosus to evaluate whether such overexpression may improve the specific productivity of the modified strain.

A strain that overexpresses phosphoketolase was created using methods similar to those described in example 8 and was then evaluated in fed-batch bioreactors. A DNA cassette based on that depicted in FIG. 1B where XPK_Ca is the GOI and G418R is the selectable marker was constructed. This cassette was transformed into C. oleaginosus via electroporation and strains containing stable integrated copies of the XPK_Ca overexpression cassette were isolated and sequence verified. In some cases, the cassette was concatenated (usually end to end) with one or more additional copies at the site of ectopic integration. The increase in phosphoketolase activity in C. oleaginosus resulted in specific productivity of 69.2 mg/g/hr after 65 hours, a 14.1% improvement relative to WT. The increase in specific productivity suggests that the XPK_Ca overexpression cassette was functional and results in modified strains that are improved in their fermentation performance. This further indicates that phosphoketolase overexpression can be targeted for improving specific performance of the C. oleaginosus organism and increasing commercial lipid production process performance.

### Example 15: Generation of C. oleaginosus Strain with Overexpression of a Malic Enzyme

A Malic Enzyme from C. oleaginosus, ME2 (Seq ID No: 37), was selected for overexpression in C. oleaginosus. Malic enzyme is thought to potentially play a role in the production of cytosolic NADPH for lipid biosynthesis. It is believed that the overexpression of malic enzyme may increase cytosolic NADPH supply as it converts more malate to pyruvate. ME2 (Seq ID No: 37) which encodes the putative C. oleaginosus malic enzyme was targeted for overexpression in C. oleaginosus to evaluate whether its overexpression would result in improved specific productivity.

A strain that overexpresses malic enzyme was created using methods similar to those described in Example 8 and was then evaluated in fed-batch bioreactors. A DNA cassette based on that depicted in FIG. 1B where ME2 is the GOI and G418R is the selectable marker was constructed. This cassette was transformed into C. oleaginosus via electroporation and strains containing stable integrated copies of the ME2 overexpression cassette were isolated and sequence verified. In some cases, the cassette was concatenated (usually end to end) with one or more additional copies at the site of integration. ME2 overexpression resulted in specific productivity of 69.2 mg/g/hr after 65 hours, a 14.1% improvement relative to WT. The increase in specific productivity suggests that the ME2 overexpression cassette was functional and results in modified strains that are improved in their fermentation performance. This further indicates that malic enzyme overexpression can be targeted for improving specific performance of the C. oleaginosus organism and increasing lipid production process performance.

### Example 16: Decreasing Viscosity of Fermentation Broth in Fed-Batch Bioreactors

Viscosity is a rheological property that in the context of a complex fermentation broth will generally follow a non-Newtonian behavior. In wild-type C. oleaginosus fermentations; as well as in other fungi the culture broth may become viscous. Increased viscosity of culture broth may result from the interactions of extracellular metabolites such as polysaccharides; the interaction of physical cell properties, such as filamentation, may also result in increased viscosity. A high viscosity broth may have limitations on mass transfer; for example, high broth viscosity may result in limitation of oxygen transfer and result in less productive fermentations for aerobic processes. Reducing broth viscosity may decrease the power needed to mix and aerate the fermentation broth; the decreased power may result in cost savings as a result of less power consumption and the need to install lower cost motors for mixing. A reduction in power utilization results in a decrease in the environmental impact of the overall process.

To decrease the viscosity of fermentation broth in fed-batch bioreactors, C. oleaginosus strains with genetic modifications were created. C. oleaginosus strains were modified to generate strains with either deletions of MNT1, AGS1, or overexpressions of VEL1 or VEL4.

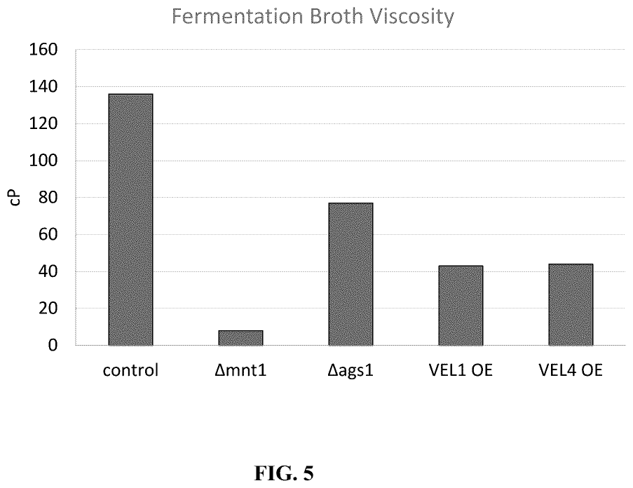

Each strain was evaluated for fermentation broth viscosity after 42-44 hours in a fed-batch bioreactor with a limiting nitrogen content of 3.2 g/L. Table 7 and FIG. 5 provide the fermentation broth viscosity of each strain and fermentation broth viscosity relative to the wild-type strain. It is observed that the strain containing an MNT 1 deletion exhibited a 94.1% decrease in viscosity as compared to the control strain, and the AGS1 deletion strain exhibited a 43.1% decrease in viscosity as compared to the control strain. It is observed that the VEL1 overexpression strain exhibited a 68% decrease in viscosity as compared to the control strain, and the VEL4 overexpression strain exhibited a 68% decrease in viscosity as compared to the control strain. The control strain viscosity is representative of the viscosity of an unmodified C. oleaginosus and/or a C. oleaginosus containing a deletion of FAD2A.

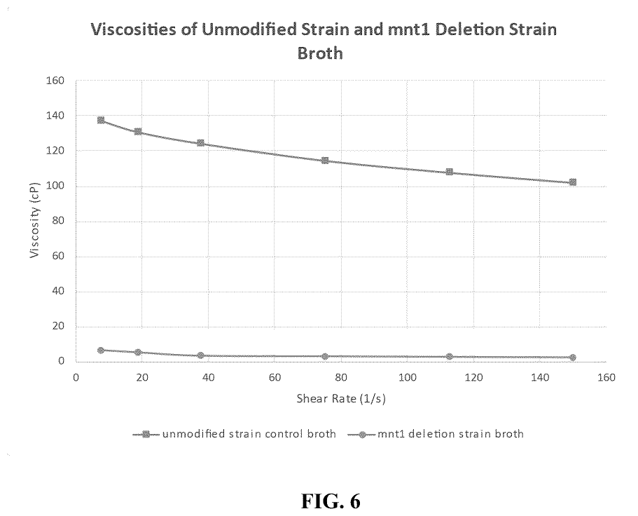

The viscosities of a fermentation broth without strain modification and a fermentation broth with the mnt1 deletion strain were measured at different shear rates (FIG. 6). The viscosities of the mnt1 deletion strain broth was found to be as little as 2% of that of the viscosity of the unmodified strain broth (as much as 98% reduction in viscosity).

TABLE 7
Fermentation broth viscosity of strains with genetic modifications

| | | Reduction (% relative |
|---|---|---|
| Strain | cP (5k rpm) | to WT) |
| WT (control) | 136 | — |
| Δmnt1 | 8 | 94.1% |
| Δags1 | 77 | 43% |
| VEL1 OE | 43 | 68% |
| VEL4 OE | 44 | 68% |

### Example 17: Increasing Specific Productivity of C. oleaginosus

Fermentation productivity is the amount of product made over time and specific productivity is the amount of product made per unit biomass per unit time. Productivity or specific productivity is in particular important with regard to lipid production as the product is intracellular and therefore limited to the amount of time it takes to reach the maximal volume available for lipid storage within a cell. High specific productivity rates translate to significant cost savings for fermentation as more lipid product may be made faster over a given time for the same size bioreactor or a smaller bioreactor may be used to achieve similar amounts of cumulative product as compared to a larger more costly unit.

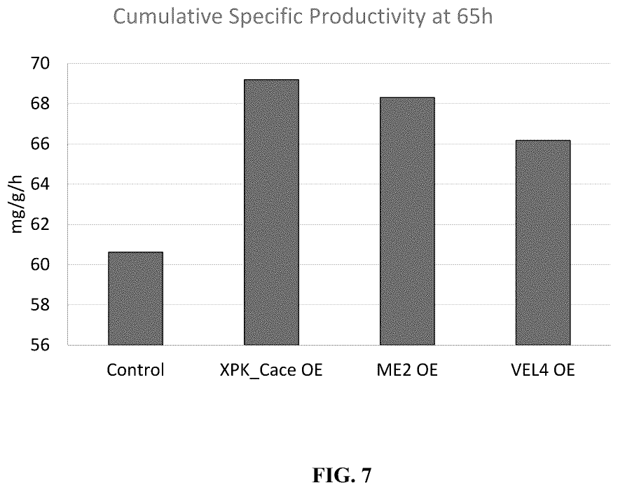

To increase the specific productivity of C. oleaginosus, strains with overexpression of either phosphoketolase, malic enzyme, or VEL4 were created. Each strain was evaluated for cumulative specific productivity after 65 hours in a fed-batch bioreactor. Table 8 and FIG. 7 provide the specific productivity of each strain and specific productivity relative to the wild-type strain. It is observed that the strain overexpressing either phosphoketolase exhibited an increase in specific productivity of lipids of 14.1% as compared to WT, the strain overexpressing malic enzyme ME2 exhibited an increase in specific productivity of lipids of 12.7% as compared to WT, and the VEL4 overexpression astrain exhibited an increase in specific productivity of lipids of 9.2% as compared to WT.

TABLE 8
Specific productivity of strains with genetic modifications

| | | Improvement (% |
|---|---|---|
| Strain | Mg/g/h | relative to WT) |
| WT (control) | 60.6 | — |
| Phosphoketolase | 69.2 | 14.1% |
| (XPK_Ca) OE | | |
| Malic enzyme | 68.3 | 12.7% |
| (ME2) OE | | |
| VEL4 OE | 66.2 | 9.2% |

### Example 18: C. oleaginous Produces Extracellular Polysaccharides (EPS) and MNT1 Deletion Reduces Extracellular Solids

EPS is typically described as a high-molecular weight carbohydrate polymer that is secreted by many organisms, including fungi. Solutions containing even low concentrations of EPS are commonly known to result in increased viscosity. Accordingly, the broth from a 3 L fermentation of a WT C. oleaginosus strain was examined for the presence of extracellular polysaccharide (EPS).

To determine whether EPS was present in C. oleaginosus fermentations the broth from 50 h of fermentation grown in nitrogen limiting media containing 4.8 g/L nitrogen content was isolated and dialyzed with a 1-kDa cutoff to generate a sample. The sample was determined to be majorly composed of mannose, representing 85.6%, with other hexoses in minor quantity. The remainder was identified as glucuronic acid (4.3% of the carbohydrate). Linkage analysis of the sample showed the sample is mainly composed of 3-linked mannose (49%). Other relevant residues included 2,3-linked mannose, along with 4-linked glucose with 4-linked glucuronic acid. The sample was composed of almost 50% carbohydrate and the average molecular weight was 4.5 MDa. Taken together we concluded that the properties of the sample are consistent with the notion that C. oleaginosus produces significant EPS.

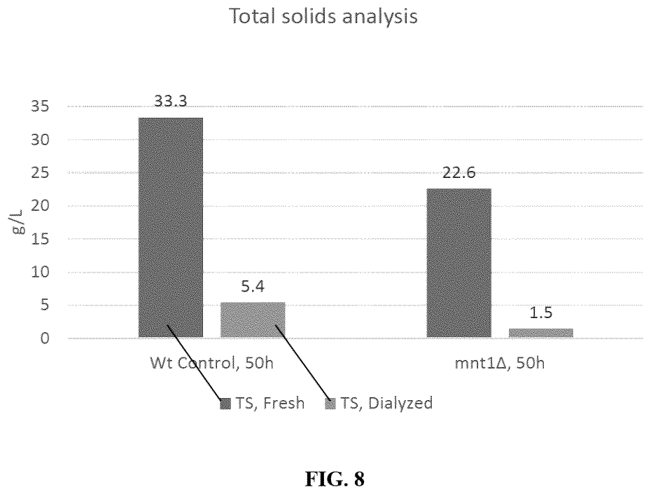

As seen in FIG. 8, the dry solids remaining before and after dialysis with a 1-kDa cutoff membrane are shown for both the WT and the mnt1 deletion strains grown under the same conditions (4.8 g/L nitrogen limited media and grown to 50 h). As EPS is expected to be the majority of the dry solids present after dialysis, the mnt1 deletion appears to have reduced EPS by more than 70% in comparison to the WT strain, from 5.4 g/L (WT) to 1.5 g/L (mnt1). This strongly suggests it is the decrease in broth EPS that results in the decrease in viscosity of broth from mnt1 deletion strains.

### Example 19: Phase Separation Using Various Solvents

400 microliters (μL) of a cell suspension consisting of oleaginous lipid-containing cells and culture media were mixed with 400 μL of either 1) water, 2) >99.5% methanol, 3) 10M H₂SO₄, or 4) acetone. Each of the four mixtures were mixed in a tube for 10 minutes before centrifuging at 10,000 relative centrifugal force (rcf) for 3 minutes each.

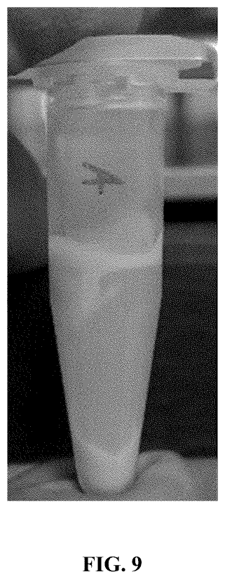

In the case of the mixture of cell suspension and water (e.g., 1), three phases were observed after centrifugation. As shown in FIG. 9, the densest phase was observed at the bottom and appeared to contain solids. A cloudy liquid phase was observed in the middle of the tube. Finally, the least dense phase was observed at the top and appeared to contain solids.

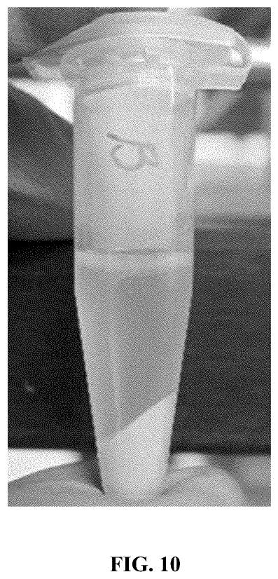

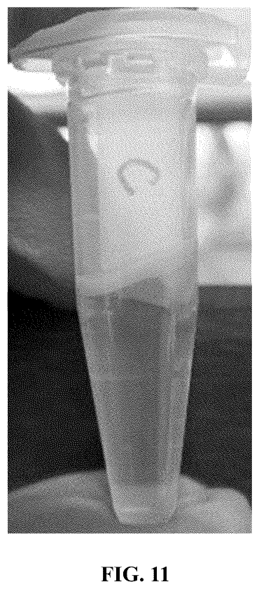

In the cases of the mixtures of cell suspension with >99.5% methanol or acetone, two phases were observed after centrifugation. As shown in FIG. 10 (>99.5% methanol) and FIG. 11 (acetone), the densest phase was observed at the bottom and appeared to contain solids. In both cases, a less dense, clear liquid phase was observed at the top of the tube.

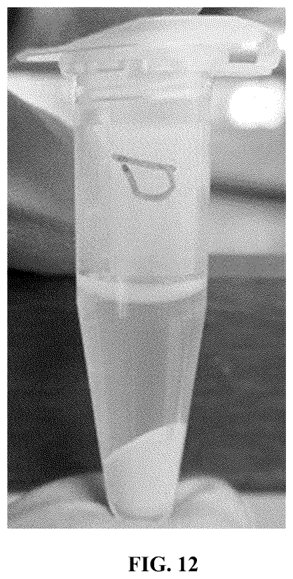

In the case of the mixtures of cell suspension with 10M H₂SO₄, as shown in FIG. 12, two phases were observed after centrifugation. A dense, clear liquid phase was observed at the bottom. A less dense phase was observed at the top of the tube and appeared to contain solids.

### Example 20: Phase Separation Using Various Solvents

400 μL of a cell suspension consisting of oleaginous lipid culture media was mixed with 900 μL of either 1) water, 2) >99.5% methanol, 3) >99.5% ethanol, 4) >99% allyl alcohol, or 5) glycerol. Additionally, 400 μL of the cell suspension was mixed with 6) 452.1 mg of xylitol. Each of the six mixtures were mixed in a tube for 10 minutes each before centrifuging at 10,000 rcf for 3 minutes each.

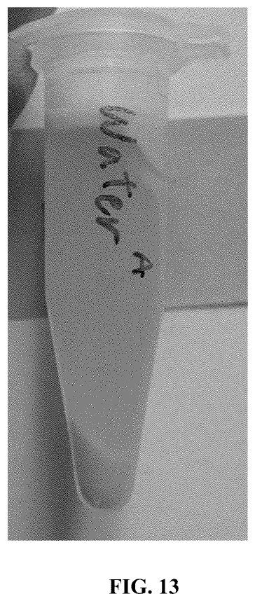

In the case of the mixture of cell suspension and water, as shown in FIG. 13, three phases were observed after centrifugation. The densest phase was observed at the bottom, verified by microscope to consist of solids which was mostly cells. A cloudy liquid phase was observed in the middle of the tube, verified by microscope to consist of little to no solids, which included cells. Finally, the least dense phase was observed at the top and was verified by microscope to consist of solids, which was also mostly cells.

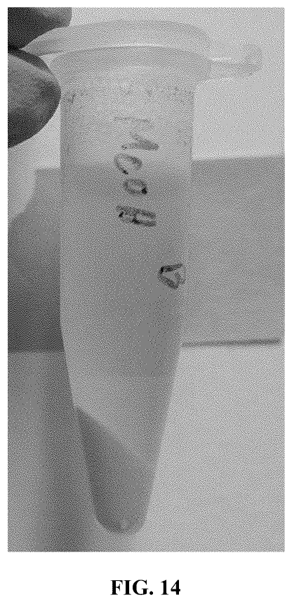

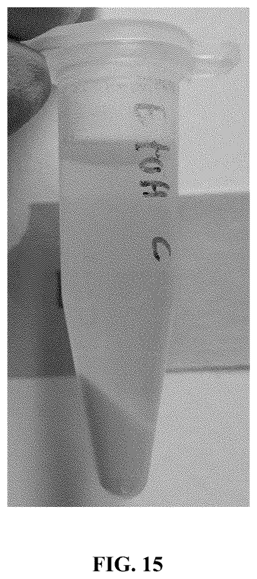

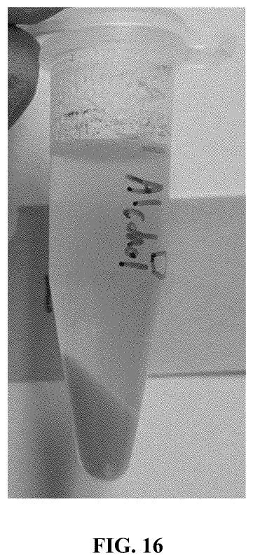

In the cases of the mixtures of cell suspension with >99.5% methanol (see FIG. 14), >99.5% ethanol (see FIG. 15), or >99% allyl alcohol (see FIG. 16), two phases were observed after centrifugation. In each case, the densest phase was observed at the bottom, verified by microscope to consist of cells and other solids. A less dense, liquid phase was observed at the top of the tube, verified by microscope to consist of little to no solids and very few cells.

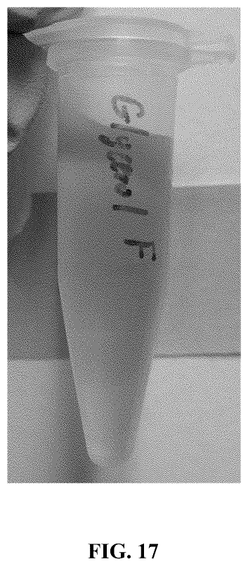

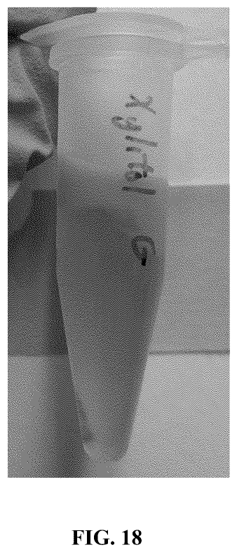

In the cases of the mixtures of cell suspension with glycerol (see FIG. 17) or xylitol (see FIG. 18), two phases were observed after centrifugation. In each case, a dense, liquid phase was observed at the bottom, verified by microscope to consist of little to no solids or cells. A less dense phase was observed at the top of the tube, verified by microscope to consist of solids, which were mostly cells.

### Example 21: Evaluation of Ethanol and Sulfuric Acid on the Sedimentation of Oleaginous Cultures

Oleaginous cultures were grown in a bioreactor under conditions to support high cellular lipid content, and the whole broth containing biomass was harvested and stored. Biomass concentration in the broth was 54 g/L, as measured as dry cell weight, and lipid content of these cells were on average 60%, as analyzed by acid hydrolysis of the biomass followed by extraction and gravimetric analysis of lipids using the Bligh-Dyer method.

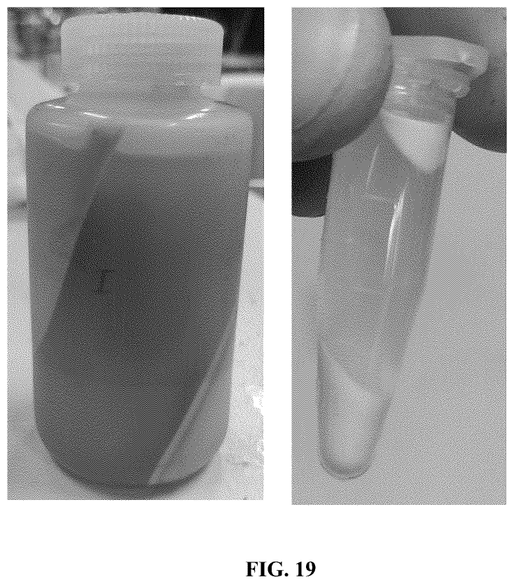

To concentrate and collect the biomass, centrifugation of the whole aqueous broth was performed in a 250 mL centrifuge bottle, at 10,000 g for 15 min. This was also repeated in 1.5 mL Eppendorf tubes, centrifuged at 12,000 rpm for 15 min. As shown in FIG. 19, the centrifugation of the culture resulted in two distinct pellets, a low-density float migrating to the top of the container, and a high-density pellet migrating to the bottom of the container. In addition, the solution between the heavy and light biomass phases was cloudy with cells having a similar density as the aqueous broth. This may be consistent with the oleaginous culture being a heterogeneous population of cells having a distribution of oil content, ranging from those with sufficient lipid to be lighter than the broth, those with enough lipid to be similar in density to the broth, and those making insufficient lipid to remain denser than the broth.

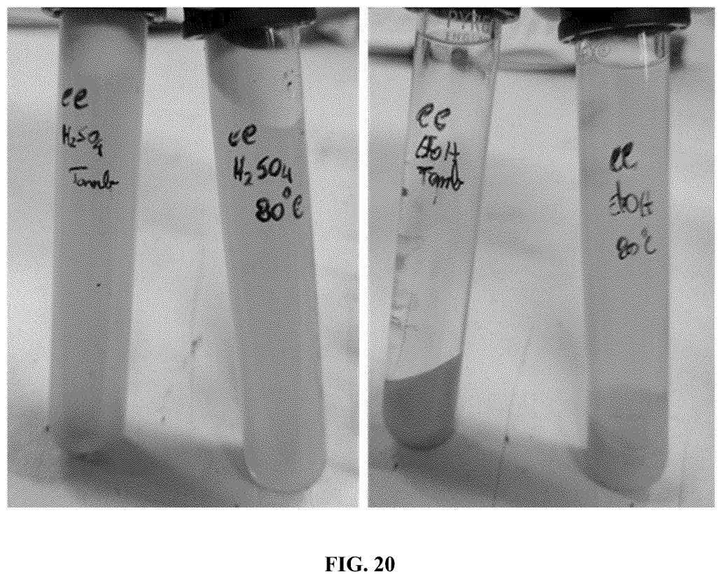

To consolidate the oil containing cells into a single pellet that could be separated from the bulk of the aqueous broth, the addition of an inorganic acid and the addition of a solvent were evaluated for the ability to increase or decrease, respectfully, the density of the broth to a level significantly above or below the average density of the oil culture population. Sulfuric Acid (H₂SO₄) was added to the broth to a final concentration of 4% (wt/vol) or ethanol was added to the broth to a final concentration of 70% (wt/vol). Each sample was centrifuged at 4000 rpm for 10 minutes. As shown in the resulting centrifuged tubes of FIG. 20, bringing the broth to 4% sulfuric acid results in all of the cells in the broth to form a floating raft at the top of the centrifuge tube, demonstrating the addition of sulfuric acid can be an effective means of increasing broth density sufficiently above that of the oil culture population, such that all of the cells can be concentrated as a light phase by centrifugation. Similarly, bringing the broth to 70% ethanol results in the cells of the broth to form a tight pellet at the bottom of the centrifuge tube, demonstrating the addition of this low-density solvent is an effective means of sufficiently decreasing the density of the broth below that of the oil culture population, such that all of the cells can be concentrated as a heavy phase by centrifugation. Experiments performed at 80° C. showed similar results, as shown in FIG. 20.

### Example 22: Evaluation of Different Ethanol Concentrations on Stability of Oleaginous Cultures

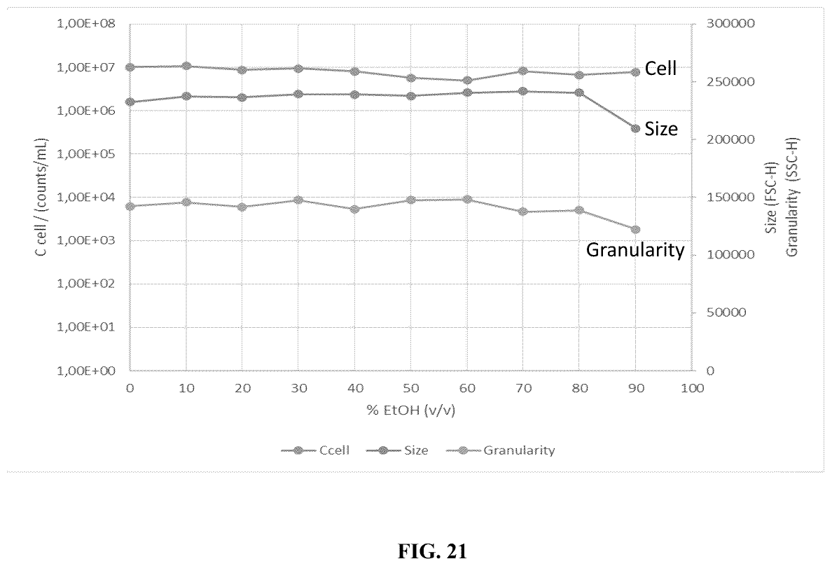

To better characterize the impact of ethanol concentration on the stability of the individual cells in oil culture, the biomass described in Example 3, was brought to different concentration of ethanol (0-90%) and characterized using flow cytometry. The biomass sample was diluted 1:10 in Ethanol:water solutions ranging from 10% to 90%, and modifications in cell concentration (Ccell), cell size (FSC-H) and morphology (SSC-H) were analyzed by flow cytometry. As shown in FIG. 21, cell concentration was maintained throughout the whole range of ethanol concentrations, indicating that the use of ethanol does not result in cell lysis and can be effectively used as a non-destructive additive in the recovery of whole oleaginous cells from aqueous suspensions.

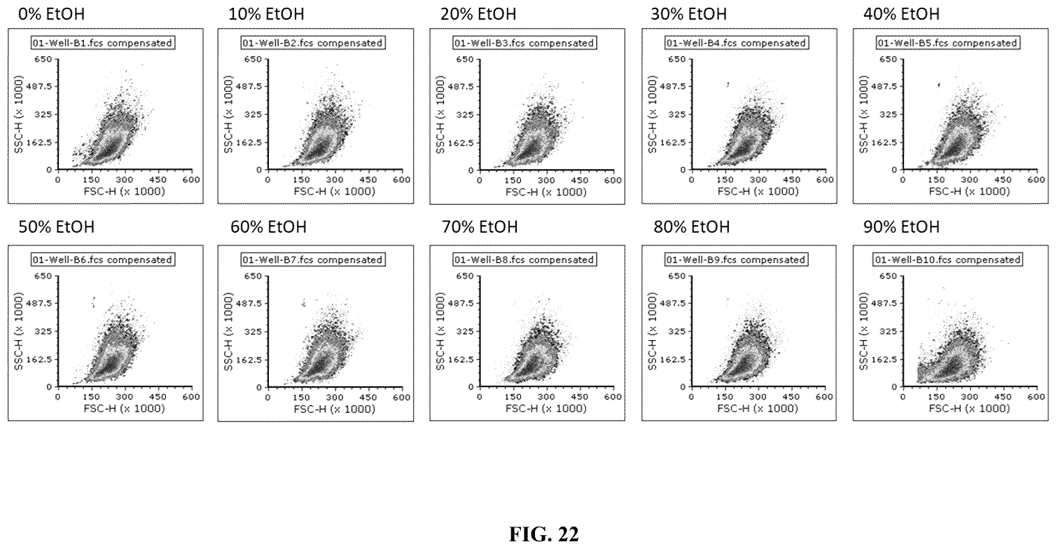

Moreover, cell morphology (FSC) and granularity (SSC) were also preserved when subjected to EtOH concentrations up to 80% (v/v), which is also evident in the population distribution from the flow cytometer analysis shown in the FIG. 22.

### Example 23: Evaluation of Ethanol Concentration on Oil Culture Sedimentation

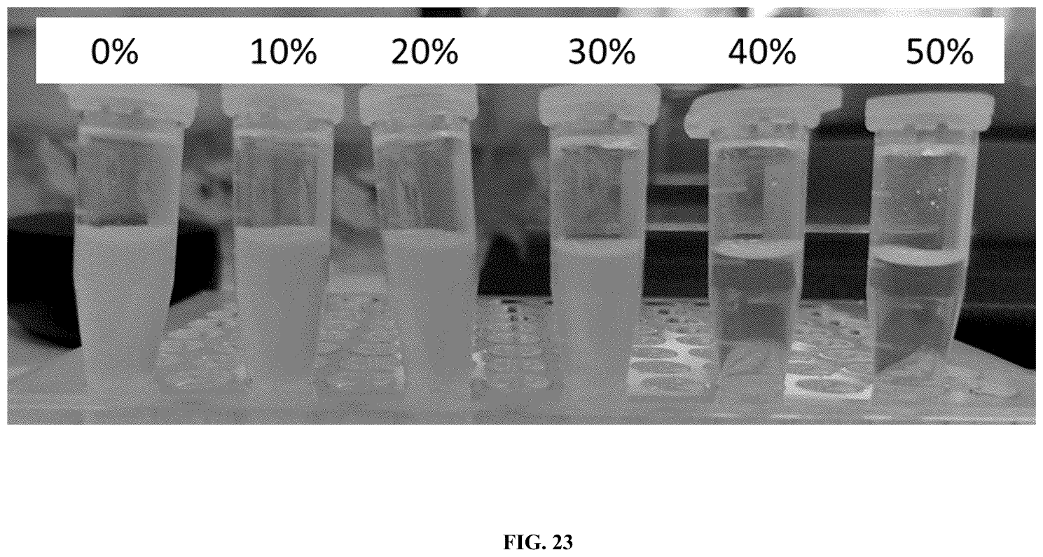

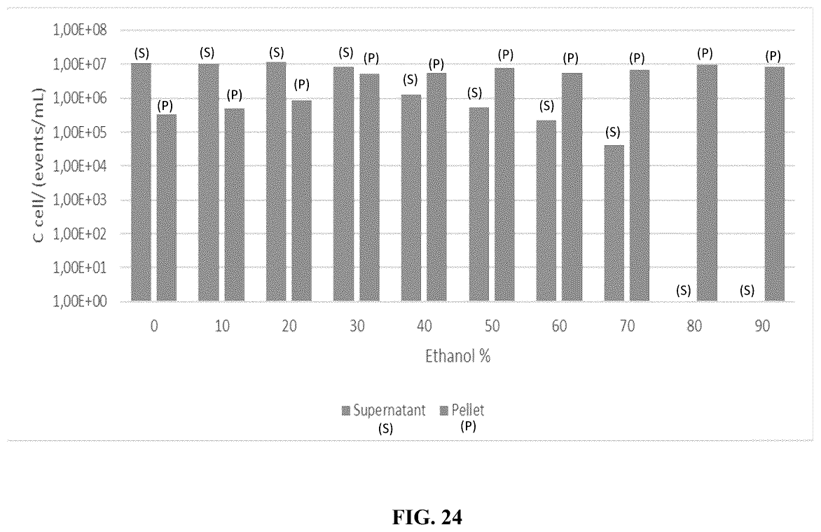

As cells are stable within a wide range of ethanol concentrations, a range of EtOH concentrations were evaluated for the ability to effectively enable the centrifugal pelleting of a high oil content oleaginous culture. An oleaginous biomass was cultured under the conditions described in Example 3, with the difference in pushing the culture to attain higher cell and lipid titers. The resulting culture contained 101 g/L of biomass, measured as dry cell weight, with the dry cells containing 75% by weight, as determined using Bligh-Dyer method. For testing the different EtOH concentrations a 100-fold dilution of the samples was performed applying different ethanol concentrations between 0 and 90% in 10% increments as previously. Dilutions were performed directly in 1.5 mL Eppendorf tubes, followed by centrifugation at 12,000 rpm for 10 min. As shown in FIG. 23, ethanol concentrations between 40-50% have a density that is below that of a high lipid content oil culture, and allows for concentration of the oil culture cells in the heavy pellet upon centrifugation. To further quantify this, the supernatant samples were collected from each centrifuged sample and analyzed directly by flow cytometry, whereas pellets were collected, washed and re-suspended in the corresponding hydroalcoholic solution before analysis by flow cytometry for characterization of cell concentration and morphology as previously. FIG. 24 illustrates the cell concentration vs the percentage of ethanol.

As shown in FIG. 24, the flow cytometry data corroborates more quantitatively the visual observations of FIG. 23. At ethanol concentration less than 40%, most of the cells remain in the supernatant or in the top solid phase due to their high lipid content and corresponding low density in comparison to the heavier liquid phase. At ethanol concentrations of 40% or above, the density of the liquid phase drops below that of the cells, and the cells pellet when centrifuged, allowing removal of upper liquid phase and collection of the concentrated lipid-rich biomass in the pellet.

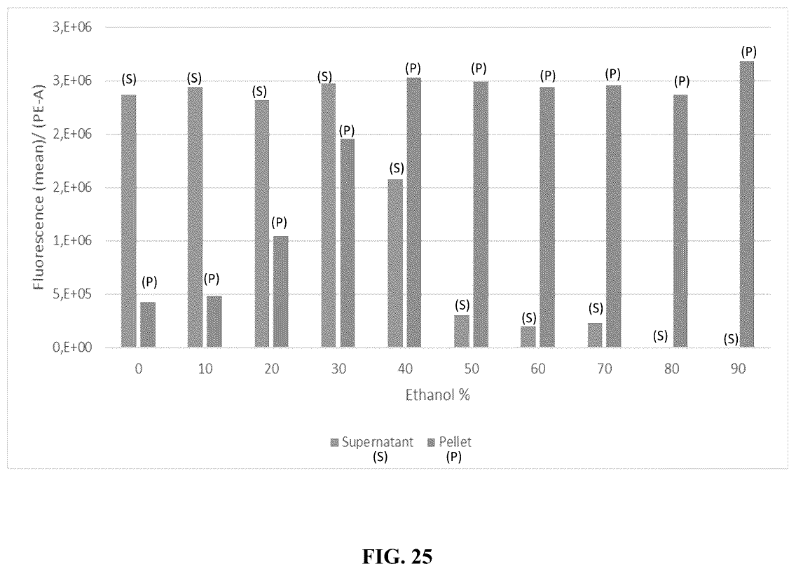

To demonstrate the correlation between lipid content, density, and the ethanol concentration of the liquid phase, cellular lipid content was also analyzed by flow cytometry. Prior to the flow cytometry, the isolated supernatants and pellets were incubated with the lipophilic fluorescent dye Nile Red. Cells containing lipid bind Nile Red and fluoresce, with total fluorescence of the cell correlating with the total lipid that it contains. FIG. 25 shows the mean values of fluorescence for the supernatant and the pellet at each concentration of ethanol.

The most fluorescent cells (those with the highest oil content and lowest density) appear to require the highest concentration of ethanol to pellet. This is consistent with increasing ethanol concentrations having decreasing liquid phase density. Indeed, the mean fluorescence of the supernatant is the highest at 50% ethanol, since most of the lower density, higher oil containing cells were pelleted under these conditions.

### Example 24: Evaluation of Cell Separation with Water and Detergent

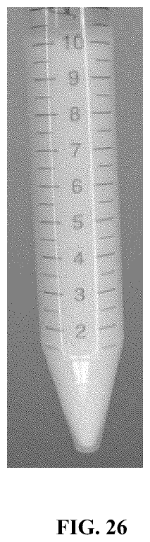

To demonstrate that water is an insufficient cell separation medium for cell separation, broth from highly oleaginous (40-90% oil cellular oil content) fermentations was exposed to water and then evaluated for the impact on the ability to separate the oil containing biomass from the aqueous broth. Approximately 10 mL of fermentation broth (consisting of cells comprised of lipids, water, and cell culture media) was mixed and poured into a 15 mL tube. The tube was then centrifuged at 4350rcf for 7 minutes. As seen in FIG. 26, no clear separation of cells from the aqueous phase was observed. The same result is seen for broths containing a diversity of cells spanning a range of densities across that of the aqueous medium as well as broths having a high viscosity or forming a stable colloid.

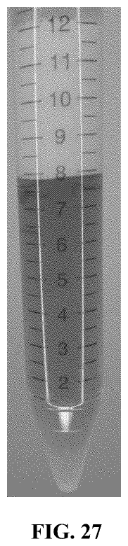

In a subsequent experiment, approximately 8 mL of the same broth was mixed with approximately 4 mL of Dawn liquid dish detergent in a 15 mL tube. The tube was then centrifuged at 4350rcf for 7 minutes. As seen in FIG. 27, two phases were observed after centrifugation. The densest phase was observed at the bottom and appeared to consist of approximately 8 mL of an aqueous phase without any solids. The lighter phase at the top appeared to be approximately 4 mL and contained all the cells and some of the aqueous phase from the cell mixture.

Based on these observations, centrifugation of fermentation broth alone is insufficient to separate water from the oil containing cells. However, surprisingly, the addition of liquid detergent changed the nature of the broth sufficiently to enable centrifugal separation of a significant portion of the aqueous broth from the oil containing cells.

### Example 25: Evaluation of Cell Separation Using Salts at Low pH

To determine the effect of lowering pH and adding salts during cell separation, broth from highly oleaginous (40-90% oil cellular oil content) fermentations was exposed to low pH and various salts and then evaluated for the impact on the ability to separate the oil containing biomass from the aqueous broth. Fermentation broth was pH-adjusted to pH=2 using 8M sulfuric acid. The initial pH of the fermentation broth was 4-6. Approximately 35 mL of the pH-adjusted fermentation broth was added to a 50 mL tube and then centrifuged at 4350rcf for 30 minutes. For each of aluminum sulfate, ferric chloride, calcium chloride, and sodium chloride, approximately 3.5 g of each salt was added to approximately 35 mL of pH-adjusted fermentation broth. The salt-broth mixtures were mixed in 50 mL tubes, and then centrifuged at 4350rcf for 30 minutes.

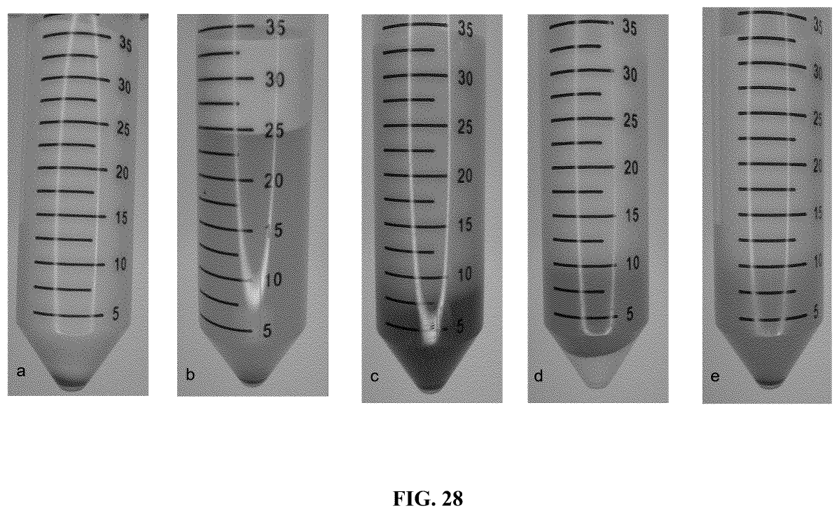

As seen in FIG. 28, no clear separation of cells from the aqueous phase was observed in the pH-adjusted broth with no salts added. In the cases where ferric chloride, calcium chloride, and sodium chloride were added to the pH-adjusted broth, a cell-containing phase was observed separate from a no- to low-solids aqueous phase; however, the aqueous phase was small compared to the cell-containing phase. In the case where aluminum sulfate was added to the pH-adjusted broth, a cell-containing phase was again observed separate from a no- to low-solids aqueous phase, but with a significant volume in the aqueous phase relative to the cell-containing phase.

Based on these observations, treatment of broth by lowering pH and mixing with aluminum sulfate surprisingly changes the nature of the broth sufficiently to enable centrifugal separation of a significant portion of the aqueous broth from the oil containing cells.

### Example 26: Evaluation of the Effect of Temperature on Cell Separation

To determine the effect of temperature on cell separation, broth from highly oleaginous (40-90% oil cellular oil content) fermentations was exposed to various temperatures and then evaluated for the impact on the ability to separate the oil containing biomass from the aqueous broth. Approximately 300 g of fermentation broth was added to a mixing vessel. This was repeated for a 2nd mixing vessel. Approximately 15 g of 99.999% sulfuric acid was added to approximately 300 g of fermentation broth for a 3rd and 4th mixing vessel. The 1st and 3rd mixing vessels were mixed at approximately 20° C. for 24 hours. The 2nd and 4th mixing vessels were mixed at 70° C. for 24 hours. After 24 hours of mixing, 10 mL of mixture was removed from each mixing vessel and dispensed into a 15 mL tube. The tubes were then centrifuged at 4350rcf for 7 minutes.

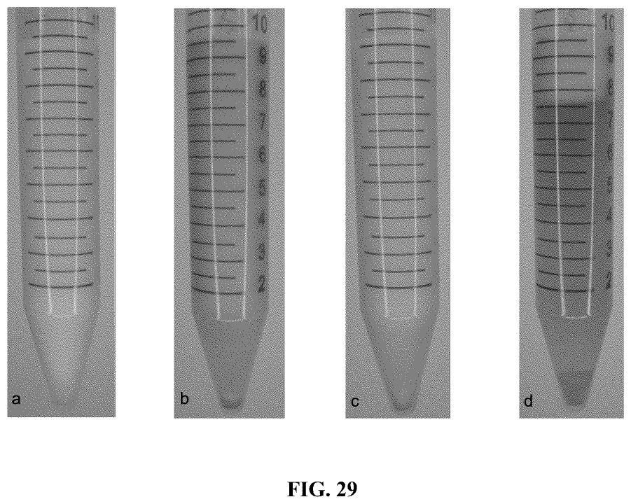

As seen in FIG. 29, no clear separation of cells from the aqueous phase was observed in the tubes containing mixtures that were mixed at 20° C. In both tubes containing 70° C. mixed material, a cell-containing phase was observed separate from a no- to low-solids aqueous phase. In the case where no sulfuric acid was added to the fermentation broth before 70° C. mixing, the aqueous phase was small compared to the cell-containing phase. In the case where sulfuric acid was added to the fermentation broth before 70° C. mixing, a significant volume of aqueous phase was observed relative to the cell-containing phase. The viscosity of each mixture is shown in Table 9.

TABLE 9
Viscosity of broth mixtures at various temperatures
with and without sulfuric acid

| | Mixing Temperature | | |
|---|---|---|---|
| Trial | (° C.) | Sulfuric Acid | Viscosity at 75 s − 1 |
| A | 20 | No | 230.5 |
| B | 70 | No | 55.92 |
| C | 20 | Yes | 94.5 |
| D | 70 | Yes | 3.27 |

Based on these observations, treatment of broth with a higher temperature without a lower pH changes the nature of the broth sufficiently to generate a separable water phase from the cells after centrifugation. When the broth is treated with both a lower pH and mixing at a higher temperature, separation of the water from the cells in the broth is enhanced synergistically, enabling centrifugal separation of a significant portion of the aqueous broth from the oil containing cells while also effectively decreasing the viscosity of the broth to very close to that of water.

### Example 27: Evaluation of the Effect of Duration on Cell Separation

To determine the effect of duration at high temperature with sulfuric acid on cell separation, broth from highly oleaginous (40-90% oil cellular oil content) fermentations was exposed to high temperature and sulfuric acid for various lengths of time and then evaluated for the impact on the ability to separate the oil containing biomass from the aqueous broth. Approximately 400 mL of fermentation broth was added to each of four mixing vessels. In the 1st mixing vessel, no sulfuric acid was added to the broth. In the 2nd mixing vessel, 0.5 g of sulfuric acid was added to every 100 g of broth. In the 3rd mixing vessel, 1 g of sulfuric acid was added to every 100 g of broth. In the 4th mixing vessel, 2 g of sulfuric acid was added to every 100 g of broth. Each vessel was mixed at 70° C. for 24 hours. After 8 hours of mixing, and then again after 24 hours of mixing, 10 mL of mixture was removed from each mixing vessel and dispensed into a 15 mL tube. The tubes were centrifuged at 4350rcf for 7 minutes.

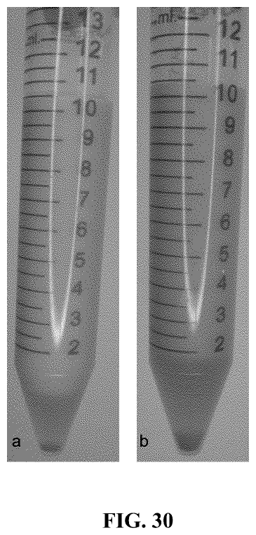

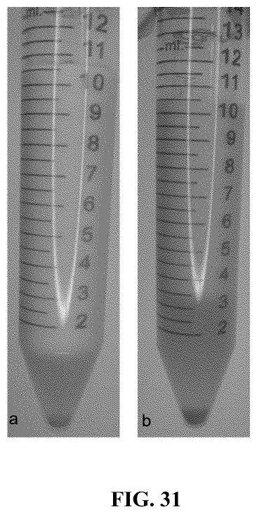

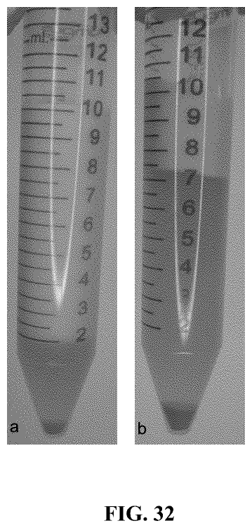

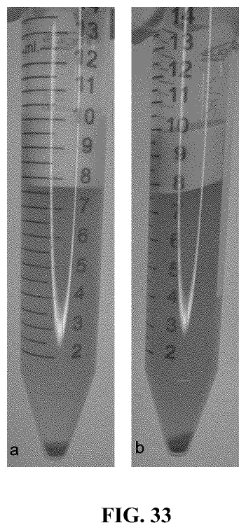

As seen in FIGS. 30-33, a cell-containing phase was observed separate from a no- to low-solids aqueous phase in all tubes. In both the 8-hour and 24-hour tubes from the 1st mixing vessel, where no sulfuric acid was added, the aqueous phase was small compared to the cell-containing phase. In both the 8-hour and 24-hour tubes from the 2nd mixing vessel, where 0.5 g of sulfuric acid was added to every 100 g of broth, the aqueous phase was small compared to the cell-containing phase. In the 8-hour tube from the 3rd mixing vessel, where 1 g of sulfuric acid was added to every 100 g of broth, the aqueous phase was small compared to the cell-containing phase. In the 24-hour tube from the 3rd mixing vessel, a significant volume of aqueous phase was observed relative to the cell-containing phase. In both the 8-hour and 24-hour tubes from the 4th mixing vessel, where 2 g of sulfuric acid was added to every 100 g of broth, a significant volume of aqueous phase was observed relative to the cell-containing phase.

Based on these observations, both the amount of sulfuric acid added to the broth and the time the broth is incubated at elevated temperature correlate with an increase in the separation of efficiency of the water and the cells in the broth. And, the treatment of broth containing oil containing cultures with elevated temperatures and/or acid are effective means of separating the oil containing cells from a significant portion of the broth aqueous fraction. Further, the benefit of exposing to acid or elevated temperature can be increased by increasing the time exposure to each of these treatments. In addition, an increase in acid concentration in a treatment significantly decreases the required time of incubation to achieve optimal separation.

### Example 28: Cell Separation Using EPS Filters Under Vacuum

In addition to centrifugation, another desired means of separating oil containing cells in a fermentation broth from the other aqueous media is the use of filtration, trangential flow filtration, dead-end filtration, etc. This may use ultra-filtration, microfiltration, filter press filtration, or other similar unit operations. To evaluate the ability of filtration to separate water from oil containing cellular biomass, fermentation broths containing oil cultures with different viscosities were applied to 1.2 μm PES filters under vacuum and the rate of filtration were compared.

Fermentation broth containing an oil culture with no strain modifications was poured over a Büchner funnel lined with a 1.2 μm PES membrane. Filtration was performed under 830 mbar of vacuum at 22° C. Under these conditions, no filtrate passed through the filter. This was consistent with the high viscosity of the broth the wild-type organism and its resistance to centrifugal separation.

Fermentation broth containing an oil culture containing a genetic modification MNT1 which results in decreased broth viscosity was poured over a Büchner funnel lined with a 1.2 μm PES membrane. Filtration was performed under 830 mbar of vacuum at 22° C. 83% of the starting broth by weight passed through the membrane as filtrate with a flux of 166.9 kg/hr-m2.

Fermentation broth containing an oil culture containing a genetic modification MNT1, but grown to higher cell density was also poured over a Büchner funnel lined with a 1.2 μm PES membrane. Filtration was performed under 830 mbar of vacuum 22° C. 77% of the starting broth by weight passed through the membrane as filtrate with a flux of 53.7 kg/hr-m2.

Hence, an effective means of treating a fermentation broth to enable efficient water removal by filtration is to identify a genetically modified strain that results in a decrease in broth viscosity. For example, such a strain could be engineered to decrease the secretion of extracellular factors the increase broth viscosity, such as proteins, polymers, polysaccharides and the like.

### Example 29: Comparison of Cell Separation Using EPS Filters Under Vacuum and Centrifugation

As strains that with lower viscosity were more effectively separated from aqueous fermentation broth by filtration, the effect of viscosity on separation by centrifugation was tested.

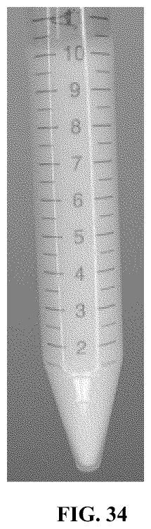

10 mL of fermentation broth from the unmodified high viscosity strain was poured into a 15 mL centrifuge tube. The tube was centrifuged at 4350rcf for 7 minutes. As seen in FIG. 34, no phase separation occurred. This was consistent with previous observations.

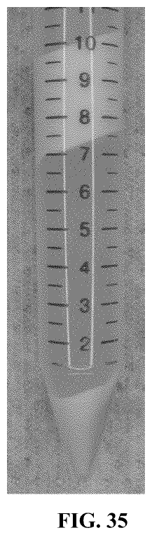

For comparison, 10 mL of broth containing an oil culture produced from a strain with the genetic modification mnt14 was poured into a 15 mL centrifuge tube. The tube was centrifuged at 4350rcf for 7 minutes. As seen in FIG. 35, 28% of the broth by volume separated as a cell layer on the top of the tube. Below it, 64% by volume separated as an aqueous phase in the middle of the tube. The remaining 7% of broth by volume was a cell layer on the bottom of the tube.

Hence, an effective means of treating a fermentation broth to enable efficient water removal by centrifugation is to identify a genetically modified strain that results in a decrease in broth viscosity. For example, such a strain could be engineered to decrease the secretion of extracellular factors that increase broth viscosity, such as proteins, polymers, polysaccharides and the like. Based on these observations, it could be expected that those chemical and physical treatments identified to be changing broth characteristics to enable effective centrifugal separation of water from oleaginous cell biomass would also decrease broth viscosity and enable effective filtration, similar to the genetic modification.

### Definitions

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “about” when referring to a number or a numerical range generally means that the number or numerical range referred to is an approximation within experimental variability (or within statistical experimental error), and thus the number or numerical range may vary from, for example, between 1% and 15% of the stated number or numerical range.

The term “sequence identity” as used herein in the context of amino acid sequences is defined as the percentage of amino acid residues in a candidate sequence that are identical with the amino acid residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. As used herein “similar” or “similarity” when referring to an amino acid sequence refers to “sequence identity” unless otherwise stated.

As used herein the “Enzyme Commission number” (or “EC number”), refers to the standard implemented by the Nomenclature Committee of the International Union of Biochemistry and Molecular Biology (IUBMB), is a numerical classification scheme for enzymes, based on the chemical reactions they catalyze.

As used herein a “deletion” or a “deletion modification” can include a genetic modification which leads to under-production, under-expression, inactivation, or negative attenuation of the gene or a protein encoded by the gene.

As used herein an “overexpression” or a “overexpression modification” can include a genetic modification which leads to overexpression, overproduction, activation, or positive attenuation of the gene or a protein encoded by the gene.

As used herein “negative attenuation” can refer to a reduction in the specific activity of a protein encoded by a gene. For example, the catalytic activity of an enzyme.

As used herein “positive attenuation” can refer to an increase in the specific activity of a protein encoded by a gene. For example, the catalytic activity of an enzyme.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. (canceled)

2. (canceled)

3. A microorganism comprising one or more genetic modifications, wherein the genetic modification comprise at least one of the following genetic modifications:
a. a MNT1 deletion, under-production, under-expression, inactivation, or negative attenuation; a deletion, under-production, under-expression, inactivation, or negative attenuation of a MNT1 homologue; a deletion, under-production, under-expression, inactivation, or negative attenuation of a protein comprising an amino acid sequence set forth in SEQ ID NO. 27, SEQ ID NO. 72, or SEQ ID NO. 73; a deletion, under-production, under-expression, inactivation, or negative attenuation of a protein comprising an amino acid sequence which is at least 70%, 75%, 80%, 85%, 90%, or 95% similar to SEQ ID NO. 27, SEQ ID NO. 72, or SEQ ID NO. 73; or a deletion, under-production, under-expression, inactivation, or negative attenuation of a protein which catalyzes a reaction defined by EC 2.4.1;
b. an AGS1 deletion, under-production, under-expression, inactivation, or negative attenuation; a deletion, under-production, under-expression, inactivation, or negative attenuation of an AGS1 homologue; a deletion, under-production, under-expression, inactivation, or negative attenuation of a protein comprising an amino acid sequence set forth in SEQ ID NO. 28, SEQ ID NO. 74, SEQ ID NO. 75, or SEQ ID NO. 76; a deletion, under-production, under-expression, inactivation, or negative attenuation of a protein comprising an amino acid sequence which is at least 70%, 75%, 80%, 85%, 90%, or 95% similar to SEQ ID NO. 28; or a deletion, under-production, under-expression, inactivation, or negative attenuation of a protein which catalyzes a reaction defined by EC 2.4.1.183;
c. a VEL1 overexpression, overproduction, activation, or positive attenuation; an overexpression, overproduction, activation, or positive attenuation of a VEL1 homologue; an overexpression, overproduction, activation, or positive attenuation of a protein comprising an amino acid sequence set forth in SEQ ID NO. 59, SEQ ID NO. 77, SEQ ID NO. 78, SEQ ID NO. 79, or SEQ ID NO. 80; or an overexpression, overproduction, activation, or positive attenuation of a protein comprising an amino acid sequence which is at least 70%, 75%, 80%, 85%, 90%, or 95% similar SEQ ID NO. 59, SEQ ID NO. 77, SEQ ID NO. 78, SEQ ID NO. 79, or SEQ ID NO. 80; or
d. a VEL4 overexpression, overproduction, activation, or positive attenuation; an overexpression, overproduction, activation, or positive attenuation of a VEL4 homologue; an overexpression, overproduction, activation, or positive attenuation of a protein comprising an amino acid sequence set forth in SEQ ID NO. 58, or SEQ ID NO. 81, SEQ ID NO. 82, SEQ ID NO. 83, or SEQ ID NO. 84; or an overexpression, overproduction, activation, or positive attenuation of a protein comprising an amino acid sequence which is at least 70%, 75%, 80%, 85%, 90%, or 95% similar SEQ ID NO. 58, or SEQ ID NO. 81, SEQ ID NO. 82, SEQ ID NO. 83, or SEQ ID NO. 84.

4. The oleaginous microorganism of claim 3, wherein the deletion, under-production, under-expression, inactivation, or negative attenuation comprises: gene deletion, replacement, mutation, or knockdown of a gene encoding a protein in a. or b.

5. The oleaginous microorganism of claim 3, wherein the overexpression, overproduction, activation, or positive attenuation comprises: heterologous expression of a gene encoding a protein in c. or d, modification of a regulatory element coupled to a gene encoding a protein in c. or d., modification of a nucleotide sequence of a gene encoding a protein in c. or d., or a mutation to an amino acid sequence of a protein in c. or d.

6-8. (canceled)

9. The oleaginous microorganism of claim 3, wherein the microorganism has an additional genetic modification comprising at least one of the following genetic modifications:
a. a FAD2A deletion, under-production, under-expression, inactivation, or negative attenuation; a deletion, under-production, under-expression, inactivation, or negative attenuation of an FAD2A homologue; a deletion, under-production, under-expression, inactivation, or negative attenuation of a protein comprising an amino acid sequence set forth in SEQ ID NO. 1; a deletion, under-production, under-expression, inactivation, or negative attenuation of a protein comprising an amino acid sequence which is at least 70%, 75%, 80%, 85%, 90%, or 95% similar to SEQ ID NO. 1; or a deletion, under-production, under-expression, inactivation, or negative attenuation of a protein which catalyzes a reaction defined by EC 1.14.19.6;
c. a overexpression, overproduction, activation, or positive attenuation of a XPK gene encoding a phosphoketolase; overexpression, overproduction, activation, or positive attenuation of a protein comprising an amino acid sequence set forth in SEQ ID NO. 40, or overexpression of a protein comprising an amino acid sequence which is at least 70%, 75%, 80%, 85%, 90%, or 95% similar SEQ ID NO. 40, overexpression of a protein which catalyzes a reaction defined by EC 4.1.2.9 or EC 4.1.2.22; or
d. an overexpression, overproduction, activation, or positive attenuation of a ME2 gene encoding a Malic Enzyme; overexpression, overproduction, activation, or positive attenuation of a protein comprising an amino acid sequence set forth in SEQ ID NO. 37; or overexpression, overproduction, activation, or positive attenuation of a protein comprising an amino acid sequence which is at least 70%, 75%, 80%, 85%, 90%, or 95% similar SEQ ID NO. 37, overexpression of a protein which catalyzes a reaction defined by EC 1.1.1.40.

10. The microorganism of claim 9, wherein the gene XPK encoding the phosphoketolase is from Clostridium acetylbutilicum.

11-17. (canceled)

18. The microorganism of claim 9, wherein the overexpression, overproduction, activation, or positive attenuation of a phosphoketolase (XPK) encoding gene, overexpression, overproduction, activation, or positive attenuation of a Malic Enzyme (ME2) encoding gene, or overexpression, overproduction, activation, or positive attenuation of a VEL4 encoding gene modifications increase production of lipids by the microorganism by at least 8, 9, 10, 11, 12, 13, or 14%.

19. The microorganism of claim 3, wherein the microorganism comprises triacylglycerides (TAGs) which TAGs comprise less than 6% polyunsaturated fatty acids.

20. The microorganism of claim 3, wherein one or more of the genetic modifications is a targeted deletion of genomic DNA comprising a heterozygous or homozygous deletion of a native gene.

21. (canceled)

22. The microorganism of claim 3, wherein the microorganism further comprises addition of exogenous DNA.

23. The microorganism of claim 22, wherein the exogenous DNA is a selectable marker and is flanked by recombination sites, or is a single recombination site and does not comprise a selectable marker.

24. (canceled)

25. The microorganism of claim 19, wherein the TAGs comprise less than 5% linoleic acid.

26. The microorganism of claim 19, wherein the TAGs comprise less than 5% PUFA and less than 4% linoleic acid.

27-30. (canceled)

31. The microorganism of claim 3, wherein the genomic DNA or exogenous DNA encodes at least a portion of a fatty acid metabolism protein.

32. The microorganism of claim 31, wherein the fatty acid metabolism protein is selected from a fatty acid desaturase, TGL3, MFE1, NADPH dependent GDH dehydrogenase, PEX10; Trioll246656, TFDH1, TOR1, OLE1, or FAD2A.

33. The microorganism of claim 3, wherein the genomic DNA encodes at least a portion of a protein selected from A12 desaturase FAD2A; TGL3-370130 (lipase); MFE1-385434 (B-oxidation); TOR1 (372351) (Target of rapamycin-signaling); GSY1; Glycogen synthase 365053 EC: 2.4.1.11; Trehalose synthase 371254 (TPS1) EC: 2.4.1.15; TFDH1 (C1-tetrahydrofolate synthase) 395209; PEX10 (lipase) 367175 E3 ubiquitin ligase; OLE1 348634 EC: 1.14.19.1; UDP-Glc pyrophosphorylase (UGP) 347999 EC: 2.7.7.9; UDP-Glc decarboxylase (UXS1) 370161 EC: 4.1.1.35; UDP-glucose 4-epimerase (GALE1) 367812 EC: 5.1.3.2; UDP-galactose transporter (UGT) 334886; Mannose pyrophosphorylase (MPP2) 370163 EC: 2.7.7.13; B-glucan synthesis (SKN1A) 306218; B-glucan synthesis (SKN1B) 333973; CAS1 (o-acetyltransferase) 177902; Chitin synthase (CHS2) 215086; Chitin synthase (CHS4) 348500; Chitin synthase (CHS1) 390231 EC: 2.4.1.16; Glycosylphosphatidylinositol mannosyltransferase (PIGB) 385852; Glycosylphosphatidylinositol mannosyltransferase (PIGS) 413175; UDP-glucose 4-epimerase 365740 (GALE2); PIGH 394303; Mannose pyrophosphorylase (MPP1) 367061 EC: 2.7.7.13; B-glucan synthesis (SKN1C) 370117

34. (canceled)

35. The microorganism of claim 31, wherein the fatty acid metabolism protein is selected from GAPN, DGAT, PTA, ME, XPK, POS5, ZWF, CTP1, CAT2, ACC1, TOR, ACL, OLE1, FAT5, ELO1, ELO2, EUTE, MPHF, ALD6, ADH2, EDD, EDA, CBBM, PRK.

36. The microorganism of claim 3, comprising three copies of a gene, wherein two of the copies are endogenous to the microorganism.

37. The microorganism of claim 36, wherein the gene encodes a fatty acid metabolism protein selected from GAPN, DGAT, PTA, ME, XPK, POS5, ZWF, CTP1, CAT2, ACC1, TOR, ACL, OLE1, FAT5, ELO1, ELO2, EUTE, MPHF, ALD6, ADH2, EDD, EDA, CBBM, PRK.

38. The microorganism of claim 3, wherein the microorganism is Cutaneotrichosporon oleaginosus.

39. A liquid culture comprising the microorganism of claim 3.

40. The liquid culture of claim 39, wherein the liquid culture comprises a viscosity of up to 15, 10, or 5 cP.

41. The liquid culture of claim 39, wherein the liquid culture is configured to increase a transfer of dissolved oxygen within the culture.

42-50. (canceled)

51. A method of altering a fatty acid profile of a diploid oleaginous microorganism, the method comprising modifying target native genomic DNA of the microorganism by homologous recombination, wherein the modifying modulates expression of a fatty acid metabolism protein in the microorganism, thereby altering the fatty acid profile of the microorganism.

52-64. (canceled)

65. The oleaginous microorganism of claim 3, wherein the microorganism comprises a genetic modification and has greater than 50% by weight of triacylglycerides (TAGs), wherein the TAGs comprise less than 6% polyunsaturated fatty acids, wherein the microorganism is genetically diploid, and wherein the genetic modification causes underexpression of a Δ12 desaturase.

66. (canceled)

67. The genetically modified oleaginous microorganism of claim 65, wherein the genetic modification is a deletion, under-production, under-expression, inactivation, or negative attenuation in a gene encoding FAD2A to eliminate linoleic acid production.

68-93. (canceled)

94. An oleaginous microorganism that has been genetically modified to underproduce one or more of the proteins set forth in Table 1 or to overproduce one or more of the proteins set forth in Table 2.

95-167. (canceled)

167. The microorganism of claim 3, wherein the yeast is selected from a group consisting of Lipomyces tetrasporus, Lypomyces starkeyi, Cutanetrichosporon oleaginosus, Rhodotorula toluroides, and Yarrowia Lypolytica.

169-227. (canceled)


SEQUENCE LISTING (26 sequences)

SEQ ID NO: 1 - protein, 446 aa, synthetic construct
MSAATLRQRNVDKPGAADKAELLREAEDLELTEGQKFVVPNFTVKQLLDAIPAHCYKRSAFKSSLYVLQDFVLLAALVYGAYHIDSFLSRFNLGSVAHTAAKIGLWFTYQVLAGMVGTGIWVIAHECGHQAYSESKTINNAVGWVLHSILLVPYHSWRISHGRHHAATGHLTRDEVFVPRTREQLGIQAPKTEEEKKGINVPAWRQAELREALEESPIGALYGAILHQLFGWPMYLIRNASGQLWYPKMTNHFQPSSIIFKPSHFWQIIASDIGVVLTAAALGVFVYYRGFAEMARIYLIPYLWVNHWLVFITFLQHTDPVLPHYSEKTWTFARGALATIDRNCLGPVGPYLFHGITETHVAHHTSSRIPHYNAWEATEALKKFLGPHYHYNPENMFVSFWKAHRYCKFIEAGEDVAFYRNAAGVAQKVGIIEENGAVSDSGVEHK

SEQ ID NO: 2 - protein, 792 aa, synthetic construct
MTAIDDIPEGVDPTLWTYNHINEEHLAAFEHALSLQDVAPIEGDMSPAMLKSPQLTPHDAPEWSKGVEKLTATSDFAPIHSRVRKRRSAGQQSLGLTYHLIRWPLLAFFFLIIWIEFSAYVLTRQVVNVFEWLFAWRGVQRVLKRKLRNAKSYEEWRATAKELDDELCFDDWKETDSDGYFDANLVRRVRATLYKLRLAKDTRALMDALSICVRPNFAGTESSRMYSETFYGTKTLVEAHIKEVAASLDYVRTASNVTLEEKRAFFRRLNKNYGTSALCLSGGASFGYYHFGVARALLDHGLLPRVITGTSAGGLIAALVCTYTDSELKQLIRPELADKITALEDPFSVWIRRWMQTGARFSALEWARKAMYFTRGSLTFKEAYERTGRALNVSVVPADRHSPTILLNHLTAPNCVIWSAIIASAAVPGILNPVVLMAKDRDGSVRPHNLGGSRFKDGSLREDIPLSSLHTQFNCNFSIVSQTNPHIHLFFFAPRGAVGRPVAHRKGRGWRGGFILSALESYIKLDLSKHFKVIRDLDLLPQLLQSDWSGVFLQRFFGDLTLIPKSRPLDWLHILDDPDRKGLARMILVGQRVTWPTIHMVHNRAKIEDAIHRGRAEVRMALARERPSAAPTGTMPLESDADSAIALRARRADGQGSGSSRRRRPDRLQIPRTQNITDVQAVTSPNELPRSNTAPSFGDTFRQMRSSPLAAFGFKNRTRSRRSISAWLGGGSDSSLSDDEVDLGGPEWRADAAATANGLDLNDDVFLDSGEVSAALPDSTSDPTQDGSASDP

SEQ ID NO: 3 - protein, 898 aa, synthetic construct
MASLDFKDLVVLITGAGGGIGAGYSHFYGARGAKVVVNDVSAQSAQKVVDEIKAAGGQAAIAVGSVTEGQKIVDQAVQAFGTVHVLINNAGILRDKSFKKMDDAQWDAVIAVHLKGAYSMTKAVWPLFRQQKFGRVINTSSPAGIYGNMGQANYSAAKMALVAFSKSLSIEGVKYDIRANTIVPLAASAMTQTVMPPEVLKGLNPDFIAPIIGALTARNGPDVRGRIFEVGGGFYSELRWERSKGHLFKTDDTFTPTAIRHEWKKITDFSKDVEYPGDLKAAGRNLMAVIEKQPGLKPNEQGPPPPVDFKGQTVIITGAGAGLGRAYAHLYSKLGANVVVNDVSAQNAEAVVKECEQLGAKAAAAVCSAEEGEKIVKVALDKFGGVQVLVCNAGVLRDKAFVNMTEEQWDTVMAVHLRGTFRCCKAVWPIFQEQKYGRILVTSSPNGIIGAHGQGNYASAKAGLIGFGRSLAIEGKKNNILTNILAPSAGTAMTATVWTEEMVKTFSPDFVAPVVAFLTSKDQKGTGQVIETYGGQVAALRWQRTHGAVFPNNRVPTPEQVVAKWDEITTFDDKATHPRNTQESGMQIFENFSNLAPEEEQKDYVDPEDPEAIVKAKRAEPKGIEYEYTERDVMLYNLGIGAHATELQWTYENDQDFQPLPTFGVIPQFTAGLGDYDFLPNYNPGKLLHGEQYLSIKGKVPTSGKLVNKARLFEVLDKGKAAAVTTVIESRDAQTNELIYENHSTVFIRDSGGFGGKRAAKDRGAATAPNAPPKRKPDAVVEEKTSPDQAALYRLSGDYNPLHIDPQFSIIGGFKKPILHGLCSMGFAGRHVLKTFGPYKDIKVRFAGTVIPGETLVTEMWKEGDKVIFQTKVKERDAPALSAAAVTLINSGAPKSKM

SEQ ID NO: 4 - protein, 2361 aa, synthetic construct
MSSQPEALENIFSRLGSKSEDVRVAAAEDLKAQINSANIEFPGDDSKKNVWKEVFHRTFELTRSNTTSERLGSIAAIDAMLEETNNDSPDRALQNNIRLYGYLRPLLTYPDSTVMVAAAHVVGNMVKLAGANLGESFFNKEVGQAIQMIDDSRQEVGRFSAVLLLHQFAVYAPSQFYPFISRVLEKIWIPLRDSRTMVRERASMLLSSCLDIMKARERSSSEIYQVIFEEAKSGLLKASSVDSILGSLLAFSAMLQNQQISMGEYYRQICEITLKYRDSKETVIRKSVITLIPSMATYDSDEFEVHYLHRSMAYLLQALNKPTDRDIAYVALGHLSIQLASKMRPFIDDIVKIIKEHLRMRGKKGAPFEAPIFQCLAMLTTAVGPMLTRQMHEILDLMFPWGLSEALYHALEVISSHIPPLLRTIQDRLLETLSVILTGHSFRPLGAPVRRAKGGEITRDVVIQASGGGQSPETIALALKVLGNFDFGGHTLNEFVQEAALPYLEHDSPEVRKEAVLASTQLFVNDPICNQTSSHAIEIVNDVLEKLLTVGITDQVPGIRQAVLESLDEKFDRHLAQAEDIRCLFVALNDEEFKIRELAIGIIGRLAHHNPAYVMPSLRKSLINLITELEYSTANKQKEESAKLLCLLIGASSSLVKSYASTILSVILRIARSPSTPATVTASCVNCIGELARVAGEELVSNAKTIMDLLLELLNDPASTVKRNAALKALGQVVSNTGEVITPYSDYPQLLGILFRILRLETSSQIRLEAIRTLGMLGALDPFKHKQLLGGDNDNTTESTGPRVTDITLLMNHNGGSNDDYYQMVVINSLVNVLNDANMKEHHYDAINSVMLIFRTQRLRCVGFLPQILPAFLGVIRMGSRDSQDVFLKQLAQLISIVKQHIRNYLSEVFALVHDYWNPNSMMQITIISLVEAIARAVEGEFKAYLPQLLQQILRTFDGDLSARNLTDQRENTLLHILKAFIVFGSSIEDYLHLVLPVIIRSFENPAAPTKLRKAALTTTAQLCRKVNFSDHASQIIHPLVRTLSTQDPELRNAAMETLCVLVLQFGPDYAIFIPMVNKALLENKIVWPMYDQMVTKLLNRERLPPDLGPVEQYAADSMNEVSVSAEQHKLPVNQQQLKSAWDCSHVATKKEWLEWLKRLGVEFMRESPSQAIRACTKLADQHGAFARELFNVAFVSCWTELYESYQEDLVHHLEQALTNDGVPSEVVNTVLNLAEFMEHDDKPLALEPRLLGDYATAFHAYAKALHYKELEFFVDASPTVVEDLISVNQKLQQSDAAWGTLEYAQNNLQMTHDVLWYEKLGRWEEALEVWNERSADPDSQFDEQALAMGKFQALHALGEWEELADVIQVRWPNAAGEEKKTIAPLGAAASWALYQWDMMDDYISAMKTDSADRNFFKAILAVHRNQFTSAKRHITKARERLDGELTTLTGESYGRAYDVIVRVQMLSELEEIISYKDHADQPERQATQRQTWHKRLAGCQRDVEVWQRILQVRSLVLTPSEDMETWISFADLCRVSDRLNLAEKTLTSLVGSSCSNLDAESRSRAPPPIVFAYYRLKWAQAVAVDSRDERVETLGYLHDFTQTLSDDMGLGPRNNQGRLILPDVKLYGEYTKLLARCHVELGKWEASMLEQGFTAAPASVLSNYALATELDPDWYQAWHTWALANFEVIGSLEVSQSGLQPAHFATYIIPAVEGFLRSIALSPGNSLQDTLRLLTLWFKYGYQSGVNQAIQQGMASVNIDVWLEVIPQIIARIQTPRPTIRELIVRLLHSIGRAHPQALIYPLTVASKSNVATRKQVAQDIMDKMREHSATIVDQADLVSTELIRAAILWHEQWHEGLEEASKHYFADHNEEAMFEVLEPLHDMIEKGPETVRETSFVQSFGHDLRLARDLCRRYRQQGDLNELNQAWDIYCATFQRLARQIKLLNVIELQYVSPKLMNVRDLDLAVPGTYQSGKPVIGIAYVLPTFTVINSKQRPRRFTMRGRDGKEYTFVLKGHEDLRQDERVMQLFGLVNTLLSADQESSKRHLSIRQFSITPLSPGAGLLGWVPHSDTLHALIKHYRDSRKILIDIETRLMNQMADDNYDSLPLLHKVEVFQYALDNTTGQDLYRILWLRSRNSESWLERRTTYTRSLALTSMVGYILGLGDRHPSNLMLDQITGEIVHIDFGDCFEVAMQRDKYPEKVPFRLTRMLIHAMEVCGITGTFSRSCEVSMEVLRANKESLMAVLEAFVYDPLINWRLTAGQGGQRQGAHDVEERGGQPYAVQRGGRADERDILVEADRPEVMNDKALQVVERVRRKLTGRDFKPNVTLDIKEQVEELVTQATSVENLCVAFVGWCSFW

SEQ ID NO: 5 - protein, 739 aa, synthetic construct
MAVQRNVHQPLLFECAWEVANKVGGIYTVIKTKVPVTVKEYGDRYTLIGPLSYKTAHIEVESEDPEPGSPMEGTLNAMRDHGVKYIYGRWLIEGAPRVLLFDTGSCYNRMDEWKGDLWNVAGIPTPPNDHETNETLVFGYMVAWFLGEFSGRCLDTAIIAHFHEWQAGLAIPLARKRKIDVTTIFTTHATLLGRYLCAGSVDFYNNLQYFDVDHEAGKRGIYHRYCIERSAAHCADVFTTVSHITAYESEHLLKRKPDGVLPNGLNVVKFAAMHEFQNLHVKAKEKINDFIRGHFYGHYDFDLDNTIYMFTAGRYEFRNKGVDMFIESLARLNHRLKAMNSKMTVVAFIIMPAATNSYTIEALKGQAVTGQLRDVVKQITDRIGNRLFEHAARYDGTHGTEVPKPEELMSAEDLVLLKRRVFALKRNSLPPVVTHNMQDDSNDPILNQIRRVQLFNRTSDRVKVIFHPEFLNSNNPILGLDYEEFVRGCHLGVFPSYYEPFGYTPAECTVMGIPSVTTNLSGFGCFMEDLLESPEDYGCYIVDRRGQGVDESVEQLTQVLTEFTTKSRRQRINQRNRTERLSELLDWKSLGLEYAKARQLALRRAYPDSFNDDEPEFTGVQRVGAPLSAPGSPRYRSGMMTPGDYATLTEEMEHLNTSDYMGAKHPWNWNKEDSDEEEGYTFPIAGLKPRGRSDSLASAISGTMTPSGKHRLNESDLAQADKALESHSSAANANGNGNK

SEQ ID NO: 6 - protein, 494 aa, synthetic construct
MNFDASYWLAYREANIRFADVVASFVRTGDVVWVQDYHLMLLPMLLRSMITGESNQGEMTRREMGRVKEGVDEAVVNDINIKMPPPNRGNGGGADEGVEMLEDVEEESNPKSPTHRQSSSTPGMTPFQKSEIQARRRGKGGVRIGFFLHTPFPSSEIYRILPVRREILLGVLQCDLIGFHTYDYARHFLSACTRILGLETQPNGIEFEGRWVQVGTFPIGIDPWQFVEGVKLPVVQERLDRLEKRFEDCKVIIGVDRLDYIKGIPQKLHAFEVFLTQHPEWIEKVILIQLAIPSRQDVEEYMNLRSCVNELVGRINGQFSTPTWTPIVFMHRSVPFEELTAMYALADVCLVTSTRDGMNLVAYEYISSQAEKHGSMILSEFAGASQSLAGSLIINPWDVDSTASAIHQALTLAPEIRAQNWTKLFAYVSNFTAQSWGLSFLNELNRSTGRRPTGPLLGGRRKSSSNLSRASSRASIKRRFTVNSSTPVAPVVAS

SEQ ID NO: 7 - protein, 958 aa, synthetic construct
MSAAKSYEGSAQLIDGNAIAKEIRQNIHAAITELQAKERSFKTPVLNIYQLGSDPASSTYIRMKLKAAEESGMKVEHIQVPADSEVEGAAKGTGVRKLLDLVHKANEDEKVAGILVQLPLPGASKEEEKKVVESISVGKDVDGFHPENIGLLSGRLTEPHFTPCTPAGIVRLIDSTGFDLKGANVVVLGRSDIVGTPTCALLRKRDATVTQCHSKTVGLPDIVRQADVVVAAIGQGQFVKGDWIRPGAIVIDVGTNFIPDASKKSGQRMVGDVDFPAASNVAGFITPVPGGVGPMTVAMLMNNTFRAARRQWESSRQRKLTPLPLNILPQVPSDIEIAVAQTPKPVADIAREIGVHDDEIESYGKYKAKIELSVLERLQERKDGKYIVVAGITPTPLGEGKSTTTIGLAQALGAHLQKTAIACVRQPSQGPTFGVKGGAAGGGYSQVIPMTEFNLHLTGDIHAVTAANNLLAAAIDARMFHESTQTDKALFNRLCPPKKGVRSFAKPMLKRLEKLGINKTNPADLTEEEAARFARLDIDPATITWNRVLDVNDRYLRKITVGQAPTEKGFTRETAFDIAVASEVMAVLALATDVADMRERLGRMVVASSKKGEPITAEDIGCAGAMAVLMKDAIKPTLMQTLEGTPVFVHAGPFANIAHGNSSIIADRIALKLAGIEEGDAEDRNGYVITEAGFGADIGMEKFCNIKTRVSGLRPNAVVLVATIRALKMHGGGPTVSPGKPLDPVYSTENLELLQKGCANLGKHIENAKKFGLKVVVAINKFVFDTEAEMKLVQDFALQAGADYAVPANHWAKGGEGAVELAQAVVEACKDESQFKFLYDVNLPLEEKMSIIAREMYGADGVELSPEAKAEVERYERQGYAHLPICMAKTALSLSDDPSRKGVPTGFTLPIRNVRLSAGASFVYPLVGDMSTMPGLTTRPGFYDIDLDPETGDILGLF

SEQ ID NO: 8 - protein, 365 aa, synthetic construct
MDSTRSVSHTIQGQSVAQPLTASAAVAGTVTPPDAFDRLQPEPASQAHILRSYQRDTAQVGRLTELVSELLRSMAGTRWLTHKQTIVDLAVRAVYLMLTFGRGRQTLGEEYTDILPTANKLAPTRRRRLLTVALLLLPTALTSPSMLHYLRGDPYDHSRWAAAKRRVARFIESPLGQSLPEIHLVMFMFGGRFYEFARRATGMVYVSDMPPLPAEHKPASYEPLGLIMALPLLYRLFPRSTGGASGASATRAVAGSMEEKVKENDYSGTATPIRNAPEDSVVVTSEEQYDLPNTYLDEEALDLPERQCTLCLESRGTGEGSGGTTAVTECGHIFCWGCLGGLDKMECPLCRQALRMERLIAAYNL

SEQ ID NO: 9 - protein, 544 aa, synthetic construct
MSASTKQAATTVAQPSGKPVTNVIDPERDDFIVPDNYVTRTVENMKMLPPVTWRNLHKNIQWISFLALTIPPAMAIYGLCTVPVQTKTFIWSVVYYFITGLGITAGYHRLWAHRSYNASKPLQYFLALAGAGSVQGSIRWWSRGHRAHHRYTDTKLDPYSAHEGFWHAHMGWMLIKPRGKIGVADISDLSKNPVVKWQHNNYVALLFFMGLAFPTLVAGLGWGDWWGGLFFAGAARLVFVHHSTFCVNSLAHWLGETPFDNKHTPKDHFITALVTVGEGYHNFHHQFPMDFRNAIKWYQYDPTKWFIWTMAQLGLASHLKKFPDNEIKKGQYTMKLMQLQEQSEKLEWPKHSNDLPVISWEDFQAESKTRALIAVHGFIHDCSSFIEDHPGGAHLIKRAIGTDSTTAFFGGVYDHSNAAHNLLAMMRVGVLDGGMEVEHLKVGRLTRSNSADSLVSDAPSSASSTVSASSILSEVEDMDKPERKPVYQPVAQVSNPWTLAVPPSEKYRIIRTSPEIRPGLISRIQSAASPSLTRAASFDAPVAP

SEQ ID NO: 10 - protein, 468 aa, synthetic construct
MRNELNRMVQGVTDPAKKKMFNAEMESFFLLFNRYLTEKAKGEKIDWEKIQPPKPEQVRPYKVLDKVDPSILNKLAVLKLNGGLGTTMGCVGPKSVIEVREGMTFLDLSVRQIEHLNEKYNVNVPFILMNSFNTDDDTARIIQKYQNHNINILTFNQSRYPRVNKESLLPCPADATSDKANWYPPGHGDIYDAINNSGLLDQLIAAGKEYIFISNVDNLGAVVDLNIFQTMIDAQAEYVMEVTDKTKADIKGGTIIDYEGKPRLLEVAQVPKDHLDEFCSTRKFKIFNTNNIWCSLKAIKRVVDNDELNLEIIVNNKVTDKGEAVIQLETAIGAAIKHFDSSIGINVPRSRFLPVKSCSDLLLIKSGLYNLQHGVLTMDKSREFGGTPVVKLGDQFKKVANFEKRFKSIPNITELDHLTVSGDVWFGKRVRLAGTCIIVATEGNKIMIPDGTDLENKLITGNLSIIDH

SEQ ID NO: 11 - protein, 417 aa, synthetic construct
MSPKTADEIAAAADATIHEVGYDRNGFDSRIDTKTLRSLYLAPAFIKAGAETTNVGSASKDYGTESVYDKDSNKIQYTTFAKFPPVRLLPDAQRKRILVTGGAGFVGSHLVDRLMLLGHEVTVLDNFFTGSRTTVSHWVGHPNFEMVRHDVVEPFMIEVDQIYHLACPASPPHYQYNAVKTIKTSFMGTLNMLGLAKRTKARFLITSTSEVYGDPEEHPQREDYWGHVNCIGPRACYDEGKRVAETLTYGYMRQDGVDVRVARIFNTFGPRMNPYDGRVVSNFIIQALKGEDMTVYGDGQQTRSFQYIHDLVDGLILLMNGEETRPVNIGSSHEFTIMEFAEAVRDIVEEVQKAEGVPNPKRVGIIYNDIPTDDPQRRRADTTRAKETLDWQPRWMVRDGVREMVLYYKQMIDQGKL

SEQ ID NO: 12 - protein, 386 aa, synthetic construct
MSRDNSQLKRVLVTGGLGYIGSHVVLSLLLSGRYLPIVIDNCHNAFPEAVNRCAEIARDEMGADAPQPIFHQVDLRNPADIDAVFEKYQADGGIWGVVHLAALKAVGESSEIPLAYYQVNVGGSVSLLQSMAKYGCNSFVFSSSATVYGTPEIIPIPETSRVQAESAYGRTKAMVENVIQDVCRSGTMEAPRKEPLKAVSVRYFNPAGAHPSGKLGEEPRGKPGNLLPLLAQMAVGREKSELKVFGNDYPGSPDGTCVRDYLHIMDLAQGHVLALDAIAVPADQPNIFDKCDPRADGFFRAFNLGRGKGQSVLTMIEAMRKATGFDYKYEIVGRRMGDVPDLTADPALAEKELGFLAKRDLPEMCRDLWNFQTKNPNGYEPSANAA

SEQ ID NO: 13 - protein, 377 aa, synthetic construct
MYSNNVERRSDADEKVLMANLGGAQRPPSPSLAAKDTPAAPSSREQILPIVNYCAASIMMTVVNKYVVSGAKFNMNFLLLAIQSSVCVLAVYSVKRAGVISFRDFDLKDAKQWYPVSCLLVAVIYTGSKSLQFLSIPVYTIFKNLTIILIAYGEVFMFSGIVTGLTLVSFALMVGSSVIAAWADISSAWSTTLDQTTGRELTAMPTIGGVSVGYFWMALNCACSAAYVLYMRKRIKVTGFKDWDSMFYNNLLSIPVLVVASLVFEDWGSKSLSDNFPAENRTILLSAIVFSGAAAVFISYSTAWCVRVCGSTTYSMVGALNKLPVAASGMLFFGDPVSFGNVSAIGVGGIAGIVYAIAKTNQARVEAAAKARQSGGP

SEQ ID NO: 14 - protein, 404 aa, synthetic construct
MANTVTKGVILVGGPSKGTRMRPLTLDCPKPLLPIGGKPMVWHPLNALAQVPGLTDIIMIGFYEDSVMAPFVRESQREFPNIRISYMREYRSLGTAGGLYHFRDLILRTPLPQHIFICNIDICSSFPFTELLAKHSAHRGVGTILGVNVPKDTATRYGCIVSDPETKQMLHYVEKPESWISNTVNGGVYLFDKSVFDEIRVAMDKKTECAAHNPLIELDETLQLEQDVIAPLAGTGKMYVCETRDFWRQIKTAASAVTASGLYLKQFQRSRPELLARAGGAIIEPVYIDGSAHIDGSAKIGPNATIGPNVVVGEGARIANAIVLDNSVIDKHAVVLNSIVGSNSRLGQWARVDGEPEPVSDVKGQISVSVLASDVHVAPETHIRSCIVLPNKNLPKNAYNQVLL

SEQ ID NO: 15 - protein, 649 aa, synthetic construct
MSLRRHSPQFGGAGAGGSGTSPLLASAQTPAGQYARLSETSLAGNDSPSSSMRYGPSAGGGARGYGPQATVSSFSSGTRYGPTADSPSSSSHGHYAAQVEPMSLRAAGAGAGTGPVAALDEADLDDQLHTFTAAEKKDLTAPFTLRSWRGWANALTLLVMLSGFVVLFAGYPIISWYYSDKNALGANQPGFNLGGVNATGQYPEIPGFPQMIDPDTPESAKKHISFEGDTWDLVFSDEFNKDGRTFFEGDDPFFTAMDIHYWPTNDFEWYDPSVPTTRDGHLVLTITQEPINGLMFKSGMIQSWNKLCFNRNAYFEVSASLPGTPRIGGFWPGVWTMGNLGRAGYGGTNDGMWPYSYSSCDIGILPNQSADGVTPEVAFTTGDPNYNMTLSYLPGQRLSSCNCPGYDHPGPEGVGRGAVEIDMIEAQMDLKVQRGMVSQSAQVAPYDDYYQFNNESDAAIIYDSEVTRYNTYLGSVYQQAVSGLSYTDTRIYSNETSLHVNESSKFGVFGVEYLADYDDVSKGYITWINMGKRAWTMWAKAVGPNPRTGVGQRLISQEPMYMIFNLGMSNNFEYVDFANLMLPNEMRIDYIRVYQKPGEGTIGCDPPDRPTASYIAKHADIYNNPNITIFPDADKGRVFPKNRMMDPNC

SEQ ID NO: 16 - protein, 525 aa, synthetic construct
MGPEEDDDLHNPTVRQSYKTGFFRSLLTVRGFTNVGCLLIITLILVVLFMGYPIITEFTERHQTTNGAFGLGGTNASGQVPLVRQPIDPDTPPEAYQKVSLETGETWDLIFSDEFNHPGRSFYPGDDPYWEAEDLHYWPTNNLEWYDPKRLTTRDGSLVITLTNEQYNRRNYTGGMMNTWNKFCFTGGYIEAAVSLPGKSNVQGLWPAVWTMGNLGRAGYGGTTDGMWPYSYDSCDVGTLKNQSLNGVPEVAQTQGDPSYNMTLSYLSGQRLSACTCPGDKSHPGPIRPDGTYVGRAAPEIDMIEAAVSDSKVGEVSLSGQWAPFNPWYGWINQTDLVTVYNVSNSHFNQYRGSIWQQATSLLAKTDQACYTHEQGCFQVFGVEWQPSKDNDNDGYISWLSDGKKAWTLREGAMGPNSDAGIGQRIVSREPMYMIVNLGISPNFGYIDFEGLDELWPVEMHVDYIRVYQDPKKRNIGCDPPDYPTADFIDKHRVAFTNPNITTFEQLQDAMPGLEWPKNRFLGEC

SEQ ID NO: 17 - protein, 888 aa, synthetic construct
MPPAQKQAQAARLNPVWYHYACATVAAALVLGNLLRWAYLDAPDNYHCGAMLSTGKWLDEGTYKNWQPEGCYQAAIKAPALAKCLTSPSAVTPVTDGLSNRRVLFVGDSTVRQLYFAAVRQVDKGTDYPKSWESDGEKHSDRKAVITTKGGQQLTIEFWWDPYLNTTQTHDYLASRPETPAAMLVMGSGLWYLRNPSSGGATAFTKTIEDTFETLRAKQGSPLAPLLNPWDAMTTTGSNWLPGLMPDSTGAGVSAGGHDFALADAVFFVPVANPVVEKLSAERAETIVPADVEMMNAEVATRLAQSNPPPIIVPSVFNELLVDEETSDGLHFSDRIMDKQAELLYSWRCNDVLRKEVSEGACCRRYTTLRPLQALLLLYLGVWAPISCLIAPRLRPGHPLLAVIPADKVAKAISSFGLAIAYLYLADRTTIFLKEQKDWDPKIFSALMIAALIAGLATVKNKGKDLGFLNRDITDEWKGWMQIAILIYHFFGASKISGIYNPIRVMVAAYLFMTGYGHFFFYFKKADYGFNRIAQVLVRLNLLSVVLPYTMNTDYAFYYFAPLVSWWYMIIYGTMFLGSKYNDKPAFLLTKLVVCAGAITAFMHQTWMMEGLFSFLNTVFRIQWSAKEWSFRVTLDLYIVWGGMLAAYAFIKFNELRIAERPFFPALRTGAIVASVLGLIWYMWFQLSRKDKFEYNGYHAAVSCIPILSFVVLRNASALLRTCTSQLFCFVGQISLETFILQFHGWLAADTKAVLLVLPATRWRPVNLVVSSIAFGWLSYKVSGATGDITSWAVGKKKPALPPPVTQGEPIFDAGQENIPLMEPAKDENGNAVNGSANASAAYAAPEKSWRDSTVMSVLGNIGVLAQTHDSVKLGLVLLVLWVANWLY

SEQ ID NO: 18 - protein, 461 aa, synthetic construct
MLYLTADKFPVFVPFGVIGFYRYLWYCIRLIARYVYRPIPLPETPTYRADEDVTIIVPTIDAGEEFREAALSWLVGNPKEIIIVTEEKMLAPLQELANSVDPTRIRVLTVPFANKRLQMAHGIRNTTTDIIVFADDDAIWPPQLLPYVLACFEDQQVGGVGTSQRVKSVGKKMTVWEVLAGFRLTIRNIEIASATHIDGGIPCLSGRTAAYRTVILKDPDFLHGFTNDLWLNKYHLNSGDDKFLTRWMVSHGWNTYVQVCKEAELLSTMKPNWRFLKQVLRWTRNTWRSDFRSVFTERYVWTKHPYVCYTMIDKFINPLTLLVGPALVCYLLYKSTKPIDQGGYHLPAWDIVVSYLVWLLCTRTLKVLPHLWDRPKDIIYVPVFIIFGYYFAIMKIYALFTLHKTGWGTRSGIGDPATATTAAENVNEKVPRPVELGGPAPSHSRPYDVEMAQNVNNPYRA

SEQ ID NO: 19 - protein, 434 aa, synthetic construct
MSSQYSHDDINGNGGSARSSQYRLPGPPPPLMDQSHLRPGKQAELLSHDRTLELYRENAKKTNDPELIFEFAVFMIDAAKSMVPPPNPDDPNDPANRPNAAALEKRDELIKEATSLLKRLADRGYPDAQYFLADCYANGIGTTRGKQDFDKAFPLFALAAKHGHPDACYRAGTCCEHGWGTRRESAKAIQFYRKAAVGLHPGAMYRLGTAELNGSLGLSKRPKEGVKWLKRSAEHATEEFPHALHELALLHERGIENIVFVDLDYSAELLAQAAELGYAPSAFKLGECYEYGKMGCPVDAALSIHYYNIAAQQGHRDACFALTAWYLVGAEGVLPQSDTEAYLWAKKAADKGLAKAQYALGYFTETGVGVPPNIQQAMKFYRQAADGGDKRAAKRLAGGVSGGGAALNRQREMDAMKEAGGMNGKDKDKDCIIM

SEQ ID NO: 20 - protein, 959 aa, synthetic construct
MDIGDVYRAPPQGQVPRRRDPYDAGHPAPPPPVPPNLRMDRNNVTPSPGPYPNYPPSSSAHENPFQHPSDHGNDWGQAPPVPPPPQPYAQLGRTDSSQSYQTPYMADNRLTSPPPLFPAPSASPGFSQSGPYGHPLDDDTNDAQPLLHHAQPHPAFNIQPMPPRPQAPYDPPIQGSAGGRVHFDDPYQTQGVNDEQERNNVHYGAPPARVLRRNRTQKRVQLFRGHLVLDVDVPSMLMDQSAIKEGNEFTKMRYTAVTCNPDEFVLDKYTLRQRLYEQPRKTELFIVVTMYNEDDVLFCRTMRGVMQNVAHLCSRSRSKTWGKDGWKKVVVCIVSDGRMKINKRTQSVLAALGVYQDGVAKNVVNNKPVTAHLYEYTTQLWIDSKGRVGPGGDNAVPVQLIFCLKEKNQKKINSHRWFFNAFGDCLRPNVCVLLDVGTQPGPDSIYHLWKAFEVNQSIGGACGEIVALKGTMGKNLLNPLVAAQNFEYKMSNILDKPLESAFGYITVLPGAFSAYRYIALQNDDNGEGPLEKYFYGEKMHDANAGIFKANMYLAEDRILCWELVSKRSSRWTLHYVKSAYAITDVPDTVPELVSQRRRWLNGSFFAAVHSIAHFHYLYRSSHSVMRMFWLHVELIYQIISNVFAWFSLGNFYIAFIVLSKSMEYAVDLAPNEPAFDEFGNRKTNKVFEYINIPLQYAYLALLLLCFLLSLGNRPAGSKWGYTISMIGFALITVYMIFAAVWLTVAGVNQIKEDGATVGELVQNPTFRNIVLSLLATYGLYIVSSLMALDPWHLVTSFVQYLLLAPSYINVLNVYAFSNVHDVSWGTKGSDKVSEDLGVVKSSGGDGEVTVDMFSEQKDLNAIYAAELQVLATKPPKEEAKVNTEQQHEDYYKNFRTNVLLSWTMTNAALAIVILRVGVDGQFVIAQTYMAVMLYTVAGLAFFRFLGAIVYLVVRLFMGE

SEQ ID NO: 21 - protein, 619 aa, synthetic construct
MACGLPRALQVALAVRALSLALPHTFFQPDEFYQAFEPAYAVVFGQGHLTWEWRDLPLPEGVAHTWWTRVVTGGRLRGWIWPGVFVLAYKLVQVLALDSTDLIVFVPRVVGVILAAVTDYYTSRLAGKVLGEGYAAGALFLSLTSLFNAHLLPRALSTSPETLLTTIGLYYYPLPAFRPASLGATCVPSQDGTAEHHEAQRTCGAMDRAEGGKTRPSSDNMLFAAAVAALSLCIRPTMLVFWAYLHLETSIRHWVTHGATATFCLTADTLLGGLTVLGLSTLLDWHMMGRLTIPTLSFFHQNVVRNVALFYGGTGLLYHLVQSLPLLLMPIWFWWGEGFLAAILPQSMQPPALKKLDIPLALSTLGRTIVVTIATLSLSPHSEWRFLHPLLPPLMLFALPPLQASYKPTPMGYHSFLTSVRQYCRLPSRPCYLILLCPIIPYLYLNSFHGAAQVAVINGLRHGDFGAASFVVALMPCHSTPWSSHLGNIPGWFLTCEPPLVPQSADVHWTQQDLFYASPVTYIQHVFPSPPAKLSDVPTMAGEERMPSHVLVFGEVLARQDTLGNSSLSVEQALAERGYHEKARLWNGFDFAQDEEKRRGGVRVWTHGESYDLVGEPAA

SEQ ID NO: 22 - protein, 584 aa, synthetic construct
MAETDPPLDAAPLLAAVNPPRSRRWAIMLCFPLFLALASPWWWTTTSIVRLPLPVERIGALESQSDPVITTLVSFTGEDAAFPKAPPGRADFPTATKLDALAAEVVKGVDGMLARDRPNVRGTRRWDVVVNGPKSVRHRVHIGVSETAKPDWPLEPYVQTSYGTADVPPGTVVIPIHPSMVSDLNLKRHYKIALVNALHNLLPAHLPPVANRALAYSPNITLSFVVVNEDAAEGAFVNDWDIDGALRDHIFPHLEPLSPVFNFSIESQVLYHAPLAFEPAQQDDGWVVDEDGMKMFVSERWTPDSLSTNNPVLKFLLFVPSAQHAPLRLAANATSFLIPQFGSVVLLNPPAEGGAYRLSLDALDAPFKQFTEHLYGLLSLPALPEKLHHCPEPASEGKTRAQRGLRDPLSLWQLDTIFRVRMAENAAEARKTLGGIVRLVDKIKEMQLGPGVRDKVLGAVRRLEELPETAQDMSPLTAFVLSRDAVGLANEAFFDPSMMGLLYFPDQHKFAVYTPLFAPIGVSIVVGLLREIANLIKKRKAARKAAQAKKDDERLDNGTQVVSEAMTTAREAPAAARLRATTTS

SEQ ID NO: 23 - protein, 293 aa, synthetic construct
MHVLLTGALGRCGMRTLSQLLDEGHTVLATDRTSPSSPLGDGAEFMAADLTLLHDIDALFKHRFDGVIHLGAIPNPVPEIDDRELHANNVVSSYNVMRTAVDHGVRRIVQASSVNAHGLSFAPPGHTAFDRFPITEAVERRDEDAYATSKHECEVQASALVRYAPGTRIASLRPHMVRDDYASAYPDTEARDIFSWVSYAAVARAAVLALTSEGWGGHEVFNIVAPEICWEGSVEEQYIDKDGEPPAHKVGTLELVRKYWPNVEVDEEYWRDRPRRAVWDSSKAERLLGWRHE

SEQ ID NO: 24 - protein, 186 aa, synthetic construct
MRSLTSPLPSHPQYSVLVVTTTAGARDAAGVVTEHRVAPRRNNLGPRTYLLPAAIVLLCWWRFPHPLVLASAVAVALALRWRVLYQSVTPLPGLGVQLATVRGFGVGPKSTARQFVPLDDISTVVIHEGLRRWSVRYYLGVVRKRGNVVVAFDAVHPHPEVLREVYHGVREALYEEYAGDASETGE

SEQ ID NO: 25 - protein, 364 aa, synthetic construct
MKALILVGGFGTRLRPLTLSWPKPLVEFCNKAMILHQIEALVKAGVKDIVLAVNYRPEVMVSVLKKTEEEYGITINFSVETEPLGTAGPLALARDILGKDDSPFFVLNSDVTCTYPFEAFRDFHLKHGCEGSIMVTKVSEPSAYGVVVTKPGSTIIDRFVEKPVEFVGNRINAGIYMFNPSVLDRIELRPTSIEKEIFPAIAADQQLHAYDLQGFWMDVGQPKDFISGTCLYLSHLTSQGSPDLADPAQNKWVHGGNVLVDPTAEIDPTALVGPNVVIGPGAKIGKGARLQRCVIMSNAEVRDHAWIASSIIGWNSTVGRWCRVENITVLGDDVSIKDELYVNGASVLPHKSISTSITEPRIVM

SEQ ID NO: 26 - protein, 606 aa, synthetic construct
MSASGDHSALLPPGTGTRSPNLDYSQRSSRRYGPAASTDPVPSTASLLGGSAGSRLSSGSRLSAPRSTSTHGTRAGATSSASDGWGWNDPEADDYLHNPDPKRDRKNDRGGSIFTLRGIVNIGCLVLLLLCLITLFAGYPIITTYTERRQSNNGAFNLGGTNATGQVPLIAGFPSLIDPDTPMDVRSRVGYDGKAYELVFSDEFNKDGRTFWPGDDPFWTAVDIHYWPTVDLEWYHPSAITTENGHLKITMTQEPINDLQFKSGMLQSWNQMCFQWSYYFEISVSLPGNNRIGGFWPGVWTLGNLGRAGYGGTTDGLWPYTYTACDLGTLANQTWPAGSGEVTPPGAITTSDGDPLSYLPGQRLSSCTCKGEDHPGPNPSVGRGAPEVDVIEGQIDTRMRMGEMSQSAQVAPFDADYQFDNSSDAVTIWATDITKWNTYLGGVYQQAVSGLVYVDNGVYTGTSGLFNIYGVEVFADKNDESKGYVTWVSGNEKSWTMEAKATGPNPLSGVSQRPITQEPMYLIMNFGMSNGFQQVDLQNLQWPAHMLIDYVRVYQIPDYGYLGCDPEDHPTAEYINSHPEPYNNANITLWSQAGYSWPKNRLVNTC